(19) United States
(12) Patent Application Publication
(10) Pub. No.: US 2026/0092334 A1
(43) Pub. Date: Apr. 2, 2026

(54) METHOD FOR BARLEY HYBRID SEED PRODUCTION

(71) Applicant: KWS SAAT SE & Co. KGaA, Einbeck (DE)

(72) Inventors: Parastoo HOSEINZADEH, Einbeck (DE); Stephanie JAHNKE, Einbeck (DE); Volker MARWEDE, Einbeck (DE); Jason WENZIG, Einbeck (DE)

(73) Assignee: KWS SAAT SE & Co. KGaA, Einbeck (DE)

(21) Appl. No.: 19/111,098

(22) PCT Filed: Sep. 11, 2023
(86) PCT No.: PCT/IB2023/000540

§ 371 Date: Mar. 12, 2025

Related U.S. Application Data
(60) Provisional application No. 17/943,483, filed on Sep. 13, 2022.

(51) Int. Cl. C12Q 1/6895; A01H 1/02; A01H 1/04; A01H 5/10; A01H 6/46

(52) U.S. Cl. CPC C12Q 1/6895; A01H 1/023; A01H 1/045; A01H 5/10; A01H 6/4624; C12Q 2600/13; C12Q 2600/156; C12Q 2600/158

(57) ABSTRACT

The present invention relates to hybrid barley seed production and marker assisted selection of barley plants in order to obtain barley plants or seeds comprising an Ant 1 and/or Ant2 gene allowing color sorting of seeds.

### SEQUENCE LISTING

The instant application contains a Sequence Listing which has been submitted electronically in XML format and is hereby incorporated by reference in its entirety. Said XML copy, created on Nov. 7, 2022, is named 245761_000188_SL.xml and is 56, 160 bytes in size.

### FIELD OF THE INVENTION

The invention relates to methods for generating barley plants as well as marker assisted identification and selection of barley plants. The invention further relates to methods for barley hybrid seed production and selection of barley hybrid seeds.

### BACKGROUND OF THE INVENTION

The development of hybrids in cereal crops such as winter barley has always been appealing for the prospects of seed yield increases due to heterosis (hybrid vigor) providing yield stability across sites, but also offers opportunities such as trait introgression. However, the production of marketable hybrids is an undertaking that takes both seed yield and quality parameters into account. Hybrid barley production can be facilitated in the field in two ways. Firstly, female and male plants are grown in alternating strips adjacent to one another. The males pollinate the females through pollen transfer by means of air movement, i.e., wind. Secondly, the most common system is the mixed seed production. In this system, a technical mixture of female seed and pollinator (restorer) seed is sown in the field. The blending ratio of female and male seed can vary, but usually is around the 85/15 ratio of female/restorer. The mixed production system is the economically preferred production scenario, as it offers the harvest of one entire field instead of only the strips of females as in the first scenario. Several environmental factors such as moisture, temperature, air movement need to be in good constellation at the stage of pollen shedding to facilitate a successful, complete pollination on females around a pollen shedding male plant. In addition, the female plants must be in the right developmental stage to accept the pollen. While the pollination of the pollinator can always be considered complete due to its self-pollinating nature, the pollination rate on the female can be subjected to a large range of success. In case of poor pollination conditions, seed set on the females will be very low and seed harvested from the field will show a strong bias towards pollinator seed. If the level of hybrid seed in such a harvest seed lot is below a level of 85% hybrid seed or hybridity level of 85%, the seed lot is non-marketable as hybrid seed and will have to be rejected. Financial losses can be significant, if hybrid seed lots slated for sales must be rejected for quality issues in larger frequencies. The presence of pollinator seed in each hybrid seed lot is not only crucial to define marketability but is also considered a disturbing side effect in terms of using seed harvested from a F1-brewing quality hybrid as a malting substrate.

It is an objective of the present invention to address one or more of the above shortcomings.

### SUMMARY OF THE INVENTION

The herein presented invention in certain aspects and embodiments enables the color sorting of unwanted pollinator seeds in a population of hybrid seeds. A selection of the pollinator seed is a necessity, derived from for instance the cytoplasmic male sterility system, used in seed production to avoid the self-pollination of female plants. The color sorting is established by identifying the genetic status of two genes mediating seed color, Ant1 and Ant2, which are used in a way that only the male or female seed carries the color, and respectively the female or male plant and seed is colorless.

The present invention in certain aspects and embodiments relates to the combination of two dominant genes in epistatic interaction, coloring the seed coat in (winter) barley, and marker technology to trace the gene(s) and their allelic status during different breeding steps in a process to develop (winter) barley hybrids. In a hybrid seed production system, the coloration is the key feature to identify and discard unwanted (male) fertile seeds from a technical seed mixture of (winter) barley hybrid seeds.

In particular, the present invention in certain aspects and embodiments addresses the need in the prior art for uniformly colored hybrid seeds which are easily selectable in the production process. Furthermore, using parental components with different seed color to enhance the hybrid seed purity in the final hybrid seed lots in mixed hybrid cereal seed production has never been reported before. The application of current developed genetic markers comprised in or linked with the Ant1 and Ant2 genes facilitates the integration of the colored seed coat traits from plant genetic resources into elite germplasm that are going to be used in barley restorer lines. This invention in certain aspects and embodiments enables that inbred line seeds coming from restorer lines are distinguished based on the seed/grain coat color from the hybrid seeds (i.e., F1 hybrid seeds) having non-colored seeds.

In particular, the present invention in certain aspects and embodiments offers a remedy to the major obstacles in current hybrid barley production and has specific advantages in prospective hybrid barley seed production:
Low-cost mixed hybrid production with no risk of a bias towards the pollinator seed in seeds harvested can be conducted as the restorer seed can be easily removed from the hybrid seed lot by routine optical seed processing.
The successful removal of restorer seed ensures 100% hybrid seed will be sold to the market.
The nature of the system will ensure that the grower will harvest only seed grown from a uniform F1-hybrid seed lot constituting F2-seed. The seed will not be contaminated by restorer seed as in the classical mixed hybrid production system described above. Therefore, farmers benefit 100% from the heterosis effect inherent in pure (F1) hybrid seeds.
The derived plant out of the germinated hybrid seed will be 100% colored, with a substernal degree of anthocyanin content, which is not present in uncolored seeds. Anthocyanins have exceptional dietary benefits for both human nutrition and animal feed.
The produced hybrid barley seeds will be rich in anthocyanin content being identified as promising ingredients for the development of whole grain functional foods which is not present in any barley line being sold currently at the market. In Germany, winter barley is mainly used in the feeding industry. Hybrid barley could help to create super food for animals' consumption as well.
In case of regrowing of the seeds harvested from hybrid plants, the farmers will observe phenotypic segregation which increases in the following years. This impedes farmers regrowing of the seeds.

The present invention is in particular captured by any one or any combination of one or more of the below numbered statements 1 to 180, with any other statement and/or embodiments.

1. A method for identifying and/or selecting a barley plant or plant part, comprising screening for the presence of a polynucleotide comprising one or more molecular marker (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

2. The method according to statement 1, comprising screening for the presence of one or more molecular marker (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

3. A method for identifying and/or selecting a barley plant or plant part, comprising screening for the presence of one or more molecular marker (alleles) selected from Ant1_HapM1, Ant1_HapM2, Ant1_HapM3, Ant1_HapM4, Ant1_HapM5, and Ant1_HapM6 (in the in the Ant1 gene and/or genome of the plant or plant part).

4. A method for identifying and/or selecting a barley plant or plant part, comprising screening for the presence of a polynucleotide comprising two or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

5. The method according to statement 4, comprising screening for the presence of one or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

6. The method according to statement 4, comprising screening for the presence of two or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

7. A method for identifying and/or selecting a barley plant or plant part, comprising screening for the presence of two or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

8. A method for identifying and/or selecting a barley plant or plant part, comprising screening for the presence of a polynucleotide comprising three or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

9. The method according to statement 8, comprising screening for the presence of one or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

10. The method according to statement 8, comprising screening for the presence of three or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

11. A method for identifying and/or selecting a barley plant or plant part, comprising screening for the presence of three or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

12. A method for identifying and/or selecting a barley plant or plant part, comprising screening for the presence of a polynucleotide comprising four or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

13. The method according to statement 12, comprising screening for the presence of one or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

14. The method according to statement 12, comprising screening for the presence of four or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

15. A method for identifying and/or selecting a barley plant or plant part, comprising screening for the presence of four or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

16. A method for identifying and/or selecting a barley plant or plant part, comprising screening for the presence of a polynucleotide comprising five or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

17. The method according to statement 16, comprising screening for the presence of one or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

18. The method according to statement 16, comprising screening for the presence of five or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

19. A method for identifying and/or selecting a barley plant or plant part, comprising screening for the presence of five or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

20. A method for identifying and/or selecting a barley plant or plant part, comprising screening for the presence of a polynucleotide comprising molecular markers (alleles) Ant1_HapM1, Ant1_HapM2, Ant1_HapM3, Ant1_HapM4, Ant1_HapM5, and Ant1_HapM6 (in the Ant1 gene and/or genome of the plant or plant part).

21. The method according to statement 20, comprising screening for the presence of one or more molecular markers (alleles) selected from Ant1_HapM1, Ant1_HapM2, Ant1_HapM3, Ant1_HapM4, Ant1_HapM5, and Ant1_HapM6 (in the Ant1 gene and/or genome of the plant or plant part).

22. The method according to statement 20, comprising screening for the presence of molecular markers (alleles) Ant1_HapM1, Ant1_HapM2, Ant1_HapM3, Ant1_HapM4, Ant1_HapM5, and Ant1_HapM6 (in the Ant1 gene and/or genome of the plant or plant part).

23. A method for identifying and/or selecting a barley plant or plant part, comprising screening for the presence of molecular markers (alleles) Ant1_HapM1, Ant1_HapM2, Ant1_HapM3, Ant1_HapM4, Ant1_HapM5, and Ant1_HapM6 (in the Ant1 gene and/or genome of the plant or plant part).

24. The method according to any of statements 1 to 23, wherein Ant1_HapM1 is or comprises a SNP at a position corresponding to position 71238822 bp (on chromosome 7H) of reference genome sequence assembly of Barley cultivar Morex v3.0 July 2020 (Morex_v3) (Mascher et al., 2021);

Ant1_HapM2 is or comprises a SNP at a position corresponding to position 71238454 bp (on chromosome 7H) of reference genome sequence assembly of Barley cultivar Morex v3.0 July 2020 (Morex_v3) (Mascher et al., 2021);

Ant1_HapM3 is or comprises a SNP at a position corresponding to position 71238052 bp (on chromosome 7H) of reference genome sequence assembly of Barley cultivar Morex v3.0 July 2020 (Morex_v3) (Mascher et al., 2021):

Ant1_HapM4 is or comprises a SNP at a position corresponding to position 71237825 bp (on chromosome 7H) of reference genome sequence assembly of Barley cultivar Morex v3.0 July 2020 (Morex_v3) (Mascher et al., 2021);

Ant1_HapM5 is or comprises a SNP at a position corresponding to position 71237633 bp (on chromosome 7H) of reference genome sequence assembly of Barley cultivar Morex v3.0 July 2020 (Morex_v3) (Mascher et al., 2021); and/or

Ant1_HapM6 is or comprises a SNP at a position corresponding to position 71237608 bp (on chromosome 7H) reference genome sequence assembly of Barley cultivar Morex v3.0 July 2020 (Morex_v3) (Mascher et al., 2021).

25. The method according to any of statements 1 to 23, wherein
Ant1_HapM1 is or comprises a SNP at a position corresponding to position 95 of SEQ ID NO: 1 or position 95 of SEQ ID NO: 7;
Ant1_HapM2 is or comprises a SNP at a position corresponding to position 463 of SEQ ID NO: 1 or position 224 of SEQ ID NO: 8;
Ant1_HapM3 is or comprises a SNP at a position corresponding to position 865 of SEQ ID NO: 1, position 219 of SEQ ID NO: 2, position 219 of a sequence encoding a protein having a sequence of SEQ ID NO: 3, or position 206 of SEQ ID NO: 9;
Ant1_HapM4 is or comprises a SNP at a position corresponding to position 1092 of SEQ ID NO: 1, position 446 of SEQ ID NO: 2, position 446 of a sequence encoding a protein having a sequence of SEQ ID NO: 3, or position 73 of SEQ ID NO: 10;
Ant1_HapM5 is or comprises a SNP at a position corresponding to position 1284 of SEQ ID NO: 1, position 638 of SEQ ID NO: 2, position 638 of a sequence encoding a protein having a sequence of SEQ ID NO: 3, or position 145 of SEQ ID NO: 11; and/or
Ant1_HapM6 is or comprises a SNP at a position corresponding to position 1309 of SEQ ID NO: 1, position 663 of SEQ ID NO: 2, position 663 of a sequence encoding a protein having a sequence of SEQ ID NO: 3, or position 50 of SEQ ID NO: 12.

26. A method for identifying and/or selecting a barley plant or plant part, comprising screening for the presence of a polynucleotide comprising one or more SNP at a position corresponding to position 71238822 bp (on chromosome 7H) of reference genome sequence assembly of Barley cultivar Morex v3.0 July 2020 (Morex_v3) (Mascher et al., 2021) (Ant1_HapM1);
position 71238454 bp (on chromosome 7H) of reference genome sequence assembly of Barley cultivar Morex v3.0 July 2020 (Morex_v3) (Mascher et al., 2021) (Ant1_HapM2);
position 71238052 bp (on chromosome 7H) of reference genome sequence assembly of Barley cultivar Morex v3.0 July 2020 (Morex_v3) (Mascher et al., 2021) (Ant1_HapM3);
position 71237825 bp (on chromosome 7H) of reference genome sequence assembly of Barley cultivar Morex v3.0 July 2020 (Morex_v3) (Mascher et al., 2021) (Ant1_HapM4);
position 71237633 bp (on chromosome 7H) of reference genome sequence assembly of Barley cultivar Morex v3.0 July 2020 (Morex_v3) (Mascher et al., 2021) (Ant1_HapM5); and/or
position 71237608 bp (on chromosome 7H) of reference genome sequence assembly of Barley cultivar Morex v3.0 July 2020 (Morex_v3) (Mascher et al., 2021) (Ant1_HapM6).

27. A method for identifying and/or selecting a barley plant or plant part, comprising screening for the presence of a polynucleotide comprising one or more SNP at a position corresponding to position 95 of SEQ ID NO: 7 or 95 of SEQ ID NO: 1 (Ant1_HapM1);
position 224 of SEQ ID NO: 8 or 463 of SEQ ID NO: 1 (Ant1_HapM2);
position 206 of SEQ ID NO: 9 or 865 of SEQ ID NO: 1 (Ant1_HapM3);
position 73 of SEQ ID NO: 10 or 1092 of SEQ ID NO: 1 (Ant1_HapM4);
position 145 of SEQ ID NO: 11 or 1284 of SEQ ID NO: 1 (Ant1_HapM5); and/or
position 50 of SEQ ID NO: 12 or 1309 of SEQ ID NO: 1 (Ant1_HapM6).

28. The method according to any of statements 24 to 27, wherein said SNP is
T in Ant1_HapM1;
C in Ant1_HapM2;
C in Ant1_HapM3;
C in Ant1_HapM4;
C in Ant1_HapM5; and/or
G in Ant1_HapM6.

29. The method according to any of statements 1 to 28, wherein
Ant1_HapM1 is, comprises, or is comprised in a polynucleotide having a sequence as set forth in SEQ ID NO: 7 or a nucleotide sequence which is at least 90% identical to SEQ ID NO: 7, the complement or reverse complement thereof;
Ant1_HapM2 is, comprises, or is comprised in a polynucleotide having a sequence as set forth in SEQ ID NO: 8 or a nucleotide sequence which is at least 90% identical to SEQ ID NO: 8, the complement or reverse complement thereof;
Ant1_HapM3 is, comprises, or is comprised in a polynucleotide having a sequence as set forth in SEQ ID NO: 9 or a nucleotide sequence which is at least 90% identical to SEQ ID NO: 9, the complement or reverse complement thereof;
Ant1_HapM4 is, comprises, or is comprised in a polynucleotide having a sequence as set forth in SEQ ID NO: 10; or a nucleotide sequence which is at least 90% identical to SEQ ID NO: 10, the complement or reverse complement thereof;
Ant1_HapM5 is, comprises, or is comprised in a polynucleotide having a sequence as set forth in SEQ ID NO: 11 or a nucleotide sequence which is at least 90% identical to SEQ ID NO: 11, the complement or reverse complement thereof; and/or
Ant1_HapM6 is, comprises, or is comprised in a polynucleotide having a sequence as set forth in SEQ ID NO: 12 or a nucleotide sequence which is at least 90% identical to SEQ ID NO: 12, the complement or reverse complement thereof.

30. The method according to any of statements 1 to 29, wherein
Ant1_HapM1, Ant1_HapM2, Ant1_HapM3, Ant1_HapM4, Ant1_HapM5, and/or Ant1_HapM6 is/are comprised in an Ant1 gene having
a genomic sequence as set forth in SEQ ID NO: 1 or 4 or a sequence which is at least 90% identical to SEQ ID NO: 1 or 4;
a coding genomic sequence as set forth in SEQ ID NO: 2 or 5 or a sequence which is at least 90% identical to SEQ ID NO: 2 or 5;
a sequence encoding a protein having a sequence as set forth in SEQ ID NO: 3 or 6 or a sequence which is at least 90% identical to SEQ ID NO: 3 or 6.

31. A method for identifying and/or selecting a barley plant or plant part, comprising screening for the presence of a polynucleotide having a sequence which is at least 80% identical to a sequence as set forth in SEQ ID NO: 1 or 4 (in the genome of the plant or plant part).

32. A method for identifying and/or selecting a barley plant or plant part, comprising screening for the presence of an Ant1 gene having
a genomic sequence as set forth in SEQ ID NO: 1 or 4 or a sequence which is at least 80% identical to SEQ ID NO: 1 or 4;
a coding genomic sequence as set forth in SEQ ID NO: 2 or 5 or a sequence which is at least 80% identical to SEQ ID NO: 2 or 5;
a sequence encoding a protein having a sequence as set forth in SEQ ID NO: 3 or 6 or a sequence which is at least 80% identical to SEQ ID NO: 3 or 6.

33. The method according to statement 31 or 32 wherein said polynucleotide or Ant1 gene comprises one or more molecular marker (alleles) selected from Ant1_HapM1, Ant1_HapM2, Ant1_HapM3, Ant1_HapM4, Ant1_HapM5, and Ant1_HapM6.

34. The method according to statement 31 or 32 wherein said polynucleotide or Ant1 gene comprises two or more molecular markers (alleles) selected from Ant1_HapM1, Ant1_HapM2, Ant1_HapM3, Ant1_HapM4, Ant1_HapM5, and Ant1_HapM6.

35. The method according to statement 31 or 32 wherein said polynucleotide or Ant1 gene comprises three or more molecular markers (alleles) selected from Ant1_HapM1, Ant1_HapM2, Ant1_HapM3, Ant1_HapM4, Ant1_HapM5, and Ant1_HapM6.

36. The method according to statement 31 or 32 wherein said polynucleotide or Ant1 gene comprises four or more molecular markers (alleles) selected from Ant1_HapM1, Ant1_HapM2, Ant1_HapM3, Ant1_HapM4, Ant1_HapM5, and Ant1_HapM6.

37. The method according to statement 31 or 32 wherein said polynucleotide or Ant1 gene comprises five or more molecular markers (alleles) selected from Ant1_HapM1, Ant1_HapM2, Ant1_HapM3, Ant1_HapM4, Ant1_HapM5, and Ant1_HapM6.

38. The method according to statement 31 or 32 wherein said polynucleotide or Ant1 gene comprises molecular markers (alleles) Ant1_HapM1, Ant1_HapM2, Ant1_HapM3, Ant1_HapM4, Ant1_HapM5, and Ant1_HapM6.

39. The method according to any of statements 31 to 38, comprising screening for the presence of any one or more of molecular markers (alleles) selected from Ant1_HapM1, Ant1_HapM2, Ant1_HapM3, Ant1_HapM4, Ant1_HapM5, and Ant1_HapM6.

40. A method for identifying and/or selecting a barley plant or plant part, comprising screening for the presence of one or more polynucleic acid comprising a sequence which is at least 80%, preferably at least 90%, more preferably at least 95% identical, most preferably at least 98% identical to a sequence as set forth in any one or more of
SEQ ID NO: 7;
SEQ ID NO: 8;
SEQ ID NO: 9;
SEQ ID NO: 10;
SEQ ID NO: 11; and/or
SEQ ID NO: 12.

41. The method according to statement 40, wherein
SEQ ID NO: 7 comprises a SNP at a position corresponding to position 95;
SEQ ID NO: 8 comprises a SNP at a position corresponding to position 224;
SEQ ID NO: 9 comprises a SNP at a position corresponding to position 206;
SEQ ID NO: 10 comprises a SNP at a position corresponding to position 73;
SEQ ID NO: 11 comprises a SNP at a position corresponding to position 145; and/or
SEQ ID NO: 12 comprises a SNP at a position corresponding to position 50.

42. The method according to any of statements 40 to 41, wherein said SNP is
T in SEQ ID NO: 7;
C in SEQ ID NO: 8;
C in SEQ ID NO: 9;
C in SEQ ID NO: 10;
C in SEQ ID NO: 11; and/or
G in SEQ ID NO: 12.
43. A method for identifying and/or selecting a barley plant or plant part, comprising screening for the presence of one or more polynucleic acid comprising a sequence as set forth in any one or more of
SEQ ID NO: 7;
SEQ ID NO: 8;
SEQ ID NO: 9;
SEQ ID NO: 10;
SEQ ID NO: 11; and/or
SEQ ID NO: 12.

44. A method for identifying and/or selecting a barley plant or plant part, comprising screening for the presence of a polynucleotide comprising one or more SNP at a position corresponding to
position 95 of SEQ ID NO: 7;
position 224 of SEQ ID NO: 8;
position 206 of SEQ ID NO: 9;
position 73 of SEQ ID NO: 10;
position 145 of SEQ ID NO: 11; and/or
position 50 of SEQ ID NO: 12.

45. The method according to statements 44, wherein said SNP is
T in SEQ ID NO: 7;
C in SEQ ID NO: 8;
C in SEQ ID NO: 9;
C in SEQ ID NO: 10;
C in SEQ ID NO: 11; and/or
G in SEQ ID NO: 12.

46. A method for identifying and/or selecting a barley plant or plant part, comprising screening for the presence of a polynucleotide comprising one or more SNP in one or more polynucleotide at a position corresponding to
position 95 of SEQ ID NO: 7;
position 224 of SEQ ID NO: 8;
position 206 of SEQ ID NO: 9;
position 73 of SEQ ID NO: 10;
position 145 of SEQ ID NO: 11; and/or
position 50 of SEQ ID NO: 12.

47. The method according to statements 46, wherein said SNP is
T in SEQ ID NO: 7;
C in SEQ ID NO: 8;
C in SEQ ID NO: 9;
C in SEQ ID NO: 10;
C in SEQ ID NO: 11; and/or
G in SEQ ID NO: 12.

48. The method according to any of statements 1 to 47, wherein said polynucleotide, molecular marker (allele), Ant1 gene, or SNP is comprised on chromosome 7H.

49. A method for identifying and/or selecting a barley plant or plant part, comprising screening for the presence of a polynucleotide comprising one or more molecular marker (alleles) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7 (in or associated with/linked to the Ant2 gene and/or in the genome of the plant or plant part).

50. The method according to statement 49, comprising screening for the presence of one or more molecular marker (alleles) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7 (in or associated with/linked to the Ant2 gene and/or in genome of the plant or plant part).

51. A method for identifying and/or selecting a barley plant or plant part, comprising screening for the presence of one or more molecular marker (alleles) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7 (in or associated with/linked to the Ant2 gene and/or in genome of the plant or plant part).

52. The method according to any of statements 1 to 48, further comprising screening for the presence of a polynucleotide comprising one or more molecular marker (alleles) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7 (in or associated with/linked to the Ant2 gene and/or in genome of the plant or plant part).

53. The method according to any of statements 1 to 48, further comprising screening for the presence of one or more molecular marker (alleles) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7 (in or associated with/linked to the Ant2 gene and/or in genome of the plant or plant part).

54. The method according to any of statements 48 to 53, wherein
Ant2_M1 is or comprises a SNP at a position corresponding to position 591324765 bp (on chromosome 2H) of reference genome sequence assembly of Barley cultivar Morex v3.0 July 2020 (Morex_v3) (Mascher et al., 2021)
Ant2_M2 is or comprises a SNP at a position corresponding to position 593492855 bp (on chromosome 2H) of reference genome sequence assembly of Barley cultivar Morex v3.0 July 2020 (Morex_v3) (Mascher et al., 2021);
Ant2_M3 is or comprises a SNP at a position corresponding to position 593493961 bp (on chromosome 2H) of reference genome sequence assembly of Barley cultivar Morex v3.0 July 2020 (Morex_v3) (Mascher et al., 2021);
Ant2_M4 is or comprises a SNP at a position corresponding to position 593494314 bp (on chromosome 2H) of reference genome sequence assembly of Barley cultivar Morex v3.0 July 2020 (Morex_v3) (Mascher et al., 2021);
Ant2_M5 is or comprises a SNP at a position corresponding to position 593494569 bp (on chromosome 2H) of reference genome sequence assembly of Barley cultivar Morex v3.0 July 2020 (Morex_v3) (Mascher et al., 2021);
Ant2_M6 is or comprises a SNP at a position corresponding to position 593495307 bp (on chromosome 2H) of reference genome sequence assembly of Barley cultivar Morex v3.0 July 2020 (Morex_v3) (Mascher et al., 2021);
Ant2_M7 is or comprises a SNP at a position corresponding to position 593496606 bp (on chromosome 2H) of reference genome sequence assembly of Barley cultivar Morex v3.0 July 2020 (Morex_v3) (Mascher et al., 2021);
Ant2_HapM1 is or comprises a SNP at a position corresponding to position 593496442 bp (on chromosome 2H) of reference genome sequence assembly of Barley cultivar Morex v3.0 July 2020 (Morex_v3) (Mascher et al., 2021);
Ant2_HapM2 is or comprises a SNP at a position corresponding to position 593492774 bp (on chromosome 2H) of reference genome sequence assembly of Barley cultivar Morex v3.0 July 2020 (Morex_v3) (Mascher et al., 2021);
Ant2_HapM3 is or comprises a SNP at a position corresponding to position 593493167 bp (on chromosome 2H) of reference genome sequence assembly of Barley cultivar Morex v3.0 July 2020 (Morex_v3) (Mascher et al., 2021);
Ant2_HapM4 is or comprises a SNP at a position corresponding to position 593494160 bp (on chromosome 2H) of reference genome sequence assembly of Barley cultivar Morex v3.0 July 2020 (Morex_v3) (Mascher et al., 2021);
Ant2_HapM5 is or comprises a SNP at a position corresponding to position 593494377 bp (on chromosome 2H) of reference genome sequence assembly of Barley cultivar Morex v3.0 July 2020 (Morex_v3) (Mascher et al., 2021);
Ant2_HapM6 is or comprises a SNP at a position corresponding to position 593495891 bp (on chromosome 2H) of reference genome sequence assembly of Barley cultivar Morex v3.0 July 2020 (Morex_v3) (Mascher et al., 2021); and/or
Ant2_HapM7 is or comprises a SNP at a position corresponding to position 593497594 bp (on chromosome 2H) of reference genome sequence assembly of Barley cultivar Morex v3.0 July 2020 (Morex_v3) (Mascher et al., 2021).

55. The method according to any of statements 48 to 53, wherein
Ant2_M1 is or comprises a SNP at a position corresponding to position 101 of SEQ ID NO: 13;
Ant2_M2 is or comprises a SNP at a position corresponding to position 491 of SEQ ID NO: 14 or position 46 of SEQ ID NO: 20;
Ant2_M3 is or comprises a SNP at a position corresponding to position 1597 of SEQ ID NO: 14 or position 96 of SEQ ID NO: 21;
Ant2_M4 is or comprises a SNP at a position corresponding to position 1950 of SEQ ID NO: 14 or position 64 of SEQ ID NO: 22;
Ant2_M5 is or comprises a SNP at a position corresponding to position 2207 of SEQ ID NO: 14 or position 73 of SEQ ID NO: 23;
Ant2_M6 is or comprises a SNP at a position corresponding to position 2946 of SEQ ID NO: 14 or position 101 of SEQ ID NO: 24;
Ant2_M7 is or comprises a SNP at a position corresponding to position 3907 of SEQ ID NO: 14, position 804 of SEQ ID NO: 15, position 804 of a sequence encoding a protein having a sequence of SEQ ID NO: 16, or position 72 of SEQ ID NO: 25;
Ant2_HapM1 is or comprises a SNP at a position corresponding to position 3743 of SEQ ID NO: 14, position 640 of SEQ ID NO: 15, position 640 of a sequence encoding a protein having a sequence of SEQ ID NO: 16, or position 84 of SEQ ID NO: 26;
Ant2_HapM2 is or comprises a SNP at a position corresponding to position 410 of SEQ ID NO: 14 or position 76 of SEQ ID NO: 27;
Ant2_HapM3 is or comprises a SNP at a position corresponding to position 803 of SEQ ID NO: 14, position 49 of SEQ ID NO: 15, position 49 of a sequence encoding a protein having a sequence of SEQ ID NO: 16, or position 94 of SEQ ID NO: 28;
Ant2_HapM4 is or comprises a SNP at a position corresponding to position 1796 of SEQ ID NO: 14 or position 88 of SEQ ID NO: 29;
Ant2_HapM5 is or comprises a SNP at a position corresponding to position 2013 of SEQ ID NO: 14 or position 52 of SEQ ID NO: 30;
Ant2_HapM6 is or comprises a SNP at a position corresponding to position 3530 of SEQ ID NO: 14 or position 80 of SEQ ID NO: 31; and/or
Ant2_HapM7 is or comprises a SNP at a position corresponding to position 4895 of SEQ ID NO: 14, position 1619 of SEQ ID NO: 15, position 1619 of a sequence encoding a protein having a sequence of SEQ ID NO: 16, or position 73 of SEQ ID NO: 32.

56. A method for identifying and/or selecting a barley plant or plant part, comprising screening for the presence of a polynucleotide comprising one or more SNP at a position corresponding to position 591324765 bp (on chromosome 2H) of reference genome sequence assembly of Barley cultivar Morex v3.0 July 2020 (Morex_v3) (Mascher et al., 2021) (Ant2_M1);
position 593492855 bp (on chromosome 2H) of reference genome sequence assembly of Barley cultivar Morex v3.0 July 2020 (Morex_v3) (Mascher et al., 2021) (Ant2_M2);
position 593493961 bp (on chromosome 2H) of reference genome sequence assembly of Barley cultivar Morex v3.0 July 2020 (Morex_v3) (Mascher et al., 2021) (Ant2_M3);
position 593494314 bp (on chromosome 2H) of reference genome sequence assembly of Barley cultivar Morex v3.0 July 2020 (Morex_v3) (Mascher et al., 2021) (Ant2_M4);
position 593494569 bp (on chromosome 2H) of reference genome sequence assembly of Barley cultivar Morex v3.0 July 2020 (Morex_v3) (Mascher et al., 2021) (Ant2_M5);
position 593495307 bp (on chromosome 2H) of reference genome sequence assembly of Barley cultivar Morex v3.0 July 2020 (Morex_v3) (Mascher et al., 2021) (Ant2_M6);
position 593496606 bp (on chromosome 2H) of reference genome sequence assembly of Barley cultivar Morex v3.0 July 2020 (Morex_v3) (Mascher et al., 2021) (Ant2_M7);
position 593496442 bp (on chromosome 2H) of reference genome sequence assembly of Barley cultivar Morex v3.0 July 2020 (Morex_v3) (Mascher et al., 2021) (Ant2_HapM1);
position 593492774 bp (on chromosome 2H) of reference genome sequence assembly of Barley cultivar Morex v3.0 July 2020 (Morex_v3) (Mascher et al., 2021) (Ant2_HapM2);
position 593493167 bp (on chromosome 2H) of reference genome sequence assembly of Barley cultivar Morex v3.0 July 2020 (Morex_v3) (Mascher et al., 2021) (Ant2_HapM3);
position 593494160 bp (on chromosome 2H) of reference genome sequence assembly of Barley cultivar Morex v3.0 July 2020 (Morex_v3) (Mascher et al., 2021) (Ant2_HapM4);
position 593494377 bp (on chromosome 2H) of reference genome sequence assembly of Barley cultivar Morex v3.0 July 2020 (Morex_v3) (Mascher et al., 2021) (Ant2_HapM5);
position 593495891 bp (on chromosome 2H) of reference genome sequence assembly of Barley cultivar Morex v3.0 July 2020 (Morex_v3) (Mascher et al., 2021) (Ant2_HapM6); and/or
position 593497594 bp (on chromosome 2H) of reference genome sequence assembly of Barley cultivar Morex v3.0 July 2020 (Morex_v3) (Mascher et al., 2021) (Ant2_HapM7);
preferably screening for the presence of a polynucleotide comprising one or more SNP at a position corresponding to
position 593492855 bp (on chromosome 2H) of reference genome sequence assembly of Barley cultivar Morex v3.0 July 2020 (Morex_v3) (Mascher et al., 2021) (Ant2_M2);
position 593493961 bp (on chromosome 2H) of reference genome sequence assembly of Barley cultivar Morex v3.0 July 2020 (Morex_v3) (Mascher et al., 2021) (Ant2_M3);
position 593494314 bp (on chromosome 2H) of reference genome sequence assembly of Barley cultivar Morex v3.0 July 2020 (Morex_v3) (Mascher et al., 2021) (Ant2_M4);
position 593494569 bp (on chromosome 2H) of reference genome sequence assembly of Barley cultivar Morex v3.0 July 2020 (Morex_v3) (Mascher et al., 2021) (Ant2_M5);
position 593495307 bp (on chromosome 2H) of reference genome sequence assembly of Barley cultivar Morex v3.0 July 2020 (Morex_v3) (Mascher et al., 2021) (Ant2_M6); and/or
position 593496606 bp (on chromosome 2H) of reference genome sequence assembly of Barley cultivar Morex v3.0 July 2020 (Morex_v3) (Mascher et al., 2021) (Ant2_M7);
or screening for the presence of a polynucleotide comprising a SNP at a position corresponding to
position 593496442 bp (on chromosome 2H) of reference genome sequence assembly of Barley cultivar Morex v3.0 July 2020 (Morex_v3) (Mascher et al., 2021) (Ant2_HapM1);
and one or more SNP at a position corresponding to
position 593492774 bp (on chromosome 2H) of reference genome sequence assembly of Barley cultivar Morex v3.0 July 2020 (Morex_v3) (Mascher et al., 2021) (Ant2_HapM2);
position 593493167 bp (on chromosome 2H) of reference genome sequence assembly of Barley cultivar Morex v3.0 July 2020 (Morex_v3) (Mascher et al., 2021) (Ant2_HapM3);
position 593494160 bp (on chromosome 2H) of reference genome sequence assembly of Barley cultivar Morex v3.0 July 2020 (Morex_v3) (Mascher et al., 2021) (Ant2_HapM4);
position 593494377 bp (on chromosome 2H) of reference genome sequence assembly of Barley cultivar Morex v3.0 July 2020 (Morex_v3) (Mascher et al., 2021) (Ant2_HapM5);
position 593495891 bp (on chromosome 2H) of reference genome sequence assembly of Barley cultivar Morex v3.0 July 2020 (Morex_v3) (Mascher et al., 2021) (Ant2_HapM6); and/or
position 593497594 bp (on chromosome 2H) of reference genome sequence assembly of Barley cultivar Morex v3.0 July 2020 (Morex_v3) (Mascher et al., 2021) (Ant2_HapM7).

57. A method for identifying and/or selecting a barley plant or plant part, comprising screening for the presence of a polynucleotide comprising one or more SNP at a position corresponding to
position 101 of SEQ ID NO: 13 (Ant2_M1)
position 46 of SEQ ID NO: 20 (Ant2_M2)
position 96 of SEQ ID NO: 21 (Ant2_M3)
position 64 of SEQ ID NO: 22 (Ant2_M4)
position 73 of SEQ ID NO: 23 (Ant2_M5)
position 101 of SEQ ID NO: 24 (Ant2_M6)
position 72 of SEQ ID NO: 25 (Ant2_M7)
position 84 of SEQ ID NO: 26 (Ant2_HapM1)
position 76 of SEQ ID NO: 27 (Ant2_HapM2)
position 94 of SEQ ID NO: 28 (Ant2_HapM3)
position 88 of SEQ ID NO: 29 (Ant2_HapM4)
position 52 of SEQ ID NO: 30 (Ant2_HapM5)
position 80 of SEQ ID NO: 31 (Ant2_HapM6) and/or
position 73 of SEQ ID NO: 32 (Ant2_HapM7);
preferably screening for the presence of a polynucleotide comprising one or more SNP at a position corresponding to
position 46 of SEQ ID NO: 20 (Ant2_M2)
position 96 of SEQ ID NO: 21 (Ant2_M3)
position 64 of SEQ ID NO: 22 (Ant2_M4)
position 73 of SEQ ID NO: 23 (Ant2_M5)
position 101 of SEQ ID NO: 24 (Ant2_M6) and/or
position 72 of SEQ ID NO: 25 (Ant2_M7);
or screening for the presence of a polynucleotide comprising a SNP at a position corresponding to
position 84 of SEQ ID NO: 26 (Ant2_HapM1)
and one or more SNP at a position corresponding to
position 76 of SEQ ID NO: 27 (Ant2_HapM2)
position 94 of SEQ ID NO: 28 (Ant2_HapM3)
position 88 of SEQ ID NO: 29 (Ant2_HapM4)
position 52 of SEQ ID NO: 30 (Ant2_HapM5)
position 80 of SEQ ID NO: 31 (Ant2_HapM6) and/or
position 73 of SEQ ID NO: 32 (Ant2_HapM7).

58. The method according to any of statements 54 to 57, wherein said SNP is
C in Ant2_M1
G in Ant2_M2
C in Ant2_M3
T in Ant2_M4
G in Ant2_M5
T in Ant2_M6
A in Ant2_M7
G in Ant2_HapM1
C in Ant2_HapM2
A in Ant2_HapM3
T in Ant2_HapM4
A in Ant2_HapM5
G in Ant2_HapM6 and/or
C in Ant2_HapM7.

59. The method according to any of statements 49 to 58, wherein
Ant2_M1 is, comprises, or is comprised in a polynucleotide having a sequence as set forth in SEQ ID NO: 13 or a nucleotide sequence which is at least 90% identical to SEQ ID NO: 13, the complement or reverse complement thereof
Ant2_M2 is, comprises, or is comprised in a polynucleotide having a sequence as set forth in SEQ ID NO: 20 or a nucleotide sequence which is at least 90% identical to SEQ ID NO: 20, the complement or reverse complement thereof
Ant2_M3 is, comprises, or is comprised in a polynucleotide having a sequence as set forth in SEQ ID NO: 21 or a nucleotide sequence which is at least 90% identical to SEQ ID NO: 21, the complement or reverse complement thereof
Ant2_M4 is, comprises, or is comprised in a polynucleotide having a sequence as set forth in SEQ ID NO: 22 or a nucleotide sequence which is at least 90% identical to SEQ ID NO: 22, the complement or reverse complement thereof
Ant2_M5 is, comprises, or is comprised in a polynucleotide having a sequence as set forth in SEQ ID NO: 23 or a nucleotide sequence which is at least 90% identical to SEQ ID NO: 23, the complement or reverse complement thereof
Ant2_M6 is, comprises, or is comprised in a polynucleotide having a sequence as set forth in SEQ ID NO: 24 or a nucleotide sequence which is at least 90% identical to SEQ ID NO: 24, the complement or reverse complement thereof
Ant2_M7 is, comprises, or is comprised in a polynucleotide having a sequence as set forth in SEQ ID NO: 25 or a nucleotide sequence which is at least 90% identical to SEQ ID NO: 25, the complement or reverse complement thereof
Ant2_HapM1 is, comprises, or is comprised in a polynucleotide having a sequence as set forth in SEQ ID NO: 26 or a nucleotide sequence which is at least 90% identical to SEQ ID NO: 26, the complement or reverse complement thereof
Ant2_HapM2 is, comprises, or is comprised in a polynucleotide having a sequence as set forth in SEQ ID NO: 27 or a nucleotide sequence which is at least 90% identical to SEQ ID NO: 27, the complement or reverse complement thereof
Ant2_HapM3 is, comprises, or is comprised in a polynucleotide having a sequence as set forth in SEQ ID NO: 28 or a nucleotide sequence which is at least 90% identical to SEQ ID NO: 28, the complement or reverse complement thereof
Ant2_HapM4 is, comprises, or is comprised in a polynucleotide having a sequence as set forth in SEQ ID NO: 29 or a nucleotide sequence which is at least 90% identical to SEQ ID NO: 29, the complement or reverse complement thereof
Ant2_HapM5 is, comprises, or is comprised in a polynucleotide having a sequence as set forth in SEQ ID NO: 30 or a nucleotide sequence which is at least 90% identical to SEQ ID NO: 30, the complement or reverse complement thereof
Ant2_HapM6 is, comprises, or is comprised in a polynucleotide having a sequence as set forth in SEQ ID NO: 31 or a nucleotide sequence which is at least 90% identical to SEQ ID NO: 31, the complement or reverse complement thereof; and/or
Ant2_HapM7 is, comprises, or is comprised in a polynucleotide having a sequence as set forth in SEQ ID NO: 32 or a nucleotide sequence which is at least 90% identical to SEQ ID NO: 32, the complement or reverse complement thereof.

60. The method according to any of statements 49 to 59, wherein Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7 is/are comprised in or are linked with or associated with an Ant2 gene having
a genomic sequence as set forth in SEQ ID NO: 14 or 17 or a sequence which is at least 90% identical to SEQ ID NO: 14 or 17;
a coding genomic sequence as set forth in SEQ ID NO: 15 or 18 or a sequence which is at least 90% identical to SEQ ID NO: 15 or 18;
a sequence encoding a protein having a sequence as set forth in SEQ ID NO: 16 or 19 or a sequence which is at least 90% identical to SEQ ID NO: 16 or 19.

61. A method for identifying and/or selecting a barley plant or plant part, comprising screening for the presence of a polynucleotide having a sequence which is at least 80% identical to a sequence as set forth in SEQ ID NO: 14 or 17 (in the genome of the plant or plant part).

62. A method for identifying and/or selecting a barley plant or plant part, comprising screening for the presence of an Ant2 gene having
a genomic sequence as set forth in SEQ ID NO: 14 or 17 or a sequence which is at least 80% identical to SEQ ID NO: 14 or 17;
a coding sequence as set forth in SEQ ID NO: 15 or 18 or a sequence which is at least 80% identical to SEQ ID NO: 15 or 18;
a sequence encoding a protein having a sequence as set forth in SEQ ID NO: 16 or 19 or a sequence which is at least 80% identical to SEQ ID NO: 16 or 19.

63. The method according to statement 61 wherein said polynucleotide Ant2 comprises molecular marker (allele) Ant2_M1 and/or wherein said polynucleotide or Ant2 gene comprises one or more of molecular marker(s) (alleles) Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably wherein said polynucleotide or Ant2 gene comprises one or more of molecular marker(s) (alleles) Ant2_M2, Ant2_M3, Ant2_M4, Ant2_M5, Ant2_M6, and/or Ant2_M7; or wherein said polynucleotide or Ant2 gene comprises molecular marker (allele) Ant2_HapM1 and one or more of molecular marker(s) (alleles) Ant2_HapM2, Ant2_HapM3, Ant2_HapM4, Ant2_HapM5, Ant2_HapM6, and/or Ant2_HapM7.

64. The method according to any of statements 61 to 63, comprising screening for the presence of one or more of marker (allele) Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably screening for the presence of one or more of marker (allele) Ant2_M2, Ant2_M3, Ant2_M4, Ant2_M5, Ant2_M6, and/or Ant2_M7; or screening for the presence of marker (allele) Ant2_HapM1 and one or more of marker(s) (alleles) Ant2_HapM2, Ant2_HapM3, Ant2_HapM4, Ant2_HapM5, Ant2_HapM6, and/or Ant2_HapM7.

65. A method for identifying and/or selecting a barley plant or plant part, comprising screening for the presence of one or more polynucleic acid comprising a sequence which is at least 80%, preferably at least 90%, more preferably at least 95% identical, most preferably at least 98% identical to a sequence as set forth in
SEQ ID NO: 13
SEQ ID NO: 20
SEQ ID NO: 21
SEQ ID NO: 22
SEQ ID NO: 23
SEQ ID NO: 24
SEQ ID NO: 25
SEQ ID NO: 26
SEQ ID NO: 27
SEQ ID NO: 28
SEQ ID NO: 29
SEQ ID NO: 30
SEQ ID NO: 31 and/or
SEQ ID NO: 32
preferably screening for the presence of one or more polynucleic acid comprising a sequence which is at least 80%, preferably at least 90%, more preferably at least 95% identical, most preferably at least 98% identical to a sequence as set forth in
SEQ ID NO: 20
SEQ ID NO: 21
SEQ ID NO: 22
SEQ ID NO: 23
SEQ ID NO: 24 and/or
SEQ ID NO: 25
or screening for the presence of a polynucleic acid comprising a sequence which is at least 80%, preferably at least 90%, more preferably at least 95% identical, most preferably at least 98% identical to a sequence as set forth in
SEQ ID NO: 26 and one or more polynucleic acid comprising a sequence which is at least 80%, preferably at least 90%, more preferably at least 95% identical, most preferably at least 98% identical to a sequence as set forth in
SEQ ID NO: 27
SEQ ID NO: 28
SEQ ID NO: 29
SEQ ID NO: 30
SEQ ID NO: 31 and/or
SEQ ID NO: 32.

66 The method according to statement 65, wherein
SEQ ID NO: 13 comprises a SNP at a position corresponding to position 101
SEQ ID NO: 20 comprises a SNP at a position corresponding to position 46
SEQ ID NO: 21 comprises a SNP at a position corresponding to position 96
SEQ ID NO: 22 comprises a SNP at a position corresponding to position 64
SEQ ID NO: 23 comprises a SNP at a position corresponding to position 73
SEQ ID NO: 24 comprises a SNP at a position corresponding to position 101
SEQ ID NO: 25 comprises a SNP at a position corresponding to position 72
SEQ ID NO: 26 comprises a SNP at a position corresponding to position 84
SEQ ID NO: 27 comprises a SNP at a position corresponding to position 76
SEQ ID NO: 28 comprises a SNP at a position corresponding to position 94
SEQ ID NO: 29 comprises a SNP at a position corresponding to position 88
SEQ ID NO: 30 comprises a SNP at a position corresponding to position 52
SEQ ID NO: 31 comprises a SNP at a position corresponding to position 80 and/or
SEQ ID NO: 32 comprises a SNP at a position corresponding to position 73.

67. The method according to any of statements 65 to 66, wherein said SNP is
C in SEQ ID NO: 13
G in SEQ ID NO: 20
C in SEQ ID NO: 21
T in SEQ ID NO: 22
G in SEQ ID NO: 23
T in SEQ ID NO: 24
A in SEQ ID NO: 25
G in SEQ ID NO: 26
C in SEQ ID NO: 27
A in SEQ ID NO: 28
T in SEQ ID NO: 29
A in SEQ ID NO: 30
G in SEQ ID NO: 31 and/or
C in SEQ ID NO: 32.

68. A method for identifying and/or selecting a barley plant or plant part, comprising screening for the presence of a polynucleic acid comprising a sequence as set forth in
SEQ ID NO: 13
SEQ ID NO: 20
SEQ ID NO: 21
SEQ ID NO: 22
SEQ ID NO: 23
SEQ ID NO: 24
SEQ ID NO: 25
SEQ ID NO: 26$
SEQ ID NO: 27
SEQ ID NO: 28
SEQ ID NO: 29
SEQ ID NO: 30
SEQ ID NO: 31 and/or
SEQ ID NO: 32;
preferably screening for the presence of a polynucleic acid comprising a sequence as set forth in
SEQ ID NO: 13
SEQ ID NO: 20
SEQ ID NO: 21
SEQ ID NO: 22
SEQ ID NO: 23
SEQ ID NO: 24 and/or
SEQ ID NO: 25;
or screening for the presence of a polynucleic acid comprising a sequence as set forth in SEQ ID NO: 26 and one or more of
SEQ ID NO: 27
SEQ ID NO: 28
SEQ ID NO: 29
SEQ ID NO: 30
SEQ ID NO: 31 and/or
SEQ ID NO: 32.

69. A method for identifying and/or selecting a barley plant or plant part, comprising screening for the presence of a polynucleotide comprising a SNP at a position corresponding to
position 101 of SEQ ID NO: 13 (Ant2_M1)
position 46 of SEQ ID NO: 20 (Ant2_M2)
position 96 of SEQ ID NO: 21 (Ant2_M3)
position 64 of SEQ ID NO: 22 (Ant2_M4)
position 73 of SEQ ID NO: 23 (Ant2_M5)
position 101 of SEQ ID NO: 24 (Ant2_M6)
position 72 of SEQ ID NO: 25 (Ant2_M7)
position 84 of SEQ ID NO: 26 (Ant2_HapM1)
position 76 of SEQ ID NO: 27 (Ant2_HapM2)
position 94 of SEQ ID NO: 28 (Ant2_HapM3)
position 88 of SEQ ID NO: 29 (Ant2_HapM4)
position 52 of SEQ ID NO: 30 (Ant2_HapM5)
position 80 of SEQ ID NO: 31 (Ant2_HapM6) and/or
position 73 of SEQ ID NO: 32 (Ant2_HapM7);
preferably screening for the presence of a polynucleotide comprising one or more SNP at a position corresponding to
position 46 of SEQ ID NO: 20 (Ant2_M2)
position 96 of SEQ ID NO: 21 (Ant2_M3)
position 64 of SEQ ID NO: 22 (Ant2_M4)
position 73 of SEQ ID NO: 23 (Ant2_M5)
position 101 of SEQ ID NO: 24 (Ant2_M6) and/or
position 72 of SEQ ID NO: 25 (Ant2_M7);
or screening for the presence of a polynucleotide comprising a SNP at a position corresponding to
position 84 of SEQ ID NO: 26 (Ant2_HapM1)
and one or more SNP at a position corresponding to
position 76 of SEQ ID NO: 27 (Ant2_HapM2)
position 94 of SEQ ID NO: 28 (Ant2_HapM3)
position 88 of SEQ ID NO: 29 (Ant2_HapM4)
position 52 of SEQ ID NO: 30 (Ant2_HapM5)
position 80 of SEQ ID NO: 31 (Ant2_HapM6) and/or
position 73 of SEQ ID NO: 32 (Ant2_HapM7).

70. The method according to statements 69, wherein said SNP is
C in SEQ ID NO: 13
G in SEQ ID NO: 20
C in SEQ ID NO: 21
T in SEQ ID NO: 22
G in SEQ ID NO: 23
T in SEQ ID NO: 24
A in SEQ ID NO: 25
G in SEQ ID NO: 26
C in SEQ ID NO: 27
A in SEQ ID NO: 28
T in SEQ ID NO: 29
A in SEQ ID NO: 30
G in SEQ ID NO: 31 and/or
C in SEQ ID NO: 32.

71. A method for identifying and/or selecting a barley plant or plant part, comprising screening for the presence of a polynucleotide comprising a SNP in a polynucleotide at a position corresponding to
position 101 of SEQ ID NO: 13 (Ant2_M1)
position 46 of SEQ ID NO: 20 (Ant2_M2)
position 96 of SEQ ID NO: 21 (Ant2_M3)
position 64 of SEQ ID NO: 22 (Ant2_M4)
position 73 of SEQ ID NO: 23 (Ant2_M5)
position 101 of SEQ ID NO: 24 (Ant2_M6)
position 72 of SEQ ID NO: 25 (Ant2_M7)
position 84 of SEQ ID NO: 26 (Ant2_HapM1)
position 76 of SEQ ID NO: 27 (Ant2_HapM2)
position 94 of SEQ ID NO: 28 (Ant2_HapM3)
position 88 of SEQ ID NO: 29 (Ant2_HapM4)
position 52 of SEQ ID NO: 30 (Ant2_HapM5)
position 80 of SEQ ID NO: 31 (Ant2_HapM6) and/or
position 73 of SEQ ID NO: 32 (Ant2_HapM7);
preferably screening for the presence of a polynucleotide comprising a SNP at a position corresponding to
position 46 of SEQ ID NO: 20 (Ant2_M2)
position 96 of SEQ ID NO: 21 (Ant2_M3)
position 64 of SEQ ID NO: 22 (Ant2_M4)
position 73 of SEQ ID NO: 23 (Ant2_M5)
position 101 of SEQ ID NO: 24 (Ant2_M6) and/or
position 72 of SEQ ID NO: 25 (Ant2_M7);
or screening for the presence of a polynucleotide comprising a SNP at a position corresponding to
position 84 of SEQ ID NO: 26 (Ant2_HapM1)
and a SNP at a position corresponding to one or more of
position 76 of SEQ ID NO: 27 (Ant2_HapM2)
position 94 of SEQ ID NO: 28 (Ant2_HapM3)
position 88 of SEQ ID NO: 29 (Ant2_HapM4)
position 52 of SEQ ID NO: 30 (Ant2_HapM5)
position 80 of SEQ ID NO: 31 (Ant2_HapM6) and/or
position 73 of SEQ ID NO: 32 (Ant2_HapM7).

72. The method according to statements 71, wherein said SNP is
C in SEQ ID NO: 13
G in SEQ ID NO: 20
C in SEQ ID NO: 21
T in SEQ ID NO: 22
G in SEQ ID NO: 23
T in SEQ ID NO: 24
A in SEQ ID NO: 25
G in SEQ ID NO: 26
C in SEQ ID NO: 27
A in SEQ ID NO: 28
T in SEQ ID NO: 29
A in SEQ ID NO: 30
G in SEQ ID NO: 31 and/or
C in SEQ ID NO: 32.

73. The method according to any of statements 49 to 64, wherein said polynucleotide, molecular marker (allele), Ant2 gene, or SNP is comprised on chromosome 2H.

74. The method according to any of statements 1 to 73, which is a method for identifying a barley plant or plant part producing or capable of producing a seed having a purple pericarp and/or hull (palea and lemma), and/or a plant grown therefrom and producing or capable of producing a seed having a purple pericarp and/or hull (palea and lemma).

75. The method according to any of statements 1 to 73, which is a method for identifying a barley plant or plant part producing or capable of producing a seed having a non-purple pericarp and/or hull (palea and lemma), and/or a plant grown therefrom and producing or capable of producing a seed having a non-purple pericarp and/or hull (palea and lemma).

76. The method according to any of statements 1 to 73, which is a method for distinguishing between a barley plant or plant part producing or capable of producing a seed having a purple pericarp and/or hull (palea and lemma) and a barley plant or plant part producing or capable of producing a seed having a non-purple pericarp and/or hull (palea and lemma), and/or a plant grown therefrom and producing or capable of producing a seed having a purple pericarp and/or hull (palea and lemma) or a non-purple pericarp and/or hull (palea and lemma).

77. The method according to any of statements 74 to 76, wherein said barley plant of plant part is identified as producing or being capable of producing a seed having a purple pericarp and/or hull (palea and lemma) and/or a plant grown therefrom and producing or capable of producing a seed having a purple pericarp and/or hull (palea and lemma) if said polynucleotide, molecular marker (allele), Ant1 gene, Ant2 gene, or SNP is present in the genome of said plant or plant part.

78. The method according to any of statements 1 to 77, wherein said polynucleotide, molecular marker (allele), Ant1 gene, Ant2 gene, and/or SNP is homozygous.

79. The method according to any of statements 1 to 77, wherein said polynucleotide, molecular marker (allele), Ant1 gene, Ant2 gene, and/or SNP is heterozygous.

80. The method according to statements 74, wherein said polynucleotide, molecular marker (allele), Ant1 gene, and/or SNP as defined in any of statements 1 to 48 is homozygous, and wherein said polynucleotide, molecular marker (allele). Ant2 gene, and/or SNP according to any of statements 49 to 73 is homozygous or heterozygous.

81. The method according to statements 75, wherein said polynucleotide, molecular marker (allele), Ant1 gene, and/or SNP as defined in any of statements 1 to 48 is absent or heterozygous and/or said polynucleotide, molecular marker (allele), Ant2 gene, and/or SNP according to any of statements 49 to 73 is absent.

82. The method according to statements 76 to 77, wherein said barley plant of plant part is identified as producing or being capable of producing a seed having a purple pericarp and/or hull (palea and lemma) and/or a plant grown therefrom and producing or capable of producing a seed having a purple pericarp and/or hull (palea and lemma) if said polynucleotide, molecular marker (allele), Ant1 gene, or SNP as defined in any of statements 1 to 48 is homozygously present in the genome of said plant or plant part, and wherein said polynucleotide, molecular marker (allele), Ant2 gene, or SNP as defined in any of statements 49 to 73 is homozygously or is heterozygously present in the genome of said plant or plant part.

83. The method according to statements 76 to 77, wherein said barley plant or plant part is identified as producing or being capable of producing a seed having a non-purple pericarp and/or hull (palea and lemma) and/or a plant grown therefrom and producing or capable of producing a seed having a non-purple pericarp and/or hull (palea and lemma) if said polynucleotide, molecular marker (allele), Ant1 gene, or SNP as defined in any of statements 1 to 48 is absent or heterozygously present in the genome of said plant or plant part and/or if said polynucleotide, molecular marker (allele), Ant2 gene, or SNP as defined in any of statements 49 to 73 is absent in the genome of said plant or plant part.

84. The method according to any of statements 1 to 83, comprising isolating (genomic) DNA from the plant or plant part.

85. The method according to any of statements 1 to 84, comprising selecting a plant or plant part comprising the polynucleotide, one or more of the (molecular) marker (allele), or the SNP.

86. The method according to any of statements 1 to 84, comprising selecting a plant or plant part comprising a homozygous polynucleotide, molecular marker (allele), Ant1 gene, or SNP as defined in any of statements 1 to 48 and a homozygous or heterozygous polynucleotide, molecular marker (allele), Ant2 gene, or SNP as defined in any of statements 49 to 73.

87. The method according to any of statements 1 to 84, comprising selecting a plant or plant part not comprising a homozygous polynucleotide, molecular marker (allele), Ant1 gene, or SNP as defined in any of statements 1 to 48 and a homozygous or heterozygous polynucleotide, molecular marker (allele), Ant2 gene, or SNP as defined in any of statements 49 to 73.

88. The method according to any of statements 1 to 87, wherein said plant part is a seed.

89. The method according to any of statements 1 to 87, wherein said plant part is not propagation material.

90. The method according to any of statements 1 to 89, wherein said polynucleotide has a length of at most 3 Mb, such as at most 1 Mb, preferably at most 500 kb, more preferably at most 200 kb, most preferably at most 100 kb, and/or wherein said polynucleotide has a length of at least 15 nucleotides, preferably at least 18 nucleotides, more preferably at least 20 nucleotides; or wherein said polynucleotide has a length of at most 10 kb, preferably at most 5 kb, and/or wherein said polynucleotide has a length of at least 15 nucleotides, preferably at least 18 nucleotides, more preferably at least 20 nucleotides.

91. A barley plant or plant part comprising the polynucleotide, molecular marker (allele), SNP, Ant1 gene, and/or Ant2 gene as defined in any of statements 1 to 87.

92. The barley plant or plant part according to statement 91, expressing or capable of expressing the Ant1 and/or Ant2 gene.

93. The barley plant or plant part according to statement 91, expressing or capable of 93. expressing the Ant1 gene on chromosome 7H.

94. The barley plant or plant part according to statement 91, expressing or capable of expressing the Ant2 gene on chromosome 2H.

95. The barley plant or plant part according to any of statements 91 to 94, wherein said polynucleotide, molecular marker (allele), SNP, Ant1 gene, and/or Ant2 gene is homozygous.

96. The barley plant or plant part according to any of statements 91 to 94, wherein said polynucleotide, molecular marker (allele), SNP, Ant1 gene, and/or Ant2 gene is heterozygous.

97. The barley plant or plant part according to any of statements 91 to 96, wherein said plant is sterile.

98. The barley plant or plant part according to any of statements 89 to 97, wherein said plant is male sterile.

99. The barley plant or plant part according to any of statements 89 to 97, wherein said plant is cytoplasmically sterile.

100. The barley plant or plant part according to any of statements 89 to 99, wherein said plant is cytoplasmically male sterile.

101. A sterile, preferably male sterile, more preferably cytoplasmically male sterile barley plant comprising a homozygous polynucleotide, molecular marker (allele), SNP, Ant1 gene as defined in any of statements 1 to 48 and comprising a homozygous or heterozygous polynucleotide, molecular marker (allele), SNP, Ant2 gene as defined in any of statements 48 to 73, or a plant part thereof.

102. The barley plant or plant part according to any of statements 89 to 101, wherein said plant or plant part is mutagenized, such as chemical mutagenization or mutagenization by radiation.

103. The barley plant or plant part according to any of statements 89 to 101, wherein said plant or plant part is transgenic or gene-edited.

104. The barley plant or plant part according to any of statements 89 to 101, wherein said plant or plant part is obtained by introgression of said one or more polynucleotide, molecular marker (allele), SNP, Ant1 gene and/or Ant2 gene.

105. A method for generating or producing a barley plant or plant part, comprising introducing into the genome of a barley plant or plant part a polynucleotide, molecular marker (allele), SNP, Ant1 gene or coding sequence thereof, and/or Ant2 gene or coding sequence thereof as defined in any of statements 1 to 73.

106. A method for generating or producing a barley plant or plant part, comprising providing a barley seed mixture harvested from a cross between a first barley parent plant (population) and a second barley parent plant (population), wherein said first and/or second barley parent plant (population) comprises a polynucleotide, molecular marker (allele), SNP, Ant1 gene, and/or Ant2 gene as defined in any of statements 1 to 73;
selecting seeds comprising a polynucleotide, molecular marker (allele), SNP, Ant1 gene, and/or Ant2 gene as defined in any of statements 1 to 73.

107. The method according to statement 106, wherein said first or second barley parent plant (population) comprises a polynucleotide, molecular marker (allele), SNP, Ant1 gene, and/or Ant2 gene as defined in any of statements 1 to 73.

108. The method according to statement 106 or 107, wherein one and only one of said first or second barley parent plant (population) comprises a homozygous polynucleotide, molecular marker (allele), SNP, or Ant1 gene as defined in any of statements 1 to 48 and a homozygous or heterozygous polynucleotide, molecular marker (allele), SNP, or Ant2 gene as defined in any of statements 49 to 73.

109. The method according to any of statements 106 to 108, wherein one and only one of said first or second barley parent plant (population) comprises a homozygous polynucleotide, molecular marker (allele), SNP, or Ant1 gene as defined in any of statements 1 to 48 and a homozygous or heterozygous polynucleotide, molecular marker (allele). SNP, or Ant2 gene as defined in any of statements 49 to 73, wherein the other plant (population) does not comprise a heterozygous polynucleotide, molecular marker (allele), SNP, or Ant1 gene as defined in any of statements 1 to 48, and wherein if both plants (populations) comprise a homozygous polynucleotide, molecular marker (allele), SNP, or Ant1 gene as defined in any of statements 1 to 48 one of the plants (populations) comprises a homozygous polynucleotide, molecular marker (allele), SNP, or Ant2 gene as defined in any of statements 49 to 73.

110. The method according to any of statements 106 to 109, wherein one and only one of said first or second barley parent plant (population) comprises a polynucleotide, molecular marker (allele), SNP, or Ant1 gene as defined in any of statements 1 to 48 (and the other one does not—i.e., neither homozygous nor heterozygous), wherein said polynucleotide, molecular marker (allele), SNP, or Ant1 gene is homozygous, and comprises a homozygous or heterozygous polynucleotide, molecular marker (allele), SNP, or Ant2 gene as defined in any of statements 49 to 73.

111. The method according to any of statements 106 to 109, wherein said first barley parent plant (population) comprises a homozygous polynucleotide, molecular marker (allele), SNP, or Ant1 gene as defined in any of statements 1 to 48 and a homozygous polynucleotide, molecular marker (allele), SNP, or Ant2 gene as defined in any of statements 49 to 73; and wherein said second barley parent plant (population) comprises a homozygous polynucleotide, molecular marker (allele), SNP, or Ant1 gene as defined in any of statements 1 to 48 and does not comprise a polynucleotide, molecular marker (allele), SNP, or Ant2 gene as defined in any of statements 49 to 73.

112. The method according to any of statements 106 to 111, further comprising selecting seeds comprising a polynucleotide, molecular marker (allele), SNP or Ant1 gene as defined in any of statements 1 to 48, and a polynucleotide, molecular marker (allele), SNP or Ant2 gene as defined in any of statements 49 to 73.

113. The method according to statement 112, wherein said seeds comprise a homozygous polynucleotide, molecular marker (allele), SNP or Ant1 gene as defined in any of statements 1 to 48, and a homozygous or heterozygous polynucleotide, molecular marker (allele), SNP or Ant2 gene as defined in any of statements 49 to 73.

114. The method according to any of statements 106 to 111, further comprising selecting seeds not comprising a homozygous polynucleotide, molecular marker (allele), SNP or Ant1 gene as defined in any of statements 1 to 48, and a homozygous or heterozygous polynucleotide, molecular marker (allele), SNP or Ant2 gene as defined in any of statements 49 to 73.

115. A method for generating or producing a barley plant or plant part, comprising introducing into the genome of a barley plant or plant part a (Ant1 encoding) polynucleotide having a sequence selected from
(1) a sequence having a sequence which is at least 80%, preferably at least 90%, more preferably at least 95%, identical to a sequence as set forth in SEQ ID NO: 1 and/or one or more sequences as set forth in SEQ ID NOs: 7 to 12;
(2) a sequence having a sequence which is at least 80%, preferably at least 90%, more preferably at least 95%, identical to a sequence as set forth in SEQ ID NO: 2; and/or
(3) a sequence encoding a protein having a sequence which is at least 80%, preferably at least 90%, more preferably at least 95%, identical to a sequence as set forth in SEQ ID NO: 3.

116. A method for generating or producing a barley plant or plant part, comprising introducing into the genome of a barley plant or plant part a (Ant2 encoding) polynucleotide having a sequence selected from
(1) a sequence having a sequence which is at least 80%, preferably at least 90%, more preferably at least 95%, identical to a sequence as set forth in SEQ ID NO: 14 and/or one or more sequences as set forth in SEQ ID NOs: 13 or 20 to 32, preferably one or more sequences as set forth in SEQ ID NOs: 20 to 25 or a sequence as set forth in SEQ ID NO: 26 and one or more sequences as set forth in SEQ ID NOs: 27 to 32;
(2) a sequence having a sequence which is at least 80%, preferably at least 90%, more preferably at least 95%, identical to a sequence as set forth in SEQ ID NO: 15; and/or
(3) a sequence encoding a protein having a sequence which is at least 80%, preferably at least 90%, more preferably at least 95%, identical to a sequence as set forth in SEQ ID NO: 16.

117. A method for generating or producing a barley plant or plant part, comprising
(A) introducing into the genome of a barley plant or plant part a (Ant1 encoding) polynucleotide having a sequence selected from
(1) a sequence having a sequence which is at least 80%, preferably at least 90%, more preferably at least 95%, identical to a sequence as set forth in SEQ ID NO: 1 and/or one or more sequences as set forth in SEQ ID NOs: 7 to 12;
(2) a sequence having a sequence which is at least 80%, preferably at least 90%, more preferably at least 95%, identical to a sequence as set forth in SEQ ID NO: 2; and/or
(3) a sequence encoding a protein having a sequence which is at least 80%, preferably at least 90%, more preferably at least 95%, identical to a sequence as set forth in SEQ ID NO: 3; and
(B) simultaneously or sequentially, in either order, introducing into the genome of a barley plant or plant part a (Ant2 encoding) polynucleotide having a sequence selected from
(1) a sequence having a sequence which is at least 80%, preferably at least 90%, more preferably at least 95%, identical to a sequence as set forth in SEQ ID NO: 14 and/or a one or more sequences as set forth in SEQ ID NOs: 13 or 20 to 32, preferably one or more sequences as set forth in SEQ ID NOs: 20 to 25 or a sequence as set forth in SEQ ID NO: 26 and one or more sequences as set forth in SEQ ID NOs: 27 to 32;
(2) a sequence having a sequence which is at least 80%, preferably at least 90%, more preferably at least 95%, identical to a sequence as set forth in SEQ ID NO: 15; and/or
(3) a sequence encoding a protein having a sequence which is at least 80%, preferably at least 90%, more preferably at least 95%, identical to a sequence as set forth in SEQ ID NO: 16.

118. The method according to any of statements 115 to 117, wherein said polynucleotide(s) is (are) homozygous.

119. The method according to any of statements 115 to 117, wherein said polynucleotide(s) is (are) heterozygous.

120. The method according to statement 117, wherein said polynucleotide in (A) is homozygous and wherein said polynucleotide in (B) is homozygous or heterozygous.

121. The method according to any of statements 118 or 120, wherein homozygosity is achieved by doubled haploid technology.

122. The method according to any of statements 106 to 121, wherein said polynucleotide has a length of at most 1 Mb, preferably at most 500 kb, more preferably at most 200 kb, most preferably at most 100 kb; or wherein said polynucleotide has a length of at most 10 kb, preferably at most 5 kb.

123. The method according to any of statements 105 to 122, wherein introducing into the genome comprises introgression.

124. The method according to any of statements 105 to 123, comprising (a) providing a first barley plant having the polynucleotide, molecular marker (allele), SNP, Ant1 gene or coding sequence thereof, and/or Ant2 gene or coding sequence thereof as defined in any of statements 1 to 73, (b) crossing said first barley plant with a second barley plant, and (c) selecting progeny plants having the polynucleotide, molecular marker (allele), SNP, Ant1 gene or coding sequence thereof, and/or Ant2 gene or coding sequence thereof as defined in any of statements 1 to 73.

125. The method according to statement 124, wherein one and only one of said first or second barley parent plant (population) comprises a homozygous polynucleotide, molecular marker (allele), SNP, or Ant1 gene as defined in any of statements 1 to 48 and a homozygous or heterozygous polynucleotide, molecular marker (allele), SNP, or Ant2 gene as defined in any of statements 49 to 73.

126. The method according to any of statements 124 to 125, wherein one and only one of said first or second barley parent plant (population) comprises a homozygous polynucleotide, molecular marker (allele), SNP, or Ant1 gene as defined in any of statements 1 to 48 and a homozygous or heterozygous polynucleotide, molecular marker (allele), SNP, or Ant2 gene as defined in any of statements 49 to 73, wherein the other plant (population) does not comprise a heterozygous polynucleotide, molecular marker (allele), SNP, or Ant1 gene as defined in any of statements 1 to 48, and wherein if both plants (populations) comprise a homozygous polynucleotide, molecular marker (allele), SNP, or Ant1 gene as defined in any of statements 1 to 48 one of the plants (populations) comprises a homozygous polynucleotide, molecular marker (allele), SNP, or Ant2 gene as defined in any of statements 49 to 73.

127. The method according to any of statements 124 to 126, wherein one and only one of said first or second barley parent plant (population) comprises a polynucleotide, molecular marker (allele), SNP, or Ant1 gene as defined in any of statements 1 to 48 (and the other one does not—i.e., neither homozygous nor heterozygous), wherein said polynucleotide, molecular marker (allele), SNP, or Ant1 gene is homozygous, and comprises a homozygous or heterozygous polynucleotide, molecular marker (allele), SNP, or Ant2 gene as defined in any of statements 49 to 73.

128. The method according to any of statements 124 to 126, wherein said first barley parent plant (population) comprises a homozygous polynucleotide, molecular marker (allele), SNP, or Ant1 gene as defined in any of statements 1 to 48 and a homozygous polynucleotide, molecular marker (allele), SNP, or Ant2 gene as defined in any of statements 49 to 73; and wherein said second barley parent plant (population) comprises a homozygous polynucleotide, molecular marker (allele), SNP, or Ant1 gene as defined in any of statements 1 to 48 and does not comprise a polynucleotide, molecular marker (allele), SNP, or Ant2 gene as defined in any of statements 49 to 73.

129. The method according to any of statements 124 to 128, further comprising (d) harvesting a plant part from said progeny.

130. The method according to any of statements 115 to 122, wherein introducing into the genome comprises mutagenesis, such as chemical mutagenization or mutagenization by radiation.

131. The method according to any of statements 115 to 122, wherein introducing into the genome comprises transgenesis or gene-editing.

132. The method according to any of 105 to 131, wherein said plant part is a plant cell, tissue, organ, or seed.

133. The method according to any of statements 105 to 132, wherein said plant part is an immature or mature embryo, an inflorescence, a protoplast or callus.

134. The method according to any of statements 105 or 115 to 122, comprising transforming a plant or plant part, preferably a plant cell, more preferably an immature or mature embryo, an inflorescence, a protoplast or callus, with said polynucleotide, molecular marker (allele), SNP, Ant1 gene or coding sequence thereof, and/or Ant2 gene or coding sequence thereof, and optionally regenerating a plant from said plant cell, preferably immature or mature embryo, inflorescence, protoplast or callus.

135 A barley plant or plant part obtainable by the method according to any of statements 105 to 134.

136. A method for producing, obtaining, or selecting a hybrid barley seed (mixture), comprising providing a barley (bulk or unselected) seed mixture harvested from a cross between a first barley parent plant (population) and a second barley parent plant (population), wherein (only) said first and/or second barley parent plant (population) comprises a polynucleotide, molecular marker (allele), SNP, Ant1 gene, and/or Ant2 gene as defined in any of statements 1 to 73, preferably as provided in Table B;
selecting seeds not having a purple pericarp and/or hull (palea and lemma).

137. A method for producing, obtaining, or selecting a hybrid barley seed (mixture), comprising providing a barley (bulk or unselected) seed mixture harvested from a cross between a first barley parent plant (population) and a second barley parent plant (population), wherein (only) said first and/or second barley parent plant (population) comprises a polynucleotide, molecular marker (allele), SNP, Ant1 gene, and/or Ant2 gene as defined in any of statements 1 to 73, preferably as provided in Table B;
selecting seeds having a purple pericarp and/or hull (palea and lemma).

138. A method for producing, obtaining, or selecting a hybrid barley seed (mixture), comprising providing a barley (bulk or unselected) seed mixture harvested from a cross between a first barley parent plant (population) and a second barley parent plant (population), wherein (only) said first or second barley parent plant (population) comprises a polynucleotide, molecular marker (allele), SNP, Ant1 gene, and/or Ant2 gene as defined in any of statements 1 to 73, preferably as provided in Table B;
selecting seeds not having a purple pericarp and/or hull (palea and lemma).

139. A method for producing, obtaining, or selecting a hybrid barley seed (mixture), comprising providing a barley (bulk or unselected) seed mixture harvested from a cross between a first barley parent plant (population) and a second barley parent plant (population), wherein (only) said first or second barley parent plant (population) comprises a polynucleotide, molecular marker (allele), SNP, Ant1 gene, and/or Ant2 gene as defined in any of statements 1 to 73, preferably as provided in Table B;
selecting seeds having a purple pericarp and/or hull (palea and lemma).

140. A method for producing, obtaining, or selecting a hybrid barley seed (mixture), comprising providing a barley (bulk or unselected) seed mixture harvested from a cross between a first barley parent plant (population) and a second barley parent plant (population), wherein (only) said first or second barley parent plant (population) comprises a polynucleotide, molecular marker (allele), SNP, or Ant1 gene as defined in any of statements 1 to 48, preferably as provided in Table B, and polynucleotide, molecular marker (allele), SNP, or Ant2 gene as defined in any of statements 49 to 73, preferably as provided in Table B;
selecting seeds not having a purple pericarp and/or hull (palea and lemma).

141. A method for producing, obtaining, or selecting a hybrid barley seed (mixture), comprising providing a barley (bulk or unselected) seed mixture harvested from a cross between a first barley parent plant (population) and a second barley parent plant (population), wherein (only) said first or second barley parent plant (population) comprises a polynucleotide, molecular marker (allele), SNP, or Ant1 gene as defined in any of statements 1 to 48, preferably as provided in Table B, and a polynucleotide, molecular marker (allele), SNP, or Ant2 gene as defined in any of statements 49 to 73, preferably as provided in Table B;
selecting seeds having a purple pericarp and/or hull (palea and lemma).

142. A method for producing hybrid barley seed, comprising
crossing a first barley parent plant (population) and a second barley parent plant (population), wherein (only) said first and/or second barley parent plant (population) comprises a polynucleotide, molecular marker (allele), SNP, Ant1 gene, and/or Ant2 gene as defined in any of statements 1 to 73, preferably as provided in Table B;
harvesting seeds resulting from said cross;
selecting seeds not having a purple pericarp and/or hull (palea and lemma).

143. A method for producing hybrid barley seed, comprising
crossing a first barley parent plant (population) and a second barley parent plant (population), wherein (only) said first and/or second barley parent plant (population) comprises a polynucleotide, molecular marker (allele), SNP, Ant1 gene, and/or Ant2 gene as defined in any of statements 1 to 73, preferably as provided in Table B;
harvesting seeds resulting from said cross;
selecting seeds having a purple pericarp and/or hull (palea and lemma).

144. A method for producing hybrid barley seed, comprising
crossing a first barley parent plant (population) and a second barley parent plant (population), wherein (only) said first or second barley parent plant (population) comprises a polynucleotide, molecular marker (allele), SNP, Ant1 gene, and/or Ant2 gene as defined in any of statements 1 to 73, preferably as provided in Table B;
harvesting seeds resulting from said cross;
selecting seeds not having a purple pericarp and/or hull (palea and lemma).

145. A method for producing hybrid barley seed, comprising
crossing a first barley parent plant (population) and a second barley parent plant (population), wherein (only) said first or second barley parent plant (population) comprises a polynucleotide, molecular marker (allele), SNP, Ant1 gene, and/or Ant2 gene as defined in any of statements 1 to 73, preferably as provided in Table B;
harvesting seeds resulting from said cross;
selecting seeds having a purple pericarp and/or hull (palea and lemma).

146. A method for producing hybrid barley seed, comprising
crossing a first barley parent plant (population) and a second barley parent plant (population), wherein (only) said first or second barley parent plant (population) comprises a polynucleotide, molecular marker (allele), SNP, or Ant1 gene as defined in any of statements 1 to 48, preferably as provided in Table B, and a polynucleotide, molecular marker (allele), SNP, or Ant2 gene as defined in any of statements 49 to 73, preferably as provided in Table B;
harvesting seeds resulting from said cross;
selecting seeds not having a purple pericarp and/or hull (palea and lemma).

147. A method for producing hybrid barley seed, comprising
crossing a first barley parent plant (population) and a second barley parent plant (population), wherein (only) said first or second barley parent plant (population) comprises a polynucleotide, molecular marker (allele), SNP, or Ant1 gene as defined in any of statements 1 to 48, preferably as provided in Table B, and a polynucleotide, molecular marker (allele), SNP, or Ant2 gene as defined in any of statements 49 to 73, preferably as provided in Table B;
harvesting seeds resulting from said cross;
selecting seeds having a purple pericarp and/or hull (palea and lemma).

148. The method according to any of statements 136 to 147, wherein one and only one of said first or second barley parent plant (population) comprises a homozygous polynucleotide, molecular marker (allele), SNP, or Ant1 gene as defined in any of statements 1 to 48, preferably as provided in Table B and a homozygous or heterozygous polynucleotide, molecular marker (allele), SNP, or Ant2 gene as defined in any of statements 49 to 73, preferably as provided in Table B.

149. The method according to any of statements 136 to 148, wherein one and only one of said first or second barley parent plant (population) comprises a homozygous polynucleotide, molecular marker (allele), SNP, or Ant1 gene as defined in any of statements 1 to 48, preferably as provided in Table B and a homozygous or heterozygous polynucleotide, molecular marker (allele), SNP, or Ant2 gene as defined in any of statements 49 to 73, preferably as provided in Table B, wherein the other plant (population) does not comprise a heterozygous polynucleotide, molecular marker (allele), SNP, or Ant1 gene as defined in any of statements 1 to 48, preferably as provided in Table B, and wherein if both plants (populations) comprise a homozygous polynucleotide, molecular marker (allele), SNP, or Ant1 gene as defined in any of statements 1 to 48, preferably as provided in Table B one of the plants (populations) comprises a homozygous polynucleotide, molecular marker (allele), SNP, or Ant2 gene as defined in any of statements 49 to 73, preferably as provided in Table B.

150. The method according to any of statements 136 to 149, wherein one and only one of said first or second barley parent plant (population) comprises a polynucleotide, molecular marker (allele), SNP, or Ant1 gene as defined in any of statements 1 to 48, preferably as provided in Table B (and the other one does not—i.e. neither homozygous nor heterozygous), wherein said polynucleotide, molecular marker (allele), SNP, or Ant1 gene is homozygous, and comprises a homozygous or heterozygous polynucleotide, molecular marker (allele), SNP, or Ant2 gene as defined in any of statements 49 to 73, preferably as provided in Table B.

151. The method according to any of statements 136 to 149, wherein said first barley parent plant (population) comprises a homozygous polynucleotide, molecular marker (allele), SNP, or Ant1 gene as defined in any of statements 1 to 48, preferably as provided in Table B and a homozygous polynucleotide, molecular marker (allele), SNP, or Ant2 gene as defined in any of statements 49 to 73, preferably as provided in Table B; and wherein said second barley parent plant (population) comprises a homozygous polynucleotide, molecular marker (allele), SNP, or Ant1 gene as defined in any of statements 1 to 48, preferably as provided in Table B and does not comprise a polynucleotide, molecular marker (allele), SNP, or Ant2 gene as defined in any of statements 49 to 73, preferably as provided in Table B.

152. The method according to any of statements 136 to 151, wherein said cross is a mixed seed production cross.

153. The method according to any of statements 136 to 147, wherein said polynucleotide, molecular marker (allele), SNP, Ant1 gene, and/or Ant2 gene is homozygous.

154. The method according to any of statements 136 to 147, wherein said polynucleotide, molecular marker (allele), SNP, Ant1 gene, and/or Ant2 gene is heterozygous.

155. The method according to any of statements 136 to 154, wherein said Ant1 gene comprises or is comprised in a polynucleotide and/or comprises a molecular marker (allele) or SNP as defined in any of statements 1 to 48.

156. The method according to any of statements 136 to 155, wherein said Ant2 gene comprises or is comprised in a polynucleotide and/or comprises a molecular marker (allele) or SNP as defined in any of statements 49 to 73.

157. The method according to any of statements 136 to 147, wherein said Ant1 gene is homozygous in said first barley parent plant (population) and said second barley parent plant (population).

158. The method according to any of statements 136 to 147, wherein said Ant2 gene is homozygous only in said first or second barley parent plant (population).

159. The method according to any of statements 136 to 147, wherein said Ant2 gene is absent in said first or second barley parent plant (population).

160. The method according to any of statements 105 to 159, wherein (only) said first or second barley parent plant (population) is (male and/or female) sterile.

161. The method according to any of statements 105 to 160, wherein (only) said first or second barley parent plant (population) is male sterile.

162. The method according to any of statements 105 to 161, wherein (only) said first or second barley parent plant (population) is genetically male sterile.

163. The method according to any of statements 105 to 161, wherein (only) said first or second barley parent plant (population) is cytoplasmically male sterile.

164. The method according to any of statements 105 to 163,
wherein said first barley parent plant (population) comprises a homozygous polynucleotide, molecular marker (allele), SNP, or Ant1 gene as defined in any of statements 1 to 48;
wherein said first barley parent plant (population) comprises a homozygous polynucleotide, molecular marker (allele), SNP, or Ant2 gene as defined in any of statements 49 to 73;
wherein said second barley parent plant (population) comprises a homozygous polynucleotide, molecular marker (allele), SNP, or Ant1 gene as defined in any of statements 1 to 48;
wherein said second barley parent plant (population) does not comprise a polynucleotide, molecular marker (allele), SNP, or Ant2 gene as defined in any of statements 49 to 73.

165. The method according to statement 164, wherein (only) said second barley parent plant (population) is (male and/or female) sterile, preferably male sterile, such as genetically or cytoplasmically male sterile.

166. A (isolated) polynucleotide comprising a polynucleotide, molecular marker (allele), SNP, Ant1 gene or coding sequence thereof, or Ant2 gene or coding sequence thereof as defined in any of statements 1 to 73, the complement or the reverse complement thereof, or a (unique) fragment thereof or of the complement or reverse complement thereof.

167. A (isolated) polynucleotide having a sequence as set forth in any of SEQ ID NOs: 1 to 32, the complement or the reverse complement thereof, or a (unique) fragment thereof or of the complement or reverse complement thereof.

168. The (isolated) polynucleotide according to statement 166 or 167, which comprises or consists of 10 to 500 nucleotides, preferably 15 to 250 nucleotides, more preferably 18 to 250 nucleotides, most preferably 20 to 250 nucleotides.

169. The (isolated) polynucleotide according to any of statements 166 to 168, in particular suitable as molecular marker, comprising at least 15, preferably at least 18, more preferably at least 20, contiguous nucleotides of any of SEQ ID NO: 7 to 13 or 20 to 32, or complementary to contiguous nucleotides of any of SEQ ID NO: 7 to 13 or 20 to 32, or reverse complementary to contiguous nucleotides of any of SEQ ID NO: 7 to 13 or 20 to 32.

170. The (isolated) polynucleotide according to any of statements 166 to 169 comprising a SNP as defined in any of statements 24 to 30, 41 to 47, 54 to 60, or 66 to 72.

171. A (isolated) polynucleotide specifically hybridizing with the polynucleotide as defined in any of statements 1 to 73, the complement or the reverse complement thereof.

172. The (isolated) polynucleotide according to any of statements 166 to 171, which is a primer or a probe.

173. The (isolated) polynucleotide according to any of statements 166 to 172, which is an allele-specific primer.

174. The (isolated) polynucleotide according to any of statements 166 to 173, which is a KASP primer.

175. A primer or probe capable of specifically detecting the polynucleotide, molecular marker (allele), SNP, Ant1 gene or coding sequence thereof, or Ant2 gene or coding sequence thereof as defined in any of statements 1 to 73.

176. A primer set capable of specifically detecting the polynucleotide, molecular marker (allele), SNP, Ant1 gene or coding sequence thereof, or Ant2 gene or coding sequence thereof as defined in any of statements 1 to 73.

177. A (isolated) polynucleotide having a sequence as set forth in any of SEQ ID NOs: 7 to 12, or a (unique) fragment thereof comprising at least 15 contiguous nucleotides, preferably at least 18 contiguous nucleotides, more preferably at least 20 contiguous nucleotides, wherein the polynucleotide comprises
position 95 of SEQ ID NO: 7, preferably wherein said nucleotide is T;
position 224 of SEQ ID NO: 8, preferably wherein said nucleotide is C;
position 206 of SEQ ID NO: 9, preferably wherein said nucleotide is C;
position 73 of SEQ ID NO: 10, preferably wherein said nucleotide is C;
position 145 of SEQ ID NO: 11, preferably wherein said nucleotide is C;
position 50 of SEQ ID NO: 12, preferably wherein said nucleotide is G; or
position 101 of SEQ ID NO: 13, preferably wherein said nucleotide is C; preferably as the most 3′ nucleotide;
the complement of any thereof, or the reverse complement of any thereof.

178. A (isolated) polynucleotide having a sequence as set forth in any of SEQ ID NOs: 13 or 20 to 32, or a (unique) fragment thereof comprising at least 15 contiguous nucleotides, preferably at least 18 contiguous nucleotides, more preferably at least 20 contiguous nucleotides, wherein the polynucleotide comprises
position 101 of SEQ ID NO: 13, preferably wherein said nucleotide is C
position 46 of SEQ ID NO: 20, preferably wherein said nucleotide is G;
position 96 of SEQ ID NO: 21, preferably wherein said nucleotide is C;
position 64 of SEQ ID NO: 22, preferably wherein said nucleotide is T;
position 73 of SEQ ID NO: 23, preferably wherein said nucleotide is G;
position 101 of SEQ ID NO: 24, preferably wherein said nucleotide is T;
position 72 of SEQ ID NO: 25, preferably wherein said nucleotide is A;
position 84 of SEQ ID NO: 26, preferably wherein said nucleotide is G;
position 76 of SEQ ID NO: 27, preferably wherein said nucleotide is C;
position 94 of SEQ ID NO: 28, preferably wherein said nucleotide is A;
position 88 of SEQ ID NO: 29, preferably wherein said nucleotide is T;
position 52 of SEQ ID NO: 30, preferably wherein said nucleotide is A;
position 80 of SEQ ID NO: 31, preferably wherein said nucleotide is G; and/or
position 73 of SEQ ID NO: 32, preferably wherein said nucleotide is C. preferably as the most 3′ nucleotide;
the complement of any thereof, or the reverse complement of any thereof.

179. Use of a polynucleotide, molecular marker (allele), SNP, Ant1 gene or coding sequence thereof, or Ant2 gene or coding sequence thereof as defined in any of statements 1 to 73, for identifying or selecting a barley plant or plant part.

180. Use of a polynucleotide, molecular marker (allele), SNP, Ant1 gene or coding sequence thereof, or Ant2 gene or coding sequence thereof as defined in any of statements 1 to 73, for generating a barley plant or plant part.

### BRIEF DESCRIPTION OF THE FIGURES

FIG. 1: Concept of seed sorting in the hybrid seed production of (winter) barley according to an embodiment of the invention. Key step is to anchor the dominant (i.e., purple color contributing) allelic status Ant1 and Ant2 genes in the barley restorer pool. This can be done by screening restorer crossing parents to prepare trait introgression. Each restorer line containing both genes in dominant status will produce colored seeds. The non-colored seed coat of the female pool consisting of male sterile lines is given by the inheritance of recessive Ant2 or ant1 allele. The allelic constellation of each male and female pool can be tested by KASP markers. In a mixed production system, the hybrid seeds, growing at the female will have non-colored seed coats (inherited from the mother side). The inbred seeds of the restorer line have purple colored seed coats which can be recognized from the non-colored hybrid seed through a color sorter device. Ant1: allele of ANT1 contributing to purple color; ant1: allele of ANT1 not contributing to purple color; Ant2: allele of ANT2 contributing to purple color; Ant2: allele of ANT2 not contributing to purple color.

FIG. 2: Picture of example grains (F2-generation of population 1 (HOR10032×HOR10033) showing a darker purple pigmentation (left-purple), a lighter purple pigmentation (middle-purple) and no pigmentation (right-light).

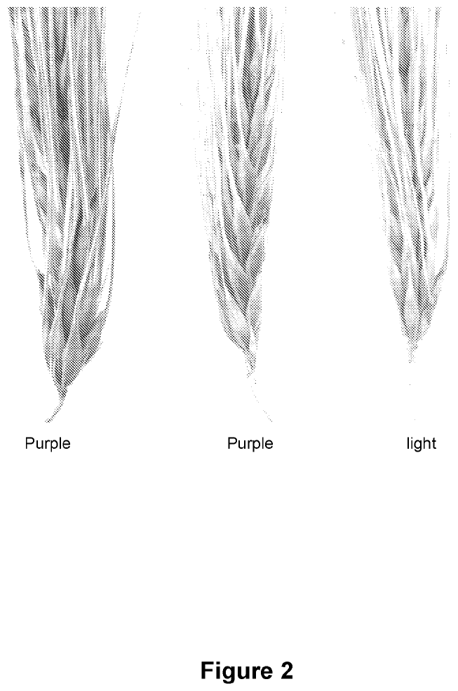

FIG. 3: Picture of example grains (F2-generation of population 9 (Titan×HOR10031) showing either no pigmentation (light) or a black pigmentation (black/pigmented) ear.

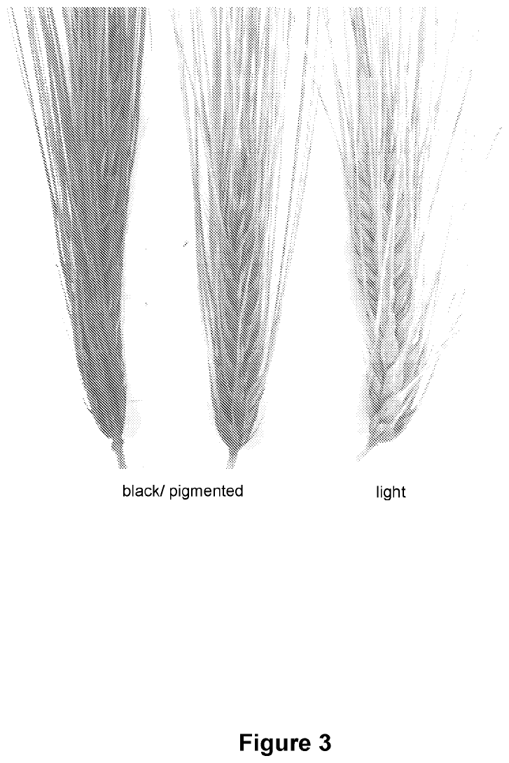

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise. As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or”

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

The recitation of numerical ranges by endpoints includes all numbers and fractions subsumed within the respective ranges, as well as the recited endpoints. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2^{nd} ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al., 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised, and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each of the statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous. It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example.

The term “plant” includes whole plants, including descendants or progeny thereof. As used herein unless clearly indicated otherwise, the term “plant” intends to mean a plant at any developmental stage. The term “plant part” includes any part or derivative of the plant, including particular plant tissues or structures, plant cells, plant protoplast, plant cell or tissue culture from which plants can be regenerated, plant calli, plant clumps and plant cells that are intact in plants or parts of plants, such as seeds, kernels, cobs, flowers, cotyledons, leaves, stems, buds, roots, root tips, stover, and the like. Plant parts may include processed plant parts or derivatives, including flower, oils, extracts etc. “Parts of a plant” are e.g., shoot vegetative organs/structures, e.g., leaves, stems and tubers; roots, flowers and floral organs/structures, e.g., bracts, sepals, petals, stamens, carpels, anthers and ovules; seed, including embryo, endosperm, and seed coat; fruit and the mature ovary; plant tissue, e.g., vascular tissue, ground tissue, and the like; and cells, e.g., guard cells, egg cells, pollen, trichomes and the like; and progeny of the same. Parts of plants may be attached to or separate from a whole intact plant. Such parts of a plant include, but are not limited to, organs, tissues, and cells of a plant, and preferably seeds. A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. “Plant cell culture” means cultures of plant units such as, for example, protoplasts, cell culture cells, cells in plant tissues, pollen, pollen tubes, ovules, embryo sacs, zygotes and embryos at various stages of development. “Plant material” refers to leaves, stems, roots, flowers or flower parts, fruits, pollen, egg cells, zygotes, seeds, cuttings, cell or tissue cultures, or any other part or product of a plant. This also includes callus or callus tissue as well as extracts (such as extracts from taproots) or samples. A “plant organ” is a distinct and visibly structured and differentiated part of a plant such as a root, stem, leaf, flower bud, or embryo. “Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In certain embodiments, the plant part or derivative is (functional) propagation material, such as germplasm, a seed, or plant embryo or other material from which a plant can be regenerated. In certain embodiments, the plant part or derivative comprises (functional) male and/or female reproductive organs.

In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, etc.

As used herein, the term plant population may be used interchangeably with population of plants. A plant population preferably comprises a multitude of individual plants, such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000.

As used herein, the term Hordeum refers to the genus Hordeum in the Poaceae family. The term Hordeum may be used herein interchangeably with barley. Non limiting species in the genus Hordeum include H. aegiceras, H. arizonicum, H. bogdanii, H. brachyantherum, H. brachyatherum, H. brevisubulatum, H. bulbosum, H. californicum, H. capense H. chilense, H. comosum, H. cordobense, H. depressum, H. distichon, H. erectifolium, H. euclaston, H. flexuosum, H. fuegianum, H. guatemalense, H. halophilum, H. intercedens, H. jubatum, H. x lagunculciforme, H. lechleri, H. marinum, H. murinum, H. muticum, H. parodii, H. patagonicum, H. x pavisii, H. procerum, H. pubiflorum, H. pusillum, H. roshevitzii, H. secalinum, H. spontaneum, H. stenostachys, H. tetraploidum, H. vulgare; or any subspecies or hybrid thereof, including all ploidy levels, such as diploid. Preferably, the Hordeum species is Hordeum vulgare. Preferably, barley as referred to herein is from the species Hordeum vulgare; or any subspecies or hybrid thereof, including all ploidy levels, such as diploid.

The term “locus” (loci plural) means a specific place or places or a site on a chromosome where for example a QTL, a gene or genetic marker is found. As used herein, the term “quantitative trait locus” or “QTL” has its ordinary meaning known in the art. By means of further guidance, and without limitation, a QTL may refer to a region of DNA that is associated with the differential expression of a quantitative phenotypic trait in at least one genetic background, e.g., in at least one breeding population. The region of the QTL encompasses or is closely linked to the gene or genes that affect the trait in question.

An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window. A QTL may encode for one or more alleles that affect the expressivity of a continuously distributed (quantitative) phenotype. In certain embodiments, the QTL as described herein may be homozygous. In certain embodiments, the QTL as described herein may be heterozygous.

As used herein, the term “allele” or “alleles” refers to one or more alternative forms, i.e., different nucleotides or nucleotide sequences, of a locus, such as a gene, marker, QTL, etc.,

As used herein, the term “mutant alleles” or “mutation” of alleles include alleles having one or more mutations, such as insertions, deletions, stop codons, base changes (e.g., transitions or transversions), or alterations in splice junctions, which may or may not give rise to altered gene products. Modifications in alleles may arise in coding or non-coding regions (e.g., promoter regions, exons, introns or splice junctions).

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background. The process of “introgressing” is often referred to as “backcrossing” when the process is repeated two or more times. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing. i.e., the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g., even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

A genetic element, an introgression fragment, a QTL or a gene or allele conferring a trait is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only pants comprising the genetic element, locus, introgression fragment, gene or allele can be used, but also progeny/descendants from such plants which have been selected to retain the genetic element, locus, introgression fragment, gene or allele, can be used and are encompassed herein. Whether a plant (or genomic DNA, cell or tissue of a plant) comprises the same genetic element, locus, introgression fragment, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic plants may also be encompassed.

“Introducing” in the meaning of the present invention includes stable or transient integration by means of transformation including Agrobacterium-mediated transformation, transfection, microinjection, biolistic bombardment, insertion using gene editing technology like CRISPR systems (e.g., CRISPR/Cas, in particular CRISPR/Cas9 or CRISPR/Cas12), CRISPR/CasX, or CRISPR/CasY), TALENs, zinc finger nucleases or meganucleases, homologous recombination optionally by means of one of the below mentioned gene editing technology including preferably a repair template, modification of endogenous gene using random or targeted mutagenesis like TILLING or above mentioned gene editing technology, etc.

As used herein the terms “genetic engineering”, “transformation” and “genetic modification” are all used herein as synonyms for the transfer of isolated and cloned genes into the DNA, usually the chromosomal DNA or genome, of another organism.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g., in a test tube). The term “transgenic” here means genetically modified by the introduction of a non-endogenous nucleic acid sequence. Typically, a species-specific nucleic acid sequence is introduced in a form, arrangement, or quantity into the cell in a location where the nucleic acid sequence does not occur naturally in the cell. This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene”, “exogene”, or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Gene editing” or “genome editing” refers to genetic engineering in which in which DNA or RNA is inserted, deleted, modified, or replaced in the genome of a living organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically introduce one or more of the molecular marker (marker allele) according to the invention as described herein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As uses herein, “hybridization” or “hybridizing” refers to a reaction in which one or more polynucleotides react to form a complex that is stabilized via hydrogen bonding between the bases of the nucleotide residues, i.e., a process in which a single-stranded nucleic acid molecule attaches itself to a complementary nucleic acid strand, i.e., agrees with this base pairing. Standard procedures for hybridization are described, for example, in Sambrook et al., (Molecular Cloning. A Laboratory Manual, Cold Spring Harbor Laboratory Press, 3rd edition 2001). The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence. Preferably this will be understood to mean an at least 50%, more preferably at least 55%, 60%, 65%, 70%, 75%, 80% or 85%, more preferably 90%, 91%, 92%, 93%, 94%, 95%, 96%, 97%, 98% or 99% of the bases of the nucleic acid strand form base pairs with the complementary nucleic acid strand. The possibility of such binding depends on the stringency of the hybridization conditions.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance, a genetic element may be replaced by gene editing in which a recombination template is provided. The DNA may be cut upstream and downstream of a sequence which needs to be replaced. As such, the sequence to be replaced is excised from the DNA. Through HDR, the excised sequence is then replaced by the template. In certain embodiments, the QTL allele of the invention as described herein may be provided on/as a template. By designing the system such that double strand breaks are introduced upstream and downstream of the corresponding region in the genome of a plant not comprising the QTL allele, this region is excised and can be replaced with the template comprising the QTL allele of the invention. In this way, introduction of the QTL allele of the invention in a plant need not involve multiple backcrossing, in particular in a plant of specific genetic background. Similarly, the polynucleic acid of the invention may be provided on/as a template. More advantageously however, the polynucleic acid of the invention may be generated without the use of a recombination template, but solely through the endonuclease action leading to a double strand DNA break which is repaired by NHEJ, resulting in the generation of indels. Further, gene editing may comprise also the exchange of single nucleotides by means of base editors. A base editor as used herein refers to a protein or a fragment thereof having the capacity to mediate a targeted base modification, i.e., the conversion of a base of interest resulting in a point mutation of interest. Preferably, the at least one base editor in the context of the present invention is temporarily or permanently fused to at least one DSBI enzyme, or optionally to a component of at least one DSBI. The fusion can be covalent and/or non-covalent. Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalysed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Goudelli et al., ((2017). Programmable base editing of A·T to G·C in genomic DNA without DNA cleavage. Nature, 551 (7681), 464.) described adenine base editors (ABEs) that mediate the conversion of A·T to G·C in genomic DNA.

In certain embodiments, the nucleic acid modification is affected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nuclease. The products are then separated by size, such as by HPLC. See also McCallum et al., “Targeted screening for induced mutations”; Nat Biotechnol. 2000 April; 18 (4): 455-7 and McCallum et al., “Targeting induced local lesions IN genomes (TILLING) for plant functional genomics”; Plant Physiol. 2000 June; 123 (2): 439-42.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. As used herein, the term “heterozygote” refers to an individual cell or plant having different alleles at one or more or all loci. When the term is used with reference to a specific locus or gene, it means at least that locus or gene has different alleles. As used herein, the term “heterozygous” means a genetic condition existing when different alleles reside at corresponding loci on homologous chromosomes. In certain embodiments, the QTL and/or one or more marker(s) as described herein is/are homozygous. In certain embodiments, the QTL and/or one or more marker(s) as described herein are heterozygous. In certain embodiments, the QTL allele and/or one or more marker(s) allele(s) as described herein is/are homozygous. In certain embodiments, the QTL allele and/or one or more marker(s) allele(s) as described herein are heterozygous.

A “marker” is a (means of finding a position on a) genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. The term marker locus is the locus (gene, sequence or nucleotide) that the marker detects. “Marker” or “molecular marker” or “marker locus” or “marker allele” may also be used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest.

The development of markers may be based either on quantitative trait loci (QTL) mapping or genome wide association studies (GWAS). A QTL mapping requires two parental lines (according to the present invention, one with purple-colored seeds and one without) that differ genetically for the barley grain color trait. These two types of mapping are referred to as low resolution mapping due to the fact that the identified chromosomal region (from now on called QTL interval) controlling the trait can contain up to several hundreds of genes within an interval size of 5-30 CM, depending on whether the QTL interval is in the telomeric or pericentromeric region. The reason for such a name is that both QTL mapping as well as GWAS generally only use rather a small size of population (100 to 200 lines), thus the size of the discovered chromosomal interval within which the gene exists becomes very large. Therefore, the markers flanking the identified chromosomal region in 100 percent of cases are not diagnostic, which means the marker score generated does not necessarily match the observed phenotype in the field. For this issue to be resolved, the mapping resolution needs to be increased, and the resolution can be increased by increasing recombination events during meiosis. To accomplish this, one has to increase the population to thousands of lines plus have different generations of selfing or back crossing that take at least four years (and are very expensive and labor-intensive). The breeder must perform high resolution mapping of fine mapping in order to have a marker nearer to the gene. In the initial screening, the thousands of individuals are screened only for initial flanking markers identified through low-resolution mapping. Such a screening aims to find recombinants. Recombination events between markers and the gene will allow to narrow down the interval and come closer to the gene. The next step is designing additional markers within the targeted interval. These may range for instance from ten to twenty markers, depending on how long an interval is. Then screen the newly identified recombinants with these 10-20 markers. This will help find the closest marker with the least number of recombination events between it and the gene, keeping in mind that the marker is only close to the gene, not within it, so there is still a possibility that the marker score does not match the observed phenotype. So, as you can see the approach requires a lot of time, efforts, and money should be invested. The use of literature-based markers is another possibility. It is more convenient and time-efficient if the information of the closet marker is already published, otherwise the publication is only beneficial to determine the corresponding chromosomal interval responsible for the trait of interest.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g., via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population. Regarding a SNP marker, allele refers to the specific nucleotide base present at that SNP locus in that individual plant. As used herein, reference to markers or marker alleles refers to markers or marker alleles associated with, linked with, or characteristic of (purple) seed color, unless explicitly referred to otherwise. Such markers or marker alleles are typically annotated as “donor” markers or marker alleles.

“Fine-mapping” refers to methods by which the position of a QTL can be determined more accurately (narrowed down) and by which the size of the introgression fragment comprising the QTL is reduced. For example, Near Isogenic Lines for the QTL (QTL-NILs) can be made, which contain different, overlapping fragments of the introgression fragment within an otherwise uniform genetic background of the recurrent parent. Such lines can then be used to map on which fragment the QTL is located and to identify a line having a shorter introgression fragment comprising the QTL.

“Marker assisted selection” (of MAS) is a process by which individual plants are selected based on marker genotypes. “Marker assisted counter-selection” is a process by which marker genotypes are used to identify plants that will not be selected, allowing them to be removed from a breeding program or planting. Marker assisted selection uses the presence of molecular markers, which are genetically linked to a particular locus or to a particular chromosome region (e.g., introgression fragment, transgene, polymorphism, mutation, etc), to select plants for the presence of the specific locus or region (introgression fragment, transgene, polymorphism, mutation, etc). For example, a marker allele genetically linked to a QTL, polynucleotide, or gene as defined herein, can be used to detect and/or select plants comprising the QTL, polynucleotide, or gene on chromosome 2H (Ant2) or 7H (Ant1). The closer the genetic linkage of the marker allele to the locus (e.g., about 10 cM, 7 cM, 6 cM, 5 cM, 4 cM, 3 cM, 2 cM, 1 cM, 0.5 cM or less), the less likely it is that the marker is dissociated from the locus through meiotic recombination. Likewise, the closer two markers are linked to each other (e.g., within 10 cM, 7 cM, 5 cM, 4 cM, 3 cM, 2 cM, 1 cM or less) the less likely it is that the two markers will be separated from one another (and the more likely they will co-segregate as a unit). A marker “within 10 cM or within 7 cM or within 5 CM, 3 CM, 2 CM, or 1 cM” of another marker refers to a marker which genetically maps to within the 10 cM or 7 cM or 5 cM, 3 CM, 2 CM, or 1 cM region flanking the marker (i.e., either side of the marker). Similarly, a marker within 10 Mb, 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of another marker refers to a marker which is physically located within the 10 Mb, 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less, of the genomic DNA region flanking the marker (i.e. either side of the marker). “LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favour the presence of linkage and a LOD score greater than 3.0 is considered evidence for linkage. A LOD score of +3 indicates 1000 to 1 odds that the linkage being observed did not occur by chance. The respective marker (alleles) of the invention as described herein are within the Ant1 or Ant2 gene or at least within 3 cM or at least within 3 Mbp from the Ant1 or Ant2 gene.

A “marker haplotype” refers to a combination of alleles at a marker locus. Suitable marker haplotypes according to the invention are the Ant1 haplotype comprising markers Ant1_HapM1, Ant1_HapM2, Ant1_HapM3, Ant1_HapM4, Ant1_HapM5, and Ant1_HapM6 and the Ant2 haplotype comprising marker Ant2_HapM1 in combination with any one or more of Ant2_HapM2, Ant2_HapM3, Ant2_HapM4, Ant2_HapM5, Ant2_HapM6, and Ant2_HapM7, such as in preferred embodiments (i) markers Ant2_HapM1 and Ant2_HapM2; (ii) markers Ant2_HapM1 and Ant2_HapM3; (iii) markers Ant2_HapM1 and Ant2_HapM4; (iv) markers Ant2_HapM1 and Ant2_HapM5; (v) markers Ant2_HapM1 and Ant2_HapM6; (vi) markers Ant2_HapM1 and Ant2_HapM7.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g., SNP technology is used in the examples provided herein.

“Genetic markers” are nucleic acids that are polymorphic in a population and where the alleles of which can be detected and distinguished by one or more analytic methods, e.g., RFLP, AFLP, isozyme, SNP, SSR, and the like. The terms “molecular marker” and “genetic marker” are used interchangeably herein. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”. The term “indel” refers to an insertion or deletion, wherein one line may be referred to as having an inserted nucleotide or piece of DNA relative to a second line, or the second line may be referred to as having a deleted nucleotide or piece of DNA relative to the first line.

“Physical distance” between loci (e.g., between molecular markers and/or between phenotypic markers) on the same chromosome is the actually physical distance expressed in bases or base pairs (bp), kilo bases or kilo base pairs (kb or kbp) or megabases or mega base pairs (Mb or Mbp).

“Genetic distance” between loci (e.g., between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

An allele “negatively” correlates with a trait when it is linked to it and when presence of the allele is an indicator that a desired trait or trait form will not occur in a plant comprising the allele. An allele “positively” correlates with a trait when it is linked to it and when presence of the allele is an indicator that the desired trait or trait form will occur in a plant comprising the allele.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to gray leaf spot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score). As used herein, the terms “linkage”, “linked”, and the like can be used interchangeably with “associated”.

The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosomal segment are also “genetically linked”, typically within a genetic recombination distance of less than or equal to 50 cM, e.g., about 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.75, 0.5, 0.25 cM or less. That is, two genetic elements within a single chromosomal segment undergo recombination during meiosis with each other at a frequency of less than or equal to about 50%, e.g., about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25% or less. “Closely linked” markers display a cross over frequency with a given marker of about 10% or less, e.g., 9%, 8%, 7%, 6%, 5%, 4%, 3%, 2%, 1%, 0.75%, 0.5%, 0.25% or less (the given marker locus is within about 10 cM of a closely linked marker locus, e.g., 9, 8, 7, 6, 5, 4, 3, 2, 1, 0.75, 0.5, 0.25 cM or less of a closely linked marker locus). Put another way, closely linked marker loci co-segregate at least about 90% the time, e.g., 91%, 92%, 93%, 94%, 95%, 96%, 97%, 98%, 99%, 99.5%, 99.75%, or more of the time.

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (BI2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the BI2seq program can be found in the readme file accompanying BLASTZ. Bl2seq performs a comparison between two sequences using either the BLASTN or BLASTP algorithm. Preferably, BLAST sequence alignments are performed according to the standard (i.e., default) settings (i.e., at the filing date of the present application).

BLASTN is used to compare nucleic acid sequences, while BLASTP is used to compare amino acid sequences. To compare two nucleic acid sequences, the options are set as follows: —i is set to a file containing the first nucleic acid sequence to be compared (e.g., C: \seq I.txt); —j is set to a file containing the second nucleic acid sequence to be compared (e.g., C:\seq2.txt); —p is set to blastn; —o is set to any desired file name (e.g., C:\output.txt); —q is set to −1; —r is set to 2; and all other options are left at their default setting. The following command will generate an output file containing a comparison between two sequences: C:\B12seq —i c:\seql.txt —j c:\seq2.txt —p blastn —o c:\output.txt —q −1 —r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the BI2seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used. The terms “nucleotide sequence(s)”, “polynucleotide(s)”, “nucleic acid sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

An “isolated nucleic acid sequence”, “isolated polynucleic acid” or “isolated DNA” refers to a nucleic acid sequence which is no longer in the natural environment from which it was isolated, e.g., the nucleic acid sequence in a bacterial host cell or in the plant nuclear or plastid genome. When referring to a “sequence” herein, it is understood that the molecule having such a sequence is referred to, e.g., the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g., minichromosome.

When reference is made to a nucleic acid sequence (e.g., DNA or genomic DNA) having “substantial sequence identity to” a reference sequence or having a sequence identity of at least 80%>, e.g., at least 85%, 90%, 95%, 98%> or 99%> nucleic acid sequence identity to a reference sequence, in one embodiment said nucleotide sequence is considered substantially identical to the given nucleotide sequence and can be identified using hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically, stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g., 100 nt) are for example those which include at least one wash in 0.2×SSC at 63° C. for 20 min, or equivalent conditions. Stringent conditions for DNA-DNA hybridisation (Southern blots using a probe of e.g., 100 nt) are for example those which include at least one wash (usually 2) in 0.2×SSC at a temperature of at least 50° C., usually about 55° C., for 20 min, or equivalent conditions. See also Sambrook et al., (1989) and Sambrook and Russell (2001). Examples of high stringent hybridization conditions are conditions under which primarily only those nucleic acid molecules that have at least 90% or at least 95% sequence identity undergo hybridization. Such high stringent hybridization conditions are, for example: 4×SSC at 65° C. and subsequent multiple washes in 0.1×SSC at 65° C. for approximately 1 hour. The term “high stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2×SSC and 0.1% SDS at 68° C. Preferably, hybridization takes place under stringent conditions. Less stringent hybridization conditions are, for example: hybridizing in 4×SSC at 37° C. and subsequent multiple washing in 1×SSC at room temperature.

It will be understood that “specifically hybridizing” means that the polynucleic acid hybridises with the (molecular) marker allele (such as under stringent hybridisation conditions, as defined herein elsewhere), but does not (substantially) hybridise with a polynucleic acid not comprising the marker allele or is (substantially) incapable of being used as a PCR primer. By means of example, in a suitable readout, the hybridization signal with the marker allele or PCR amplification of the marker allele is at least 5 times, preferably at least 10 times stronger or more than the hybridisation signal with a non-marker allele, or any other sequence.

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The term polypeptide also refers to, and does not exclude, modifications of the polypeptide, e.g., glycosylation, acetylation, phosphorylation and the like. Such modifications are well described in basic texts and in more detailed monographs, as well as in the research literature.

Amino acid substitutions encompass amino acid alterations in which an amino acid is replaced with a different naturally-occurring amino acid residue. Such substitutions may be classified as “conservative<1>, in which an amino acid residue contained in the wild-type protein is replaced with another naturally-occurring amino acid of similar character, for example Gly<→Ala, Val<→Ile<→Leu, Asp<→Glu, Lys<→Arg, Asn<→Gln or Phe<→Trp<→Tyr. Substitutions encompassed by the present invention may also be “non-conservative”, in which an amino acid residue which is present in the wild-type protein is substituted with an amino acid with different properties, such as a naturally-occurring amino acid from a different group (e.g., substituting a charged or hydrophobic amino acid with alanine. “Similar amino acids”, as used herein, refers to amino acids that have similar amino acid side chains, i.e., amino acids that have polar, non-polar or practically neutral side chains. “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the center of the protein where they can interact with similar non-polar neighbours (“hydrophobic” amino acids”). Examples of amino acids that have polar side chains are arginine, asparagine, aspartate, cysteine, glutamine, glutamate, histidine, lysine, serine, and threonine (all hydrophilic, except for cysteine which is hydrophobic). Examples of amino acids that have non-polar side chains are alanine, glycine, isoleucine, leucine, methionine, phenylalanine. proline, and tryptophan (all hydrophobic, except for glycine which is neutral).

The term “gene” when used herein refers to a polymeric form of nucleotides of any length, either ribonucleotides or deoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleic acid sequences or genomic DNA, while introns may be present as well under certain circumstances.

As used herein, the term “endogenous” refers to a gene or allele which is present in its natural genomic location. The term “endogenous” can be used interchangeably with “native” or “wild-type”. This does not however exclude the presence of one or more nucleic acid differences with the wild-type allele. In particular embodiments, the difference with a wild-type allele can be limited to less than 9 preferably less than 6, more particularly less than 3 nucleotide differences, such as 0 nucleotides difference. More particularly, the difference with the wildtype sequence can be in only one nucleotide. Preferably, the endogenous allele encodes a modified protein having less than 9, preferably less than 6, more particularly less than 3 and even more preferably only one or no amino acid difference with the wild-type protein.

A used herein, the term “exogenous polynucleotide” refers to a polynucleotide, such as a gene (or cDNA) or allele which is or has been recombinantly introduced in a cell (or plant). The exogenous polynucleotide may be episomally or genomically integrated. Integration may be random or site-directed. Integration may include replacement of a corresponding endogenous polynucleotide. It will be understood that an exogenous polynucleotide is not naturally present in the cell or plant.

The term “hybrid”, “hybrid plant”, or hybrid seed” as used in the context of the present invention has its ordinary meaning known in the art. By means of further guidance, and without limitation in the context of the present invention this term refers to the offspring of two (genetically distinct or different) parent plants, which may be different plant lines, cultivars, or varieties. It will be understood that according to the present invention, the parents of a hybrid plant preferably are from the same genus, preferably the same species. Preferably, the parents of a hybrid each are stable populations, having a high degree of homozygosity. The parents typically differ from each other in one or more traits or (agronomic, physiologic, or quality) characteristics. The hybrid therefore typically is heterozygous for such trait or (agronomic, physiologic, or quality) characteristic. According to the present invention, hybrids preferably are the F1 hybrids, i.e., the first generation of offspring resulting from the two parents (e.g., the two parental lines, cultivars, or varieties). The seed produced by crossing two parents is therefore the F1 hybrid seed.

In an aspect, the invention relates to a method for identifying and/or selecting a barley plant or plant part, comprising screening for the presence of a polynucleotide comprising one or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of a polynucleotide comprising one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably one or more of Ant2_M2, Ant2_M3, Ant2_M4, Ant2_M5, Ant2_M6, and/or Ant2_M7; or Ant2_HapM1 in combination with one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In an aspect, the invention relates to a method for identifying and/or selecting a barley plant or plant part, comprising screening for the presence of a polynucleotide comprising two or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of a polynucleotide comprising one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably one or more of Ant2_M2, Ant2_M3, Ant2_M4, Ant2_M5, Ant2_M6, and/or Ant2_M7; or Ant2_HapM1 in combination with one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In an aspect, the invention relates to a method for identifying and/or selecting a barley plant or plant part, comprising screening for the presence of a polynucleotide comprising three or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of a polynucleotide comprising one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably one or more of Ant2_M2, Ant2_M3, Ant2_M4, Ant2_M5, Ant2_M6, and/or Ant2_M7; or Ant2_HapM1 in combination with one or more of Ant2_HapM2, Ant2_HapM3, Ant2_HapM4, Ant2_HapM5, Ant2_HapM6, and/or Ant2_HapM7 (in or associated with or linked with the Ant1 gene and/or in the genome of the plant or plant part). In an aspect, the invention relates to a method for identifying and/or selecting a barley plant or plant part, comprising screening for the presence of a polynucleotide comprising four or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of a polynucleotide comprising one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably one or more of Ant2_M2, Ant2_M3, Ant2_M4, Ant2_M5, Ant2_M6, and/or Ant2_M7; or Ant2_HapM1 in combination with one or more of Ant2_HapM2, Ant2_HapM3, Ant2_HapM4, Ant2_HapM5, Ant2_HapM6, and/or Ant2_HapM7 (in or associated with or linked with the Ant1 gene and/or in the genome of the plant or plant part). In an aspect, the invention relates to a method for identifying and/or selecting a barley plant or plant part, comprising screening for the presence of a polynucleotide comprising five or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of a polynucleotide comprising one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably one or more of Ant2_M2, Ant2_M3, Ant2_M4, Ant2_M5, Ant2_M6, and/or Ant2_M7; or Ant2_HapM1 in combination with one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In an aspect, the invention relates to a method for identifying and/or selecting a barley plant or plant part, comprising screening for the presence of a polynucleotide comprising molecular marker(s) (allele(s)) Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of a polynucleotide comprising one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably one or more of Ant2_M2, Ant2_M3, Ant2_M4, Ant2_M5, Ant2_M6, and/or Ant2_M7; or Ant2_HapM1 in combination with one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part).

Screening for the presence of a polynucleic acid comprising any one or more of molecular marker (allele) selected from Ant1_HapM1, Ant1_HapM2, Ant1_HapM3, Ant1_HapM4, Ant1_HapM5, and Ant1_HapM6 may comprise screening for the presence of any one or more molecular marker (allele) selected from Ant1_HapM1, Ant1_HapM2, Ant1_HapM3, Ant1_HapM4, Ant1_HapM5, and Ant1_HapM6. Given their close linkage, screening for the presence of one particular marker may automatically identify the remaining markers if said particular marker is identified. Alternatively, screening for the presence of any one or more of molecular marker selected from Ant1_HapM1, Ant1_HapM2, Ant1_HapM3, Ant1_HapM4, Ant1_HapM5, and Ant1_HapM6 may encompass screening for, yet another closely linked marker, which if identified automatically identifies molecular marker (allele) Ant1_HapM1, Ant1_HapM2, Ant1_HapM3, Ant1_HapM4, Ant1_HapM5, and Ant1_HapM6.

Screening for the presence of a polynucleic acid comprising any one or more of molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 may comprise screening for the presence of any one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7. Given their close linkage, screening for the presence of one particular marker may automatically identify the remaining markers if said particular marker is identified. Alternatively, screening for the presence of any one or more of molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 may encompass screening for yet another closely linked marker, which if identified automatically identifies molecular marker (allele) Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7.

In certain embodiments, said polynucleic acid comprises a sequence which is at least 80%, preferably at least 90%, more preferably at least 95% identical to a sequence as set forth in SEQ ID NO: 1 or 4. In certain embodiments, said polynucleic acid comprises a sequence which is at least 80%, preferably at least 90% identical to a sequence as set forth in SEQ ID NO: 1 or 4, and encoding a protein having a sequence which is at least 80%, preferably at least 90%, more preferably at least 95% identical to a sequence as set forth in SEQ ID NO: 3 or 6. In certain embodiments, said polynucleic acid comprises a sequence which is at least 80%, preferably at least 90%, more preferably at least 95% identical to a sequence as set forth in SEQ ID NO: 1 or 4 and comprising (respectively) T at or corresponding to position 95, C at or corresponding to position 463, C at or corresponding to position 865, C at or corresponding to position 1092, C at or corresponding to position 1284, and/or G at or corresponding to position 1309 of SEQ ID NO: 1, preferably all, or alternatively not comprising one or more of (respectively) T at or corresponding to position 95, C at or corresponding to position 463, C at or corresponding to position 865, C at or corresponding to position 1092, C at or corresponding to position 1284, and/or G at or corresponding to position 1309 of SEQ ID NO: 1, or alternatively comprising one or more of V or G at or corresponding to position 95, D or G at or corresponding to position 464, D or G at or corresponding to position 866, D or T at or corresponding to position 1093, D or A at or corresponding to position 1285, and/or H or A at or corresponding to position 1310 of SEQ ID NO: 4, or alternatively not comprising one or more of T at or corresponding to position 95, C at or corresponding to position 464, C at or corresponding to position 866, C at or corresponding to position 1093, C at or corresponding to position 1285, and/or G at or corresponding to position 1310 of SEQ ID NO: 4. In certain embodiments, said polynucleic acid comprises a sequence which is at least 80%, preferably at least 90%, more preferably at least 95% identical to a sequence as set forth in SEQ ID NO: 1 or 4 and comprising (respectively) T at or corresponding to position 95, C at or corresponding to position 463, C at or corresponding to position 865, C at or corresponding to position 1092, C at or corresponding to position 1284, and/or G at or corresponding to position 1309 of SEQ ID NO: 1, preferably all, or alternatively not comprising one or more of comprising (respectively) T at or corresponding to position 95, C at or corresponding to position 463, C at or corresponding to position 865, C at or corresponding to position 1092, C at or corresponding to position 1284, and/or G at or corresponding to position 1309 of SEQ ID NO: 1, or alternatively comprising one or more of V or G at or corresponding to position 95, D or G at or corresponding to position 464, D or G at or corresponding to position 866. D or T at or corresponding to position 1093, D or A at or corresponding to position 1285, and/or H or A at or corresponding to position 1310 of SEQ ID NO: 4, or alternatively not comprising one or more of T at or corresponding to position 95, C at or corresponding to position 464, C at or corresponding to position 866, C at or corresponding to position 1093, C at or corresponding to position 1285, and/or G at or corresponding to position 1310 of SEQ ID NO: 4, and encoding a protein having a sequence which is at least 80%, preferably at least 90%, more preferably at least 95% identical to a sequence as set forth (respectively) in SEQ ID NO: 3 or 6.

In certain embodiments, said polynucleic acid comprises a sequence as set forth in SEQ ID NO: 1 or 4.

In certain embodiments, said polynucleic acid comprises a sequence which is at least 80%, preferably at least 90%, more preferably at least 95% identical to a sequence as set forth in SEQ ID NO: 2 or 5 In certain embodiments, said polynucleic acid comprises a sequence which is at least 80%, preferably at least 90% identical to a sequence as set forth in SEQ ID NO: 2 or 5, and encoding a protein having a sequence which is at least 80%, preferably at least 90%, more preferably at least 95% identical to a sequence as set forth in SEQ ID NO: 3 or 6. In certain embodiments, said polynucleic acid comprises a sequence which is at least 80%, preferably at least 90%, more preferably at least 95% identical to a sequence as set forth in SEQ ID NO: 2 or 5 and comprising (respectively) C at or corresponding to position 219, C at or corresponding to position 446, C at or corresponding to position 638, and/or G at or corresponding to position 663, preferably all, or alternatively not comprising one or more of comprising (respectively) C at or corresponding to position 219, C at or corresponding to position 446, C at or corresponding to position 638, and/or G at or corresponding to position 663, or alternatively comprising one or more of D or G at or corresponding to position 219, D or T at or corresponding to position 446, D or A at or corresponding to position 638, and/or H or A at or corresponding to position 663. In certain embodiments, said polynucleic acid comprises a sequence which is at least 80%, preferably at least 90%, more preferably at least 95% identical to a sequence as set forth in SEQ ID NO: 2 or 5 and comprising (respectively) C at or corresponding to position 219, C at or corresponding to position 446, C at or corresponding to position 638, and/or G at or corresponding to position 663, preferably all, or alternatively not comprising one or more of comprising (respectively) C at or corresponding to position 219, C at or corresponding to position 446, C at or corresponding to position 638, and/or G at or corresponding to position 663, or alternatively comprising one or more of D or G at or corresponding to position 219, D or T at or corresponding to position 446, D or A at or corresponding to position 638, and/or H or A or corresponding to at position 663, and encoding a protein having a sequence which is at least 80%, preferably at least 90%, more preferably at least 95% identical (respectively) to a sequence as set forth in SEQ ID NO: 3 or 6.

In certain embodiments, said polynucleic acid comprises a sequence as set forth in SEQ ID NO: 2 or 5.

In certain embodiments, said polynucleic acid comprises a sequence encoding a protein having a sequence which is at least 80%, preferably at least 90%, more preferably at least 95% identical to a sequence as set forth in SEQ ID NO: 3 or 6. In certain embodiments, said polynucleic acid comprises a sequence encoding a protein having a sequence which is at least 80%, preferably at least 90%, more preferably at least 95% identical to a sequence as set forth in SEQ ID NO: 3 or 6 and comprising (respectively) C at or corresponding to position 219, C at or corresponding to position 446, C at or corresponding to position 638, and/or G at or corresponding to position 663, preferably all, or alternatively not comprising one or more of comprising (respectively) C at or corresponding to position 219, C at or corresponding to position 446, C at or corresponding to position 638, and/or G at or corresponding to position 663, or alternatively comprising one or more of D or G at or corresponding to position 219, D or T at or corresponding to position 446, D or A at or corresponding to position 638, and/or H or A at or corresponding to position 663. The skilled person will understand that the indicated SNP/single nucleotide positions correspond to the positions of the coding sequence encoding the protein.

In certain embodiments, said polynucleic acid encodes for a protein having a sequence as set forth in SEQ ID NO: 3 or 6.

In certain embodiments, said polynucleic acid comprises a sequence which is at least 80%, preferably at least 90%, more preferably at least 95% identical to a sequence as set forth in any of SEQ ID NOs: 7 to 12. In certain embodiments, the polynucleic acid comprising Ant1_HapM1 has a sequence which is at least 80%, preferably at least 90%, more preferably at least 95% identical to a sequence as set forth in SEQ ID NO: 7. In certain embodiments, the polynucleic acid comprising Ant1_HapM2 has a sequence which is at least 80%, preferably at least 90%, more preferably at least 95% identical to a sequence as set forth in SEQ ID NO: 8. In certain embodiments, the polynucleic acid comprising Ant1_HapM3 has a sequence which is at least 80%, preferably at least 90%, more preferably at least 95% identical to a sequence as set forth in SEQ ID NO: 9. In certain embodiments, the polynucleic acid comprising Ant1_HapM4 has a sequence which is at least 80%, preferably at least 90%, more preferably at least 95% identical to a sequence as set forth in SEQ ID NO: 10. In certain embodiments, the polynucleic acid comprising Ant1_HapM5 has a sequence which is at least 80%, preferably at least 90%, more preferably at least 95% identical to a sequence as set forth in SEQ ID NO: 11. In certain embodiments, the polynucleic acid comprising Ant1_HapM6 has a sequence which is at least 80%, preferably at least 90%, more preferably at least 95% identical to a sequence as set forth in SEQ ID NO: 12. In certain embodiments, said polynucleic acid comprises a sequence which is at least 80%, preferably at least 90%, more preferably at least 95% identical to a sequence as set forth in any of SEQ ID NOs: 7 to 12 and comprising (respectively) T at or corresponding to position 95 of SEQ ID NO: 7, C at or corresponding to position 224 of SEQ ID NO: 8, C at or corresponding to position 206 of SEQ ID NO: 9, C at or corresponding to position 73 of SEQ ID NO: 10, C at or corresponding to position 145 of SEQ ID NO: 11, and/or G at or corresponding to position 50 of SEQ ID NO: 12, preferably all, or alternatively not comprising one or more of (respectively) T at or corresponding to position 95 of SEQ ID NO: 7, C at or corresponding to position 224 of SEQ ID NO: 8, C at or corresponding to position 206 of SEQ ID NO: 9, C at or corresponding to position 73 of SEQ ID NO: 10, C at or corresponding to position 145 of SEQ ID NO: 11, and/or G at or corresponding to position 50 of SEQ ID NO: 12, or alternatively comprising one or more of V or T at or corresponding to position 95 of SEQ ID NO: 7, D or G at or corresponding to position 224 of SEQ ID NO: 8, D or G at or corresponding to position 206 of SEQ ID NO: 9, D or T at or corresponding to position 73 of SEQ ID NO: 10, D or A at or corresponding to position 145 of SEQ ID NO: 11, and/or H or A at or corresponding to position 50 of SEQ ID NO: 12.

In certain embodiments, said polynucleic acid comprises a sequence as set forth in any of SEQ ID NOs: 7 to 12. In certain embodiments, the polynucleic acid comprising Ant1_HapM1 has a sequence as set forth in SEQ ID NO: 7. In certain embodiments, the polynucleic acid comprising Ant1_HapM2 has a sequence as set forth in SEQ ID NO: 8. In certain embodiments, the polynucleic acid comprising Ant1_HapM3 has a sequence as set forth in SEQ ID NO: 9. In certain embodiments, the polynucleic acid comprising Ant1_HapM4 has a sequence as set forth in SEQ ID NO: 10. In certain embodiments, the polynucleic acid comprising Ant1_HapM5 has a sequence as set forth in SEQ ID NO: 11. In certain embodiments, the polynucleic acid comprising Ant1_HapM6 has a sequence as set forth in SEQ ID NO: 12.

In certain embodiments, the method comprises screening for one or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e. in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In certain embodiments, the method comprises screening for two or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In certain embodiments, the method comprises screening for three or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In certain embodiments, the method comprises screening for four or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In certain embodiments, the method comprises screening for five or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In certain embodiments, the method comprises screening for molecular marker(s) (allele(s)) Ant1_HapM1, Ant1_HapM2, Ant1_HapM3, Ant1_HapM4, Ant1_HapM5, and Ant1_HapM6 (in the Ant2 gene and/or genome of the plant or plant part), optionally further comprising screening for one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part).

In an aspect, the invention relates to a method for identifying and/or selecting a barley plant or plant part, comprising screening for the presence of one or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e. in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In an aspect, the invention relates to a method for identifying and/or selecting a barley plant or plant part, comprising screening for the presence of two or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In an aspect, the invention relates to a method for identifying and/or selecting a barley plant or plant part, comprising screening for the presence of three or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In an aspect, the invention relates to a method for identifying and/or selecting a barley plant or plant part, comprising screening for the presence of four or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In an aspect, the invention relates to a method for identifying and/or selecting a barley plant or plant part, comprising screening for the presence of five or more molecular marker (allele) selected from Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part). In an aspect, the invention relates to a method for identifying and/or selecting a barley plant or plant part, comprising screening for the presence of molecular marker(s) (allele(s)) Ant1_HapM1, Ant1_HapM2, Ant1_HapM3, Ant1_HapM4, Ant1_HapM5, and Ant1_HapM6 (in the Ant1 gene and/or genome of the plant or plant part), optionally further comprising screening for the presence of one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part).

In an aspect, the invention relates to a method for identifying and/or selecting a barley plant or plant part, comprising screening for the presence of a polynucleotide comprising one or more molecular marker (allele) selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2. Ant2_M3, Ant2_M4, Ant2_M5, Ant2_M6, and/or Ant2_M7; or screening for molecular marker (allele) Ant2_HapM1 and (i.e., in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part), optionally further comprising screening for the presence of a polynucleotide comprising molecular marker(s) (allele(s)) Ant1_HapM1, Ant1_HapM2, Ant1_HapM3, Ant1_HapM4, Ant1_HapM5, and/or Ant1_HapM6 (in the Ant1 gene and/or genome of the plant or plant part), preferably all.

Screening for the presence of a polynucleic acid comprising molecular marker (allele) Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 may comprise screening for the presence of molecular marker (allele) Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7. Alternatively, screening for the presence of Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 may encompass screening for another closely linked marker, which if identified automatically identifies molecular marker (allele) Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7.

In certain embodiments, said polynucleic acid comprises a sequence which is at least 80%, preferably at least 90%, more preferably at least 95% identical to a sequence as set forth in SEQ ID NO: 14 or 17. In certain embodiments, said polynucleic acid comprises a sequence which is at least 80%, preferably at least 90% identical to a sequence as set forth in SEQ ID NO: 14 or 17, and encoding a protein having a sequence which is at least 80%, preferably at least 90%, more preferably at least 95% identical to a sequence as set forth in SEQ ID NO: 16 or 19. In certain embodiments, said polynucleic acid comprises a sequence which is at least 80%, preferably at least 90%, more preferably at least 95% identical to a sequence as set forth in SEQ ID NO: 14 or 17 and comprising or associated or linked with (respectively) C at or corresponding to position 101 of SEQ ID NO: 13, or alternatively not associated or linked with C at or corresponding to position 101 of SEQ ID NO: 13, or alternatively associated or linked with D or T at or corresponding to position 101 of SEQ ID NO: 13. In certain embodiments, said polynucleic acid comprises a sequence which is at least 80%, preferably at least 90%, more preferably at least 95% identical to a sequence as set forth in SEQ ID NO: 14 or 17 and associated or linked with C at or corresponding to position 101 of SEQ ID NO: 13, and encoding a protein having a sequence which is at least 80%, preferably at least 90%, more preferably at least 95% identical to a sequence as set forth in SEQ ID NO: 16 or 19. The skilled person will understand that the indicated SNP/single nucleotide positions correspond to the positions of the associated Ant2_M1 marker. In certain embodiments, said polynucleic acid comprises a sequence which is at least 80%, preferably at least 90%, more preferably at least 95% identical to a sequence as set forth in SEQ ID NO: 14 or 17 and comprising (respectively) G at or corresponding to position 491, C at or corresponding to position 1597, T at or corresponding to position 1950, G at or corresponding to position 2207, T at or corresponding to position 2946, A at or corresponding to position 3907, G at or corresponding to position 3743, C at or corresponding to position 410, A at or corresponding to position 803, T at or corresponding to position 1796, A at or corresponding to position 2013, G at or corresponding to position 3530, and/or C at or corresponding to position 4895 of SEQ ID NO: 14, or alternatively not comprising one or more of (respectively) G at or corresponding to position 491, C at or corresponding to position 1597, T at or corresponding to position 1950, G at or corresponding to position 2207, T at or corresponding to position 2946, A at or corresponding to position 3907, G at or corresponding to position 3743, C at or corresponding to position 410, A at or corresponding to position 803, T at or corresponding to position 1796, A at or corresponding to position 2013, G at or corresponding to position 3530, and/or C at or corresponding to position 4895 of SEQ ID NO: 14, or alternatively comprising one or more of H or T at or corresponding to position 1284, D or T at or corresponding to position 2381, V or G at or corresponding to position 2734, H or A at or corresponding to position 2989, V or C at or corresponding to position 3729, B or C at or corresponding to position 4690, H or A at or corresponding to position 4526, D or G at or corresponding to position 1203, B or G at or corresponding to position 1596, V or C at or corresponding to position 2580, B or T at or corresponding to position 2797, H or A at or corresponding to position 4313, and/or D or G at or corresponding to position 5676 of SEQ ID NO: 17, or alternatively not comprising one or more of G at or corresponding to position 1284, C at or corresponding to position 2381, T at or corresponding to position 2734, G at or corresponding to position 2989, T at or corresponding to position 3729, A at or corresponding to position 4690, G at or corresponding to position 4526, C at or corresponding to position 1203, A at or corresponding to position 1596, T at or corresponding to position 2580, A at or corresponding to position 2797, G at or corresponding to position 4313, and/or C at or corresponding to position 5676 of SEQ ID NO: 17. In certain embodiments, said polynucleic acid comprises a sequence which is at least 80%, preferably at least 90%, more preferably at least 95% identical to a sequence as set forth in SEQ ID NO: 14 or 17 and comprising (respectively) G at or corresponding to position 491, C at or corresponding to position 1597, T at or corresponding to position 1950, G at or corresponding to position 2207, T at or corresponding to position 2946, A at or corresponding to position 3907, G at or corresponding to position 3743, C at or corresponding to position 410, A at or corresponding to position 803, T at or corresponding to position 1796, A at or corresponding to position 2013, G at or corresponding to position 3530, and/or C at or corresponding to position 4895 of SEQ ID NO: 14, or alternatively not comprising one or more of (respectively) G at or corresponding to position 491, C at or corresponding to position 1597, T at or corresponding to position 1950, G at or corresponding to position 2207, T at or corresponding to position 2946, A at or corresponding to position 3907, G at or corresponding to position 3743, C at or corresponding to position 410, A at or corresponding to position 803, T at or corresponding to position 1796, A at or corresponding to position 2013, G at or corresponding to position 3530, and/or C at or corresponding to position 4895 of SEQ ID NO: 14, or alternatively comprising one or more of H or T at or corresponding to position 1284, D or T at or corresponding to position 2381, V or G at or corresponding to position 2734, H or A at or corresponding to position 2989, V or C at or corresponding to position 3729, B or C at or corresponding to position 4690, H or A at or corresponding to position 4526, D or G at or corresponding to position 1203, B or G at or corresponding to position 1596, V or C at or corresponding to position 2580, B or T at or corresponding to position 2797, H or A at or corresponding to position 4313, and/or D or G at or corresponding to position 5676 of SEQ ID NO: 17, or alternatively not comprising one or more of G at or corresponding to position 1284, C at or corresponding to position 2381, T at or corresponding to position 2734, G at or corresponding to position 2989, T at or corresponding to position 3729, A at or corresponding to position 4690, G at or corresponding to position 4526, C at or corresponding to position 1203, A at or corresponding to position 1596, T at or corresponding to position 2580, A at or corresponding to position 2797, G at or corresponding to position 4313, and/or C at or corresponding to position 5676 of SEQ ID NO: 17, and encoding a protein having a sequence which is at least 80%, preferably at least 90%, more preferably at least 95% identical to a sequence as set forth (respectively) in SEQ ID NO: 16 or 19.

In certain embodiments, said polynucleic acid comprises a sequence as set forth in SEQ ID NO: 14 or 17.

In certain embodiments, said polynucleic acid comprises a sequence which is at least 80%, preferably at least 90%, more preferably at least 95% identical to a sequence as set forth in SEQ ID NO: 15 or 18. In certain embodiments, said polynucleic acid comprises a sequence which is at least 80%, preferably at least 90% identical to a sequence as set forth in SEQ ID NO: 15 or 18, and encoding a protein having a sequence which is at least 80%, preferably at least 90%, more preferably at least 95% identical to a sequence as set forth in SEQ ID NO: 16 or 19. In certain embodiments, said polynucleic acid comprises a sequence which is at least 80%, preferably at least 90%, more preferably at least 95% identical to a sequence as set forth in SEQ ID NO: 15 or 18 and associated or linked with C at or corresponding to position 101 of SEQ ID NO: 13, or alternatively not associated or linked with C at or corresponding to position 101 of SEQ ID NO: 13, or alternatively associated or linked with D or T at or corresponding to position 101 of SEQ ID NO: 13. In certain embodiments, said polynucleic acid comprises a sequence which is at least 80%, preferably at least 90%, more preferably at least 95% identical to a sequence as set forth in SEQ ID NO: 15 or 18 and associated or linked with C at or corresponding to position 101 of SEQ ID NO: 13, or alternatively not associated or linked with C at or corresponding to position 101 of SEQ ID NO: 13, or alternatively associated or linked with D or T at or corresponding to position 101 of SEQ ID NO: 13, and encoding a protein having a sequence which is at least 80%, preferably at least 90%, more preferably at least 95% identical to a sequence as set forth in SEQ ID NO: 16 or 19. The skilled person will understand that the indicated SNP/single nucleotide positions correspond to the positions of the associated Ant2_M1 marker. In certain embodiments, said polynucleic acid comprises a sequence which is at least 80%, preferably at least 90%, more preferably at least 95% identical to a sequence as set forth in SEQ ID NO: 15 or 18 and comprising (respectively) A at or corresponding to position 804, G at or corresponding to position 640, A at or corresponding to position 49 and/or C at or corresponding to position 1619 of SEQ ID NO: 15, or alternatively not comprising one or more of (respectively) A at or corresponding to position 804, G at or corresponding to position 640, A at or corresponding to position 49 and/or C at or corresponding to position 1619 of SEQ ID NO: 15, or alternatively comprising one or more of B or C at or corresponding to position 804, H or A at or corresponding to position 640, B or G at or corresponding to position 49, and/or D or G at or corresponding to position 1619 of SEQ ID NO: 18, or alternatively not comprising one or more of A at or corresponding to position 804, G at or corresponding to position 640, A at or corresponding to position 49, and/or C at or corresponding to position 1619 of SEQ ID NO: 18. In certain embodiments, said polynucleic acid comprises a sequence which is at least 80%, preferably at least 90%, more preferably at least 95% identical to a sequence as set forth in SEQ ID NO: 15 or 18 and comprising (respectively) A at or corresponding to position 804, G at or corresponding to position 640, A at or corresponding to position 49 and/or C at or corresponding to position 1619 of SEQ ID NO: 15, or alternatively not comprising one or more of (respectively) A at or corresponding to position 804, G at or corresponding to position 640, A at or corresponding to position 49 and/or C at or corresponding to position 1619 of SEQ ID NO: 15, or alternatively comprising one or more of B or C at or corresponding to position 804, H or A at or corresponding to position 640, B or G at or corresponding to position 49, and/or D or G at or corresponding to position 1619 of SEQ ID NO: 18, or alternatively not comprising one or more of A at or corresponding to position 804, G at or corresponding to position 640, A at or corresponding to position 49, and/or C at or corresponding to position 1619 of SEQ ID NO: 18, and encoding a protein having a sequence which is at least 80%, preferably at least 90%, more preferably at least 95% identical (respectively) to a sequence as set forth in SEQ ID NO: 16 or 19.

In certain embodiments, said polynucleic acid comprises a sequence as set forth in SEQ ID NO: 15 or 18.

In certain embodiments, said polynucleic acid comprises a sequence encoding a protein sequence which is at least 80%, preferably at least 90%, more preferably at least 95% identical to a sequence as set forth in SEQ ID NO: 16 or 19. In certain embodiments, said polynucleic acid comprises a sequence encoding a protein having a sequence which is at least 80%, preferably at least 90%, more preferably at least 95% identical to a sequence as set forth in SEQ ID NO: 16 or 19 and associated or linked with C at or corresponding to position 101 of SEQ ID NO: 13, or alternatively not associated or linked with C at or corresponding to position 101 of SEQ ID NO: 13, or alternatively associated or linked with D or T at or corresponding to position 101 of SEQ ID NO: 13. The skilled person will understand that the indicated SNP/single nucleotide positions correspond to the positions of the associated Ant2_M1marker. In certain embodiments, said polynucleic acid comprises a sequence encoding a protein having a sequence which is at least 80%, preferably at least 90%, more preferably at least 95% identical to a sequence as set forth in SEQ ID NO: 16 or 19 and comprising (respectively) A at or corresponding to position 804, G at or corresponding to position 640, A at or corresponding to position 49 and/or C at or corresponding to position 1619, or alternatively not comprising one or more of (respectively) A at or corresponding to position 804, G at or corresponding to position 640, A at or corresponding to position 49 and/or C at or corresponding to position 1619, or alternatively comprising one or more of B or C at or corresponding to position 804, H or A at or corresponding to position 640, B or G at or corresponding to position 49, and/or D or G at or corresponding to position 1619. The skilled person will understand that the indicated SNP/single nucleotide positions correspond to the positions of the coding sequence encoding the protein.

In certain embodiments, said polynucleic acid encodes for a protein having a sequence as set forth in SEQ ID NO: 16 or 19.

In certain embodiments, said polynucleic acid comprises a sequence which is at least 80%, preferably at least 90%, more preferably at least 95% identical to a sequence as set forth in any of SEQ ID NOs: 13 or 20 to 32. In certain embodiments, the polynucleic acid comprising Ant2_M1 has a sequence which is at least 80%, preferably at least 90%, more preferably at least 95% identical to a sequence as set forth in SEQ ID NO: 13. In certain embodiments, the polynucleic acid comprising Ant2_M2 has a sequence which is at least 80%, preferably at least 90%, more preferably at least 95% identical to a sequence as set forth in SEQ ID NO: 20. In certain embodiments, the polynucleic acid comprising Ant2_M3 has a sequence which is at least 80%, preferably at least 90%, more preferably at least 95% identical to a sequence as set forth in SEQ ID NO: 21. In certain embodiments, the polynucleic acid comprising Ant2_M4 has a sequence which is at least 80%, preferably at least 90%, more preferably at least 95% identical to a sequence as set forth in SEQ ID NO: 22. In certain embodiments, the polynucleic acid comprising Ant2_M5 has a sequence which is at least 80%, preferably at least 90%, more preferably at least 95% identical to a sequence as set forth in SEQ ID NO: 23. In certain embodiments, the polynucleic acid comprising Ant2_M6 has a sequence which is at least 80%, preferably at least 90%, more preferably at least 95% identical to a sequence as set forth in SEQ ID NO: 24. In certain embodiments, the polynucleic acid comprising Ant2_M7 has a sequence which is at least 80%, preferably at least 90%, more preferably at least 95% identical to a sequence as set forth in SEQ ID NO: 25. In certain embodiments, the polynucleic acid comprising Ant2_HapM1 has a sequence which is at least 80%, preferably at least 90%, more preferably at least 95% identical to a sequence as set forth in SEQ ID NO: 26. In certain embodiments, the polynucleic acid comprising Ant2_HapM2 has a sequence which is at least 80%, preferably at least 90%, more preferably at least 95% identical to a sequence as set forth in SEQ ID NO: 27. In certain embodiments, the polynucleic acid comprising Ant2_HapM3 has a sequence which is at least 80%, preferably at least 90%, more preferably at least 95% identical to a sequence as set forth in SEQ ID NO: 28. In certain embodiments, the polynucleic acid comprising Ant2_HapM4 has a sequence which is at least 80%, preferably at least 90%, more preferably at least 95% identical to a sequence as set forth in SEQ ID NO: 29. In certain embodiments, the polynucleic acid comprising Ant2_HapM5 has a sequence which is at least 80%, preferably at least 90%, more preferably at least 95% identical to a sequence as set forth in SEQ ID NO: 30. In certain embodiments, the polynucleic acid comprising Ant2_HapM6 has a sequence which is at least 80%, preferably at least 90%, more preferably at least 95% identical to a sequence as set forth in SEQ ID NO: 31. In certain embodiments, the polynucleic acid comprising Ant2_HapM7 has a sequence which is at least 80%, preferably at least 90%, more preferably at least 95% identical to a sequence as set forth in SEQ ID NO: 32.

In certain embodiments, said polynucleic acid comprises a sequence which is at least 80%, preferably at least 90%, more preferably at least 95% identical to a sequence as set forth in any of SEQ ID NOs: 13 or 20 to 32 and comprising (respectively) C at or corresponding to position 101 of SEQ ID NO: 13, G at or corresponding to position 46 of SEQ ID NO: 20, C at or corresponding to position 96 of SEQ ID NO: 21, T at or corresponding to position 64 of SEQ ID NO: 22, G at or corresponding to position 73 of SEQ ID NO: 23, T at or corresponding to position 101 of SEQ ID NO: 24, A at or corresponding to position 72 of SEQ ID NO: 25, G at or corresponding to position 84 of SEQ ID NO: 26, C at or corresponding to position 76 of SEQ ID NO: 27, A at or corresponding to position 94 of SEQ ID NO: 28, T at or corresponding to position 88 of SEQ ID NO: 29, A at or corresponding to position 52 of SEQ ID NO: 30, G at or corresponding to position 80 of SEQ ID NO: 31, and/or C at or corresponding to position 73 of SEQ ID NO: 32, or alternatively not comprising C at or corresponding to position 101 of SEQ ID NO: 13, G at or corresponding to position 46 of SEQ ID NO: 20, C at or corresponding to position 96 of SEQ ID NO: 21, T at or corresponding to position 64 of SEQ ID NO: 22, G at or corresponding to position 73 of SEQ ID NO: 23, T at or corresponding to position 101 of SEQ ID NO: 24, A at or corresponding to position 72 of SEQ ID NO: 25, G at or corresponding to position 84 of SEQ ID NO: 26, C at or corresponding to position 76 of SEQ ID NO: 27, A at or corresponding to position 94 of SEQ ID NO: 28, T at or corresponding to position 88 of SEQ ID NO: 29, A at or corresponding to position 52 of SEQ ID NO: 30, G at or corresponding to position 80 of SEQ ID NO: 31, and/or C at or corresponding to position 73 of SEQ ID NO: 32, or alternatively comprising D or T at position 101 of SEQ ID NO: 13, H or T at or corresponding to position 46 of SEQ ID NO: 20, D or T at or corresponding to position 96 of SEQ ID NO: 21, V or G at or corresponding to position 64 of SEQ ID NO: 22, H or A at or corresponding to position 73 of SEQ ID NO: 23, V or C at or corresponding to position 101 of SEQ ID NO: 24, B or C at or corresponding to position 72 of SEQ ID NO: 25, H or A at or corresponding to position 84 of SEQ ID NO: 26, D or G at or corresponding to position 76 of SEQ ID NO: 27, B or G at or corresponding to position 94 of SEQ ID NO: 28, V or C at or corresponding to position 88 of SEQ ID NO: 29, B or T at or corresponding to position 52 of SEQ ID NO: 30, H or A at or corresponding to position 80 of SEQ ID NO: 31, and/or D or G at or corresponding to position 73 of SEQ ID NO: 32.

In certain embodiments, said polynucleic acid comprises a sequence as set forth in any of SEQ ID NOs: 13 or 20 to 32. In certain embodiments, the polynucleic acid comprising Ant2_M1 has a sequence as set forth in SEQ ID NO: 13. In certain embodiments, the polynucleic acid comprising Ant2_M2 has a sequence as set forth in SEQ ID NO: 20. In certain embodiments, the polynucleic acid comprising Ant2_M3 has a sequence as set forth in SEQ ID NO: 21. In certain embodiments, the polynucleic acid comprising Ant2_M4 has a sequence as set forth in SEQ ID NO: 22. In certain embodiments, the polynucleic acid comprising Ant2_M5 has a sequence as set forth in SEQ ID NO: 23. In certain embodiments, the polynucleic acid comprising Ant2_M6 has a sequence as set forth in SEQ ID NO: 24. In certain embodiments, the polynucleic acid comprising Ant2_M7 has a sequence as set forth in SEQ ID NO: 25. In certain embodiments, the polynucleic acid comprising Ant2_HapM1 has a sequence as set forth in SEQ ID NO: 26. In certain embodiments, the polynucleic acid comprising Ant2_HapM2 has a sequence as set forth in SEQ ID NO: 27. In certain embodiments, the polynucleic acid comprising Ant2_HapM3 has a sequence as set forth in SEQ ID NO: 28. In certain embodiments, the polynucleic acid comprising Ant2_HapM4 has a sequence as set forth in SEQ ID NO: 29. In certain embodiments, the polynucleic acid comprising Ant2_HapM5 has a sequence as set forth in SEQ ID NO: 30. In certain embodiments, the polynucleic acid comprising Ant2_HapM6 has a sequence as set forth in SEQ ID NO: 31. In certain embodiments, the polynucleic acid comprising Ant2_HapM7 has a sequence as set forth in SEQ ID NO: 32.

In certain embodiments, the method comprises screening for the presence of molecular marker (allele) Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e. in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part), optionally further comprising screening for the presence of molecular marker(s) (allele(s)) Ant1_HapM1, Ant1_HapM2, Ant1_HapM3, Ant1_HapM4, Ant1_HapM5, and/or Ant1_HapM6 (in the Ant1 gene and/or genome of the plant or plant part), preferably all.

In an aspect, the invention relates to a method for identifying and/or selecting a barley plant or plant part, comprising screening for the presence of molecular marker (allele) Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably comprising screening for one or more molecular marker (allele) selected from one or more of Ant2_M2, Ant2_M3, Ant2_M4, Ant2_M5, Ant2_M6, and/or Ant2_M7; or screening for molecular marker (allele) Ant2_HapM1 and (i.e. in combination with) one or more of Ant2_HapM2, Ant2_HapM3, Ant2_HapM4, Ant2_HapM5, Ant2_HapM6, and/or Ant2_HapM7 (in or associated with or linked with the Ant2 gene and/or in the genome of the plant or plant part), optionally further comprising screening for the presence of molecular marker(s) (allele(s)) Ant1_HapM1, Ant1_HapM2, Ant1_HapM3, Ant1_HapM4, Ant1_HapM5, and/or Ant1_HapM6 (in the Ant1 gene and/or genome of the plant or plant part), preferably all.

Methods for screening for or detecting the presence of a locus (allele), polynucleic acid/polynucleotide, QTL (allele), (molecular) marker (allele), gene (allele), SNP (allele), haplotype, or SEQ ID NO as described herein are known in the art. Without limitation, screening or detecting may encompass or comprise sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay), etc. Preferably the one or more locus (allele), polynucleic acid/polynucleotide, QTL (allele), (molecular) marker (allele), gene (allele), SNP (allele), haplotype, or SEQ ID NO is detectable by a polynucleic acid, such as an allele specific polynucleic acid (molecular marker), suitable for hybridization as a probe or forward primer or reverse primer to such locus (allele), polynucleic acid/polynucleotide, QTL (allele), (molecular) marker (allele), gene (allele), SNP (allele), haplotype, or SEQ ID NO, such as on a chromosomal interval within the genome.

It will be understood that the plant or plant part as referred to herein is identified in case the locus (allele), polynucleic acid/polynucleotide, QTL (allele), (molecular) marker (allele), gene (allele), SNP (allele), haplotype and/or SEQ ID NO is detected, such as preferably detected in the genome of a plant of plant part.

In certain embodiments, Ant1_HapM1 is or comprises a SNP at a position corresponding to position 71238822 (on chromosome 7H) of barley, preferably the barley reference genome Morex (version 3.0). In certain embodiments, Ant1_HapM1 is or comprises a SNP corresponding to position 68464237 (on chromosome 7H) of barley, preferably the barley reference genome Morex (version 2.0). In certain embodiments, Ant1_HapM1 is or comprises a SNP at a position corresponding to position 95 of SEQ ID NO: 1, or a sequence which is at least 80%, preferably at least 90%, more preferably at least 95% identical to SEQ ID NO: 1. In certain embodiments, Ant1_HapM1 is or comprises a SNP at a position corresponding to position 95 of SEQ ID NO: 7 or a sequence which is at least 80%, preferably at least 90%, more preferably at least 95% identical to SEQ ID NO: 7.

In certain embodiments, Ant1_HapM2 is or comprises a SNP at a position corresponding to position 71238454 (on chromosome 7H) of barley, preferably the barley reference genome Morex (version 3.0). In certain embodiments, Ant1_HapM2 is or comprises a SNP corresponding to position 68463869 (on chromosome 7H) of barley, preferably the barley reference genome Morex (version 2.0). In certain embodiments, Ant1_HapM2 is or comprises a SNP at a position corresponding to position 463 of SEQ ID NO: 1 or a sequence which is at least 80%, preferably at least 90%, more preferably at least 95% identical to SEQ ID NO: 1. In certain embodiments, Ant1_HapM2 is or comprises a SNP at a position corresponding to position 224 of SEQ ID NO: 8 or a sequence which is at least 80%, preferably at least 90%, more preferably at least 95% identical to SEQ ID NO: 8.

In certain embodiments, Ant1_HapM3 is or comprises a SNP at a position corresponding to position 71238052 (on chromosome 7H) of barley, preferably the barley reference genome Morex (version 3.0). In certain embodiments, Ant1_HapM3 is or comprises a SNP corresponding to position 68463467 (on chromosome 7H) of barley, preferably the barley reference genome Morex (version 2.0). In certain embodiments, Ant1_HapM3 is or comprises a SNP at a position corresponding to position 865 of SEQ ID NO: 1 or a sequence which is at least 80%, preferably at least 90%, more preferably at least 95% identical to SEQ ID NO: 1. In certain embodiments, Ant1_HapM3 is or comprises a SNP at a position corresponding to position 219 of SEQ ID NO: 2 or a sequence which is at least 80%, preferably at least 90%, more preferably at least 95% identical to SEQ ID NO: 2. In certain embodiments, Ant1_HapM3 is or comprises a SNP at a position corresponding to position 219 of a sequence encoding a protein having a sequence as set forth in SEQ ID NO: 3 or a sequence which is at least 80%, preferably at least 90%, more preferably at least 95% identical to SEQ ID NO: 3. In certain embodiments, Ant1_HapM3 is or comprises a SNP at a position corresponding to position 206 of SEQ ID NO: 9 or a sequence which is at least 80%, preferably at least 90%, more preferably at least 95% identical to SEQ ID NO: 9.

In certain embodiments, Ant1_HapM4 is or comprises a SNP at a position corresponding to position 71237825 (on chromosome 7H) of barley, preferably the barley reference genome Morex (version 3.0). In certain embodiments, Ant1_HapM4 is or comprises a SNP corresponding to position 68463240 (on chromosome 7H) of barley, preferably the barley reference genome Morex (version 2.0). In certain embodiments, Ant1_HapM4 is or comprises a SNP at a position corresponding to position 1092 of SEQ ID NO: 1 or a sequence which is at least 80%, preferably at least 90%, more preferably at least 95% identical to SEQ ID NO: 1. In certain embodiments, Ant1_HapM4 is or comprises a SNP at a position corresponding to position 446 of SEQ ID NO: 2 or a sequence which is at least 80%, preferably at least 90%, more preferably at least 95% identical to SEQ ID NO: 2. In certain embodiments, Ant1_HapM4 is or comprises a SNP at a position corresponding to position 446 of a sequence encoding a protein having a sequence as set forth in SEQ ID NO: 3 or a sequence which is at least 80%, preferably at least 90%, more preferably at least 95% identical to SEQ ID NO: 3. In certain embodiments, Ant1_HapM4 is or comprises a SNP at a position corresponding to position 73 of SEQ ID NO: 10 or a sequence which is at least 80%, preferably at least 90%, more preferably at least 95% identical to SEQ ID NO: 10.

In certain embodiments, Ant1_HapM5 is or comprises a SNP at a position corresponding to position 71237633 (on chromosome 7H) of barley, preferably the barley reference genome Morex (version 3.0). In certain embodiments, Ant1_HapM5 is or comprises a SNP corresponding to position 68463048 (on chromosome 7H) of barley, preferably the barley reference genome Morex (version 2.0). In certain embodiments, Ant1_HapM5 is or comprises a SNP at a position corresponding to position 1284 of SEQ ID NO: 1 or a sequence which is at least 80%, preferably at least 90%, more preferably at least 95% identical to SEQ ID NO: 1. In certain embodiments, Ant1_HapM5 is or comprises a SNP at a position corresponding to position 638 of SEQ ID NO: 2 or a sequence which is at least 80%, preferably at least 90%, more preferably at least 95% identical to SEQ ID NO: 2. In certain embodiments, Ant1_HapM5 is or comprises a SNP at a position corresponding to position 638 of a sequence encoding a protein having a sequence as set forth in SEQ ID NO: 3 or a sequence which is at least 80%, preferably at least 90%, more preferably at least 95% identical to SEQ ID NO: 3. In certain embodiments, Ant1_HapM5 is or comprises a SNP at a position corresponding to position 145 of SEQ ID NO: 11 or a sequence which is at least 80%, preferably at least 90%, more preferably at least 95% identical to SEQ ID NO: 11.

In certain embodiments, Ant1_HapM6 is or comprises a SNP at a position corresponding to position 71237608 (on chromosome 7H) of barley, preferably the barley reference genome Morex (version 3.0). In certain embodiments, Ant1_HapM6 is or comprises a SNP corresponding to position 68463023 (on chromosome 7H) of barley, preferably the barley reference genome Morex (version 2.0). In certain embodiments, Ant1_HapM6 is or comprises a SNP at a position corresponding to position 1309 of SEQ ID NO: 1 or a sequence which is at least 80%, preferably at least 90%, more preferably at least 95% identical to SEQ ID NO: 1. In certain embodiments, Ant1_HapM6 is or comprises a SNP at a position corresponding to position 663 of SEQ ID NO: 2 or a sequence which is at least 80%, preferably at least 90%, more preferably at least 95% identical to SEQ ID NO: 2. In certain embodiments, Ant1_HapM6 is or comprises a SNP at a position corresponding to position 663 of a sequence encoding a protein having a sequence as set forth in SEQ ID NO: 3 or a sequence which is at least 80%, preferably at least 90%, more preferably at least 95% identical to SEQ ID NO: 3. In certain embodiments, Ant1_HapM6 is or comprises a SNP at a position corresponding to position 50 of SEQ ID NO: 12 or a sequence which is at least 80%, preferably at least 90%, more preferably at least 95% identical to SEQ ID NO: 12.

In certain embodiments, Ant2_M1 is or comprises a SNP at a position corresponding to position 591324765 (on chromosome 2H) of barley, preferably the barley reference genome Morex (version 3.0). In certain embodiments, Ant2_M1 is or comprises a SNP corresponding to position 599993462 (on chromosome 2H) of barley, preferably the barley reference genome Morex (version 2.0). Ant2Ant2Ant2In certain embodiments, Ant2_M1 is or comprises a SNP at a position corresponding to position 101 of SEQ ID NO: 13 or a sequence which is at least 80%, preferably at least 90%, more preferably at least 95% identical to SEQ ID NO: 13.

In certain embodiments, Ant2_M2 is or comprises a SNP at a position corresponding to position 593492855 (on chromosome 2H) of barley, preferably the barley reference genome Morex (version 3.0). In certain embodiments, Ant_M2 is or comprises a SNP at a position corresponding to position 491 of SEQ ID NO: 14, or a sequence which is at least 80%, preferably at least 90%, more preferably at least 95% identical to SEQ ID NO: 14. In certain embodiments, Ant2_M2 is or comprises a SNP at a position corresponding to position 46 of SEQ ID NO: 20 or a sequence which is at least 80%, preferably at least 90%, more preferably at least 95% identical to SEQ ID NO: 20.

In certain embodiments, Ant2_M3 is or comprises a SNP at a position corresponding to position 593493961 (on chromosome 2H) of barley, preferably the barley reference genome Morex (version 3.0). In certain embodiments, Ant_M3 is or comprises a SNP at a position corresponding to position 1597 of SEQ ID NO: 14, or a sequence which is at least 80%, preferably at least 90%, more preferably at least 95% identical to SEQ ID NO: 14. In certain embodiments, Ant2_M3 is or comprises a SNP at a position corresponding to position 96 of SEQ ID NO: 21 or a sequence which is at least 80%, preferably at least 90%, more preferably at least 95% identical to SEQ ID NO: 21.

In certain embodiments, Ant2_M4 is or comprises a SNP at a position corresponding to position 593494314 (on chromosome 2H) of barley, preferably the barley reference genome Morex (version 3.0). In certain embodiments, Ant_M4 is or comprises a SNP at a position corresponding to position 1950 of SEQ ID NO: 14, or a sequence which is at least 80%, preferably at least 90%, more preferably at least 95% identical to SEQ ID NO: 14. In certain embodiments, Ant2_M4 is or comprises a SNP at a position corresponding to position 64 of SEQ ID NO: 22 or a sequence which is at least 80%, preferably at least 90%, more preferably at least 95% identical to SEQ ID NO: 22.

In certain embodiments, Ant2_M5 is or comprises a SNP at a position corresponding to position 593494569 (on chromosome 2H) of barley, preferably the barley reference genome Morex (version 3.0). In certain embodiments, Ant_M5 is or comprises a SNP at a position corresponding to position 2207 of SEQ ID NO: 14, or a sequence which is at least 80%, preferably at least 90%, more preferably at least 95% identical to SEQ ID NO: 14. In certain embodiments, Ant2_M5 is or comprises a SNP at a position corresponding to position 73 of SEQ ID NO: 23 or a sequence which is at least 80%, preferably at least 90%, more preferably at least 95% identical to SEQ ID NO: 23.

In certain embodiments, Ant2_M6 is or comprises a SNP at a position corresponding to position 593495307 (on chromosome 2H) of barley, preferably the barley reference genome Morex (version 3.0). In certain embodiments, Ant_M6 is or comprises a SNP at a position corresponding to position 2946 of SEQ ID NO: 14, or a sequence which is at least 80%, preferably at least 90%, more preferably at least 95% identical to SEQ ID NO: 14. In certain embodiments, Ant2_M6 is or comprises a SNP at a position corresponding to position 101 of SEQ ID NO: 24 or a sequence which is at least 80%, preferably at least 90%, more preferably at least 95% identical to SEQ ID NO: 24.

In certain embodiments, Ant2_M7 is or comprises a SNP at a position corresponding to position 593496606 (on chromosome 2H) of barley, preferably the barley reference genome Morex (version 3.0). In certain embodiments, Ant_M7 is or comprises a SNP at a position corresponding to position 3907 of SEQ ID NO: 14, or a sequence which is at least 80%, preferably at least 90%, more preferably at least 95% identical to SEQ ID NO: 14. In certain embodiments, Ant2_M7 is or comprises a SNP at a position corresponding to position 804 of SEQ ID NO: 15 or a sequence which is at least 80%, preferably at least 90%, more preferably at least 95% identical to SEQ ID NO: 15. In certain embodiments, Ant2_M7 is or comprises a SNP at a position corresponding to position 804 of a sequence encoding a protein having a sequence as set forth in SEQ ID NO: 16 or a sequence which is at least 80%, preferably at least 90%, more preferably at least 95% identical to SEQ ID NO: 16. In certain embodiments, Ant2_M7 is or comprises a SNP at a position corresponding to position 72 of SEQ ID NO: 25 or a sequence which is at least 80%, preferably at least 90%, more preferably at least 95% identical to SEQ ID NO: 25.

In certain embodiments, Ant2_HapM1 is or comprises a SNP at a position corresponding to position 593496442 (on chromosome 2H) of barley, preferably the barley reference genome Morex (version 3.0). In certain embodiments, Ant_HapM1 is or comprises a SNP at a position corresponding to position 3743 of SEQ ID NO: 14, or a sequence which is at least 80%, preferably at least 90%, more preferably at least 95% identical to SEQ ID NO: 14. In certain embodiments, Ant2_HapM1 is or comprises a SNP at a position corresponding to position 640 of SEQ ID NO: 15 or a sequence which is at least 80%, preferably at least 90%, more preferably at least 95% identical to SEQ ID NO: 15. In certain embodiments, Ant2_HapM1 is or comprises a SNP at a position corresponding to position 640 of a sequence encoding a protein having a sequence as set forth in SEQ ID NO: 16 or a sequence which is at least 80%, preferably at least 90%, more preferably at least 95% identical to SEQ ID NO: 16. In certain embodiments, Ant2_HapM1 is or comprises a SNP at a position corresponding to position 84 of SEQ ID NO: 26 or a sequence which is at least 80%, preferably at least 90%, more preferably at least 95% identical to SEQ ID NO: 26.

In certain embodiments, Ant2_HapM2 is or comprises a SNP at a position corresponding to position 593492774 (on chromosome 2H) of barley, preferably the barley reference genome Morex (version 3.0). In certain embodiments, Ant_HapM2 is or comprises a SNP at a position corresponding to position 410 of SEQ ID NO: 14, or a sequence which is at least 80%, preferably at least 90%, more preferably at least 95% identical to SEQ ID NO: 14. In certain embodiments, Ant2_HapM2 is or comprises a SNP at a position corresponding to position 76 of SEQ ID NO: 27 or a sequence which is at least 80%, preferably at least 90%, more preferably at least 95% identical to SEQ ID NO: 27.

In certain embodiments, Ant2_HapM3 is or comprises a SNP at a position corresponding to position 593493167 (on chromosome 2H) of barley, preferably the barley reference genome Morex (version 3.0). In certain embodiments, Ant_HapM3 is or comprises a SNP at a position corresponding to position 803 of SEQ ID NO: 14, or a sequence which is at least 80%, preferably at least 90%, more preferably at least 95% identical to SEQ ID NO: 14. In certain embodiments, Ant2_HapM3 is or comprises a SNP at a position corresponding to position 49 of SEQ ID NO: 15 or a sequence which is at least 80%, preferably at least 90%, more preferably at least 95% identical to SEQ ID NO: 15. In certain embodiments, Ant2_HapM3 is or comprises a SNP at a position corresponding to position 49 of a sequence encoding a protein having a sequence as set forth in SEQ ID NO: 16 or a sequence which is at least 80%, preferably at least 90%, more preferably at least 95% identical to SEQ ID NO: 16. In certain embodiments, Ant2_HapM3 is or comprises a SNP at a position corresponding to position 94 of SEQ ID NO: 28 or a sequence which is at least 80%, preferably at least 90%, more preferably at least 95% identical to SEQ ID NO: 28.

In certain embodiments, Ant2_HapM4 is or comprises a SNP at a position corresponding to position 593494160 (on chromosome 2H) of barley, preferably the barley reference genome Morex (version 3.0). In certain embodiments, Ant_HapM4 is or comprises a SNP at a position corresponding to position 1796 of SEQ ID NO: 14, or a sequence which is at least 80%, preferably at least 90%, more preferably at least 95% identical to SEQ ID NO: 14. In certain embodiments, Ant2_HapM4 is or comprises a SNP at a position corresponding to position 88 of SEQ ID NO: 29 or a sequence which is at least 80%, preferably at least 90%, more preferably at least 95% identical to SEQ ID NO: 29.

In certain embodiments, Ant2_HapM5 is or comprises a SNP at a position corresponding to position 593494377 (on chromosome 2H) of barley, preferably the barley reference genome Morex (version 3.0). In certain embodiments, Ant_HapM5 is or comprises a SNP at a position corresponding to position 2013 of SEQ ID NO: 14, or a sequence which is at least 80%, preferably at least 90%, more preferably at least 95% identical to SEQ ID NO: 14. In certain embodiments, Ant2_HapM5 is or comprises a SNP at a position corresponding to position 52 of SEQ ID NO: 30 or a sequence which is at least 80%, preferably at least 90%, more preferably at least 95% identical to SEQ ID NO: 30.

In certain embodiments, Ant2_HapM6 is or comprises a SNP at a position corresponding to position 593495891 (on chromosome 2H) of barley, preferably the barley reference genome Morex (version 3.0). In certain embodiments, Ant_HapM6 is or comprises a SNP at a position corresponding to position 3530 of SEQ ID NO: 14, or a sequence which is at least 80%, preferably at least 90%, more preferably at least 95% identical to SEQ ID NO: 14. In certain embodiments, Ant2_HapM6 is or comprises a SNP at a position corresponding to position 80 of SEQ ID NO: 31 or a sequence which is at least 80%, preferably at least 90%, more preferably at least 95% identical to SEQ ID NO: 31.

In certain embodiments, Ant2_HapM7 is or comprises a SNP at a position corresponding to position 593497594 (on chromosome 2H) of barley, preferably the barley reference genome Morex (version 3.0). In certain embodiments, Ant_HapM7 is or comprises a SNP at a position corresponding to position 4895 of SEQ ID NO: 14, or a sequence which is at least 80%, preferably at least 90%, more preferably at least 95% identical to SEQ ID NO: 14. In certain embodiments, Ant2_HapM7 is or comprises a SNP at a position corresponding to position 1619 of SEQ ID NO: 15 or a sequence which is at least 80%, preferably at least 90%, more preferably at least 95% identical to SEQ ID NO: 15. In certain embodiments, Ant2_HapM7 is or comprises a SNP at a position corresponding to position 1619 of a sequence encoding a protein having a sequence as set forth in SEQ ID NO: 16 or a sequence which is at least 80%, preferably at least 90%, more preferably at least 95% identical to SEQ ID NO: 16. In certain embodiments, Ant2_HapM7 is or comprises a SNP at a position corresponding to position 73 of SEQ ID NO: 32 or a sequence which is at least 80%, preferably at least 90%, more preferably at least 95% identical to SEQ ID NO: 32.

The barley Morex version 3.0 (v3.0 or v3) reference genome are referred to herein (also called barley Morex v3 pseudomolecules reference sequence or reference genome sequence assembly of Barley cultivar Morex v3.0 (July 2020)) was published in Mascher et al., (2021) “Long-read sequence assembly: a technical evaluation in barley”; Plant Cell; 33 (6): 1888-1906 (doi.org/10.1093/plcell/koab077). The sequence can for instance be consulted in the GrainGenes database (wheat.pw.usda.gov/GG3/or wheat.pw.usda.gov/jb/).

The barley Morex version 2.0 (v2.0 or v2) reference genome (also called barley Morex v2 pseudomolecules reference sequence or reference genome sequence assembly of Barley cultivar Morex v2.0) was published in Monat et al., (2019) “TRITEX: chromosome-scale sequence assembly of Triticeae genomes with open-source tools”; Genome Biol; 20, 284 (doi.org/10.1186/s13059-019-1899-5). The sequence can for instance be consulted in the GrainGenes database (wheat.pw.usda.gov/GG3/or wheat.pw.usda.gov/jb/).

In an aspect, the invention relates to a method for identifying and/or selecting a barley plant or plant part, comprising screening for the presence of (a polynucleotide comprising) a SNP (Ant1_HapM1) at a position corresponding to position 71238822 (on chromosome 7H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant1_HapM1) at a position corresponding to position 68464237 (on chromosome 7H) of barley, preferably the barley reference genome Morex (version 2.0). In an aspect, the invention relates to a method for identifying and/or selecting a barley plant or plant part, comprising screening for the presence of (a polynucleotide comprising) a SNP (Ant1_HapM1) at a position corresponding to position 95 of SEQ ID NO: 1, or a sequence which is at least 80%, preferably at least 90%, more preferably at least 95% identical to SEQ ID NO: 1. In an aspect, the invention relates to a method for identifying and/or selecting a barley plant or plant part, comprising screening for the presence of (a polynucleotide comprising) a SNP (Ant1_HapM1) at a position corresponding to position 95 of SEQ ID NO: 7 or a sequence which is at least 80%, preferably at least 90%, more preferably at least 95% identical to SEQ ID NO: 7. In certain embodiments, the methods above further comprise screening for an Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 SNP, preferably comprising screening for one or more molecular marker (allele) selected from one or more of Ant2_M2, Ant2_M3, Ant2_M4, Ant2_M5, Ant2_M6, and/or Ant2_M7 SNP; or screening for molecular marker (allele) Ant2_HapM1 SNP and (i.e. in combination with) one or more of Ant2_HapM2, Ant2_HapM3, Ant2_HapM4, Ant2_HapM5, Ant2_HapM6, and/or Ant2_HapM7 SNP, as described herein elsewhere.

In an aspect, the invention relates to a method for identifying and/or selecting a barley plant or plant part, comprising screening for the presence of (a polynucleotide comprising) a SNP (Ant1_HapM2) at a position corresponding to position 71238454 (on chromosome 7H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant1_HapM2) at a position corresponding to position 68463869 (on chromosome 7H) of barley, preferably the barley reference genome Morex (version 2.0). In an aspect, the invention relates to a method for identifying and/or selecting a barley plant or plant part, comprising screening for the presence of (a polynucleotide comprising) a SNP (Ant1_HapM2) at a position corresponding to position 463 of SEQ ID NO: 1 or a sequence which is at least 80%, preferably at least 90%, more preferably at least 95% identical to SEQ ID NO: 1. In an aspect, the invention relates to a method for identifying and/or selecting a barley plant or plant part, comprising screening for the presence of (a polynucleotide comprising) a SNP (Ant1_HapM2) at a position corresponding to position 224 of SEQ ID NO: 8 or a sequence which is at least 80%, preferably at least 90%, more preferably at least 95% identical to SEQ ID NO: 8. In certain embodiments, the methods above further comprise screening for an Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 SNP, preferably comprising screening for one or more molecular marker (allele) selected from one or more of Ant2_M2, Ant2_M3, Ant2_M4, Ant2_M5, Ant2_M6, and/or Ant2_M7 SNP; or screening for molecular marker (allele) Ant2_HapM1 SNP and (i.e. in combination with) one or more of Ant2_HapM2, Ant2_HapM3, Ant2_HapM4, Ant2_HapM5, Ant2_HapM6, and/or Ant2_HapM7 SNP, as described herein elsewhere.

In an aspect, the invention relates to a method for identifying and/or selecting a barley plant or plant part, comprising screening for the presence of (a polynucleotide comprising) a SNP (Ant1_HapM3) at a position corresponding to position 71238052 (on chromosome 7H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant1_HapM3) at a position corresponding to position 68463467 (on chromosome 7H) of barley, preferably the barley reference genome a SNP (Ant1_HapM3) at a position corresponding to position 865 of SEQ ID NO: 1 or a sequence which is at least 80%, preferably at least 90%, more preferably at least 95% identical to SEQ ID NO: 1. In an aspect, the invention relates to a method for identifying and/or selecting a barley plant or plant part, comprising screening for the presence of (a polynucleotide comprising) a SNP (Ant1_HapM3) at a position corresponding to position 219 of SEQ ID NO: 2 or a sequence which is at least 80%, preferably at least 90%, more preferably at least 95% identical to SEQ ID NO: 2. In an aspect, the invention relates to a method for identifying and/or selecting a barley plant or plant part, comprising screening for the presence of (a polynucleotide comprising) a SNP (Ant1_HapM3) at a position corresponding to position 219 of a sequence encoding a protein having a sequence as set forth in SEQ ID NO: 3 or a sequence which is at least 80%, preferably at least 90%, more preferably at least 95% identical to SEQ ID NO: 3. In an aspect, the invention relates to a method for identifying and/or selecting a barley plant or plant part, comprising screening for the presence of (a polynucleotide comprising) a SNP (Ant1_HapM3) at a position corresponding to position 206 of SEQ ID NO: 9 or a sequence which is at least 80%, preferably at least 90%, more preferably at least 95% identical to SEQ ID NO: 9. In certain embodiments, the methods above further comprise screening for an Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 SNP, preferably comprising screening for one or more molecular marker (allele) selected from one or more of Ant2_M2, Ant2_M3, Ant2_M4, Ant2_M5, Ant2_M6, and/or Ant2_M7 SNP; or screening for molecular marker (allele) Ant2_HapM1 SNP and (i.e. in combination with) one or more of Ant2_HapM2, Ant2_HapM3, Ant2_HapM4, Ant2_HapM5, Ant2_HapM6, and/or Ant2_HapM7 SNP, as described herein elsewhere.

In an aspect, the invention relates to a method for identifying and/or selecting a barley plant or plant part, comprising screening for the presence of (a polynucleotide comprising) a SNP (Ant1_HapM4) at a position corresponding to position 71237825 (on chromosome 7H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant1_HapM4) at a position corresponding to position 68463240 (on chromosome 7H) of barley, preferably the barley reference genome Morex (version 2.0). In an aspect, the invention relates to a method for identifying and/or selecting a SNP (Ant1_HapM4) at a position corresponding to position 1092 of SEQ ID NO: 1 or a sequence which is at least 80%, preferably at least 90%, more preferably at least 95% identical to SEQ ID NO: 1. In an aspect, the invention relates to a method for identifying and/or selecting a barley plant or plant part, comprising screening for the presence of (a polynucleotide comprising) a SNP (Ant1_HapM4) at a position corresponding to position 446 of SEQ ID NO: 2 or a sequence which is at least 80%, preferably at least 90%, more preferably at least 95% identical to SEQ ID NO: 2. In an aspect, the invention relates to a method for identifying and/or selecting a barley plant or plant part, comprising screening for the presence of (a polynucleotide comprising) a SNP (Ant1_HapM4) at a position corresponding to position 446 of a sequence encoding a protein having a sequence as set forth in SEQ ID NO: 3 or a sequence which is at least 80%, preferably at least 90%, more preferably at least 95% identical to SEQ ID NO: 3. In an aspect, the invention relates to a method for identifying and/or selecting a barley plant or plant part, comprising screening for the presence of (a polynucleotide comprising) a SNP (Ant1_HapM4) at a position corresponding to position 73 of SEQ ID NO: 10 or a sequence which is at least 80%, preferably at least 90%, more preferably at least 95% identical to SEQ ID NO: 10. In certain embodiments, the methods above further comprise screening for an Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 SNP, preferably comprising screening for one or more molecular marker (allele) selected from one or more of Ant2_M2, Ant2_M3, Ant2_M4, Ant2_M5, Ant2_M6, and/or Ant2_M7 SNP; or screening for molecular marker (allele) Ant2_HapM1 SNP and (i.e. in combination with) one or more of Ant2_HapM2, Ant2_HapM3, Ant2_HapM4, Ant2_HapM5, Ant2_HapM6, and/or Ant2_HapM7 SNP, as described herein elsewhere.

In an aspect, the invention relates to a method for identifying and/or selecting a barley plant or plant part, comprising screening for the presence of (a polynucleotide comprising) a SNP (Ant1_HapM5) at a position corresponding to position 71237633 (on chromosome 7H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant1_HapM5) at a position corresponding to position 68463048 (on chromosome 7H) of barley, preferably the barley reference genome a SNP (Ant1_HapM5) at a position corresponding to position 1284 of SEQ ID NO: 1 or a sequence which is at least 80%, preferably at least 90%, more preferably at least 95% identical to SEQ ID NO: 1. In an aspect, the invention relates to a method for identifying and/or selecting a barley plant or plant part, comprising screening for the presence of (a polynucleotide comprising) a SNP (Ant1_HapM5) at a position corresponding to position 638 of SEQ ID NO: x2 or a sequence which is at least 80%, preferably at least 90%, more preferably at least 95% identical to SEQ ID NO: 2. In an aspect, the invention relates to a method for identifying and/or selecting a barley plant or plant part, comprising screening for the presence of (a polynucleotide comprising) a SNP (Ant1_HapM5) at a position corresponding to position 638 of a sequence encoding a protein having a sequence as set forth in SEQ ID NO: 3 or a sequence which is at least 80%, preferably at least 90%, more preferably at least 95% identical to SEQ ID NO: 3. In an aspect, the invention relates to a method for identifying and/or selecting a barley plant or plant part, comprising screening for the presence of (a polynucleotide comprising) a SNP (Ant1_HapM5) at a position corresponding to position 145 of SEQ ID NO: 11 or a sequence which is at least 80%, preferably at least 90%, more preferably at least 95% identical to SEQ ID NO: 11. In certain embodiments, the methods above further comprise screening for an Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 SNP, preferably comprising screening for one or more molecular marker (allele) selected from one or more of Ant2_M2, Ant2_M3, Ant2_M4, Ant2_M5, Ant2_M6, and/or Ant2_M7 SNP; or screening for molecular marker (allele) Ant2_HapM1 SNP and (i.e. in combination with) one or more of Ant2_HapM2, Ant2_HapM3, Ant2_HapM4, Ant2_HapM5, Ant2_HapM6, and/or Ant2_HapM7 SNP, as described herein elsewhere.

In an aspect, the invention relates to a method for identifying and/or selecting a barley plant or plant part, comprising screening for the presence of (a polynucleotide comprising) a SNP (Ant1_HapM6) at a position corresponding to position 71237608 (on chromosome 7H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant1_HapM6) at a position corresponding to position 68463023 (on chromosome 7H) of barley, preferably the barley reference genome a SNP (Ant1_HapM6) at a position corresponding to position 1309 of SEQ ID NO: 1 or a sequence which is at least 80%, preferably at least 90%, more preferably at least 95% identical to SEQ ID NO: 1. In an aspect, the invention relates to a method for identifying and/or selecting a barley plant or plant part, comprising screening for the presence of (a polynucleotide comprising) a SNP (Ant1_HapM6) at a position corresponding to position 663 of SEQ ID NO: 2 or a sequence which is at least 80%, preferably at least 90%, more preferably at least 95% identical to SEQ ID NO: 2. In an aspect, the invention relates to a method for identifying and/or selecting a barley plant or plant part, comprising screening for the presence of (a polynucleotide comprising) a SNP (Ant1_HapM6) at a position corresponding to position 663 of a sequence encoding a protein having a sequence as set forth in SEQ ID NO: 3 or a sequence which is at least 80%, preferably at least 90%, more preferably at least 95% identical to SEQ ID NO: 3. In an aspect, the invention relates to a method for identifying and/or selecting a barley plant or plant part, comprising screening for the presence of (a polynucleotide comprising) a SNP (Ant1_HapM6) at a position corresponding to position 50 of SEQ ID NO: 12 or a sequence which is at least 80%, preferably at least 90%, more preferably at least 95% identical to SEQ ID NO: 12. In certain embodiments, the methods above further comprise screening for an Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 SNP, preferably comprising screening for one or more molecular marker (allele) selected from one or more of Ant2_M2, Ant2_M3, Ant2_M4, Ant2_M5, Ant2_M6, and/or Ant2_M7 SNP; or screening for molecular marker (allele) Ant2_HapM1 SNP and (i.e. in combination with) one or more of Ant2_HapM2, Ant2_HapM3, Ant2_HapM4, Ant2_HapM5, Ant2_HapM6, and/or Ant2_HapM7 SNP, as described herein elsewhere.

In an aspect, the invention relates to a method for identifying and/or selecting a barley plant or plant part, comprising screening for the presence of (a polynucleotide comprising) a SNP (Ant2_M1) at a position corresponding to position 591324765 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_M1) at a position corresponding to position 599993462 (on chromosome 2H) of barley, preferably the barley reference genome Morex (version 2.0). Ant2Ant2Ant2In an aspect, the invention relates to a method for identifying and/or selecting a barley plant or plant part, comprising screening for the presence of (a polynucleotide comprising) a SNP (Ant2_M1) at a position corresponding to position 101 of SEQ ID NO: 13 or a sequence which is at least 80%, preferably at least 90%, more preferably at least 95% identical to SEQ ID NO: 13. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_M2) at a position corresponding to position 593492855 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_M2) at a position corresponding to position 491 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_M2) at a position corresponding to position 46 of SEQ ID NO: 20 or a sequence which is at least 80%, preferably at least 90%, more preferably at least 95% identical to SEQ ID NO: 20. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_M3) at a position corresponding to position 593493961 (on chromosome 2H) of barley, preferably the barley reference genome Morex (version 3.0 In an aspect, the invention relates to a method for identifying and/or selecting a barley plant or plant part, comprising screening for the presence of (a polynucleotide comprising) a SNP (Ant2_M3) at a position corresponding to position 1597 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_M3) at a position corresponding to position 96 of SEQ ID NO: 21 or a sequence which is at least 80%, preferably at least 90%, more preferably at least 95% identical to SEQ ID NO: 21. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_M4) at a position corresponding to position 593494314 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_M4) at a position corresponding to position 1950 of SEQ ID NO: 14, or a sequence which is at least 80%, preferably at least 90%, more preferably at least 95% identical to SEQ ID NO: 14 In an aspect, the invention relates to a method for identifying and/or selecting a barley plant or plant part, comprising screening for the presence of (a polynucleotide comprising) a SNP (Ant2_M4) at a position corresponding to position 64 of SEQ ID NO: 22 or a sequence which is at least 80%, preferably at least 90%, more preferably at least 95% identical to SEQ ID NO: 22. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_M5) at a position corresponding to position 593494569 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_M5) at a position corresponding to position 2207 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_M5) at a position corresponding to position 73 of SEQ ID NO: 23 or a sequence which is at least 80%, preferably at least 90%, more preferably at least 95% identical to SEQ ID NO: 23. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_M6) at a position corresponding to position 593495307 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_M6) at a position corresponding to position 2946 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_M6) at a position corresponding to position 101 of SEQ ID NO: 24 or a sequence which is at least 80%, preferably at least 90%, more preferably at least 95% identical to SEQ ID NO: 24. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_M7) at a position corresponding to position 593496606 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_M7) at a position corresponding to position 3907 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_M7) at a position corresponding to position 804 of SEQ ID NO: 15 or a sequence which is at least 80%, preferably at least 90%, more preferably at least 95% identical to SEQ ID NO: 15. In an aspect, the invention relates to a method for identifying and/or selecting a barley plant or plant part, comprising screening for the presence of (a polynucleotide comprising) a SNP (Ant2_M7) at a position corresponding to position 804 of a sequence encoding a protein having a sequence as set forth in SEQ ID NO: 16 or a sequence which is at least 80%, preferably at least 90%, more preferably at least 95% identical to SEQ ID NO: 16. In an aspect, the invention relates to a method for identifying and/or selecting a barley plant or plant part, comprising screening for the presence of (a polynucleotide comprising) a SNP (Ant2_M7) at a position corresponding to position 72 of SEQ ID NO: 25 or a sequence which is at least 80%, preferably at least 90%, more preferably at least 95% identical to SEQ ID NO: 25. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_HapM1) at a position corresponding to position 593496442 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_HapM1) at a position corresponding to position 3743 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_HapM1) at a position corresponding to position 640 of SEQ ID NO: 15 or a sequence which is at least 80%, preferably at least 90%, more preferably at least 95% identical to SEQ ID NO: 15. In an aspect, the invention relates to a method for identifying and/or selecting a barley plant or plant part, comprising screening for the presence of (a polynucleotide comprising) a SNP (Ant2_HapM1) at a position corresponding to position 640 of a sequence encoding a protein having a sequence as set forth in SEQ ID NO: 16 or a sequence which is at least 80%, preferably at least 90%, more preferably at least 95% identical to SEQ ID NO: 16. In an aspect, the invention relates to a method for identifying and/or selecting a barley plant or plant part, comprising screening for the presence of (a polynucleotide comprising) a SNP (Ant2_HapM1) at a position corresponding to position 84 of SEQ ID NO: 26 or a sequence which is at least 80%, preferably at least 90%, more preferably at least 95% identical to SEQ ID NO: 26. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_HapM2) at a position corresponding to position 593492774 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_HapM2) at a position corresponding to position 410 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_HapM2) at a position corresponding to position 76 of SEQ ID NO: 27 or a sequence which is at least 80%, preferably at least 90%, more preferably at least 95% identical to SEQ ID NO: 27. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_HapM3) at a position corresponding to position 593493167 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_HapM3) at a position corresponding to position 803 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_Hap31) at a position corresponding to position 49 of SEQ ID NO: 15 or a sequence which is at least 80%, preferably at least 90%, more preferably at least 95% identical to SEQ ID NO: 15. In an aspect, the invention relates to a method for identifying and/or selecting a barley plant or plant part, comprising screening for the presence of (a polynucleotide comprising) a SNP (Ant2_HapM3) at a position corresponding to position 49 of a sequence encoding a protein having a sequence as set forth in SEQ ID NO: 16 or a sequence which is at least 80%, preferably at least 90%, more preferably at least 95% identical to SEQ ID NO: 16. In an aspect, the invention relates to a method for identifying and/or selecting a barley plant or plant part, comprising screening for the presence of (a polynucleotide comprising) a SNP (Ant2_HapM3) at a position corresponding to position 94 of SEQ ID NO: 28 or a sequence which is at least 80%, preferably at least 90%, more preferably at least 95% identical to SEQ ID NO: 28. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_HapM4) at a position corresponding to position 593494160 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_HapM4) at a position corresponding to position 1796 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_HapM4) at a position corresponding to position 88 of SEQ ID NO: 29 or a sequence which is at least 80%, preferably at least 90%, more preferably at least 95% identical to SEQ ID NO: 29. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_HapM5) at a position corresponding to position 593494377 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_HapM5) at a position corresponding to position 2013 of SEQ ID NO: 14, or a sequence which is at least 80%, preferably at least 90%, more preferably at least 95% identical to SEQ ID NO: 14 In an aspect, the invention relates to a method for identifying and/or selecting a barley plant or plant part, comprising screening for the presence of (a polynucleotide comprising) a SNP (Ant2_HapM5) at a position corresponding to position 52 of SEQ ID NO: 30 or a sequence which is at least 80%, preferably at least 90%, more preferably at least 95% identical to SEQ ID NO: 30. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_HapM6) at a position corresponding to position 593495891 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_HapM6) at a position corresponding to position 3530 of SEQ ID NO: 14, or a sequence which is at least 80%, preferably at least 90%, more preferably at least 95% identical to SEQ ID NO: 14 In an aspect, the invention relates to a method for identifying and/or selecting a barley plant or plant part, comprising screening for the presence of (a polynucleotide comprising) a SNP (Ant2_HapM6) at a position corresponding to position 80 of SEQ ID NO: 31 or a sequence which is at least 80%, preferably at least 90%, more preferably at least 95% identical to SEQ ID NO: 31. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In an aspect, the invention relates to a method for identifying and/or selecting a barley plant or plant part, comprising screening for the presence of (a polynucleotide comprising) a SNP (Ant2_HapM7) at a position corresponding to position 593497594 (on chromosome 2H) of barley, preferably the barley reference genome Morex (version 3.0). In an aspect, the invention relates to a method for identifying and/or selecting a barley plant or plant part, comprising screening for the presence of (a polynucleotide comprising) a SNP (Ant2_HapM7) at a position corresponding to position 4895 of SEQ ID NO: 14, or a sequence which is at least 80%, preferably at least 90%, more preferably at least 95% identical to SEQ ID NO: 14. In an aspect, the invention relates to a method for identifying and/or selecting a barley plant or plant part, comprising screening for the presence of (a polynucleotide comprising) a SNP (Ant2_HapM7) at a position corresponding to position 1619 of SEQ ID NO: 15 or a sequence which is at least 80%, preferably at least 90%, more preferably at least 95% identical to SEQ ID NO: 15. In an aspect, the invention relates to a method for identifying and/or selecting a barley plant or plant part, comprising screening for the presence of (a polynucleotide comprising) a SNP (Ant2_HapM7) at a position corresponding to position 1619 of a sequence encoding a protein having a sequence as set forth in SEQ ID NO: 16 or a sequence which is at least 80%, preferably at least 90%, more preferably at least 95% identical to SEQ ID NO: 16. In an aspect, the invention relates to a method for identifying and/or selecting a barley plant or plant part, comprising screening for the presence of (a polynucleotide comprising) a SNP (Ant2_HapM7) at a position corresponding to position 73 of SEQ ID NO: 32 or a sequence which is at least 80%, preferably at least 90%, more preferably at least 95% identical to SEQ ID NO: 32. In certain embodiments, the methods above further comprise screening for any one or more, preferably all, of an Ant1_HapM1, Ant1_HapM2, Ant1_HapM3, Ant1_HapM4, Ant1_HapM5, and/or Ant1_HapM6 SNP, as described herein elsewhere.

In certain embodiments, molecular markers Ant1_HapM1, Ant1_HapM2, Ant1_HapM3, Ant1_HapM4, Ant1_HapM5, Ant1_HapM6, Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are SNPs. In certain embodiments, Ant1_HapM1, Ant1_HapM2, Ant1_HapM3, Ant1_HapM4, Ant1_HapM5, Ant1_HapM6, Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are oligonucleotides or polynucleotides. In certain embodiments, Ant1_HapM1, Ant1_HapM2, Ant1_HapM3, Ant1_HapM4, Ant1_HapM5, Ant1_HapM6, Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are (SNPs) comprised in oligonucleotides or polynucleotides. The skilled person will understand that if the molecular markers are SNPs, the identification of the SNPs will encompass the nucleotide context in which the SNPs occur, i.e., the (naturally occurring) upstream and/or downstream nucleotides are taken into account. An SNP is never evaluated in isolation, but is evaluated in the context of a larger sequence, which allows to unambiguously identify the SNP, i.e., contiguous (naturally occurring) upstream or downstream nucleotides, preferably, at least 10, more preferably at least 15, most preferably at least 18 upstream and/or downstream nucleotides (either upstream or downstream or combined upstream and downstream). Contiguous upstream or downstream nucleotides can be and preferably are nucleotides occurring naturally upstream or downstream of the SNP, such as for instance genomic sequences contiguously upstream or downstream of the genomic location of the SNP. Alternatively, in case of a SNP by reference to for instance a coding sequence or cDNA, the contiguous upstream or downstream nucleotides need not necessarily be genomically naturally occurring upstream or downstream of the SNP, in particular in case of exon-bridging nucleotides. In such case, the contiguous nucleotides are to be regarded contiguous and adjacent to the SNP in the coding sequence only, but not (necessarily) in the genomic sequence.

In certain embodiments, Ant1_HapM1, Ant1_HapM2, Ant1_HapM3, Ant1_HapM4, Ant1_HapM5, Ant1_HapM6, Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are comprised in oligonucleotides or polynucleotides of at least 10 nucleotides, preferably at least 15 nucleotides, more preferably at least 18 nucleotides. In certain embodiments, Ant1_HapM1, Ant1_HapM2, Ant1_HapM3, Ant1_HapM4, Ant1_HapM5, Ant1_HapM6, Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are comprised in oligonucleotides or polynucleotides of at most 20000 nucleotides, preferably at most 10000 nucleotides, more preferably at most 5000 nucleotides. In certain embodiments, Ant1_HapM1, Ant1_HapM2, Ant1_HapM3, Ant1_HapM4, Ant1_HapM5, Ant1_HapM6, Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are comprised in oligonucleotides or polynucleotides of at most 250 nucleotides, preferably at most 100 nucleotides, more preferably at most 50 nucleotides.

In certain embodiments, Ant1_HapM1, Ant1_HapM2, Ant1_HapM3, Ant1_HapM4, Ant1_HapM5, Ant1_HapM6, Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are comprised in oligonucleotides or polynucleotides of at least 10 nucleotides, preferably at least 15 nucleotides, more preferably at least 18 nucleotides; and are comprised in oligonucleotides or polynucleotides of at most 20000 nucleotides, preferably at most 10000 nucleotides, more preferably at most 5000 nucleotides.

In certain embodiments, Ant1_HapM1, Ant1_HapM2, Ant1_HapM3, Ant1_HapM4, Ant1_HapM5, Ant1_HapM6, Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are comprised in oligonucleotides or polynucleotides of at least 10 nucleotides, preferably at least 15 nucleotides, more preferably at least 18 nucleotides; and are comprised in oligonucleotides or polynucleotides of at most 250 nucleotides, preferably at most 100 nucleotides, more preferably at most 50 nucleotides.

In certain embodiments, the polynucleic acids/polynucleotides as described herein have a length of at most 1 Mb, preferably at most 500 kb, more preferably at most 200 kb, most preferably at most 100 kb. In certain embodiments, the polynucleic acids/polynucleotides as described herein have a length of at most 20 kb, preferably at most 10 kb, more preferably at most 5 kb. In certain embodiments, the polynucleic acids/polynucleotides as described herein have a length of at most 250 bp, preferably at most 100 bp, more preferably at most 50 bp.

In certain embodiments, the polynucleic acids/polynucleotides as described herein have a length of a at least 15 nucleotides, preferably at least 18 nucleotides, more preferably at least 20 nucleotides.

In certain embodiments, the polynucleic acids/polynucleotides as described herein have a length of at most 1 Mb, preferably at most 500 kb, more preferably at most 200 kb, most preferably at most 100 kb, and at least 15 nucleotides, preferably at least 18 nucleotides, more preferably at least 20 nucleotides. In certain embodiments, the polynucleic acids/polynucleotides as described herein have a length of at most 20 kb, preferably at most 10 kb, more preferably at most 5 kb, and at least 15 nucleotides, preferably at least 18 nucleotides, more preferably at least 20 nucleotides. In certain embodiments, the polynucleic acids/polynucleotides as described herein have a length of at most 250 bp, preferably at most 100 bp, more preferably at most 50 bp, and at least 15 nucleotides, preferably at least 18 nucleotides, more preferably at least 20 nucleotides.

In preferred embodiments, the polynucleotides, loci, gene, marker etc. comprising Ant1_HapM1, Ant1_HapM2, Ant1_HapM3, Ant1_HapM4, Ant1_HapM5, and/or Ant1_HapM6 are located on barley chromosome 7H, in particular when referring to native locations of such polynucleotides.

This need however not always be the case. For instance, in case of transgenic introduction of the polynucleotides, loci, gene, etc., a different or non-native location may result.

In preferred embodiments, the polynucleotides, loci, gene, marker etc. comprising Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7 are located on barley chromosome 2H, in particular when referring to native locations of such polynucleotides. This need however not always be the case. For instance, in case of transgenic introduction of the polynucleotides, loci, gene, etc., a different or non-native location may result.

In certain embodiments, the SNPs as referred to herein may be as indicated in Table A below. The SNP identity associated with “Purple” corresponds to the SNP in an Ant1 gene allele or in or associated/linked with the Ant2 gene allele (or coding sequence thereof) responsible for or required for purple-grained barley. The SNP identity associated with “non-purple” corresponds to the SNP in an Ant1 gene allele or in or associated/linked with the Ant2 gene allele (or coding sequence thereof) responsible for or required for the absence of purple grains in barley.

TABLE A
(V = not T; D = not C; H = not G). Positions on Morex v2/v3 for Ant1
markers are on chromosome 7H. Positions on Morex v2/v3 for Ant2 markers
are on chromosome 2H. This table includes sequences which are at least 80%,
preferably at least 90%, more preferably at least 95% identical to the indicated
SEQ ID NOs, preferably either comprising one or more, preferably all (i.e. the
Ant1 “haplotype), of the Ant1 SNPs or the Ant2 SNPs, or any one or more of
the SNPs of Ant2_M2, Ant2_M3, Ant2_M4, Ant2_M5, Ant2_M6, and
Ant2_M7, or the SNP of Ant2_HapM1 combined with any one or more SNP
of Ant2_HapM2, Ant2_HapM3, Ant2_HapM4, Ant2_HapM5, Ant2_HapM6,
or Ant2_HapM7 (i.e. Ant2 “haplotypes”).

| | SNP | | | |
|---|---|---|---|---|
| | Purple | | Non-purple | |
| Marker | identity | position | identity | position |
| Ant1_HapM1 | T | 71238822 of Morex v3 | V or G | 71238822 of Morex v3 |
| | | 68464237of Morex v2 | | 68464237 of Morex v2 |
| | | 95 of SEQ ID NO: 1 | | 95 of SEQ ID NO: 4 |
| | | 95 of SEQ ID NO: 7 | | 95 of SEQ ID NO: 7 |
| Ant1_HapM2 | C | 71238454 of Morex v3 | D or G | 71238454 of Morex v3 |
| | | 68463869 of Morex v2 | | 68463869 of Morex v2 |
| | | 463 of SEQ ID NO: 1 | | 464 of SEQ ID NO: 4 |
| | | 224 of SEQ ID NO: 8 | | 224 of SEQ ID NO: 8 |
| Ant1_HapM3 | C | 71238052 of Morex v3 | D or G | 71238052 of Morex v3 |
| | | 68463467 of Morex v2 | | 68463467 of Morex v2 |
| | | 865 of SEQ ID NO: 1 | | 866 of SEQ ID NO: 4 |
| | | 219 of SEQ ID NO: 2 | | 219 of SEQ ID NO: 5 |
| | | 206 of SEQ ID NO: 9 | | 206 of SEQ ID NO: 9 |
| Ant1_HapM4 | C | 71237825 of Morex v3 | D or T | 71237825 of Morex v3 |
| | | 68463240 of Morex v2 | | 68463240 of Morex v2 |
| | | 1092 of SEQ ID NO: 1 | | 1093 of SEQ ID NO: 4 |
| | | 446 of SEQ ID NO: 2 | | 446 of SEQ ID NO: 5 |
| | | 73 of SEQ ID NO: 10 | | 73 of SEQ ID NO: 10 |
| Ant1_HapM5 | C | 71237633 of Morex v3 | D or A | 71237633 of Morex v3 |
| | | 68463048 of Morex v2 | | 68463048 of Morex v2 |
| | | 1284 of SEQ ID NO: 1 | | 1285 of SEQ ID NO: 4 |
| | | 638 of SEQ ID NO: 2 | | 638 of SEQ ID NO: 5 |
| | | 145 of SEQ ID NO: 11 | | 145 of SEQ ID NO: 11 |
| Ant1_HapM6 | G | 71237608 of Morex v3 | H or A | 71237608 of Morex v3 |
| | | 68463023 of Morex v2 | | 68463023 of Morex v2 |
| | | 1309 of SEQ ID NO: 1 | | 1310 of SEQ ID NO: 4 |
| | | 663 of SEQ ID NO: 2 | | 663 of SEQ ID NO: 5 |
| | | 50 of SEQ ID NO: 12 | | 50 of SEQ ID NO: 12 |
| Ant2_M1 | C | 591324765 of Morex v3 | D or T | 591324765 of Morex v3 |
| | | 599993462 of Morex v2 | | 599993462 of Morex v2 |
| | | 101 of SEQ ID NO: 13 | | 101 of SEQ ID NO: 13 |
| Ant2_M2 | G | 593492855 of Morex v3 | H or T | 593492855 of Morex v3 |
| | | 491 of SEQ ID NO: 14 | | 1284 of SEQ ID NO: 17 |
| | | 46 of SEQ ID NO: 20 | | 46 of SEQ ID NO: 20 |
| Ant2_M3 | C | 593493961 of Morex v3 | D or T | 593493961 of Morex v3 |
| | | 1597 of SEQ ID NO: 14 | | 2381 of SEQ ID NO: 17 |
| | | 96 of SEQ ID NO: 21 | | 96 of SEQ ID NO: 21 |
| Ant2_M4 | T | 593494314 of Morex v3 | V or G | 593494314 of Morex v3 |
| | | 1950 of SEQ ID NO: 14 | | 2734 of SEQ ID NO: 17 |
| | | 64 of SEQ ID NO: 22 | | 64 of SEQ ID NO: 22 |
| Ant2_M5 | G | 593494569 of Morex v3 | H or A | 593494569 of Morex v3 |
| | | 2207 of SEQ ID NO: 14 | | 2989 of SEQ ID NO: 17 |
| | | 73 of SEQ ID NO: 23 | | 73 of SEQ ID NO: 23 |
| Ant2_M6 | T | 593495307 of Morex v3 | V or C | 593495307 of Morex v3 |
| | | 2946 of SEQ ID NO: 14 | | 3729 of SEQ ID NO: 17 |
| | | 101 of SEQ ID NO: 24 | | 101 of SEQ ID NO: 24 |
| Ant2_M7 | A | 593496606 of Morex v3 | B or C | 593496606 of Morex v3 |
| | | 3907 of SEQ ID NO: 14 | | 4690 of SEQ ID NO: 17 |
| | | 804 of SEQ ID NO: 15 | | 804 of SEQ ID NO: 18 |
| | | 72 of SEQ ID NO: 25 | | 72 of SEQ ID NO: 25 |
| Ant2_HapM1 | G | 593496442 of Morex v3 | H or A | 593496442 of Morex v3 |
| | | 3743 of SEQ ID NO: 14 | | 4526 of SEQ ID NO: 17 |
| | | 640 of SEQ ID NO: 15 | | 640 of SEQ ID NO: 18 |
| | | 84 of SEQ ID NO: 26 | | 84 of SEQ ID NO: 26 |
| Ant2_HapM2 | C | 593492774 of Morex v3 | D or G | 593492774 of Morex v3 |
| | | 410 of SEQ ID NO: 14 | | 1203 of SEQ ID NO: 17 |
| | | 76 of SEQ ID NO: 27 | | 76 of SEQ ID NO: 27 |
| Ant2_HapM3 | A | 593493167 of Morex v3 | B or G | 593493167 of Morex v3 |
| | | 803 of SEQ ID NO: 14 | | 1596 of SEQ ID NO: 17 |
| | | 49 of SEQ ID NO: 15 | | 49 of SEQ ID NO: 18 |
| | | 94 of SEQ ID NO: 28 | | 94 of SEQ ID NO: 28 |
| Ant2_HapM4 | T | 593494160 of Morex v3 | V or C | 593494160 of Morex v3 |
| | | 1796 of SEQ ID NO: 14 | | 2580 of SEQ ID NO: 17 |
| | | 88 of SEQ ID NO: 29 | | 88 of SEQ ID NO: 29 |
| Ant2_HapM5 | A | 593494377 of Morex v3 | B or T | 593494377 of Morex v3 |
| | | 2013 of SEQ ID NO: 14 | | 2797 of SEQ ID NO: 17 |
| | | 52 of SEQ ID NO: 30 | | 52 of SEQ ID NO: 30 |
| Ant2_HapM6 | G | 593495891 of Morex v3 | H or A | 593495891 of Morex v3 |
| | | 3530 of SEQ ID NO: 14 | | 4313 of SEQ ID NO: 17 |
| | | 80 of SEQ ID NO: 31 | | 80 of SEQ ID NO: 31 |
| Ant2_HapM7 | C | 593497594 of Morex v3 | D or G | 593497594 of Morex v3 |
| | | 4895 of SEQ ID NO: 14 | | 5676 of SEQ ID NO: 17 |
| | | 1619 of SEQ ID NO: 15 | | 1619 of SEQ ID NO: 18 |
| | | 73 of SEQ ID NO: 32 | | 73 of SEQ ID NO: 32 |

A barley plant or plant part may therefore be identified as carrying an Ant1 allele responsible for or required for purple grained barley (which may be genotypically present, but may or may not be phenotypically present, as grain color. In particular pericarp and hull (i.e., palea and lemma) color is maternally inherited, and hence depends on the genotype of the female parent plant, as described herein elsewhere) if one or more of a “Purple” Ant1 SNP (allele), Ant1 gene (allele) or coding sequence thereof, or molecular marker (allele). QTL (allele), or locus (allele) of Table A, preferably all, is present or identified. A barley plant or plant part may therefore be identified as carrying an Ant1 allele responsible for or required for non-purple grained barley (which may be genotypically present, but may or may not be phenotypically present, as grain color, in particular pericarp and hull (i.e., palea and lemma) color is maternally inherited, and hence depends on the genotype of the female parent plant, as described herein elsewhere) if one or more of a “Non-purple” Ant1 SNP of Table A is identified (a single SNP may suffice). A barley plant or plant part may also be identified as carrying an Ant2 allele responsible for or required for purple grained barley if a “Purple” Ant2 SNP of Table A is present or identified. A barley plant or plant part may therefore be identified as carrying an Ant2 allele responsible for or required for non-purple grained barley if a “Non-purple” Ant2 SNP of Table A is identified.

Preferably, reference herein to a polynucleotide, molecular marker (allele), Ant1 gene (allele) (or coding sequence thereof), Ant2 gene (allele) (or coding sequence thereof), haplotype, or SNP (allele) according to the invention relates to “purple” Ant1 or Ant2 (associated) polynucleotides, sequences, markers, haplotype, or SNPs, as provided in Table A. Such polynucleotide, molecular marker (allele), Ant1 gene (allele) (or coding sequence thereof), Ant2 gene (allele) (or coding sequence thereof), haplotype, or SNP (allele) are also indicated in Table B.

Collectively, the sequences or SNPs listed in Table A as “Purple” may be referred to as polynucleotides or comprised in polynucleotides, genes. QTLs, loci, Molecular markers, etc. associated with Ant1 or Ant2 purple color alleles. Collectively, the sequences or SNPs listed in Table A as “Non-purple” may be referred to as polynucleotides or comprised in polynucleotides, genes, QTLs, loci, Molecular markers, etc. associated with Ant1 or Ant2 non-purple color alleles.

In certain embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
T at a position corresponding to position 71238822 bp (on chromosome 7H) of reference genome Barley Morex v3.0;
C at a position corresponding to position 71238454 bp (on chromosome 7H) of reference genome Barley Morex v3.0;
C at a position corresponding to position 71238052 bp (on chromosome 7H) of reference genome Barley Morex v3.0;
C at a position corresponding to position 71237825 bp (on chromosome 7H) of reference genome Barley Morex v3.0;
C at a position corresponding to position 71237633 bp (on chromosome 7H) of reference genome Barley Morex v3.0; and/or
G at a position corresponding to position 71237608 bp (on chromosome 7H) of reference genome Barley Morex v3.0.

In certain embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
T at a position corresponding to position 68464237 bp (on chromosome 7H) of reference genome Barley Morex v2.0;
C at a position corresponding to position 68463869 bp (on chromosome 7H) of reference genome Barley Morex v2.0;
C at a position corresponding to position 68463467 bp (on chromosome 7H) of reference genome Barley Morex v2.0;
C at a position corresponding to position 68463240 bp (on chromosome 7H) of reference genome Barley Morex v2.0;
C at a position corresponding to position 68463048 bp (on chromosome 7H) of reference genome Barley Morex v2.0; and/or
G at a position corresponding to position 68463023 bp (on chromosome 7H) of reference genome Barley Morex v2.0.

In certain embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
T at a position corresponding to position 95 of SEQ ID NO: 1;
C at a position corresponding to position 463 of SEQ ID NO: 1;
C at a position corresponding to position 865 of SEQ ID NO: 1;
C at a position corresponding to position 1092 of SEQ ID NO: 1;
C at a position corresponding to position 1284 of SEQ ID NO: 1; and/or
G at a position corresponding to position 1309 of SEQ ID NO: 1.

In certain other embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises or lacks one or more SNP which is
C at a position corresponding to position 219 of SEQ ID NO: 2;
C at a position corresponding to position 446 of SEQ ID NO: 2;
C at a position corresponding to position 638 of SEQ ID NO: 2; and/or
G at a position corresponding to position 663 of SEQ ID NO: 2.

In certain other embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
C at a position corresponding to position 219 of a coding sequence encoding a protein having a sequence as set forth in SEQ ID NO: 3;
C at a position corresponding to position 446 of a coding sequence encoding a protein having a sequence as set forth in SEQ ID NO: 3;
C at a position corresponding to position 638 of a coding sequence encoding a protein having a sequence as set forth in SEQ ID NO: 3; and/or
G at a position corresponding to position 663 of a coding sequence encoding a protein having a sequence as set forth in SEQ ID NO: 3.

In certain other embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
T at a position corresponding to position 95 of SEQ ID NO: 7;
C at a position corresponding to position 224 of SEQ ID NO: 8;
C at a position corresponding to position 206 of SEQ ID NO: 9;
C at a position corresponding to position 73 of SEQ ID NO: 10;
C at a position corresponding to position 145 of SEQ ID NO: 11; and/or
G at a position corresponding to position 50 of SEQ ID NO: 12.

In certain embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) does not comprise or lacks one or more SNP which is
T at a position corresponding to position 71238822 bp (on chromosome 7H) of reference genome Barley Morex v3.0;
C at a position corresponding to position 71238454 bp (on chromosome 7H) of reference genome Barley Morex v3.0;
C at a position corresponding to position 71238052 bp (on chromosome 7H) of reference genome Barley Morex v3.0;
C at a position corresponding to position 71237825 bp (on chromosome 7H) of reference genome Barley Morex v3.0;
C at a position corresponding to position 71237633 bp (on chromosome 7H) of reference genome Barley Morex v3.0; and/or
G at a position corresponding to position 71237608 bp (on chromosome 7H) of reference genome Barley Morex v3.0.

In certain embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) does not comprise or lacks one or more SNP which is
T at a position corresponding to position 68464237 bp (on chromosome 7H) of reference genome Barley Morex v2.0;
C at a position corresponding to position 68463869 bp (on chromosome 7H) of reference genome Barley Morex v2.0;
C at a position corresponding to position 68463467 bp (on chromosome 7H) of reference genome Barley Morex v2.0;
C at a position corresponding to position 68463240 bp (on chromosome 7H) of reference genome Barley Morex v2.0;
C at a position corresponding to position 68463048 bp (on chromosome 7H) of reference genome Barley Morex v2.0; and/or
G at a position corresponding to position 68463023 bp (on chromosome 7H) of reference genome Barley Morex v2.0.

In certain embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) does not comprise or lacks one or more SNP which is
T at a position corresponding to position 95 of SEQ ID NO: 1;
C at a position corresponding to position 463 of SEQ ID NO: 1;
C at a position corresponding to position 865 of SEQ ID NO: 1;
C at a position corresponding to position 1092 of SEQ ID NO: 1;
C at a position corresponding to position 1284 of SEQ ID NO: 1; and/or
G at a position corresponding to position 1309 of SEQ ID NO: 1.

In certain other embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) does not comprise or lacks one or more SNP which is
C at a position corresponding to position 219 of SEQ ID NO: 2;
C at a position corresponding to position 446 of SEQ ID NO: 2;
C at a position corresponding to position 638 of SEQ ID NO: 2; and/or
G at a position corresponding to position 663 of SEQ ID NO: 2.

In certain other embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) does not comprise or lacks one or more SNP which is
C at a position corresponding to position 219 of a coding sequence encoding a protein having a sequence as set forth in SEQ ID NO: 3;
C at a position corresponding to position 446 of a coding sequence encoding a protein having a sequence as set forth in SEQ ID NO: 3;
C at a position corresponding to position 638 of a coding sequence encoding a protein having a sequence as set forth in SEQ ID NO: 3; and/or
G at a position corresponding to position 663 of a coding sequence encoding a protein having a sequence as set forth in SEQ ID NO: 3.

In certain other embodiments, in the methods according to the invention as described herein (such as the methods for identifying or generating a barley plant or plant part) or in the polynucleic acids according to the invention as described herein, the (purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) does not comprise or lacks one or more SNP which is
T at a position corresponding to position 95 of SEQ ID NO: 7;
C at a position corresponding to position 224 of SEQ ID NO: 8;
C at a position corresponding to position 206 of SEQ ID NO: 9;
C at a position corresponding to position 73 of SEQ ID NO: 10;
C at a position corresponding to position 145 of SEQ ID NO: 11; and/or
G at a position corresponding to position 50 of SEQ ID NO: 12.

In certain embodiments, in the methods according to the invention as described herein (such as the methods for identifying or generating a barley plant or plant part) or in the polynucleic acids according to the invention as described herein, the (non-purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
V, preferably G at a position corresponding to position 71238822 bp (on chromosome 7H) of reference genome Barley Morex v3.0;
D, preferably G at a position corresponding to position 71238454 bp (on chromosome 7H) of reference genome Barley Morex v3.0;
D, preferably G at a position corresponding to position 71238052 bp (on chromosome 7H) of reference genome Barley Morex v3.0;
D, preferably T at a position corresponding to position 71237825 bp (on chromosome 7H) of reference genome Barley Morex v3.0;
D, preferably A at a position corresponding to position 71237633 bp (on chromosome 7H) of reference genome Barley Morex v3.0; and/or
H, preferably A at a position corresponding to position 71237608 bp (on chromosome 7H) of reference genome Barley Morex v3.0.

In certain embodiments, in the methods according to the invention as described herein (such as the methods for identifying or generating a barley plant or plant part) or in the polynucleic acids according to the invention as described herein, the (non-purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
V, preferably G at a position corresponding to position 68464237 bp (on chromosome 7H) of reference genome Barley Morex v2.0;
D, preferably G at a position corresponding to position 68463869 bp (on chromosome 7H) of reference genome Barley Morex v2.0;
D, preferably G at a position corresponding to position 68463467 bp (on chromosome 7H) of reference genome Barley Morex v2.0;
D, preferably T at a position corresponding to position 68463240 bp (on chromosome 7H) of reference genome Barley Morex v2.0;
D, preferably A at a position corresponding to position 68463048 bp (on chromosome 7H) of reference genome Barley Morex v2.0; and/or
H, preferably A at a position corresponding to position 68463023 bp (on chromosome 7H) of reference genome Barley Morex v2.0.

In certain embodiments, in the methods according to the invention as described herein (such as the methods for identifying or generating a barley plant or plant part) or in the polynucleic acids according to the invention as described herein, the (non-purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
V, preferably G at a position corresponding to position 95 of SEQ ID NO: 4;
D, preferably G at a position corresponding to position 464 of SEQ ID NO: 4;
D, preferably G at a position corresponding to position 866 of SEQ ID NO: 4;
D, preferably T at a position corresponding to position 1093 of SEQ ID NO: 4;
D, preferably A at a position corresponding to position 1285 of SEQ ID NO: 4; and/or
H, preferably A at a position corresponding to position 1310 of SEQ ID NO: 4.

The skilled person will understand that the corresponding positions can be easily identified on SEQ ID NO: 1.

In certain other embodiments, in the methods according to the invention as described herein (such as the methods for identifying or generating a barley plant or plant part) or in the polynucleic acids according to the invention as described herein, the (non-purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises or lacks one or more SNP which is
D, preferably G at a position corresponding to position 219 of SEQ ID NO: 5;
D, preferably T at a position corresponding to position 446 of SEQ ID NO: 5;
D, preferably A at a position corresponding to position 638 of SEQ ID NO: 5; and/or
H, preferably A at a position corresponding to position 663 of SEQ ID NO: 5.

The skilled person will understand that the corresponding positions can be easily identified on SEQ ID NO: 2.

In certain other embodiments, in the methods according to the invention as described herein (such as the methods for identifying or generating a barley plant or plant part) or in the polynucleic acids according to the invention as described herein, the (non-purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
D, preferably G at a position corresponding to position 219 of a coding sequence encoding a protein having a sequence as set forth in SEQ ID NO: 6;
D, preferably T at a position corresponding to position 446 of a coding sequence encoding a protein having a sequence as set forth in SEQ ID NO: 6;
D, preferably A at a position corresponding to position 638 of a coding sequence encoding a protein having a sequence as set forth in SEQ ID NO: 6; and/or
H, preferably A at a position corresponding to position 663 of a coding sequence encoding a protein having a sequence as set forth in SEQ ID NO: 6.

The skilled person will understand that the corresponding positions can be easily identified for SEQ ID NO: 3.

In certain other embodiments, in the methods according to the invention as described herein (such as the methods for identifying or generating a barley plant or plant part) or in the polynucleic acids according to the invention as described herein, the (non-purple) (Ant1-associated) polynucleic acid, molecular marker (allele), Ant1 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
V, preferably G at a position corresponding to position 95 of SEQ ID NO: 7;
D, preferably G at a position corresponding to position 224 of SEQ ID NO: 8;
D, preferably G at a position corresponding to position 206 of SEQ ID NO: 9;
D, preferably T at a position corresponding to position 73 of SEQ ID NO: 10;
D, preferably A at a position corresponding to position 145 of SEQ ID NO: 11; and/or
H, preferably A at a position corresponding to position 50 of SEQ ID NO: 12.

In certain embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), haplotype, SNP (allele) etc. or plant or plant part (homozygous or heterozygous, as described herein elsewhere) comprises one or more SNP which is
C at a position corresponding to position 591324765 bp (on chromosome 2H) of reference genome Barley Morex v3.0;
G at a position corresponding to position 593492855 bp (on chromosome 2H) of reference genome Barley Morex v3.0;
C at a position corresponding to position 593493961 bp (on chromosome 2H) of reference genome Barley Morex v3.0;
T at a position corresponding to position 593494314 bp (on chromosome 2H) of reference genome Barley Morex v3.0;
G at a position corresponding to position 593494569 bp (on chromosome 2H) of reference genome Barley Morex v3.0;
T at a position corresponding to position 593495307 bp (on chromosome 2H) of reference genome Barley Morex v3.0;
A at a position corresponding to position 593496606 bp (on chromosome 2H) of reference genome Barley Morex v3.0;
G at a position corresponding to position 593496442 bp (on chromosome 2H) of reference genome Barley Morex v3.0;
C at a position corresponding to position 593492774 bp (on chromosome 2H) of reference genome Barley Morex v3.0;
A at a position corresponding to position 593493167 bp (on chromosome 2H) of reference genome Barley Morex v3.0;
T at a position corresponding to position 593494160 bp (on chromosome 2H) of reference genome Barley Morex v3.0;
A at a position corresponding to position 593494377 bp (on chromosome 2H) of reference genome Barley Morex v3.0;
G at a position corresponding to position 593495891 bp (on chromosome 2H) of reference genome Barley Morex v3.0; and/or
C at a position corresponding to position 593497594 bp (on chromosome 2H) of reference genome Barley Morex v3.0;
preferably one or more of
G at a position corresponding to position 593492855 bp (on chromosome 2H) of reference genome Barley Morex v3.0;
C at a position corresponding to position 593493961 bp (on chromosome 2H) of reference genome Barley Morex v3.0;
T at a position corresponding to position 593494314 bp (on chromosome 2H) of reference genome Barley Morex v3.0;
G at a position corresponding to position 593494569 bp (on chromosome 2H) of reference genome Barley Morex v3.0;
T at a position corresponding to position 593495307 bp (on chromosome 2H) of reference genome Barley Morex v3.0; and/or
A at a position corresponding to position 593496606 bp (on chromosome 2H) of reference genome Barley Morex v3.0;
or
G at a position corresponding to position 593496442 bp (on chromosome 2H) of reference genome Barley Morex v3.0; and (i.e. in combination with) one or more of
C at a position corresponding to position 593492774 bp (on chromosome 2H) of reference genome Barley Morex v3.0;
A at a position corresponding to position 593493167 bp (on chromosome 2H) of reference genome Barley Morex v3.0;
T at a position corresponding to position 593494160 bp (on chromosome 2H) of reference genome Barley Morex v3.0;
A at a position corresponding to position 593494377 bp (on chromosome 2H) of reference genome Barley Morex v3.0;
G at a position corresponding to position 593495891 bp (on chromosome 2H) of reference genome Barley Morex v3.0; and/or
C at a position corresponding to position 593497594 bp (on chromosome 2H) of reference genome Barley Morex v3.0.

In certain embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises a SNP which is C at a position corresponding to position 599993462 bp (on chromosome 2H) of reference genome Barley Morex v2.0.

In certain embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises one or more SNP which is
G at a position corresponding to position 491 of SEQ ID NO: 14;
C at a position corresponding to position 1597 of SEQ ID NO: 14;
T at a position corresponding to position 1950 of SEQ ID NO: 14;
G at a position corresponding to position 2207 of SEQ ID NO: 14;
T at a position corresponding to position 2946 of SEQ ID NO: 14;
A at a position corresponding to position 3907 of SEQ ID NO: 14;
G at a position corresponding to position 3743 of SEQ ID NO: 14;
C at a position corresponding to position 410 of SEQ ID NO: 14;
A at a position corresponding to position 803 of SEQ ID NO: 14;
T at a position corresponding to position 1796 of SEQ ID NO: 14;
A at a position corresponding to position 2013 of SEQ ID NO: 14;
G at a position corresponding to position 3530 of SEQ ID NO: 14; and/or
C at a position corresponding to position 4895 of SEQ ID NO: 14;
preferably one or more of
G at a position corresponding to position 491 of SEQ ID NO: 14;
C at a position corresponding to position 1597 of SEQ ID NO: 14;
T at a position corresponding to position 1950 of SEQ ID NO: 14;
G at a position corresponding to position 2207 of SEQ ID NO: 14;
T at a position corresponding to position 2946 of SEQ ID NO: 14; and/or
A at a position corresponding to position 3907 of SEQ ID NO: 14;
or
G at a position corresponding to position 3743 of SEQ ID NO: 14 and (i.e., in combination with) one or more of
C at a position corresponding to position 410 of SEQ ID NO: 14;
A at a position corresponding to position 803 of SEQ ID NO: 14;
T at a position corresponding to position 1796 of SEQ ID NO: 14;
A at a position corresponding to position 2013 of SEQ ID NO: 14;
G at a position corresponding to position 3530 of SEQ ID NO: 14; and/or
C at a position corresponding to position 4895 of SEQ ID NO: 14.

In certain other embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises or lacks one or more SNP which is
A at a position corresponding to position 804 of SEQ ID NO: 15;
G at a position corresponding to position 640 of SEQ ID NO: 15;
A at a position corresponding to position 49 of SEQ ID NO: 15; and/or
C at a position corresponding to position 1619 of SEQ ID NO: 15;
preferably
A at a position corresponding to position 804 of SEQ ID NO: 15;
or
G at a position corresponding to position 640 of SEQ ID NO: 15 and (i.e., in combination with) one or more of
A at a position corresponding to position 49 of SEQ ID NO: 15; and/or
C at a position corresponding to position 1619 of SEQ ID NO: 15.

In certain other embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises one or more SNP which is
A at a position corresponding to position 804 of a coding sequence encoding a protein having a sequence as set forth in SEQ ID NO: 16;
G at a position corresponding to position 640 of a coding sequence encoding a protein having a sequence as set forth in SEQ ID NO: 16;
A at a position corresponding to position 49 of a coding sequence encoding a protein having a sequence as set forth in SEQ ID NO: 16; and/or
C at a position corresponding to position 1619 of a coding sequence encoding a protein having a sequence as set forth in SEQ ID NO: 16;
preferably
A at a position corresponding to position 804 of a coding sequence encoding a protein having a sequence as set forth in SEQ ID NO: 16;
or
G at a position corresponding to position 640 of a coding sequence encoding a protein having a sequence as set forth in SEQ ID NO: 16 and (i.e., in combination with) one or more of
A at a position corresponding to position 49 of a coding sequence encoding a protein having a sequence as set forth in SEQ ID NO: 16; and/or
C at a position corresponding to position 1619 of a coding sequence encoding a protein having a sequence as set forth in SEQ ID NO: 16.

In certain other embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), haplotype, SNP (allele) etc. or plant or plant part (homozygous or heterozygous, as described herein elsewhere) comprises a SNP which is
C at a position corresponding to position 101 of SEQ ID NO: 13
G at a position corresponding to position 46 of SEQ ID NO: 20;
C at a position corresponding to position 96 of SEQ ID NO: 21;
T at a position corresponding to position 64 of SEQ ID NO: 22;
G at a position corresponding to position 73 of SEQ ID NO: 23;
T at a position corresponding to position 101 of SEQ ID NO: 24;
A at a position corresponding to position 72 of SEQ ID NO: 25;
G at a position corresponding to position 84 of SEQ ID NO: 26;
C at a position corresponding to position 76 of SEQ ID NO: 27;
A at a position corresponding to position 94 of SEQ ID NO: 28;
T at a position corresponding to position 88 of SEQ ID NO: 29;
A at a position corresponding to position 52 of SEQ ID NO: 30;
G at a position corresponding to position 80 of SEQ ID NO: 31; and/or
C at a position corresponding to position 70 of SEQ ID NO: 32;
preferably one or more of
G at a position corresponding to position 46 of SEQ ID NO: 20;
C at a position corresponding to position 96 of SEQ ID NO: 21;
T at a position corresponding to position 64 of SEQ ID NO: 22;
G at a position corresponding to position 73 of SEQ ID NO: 23;
T at a position corresponding to position 101 of SEQ ID NO: 24; and/or
A at a position corresponding to position 72 of SEQ ID NO: 25;
or
G at a position corresponding to position 84 of SEQ ID NO: 26 and (i.e., in combination with) one or more of
C at a position corresponding to position 76 of SEQ ID NO: 27;
A at a position corresponding to position 94 of SEQ ID NO: 28;
T at a position corresponding to position 88 of SEQ ID NO: 29;
A at a position corresponding to position 52 of SEQ ID NO: 30;
G at a position corresponding to position 80 of SEQ ID NO: 31; and/or
C at a position corresponding to position 73 of SEQ ID NO: 32.

In certain embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) does not comprise or lacks one or more SNP which is
C at a position corresponding to position 591324765 bp (on chromosome 2H) of reference genome Barley Morex v3.0
G at a position corresponding to position 593492855 bp (on chromosome 2H) of reference genome Barley Morex v3.0;
C at a position corresponding to position 593493961 bp (on chromosome 2H) of reference genome Barley Morex v3.0;
T at a position corresponding to position 593494314 bp (on chromosome 2H) of reference genome Barley Morex v3.0;
G at a position corresponding to position 593494569 bp (on chromosome 2H) of reference genome Barley Morex v3.0;
T at a position corresponding to position 593495307 bp (on chromosome 2H) of reference genome Barley Morex v3.0;
A at a position corresponding to position 593496606 bp (on chromosome 2H) of reference genome Barley Morex v3.0;
G at a position corresponding to position 593496442 bp (on chromosome 2H) of reference genome Barley Morex v3.0;
C at a position corresponding to position 593492774 bp (on chromosome 2H) of reference genome Barley Morex v3.0;
A at a position corresponding to position 593493167 bp (on chromosome 2H) of reference genome Barley Morex v3.0;
T at a position corresponding to position 593494160 bp (on chromosome 2H) of reference genome Barley Morex v3.0;
A at a position corresponding to position 593494377 bp (on chromosome 2H) of reference genome Barley Morex v3.0;
G at a position corresponding to position 593495891 bp (on chromosome 2H) of reference genome Barley Morex v3.0; and/or
C at a position corresponding to position 593497594 bp (on chromosome 2H) of reference genome Barley Morex v3.0;
preferably does not comprise or lacks one or more of
G at a position corresponding to position 593492855 bp (on chromosome 2H) of reference genome Barley Morex v3.0;
C at a position corresponding to position 593493961 bp (on chromosome 2H) of reference genome Barley Morex v3.0;
T at a position corresponding to position 593494314 bp (on chromosome 2H) of reference genome Barley Morex v3.0;
G at a position corresponding to position 593494569 bp (on chromosome 2H) of reference genome Barley Morex v3.0;
T at a position corresponding to position 593495307 bp (on chromosome 2H) of reference genome Barley Morex v3.0; and/or
A at a position corresponding to position 593496606 bp (on chromosome 2H) of reference genome Barley Morex v3.0;
or does not comprise or lacks.
G at a position corresponding to position 593496442 bp (on chromosome 2H) of reference genome Barley Morex v3.0 in combination with one or more of
C at a position corresponding to position 593492774 bp (on chromosome 2H) of reference genome Barley Morex v3.0;
A at a position corresponding to position 593493167 bp (on chromosome 2H) of reference genome Barley Morex v3.0;
T at a position corresponding to position 593494160 bp (on chromosome 2H) of reference genome Barley Morex v3.0;
A at a position corresponding to position 593494377 bp (on chromosome 2H) of reference genome Barley Morex v3.0;
G at a position corresponding to position 593495891 bp (on chromosome 2H) of reference genome Barley Morex v3.0; and/or
C at a position corresponding to position 593497594 bp (on chromosome 2H) of reference genome Barley Morex v3.0.

In certain embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) does not comprise or lacks one or more SNP which is
G at a position corresponding to position 491 of SEQ ID NO: 14;
C at a position corresponding to position 1597 of SEQ ID NO: 14;
T at a position corresponding to position 1950 of SEQ ID NO: 14;
G at a position corresponding to position 2207 of SEQ ID NO: 14;
T at a position corresponding to position 2946 of SEQ ID NO: 14;
A at a position corresponding to position 3907 of SEQ ID NO: 14;
G at a position corresponding to position 3743 of SEQ ID NO: 14;
C at a position corresponding to position 410 of SEQ ID NO: 14;
A at a position corresponding to position 803 of SEQ ID NO: 14;
T at a position corresponding to position 1796 of SEQ ID NO: 14;
A at a position corresponding to position 2013 of SEQ ID NO: 14;
G at a position corresponding to position 3530 of SEQ ID NO: 14; and/or
C at a position corresponding to position 4895 of SEQ ID NO: 14;
preferably does not comprise or lacks one or more of
G at a position corresponding to position 491 of SEQ ID NO: 14;
C at a position corresponding to position 1597 of SEQ ID NO: 14;
T at a position corresponding to position 1950 of SEQ ID NO: 14;
G at a position corresponding to position 2207 of SEQ ID NO: 14;
T at a position corresponding to position 2946 of SEQ ID NO: 14; and/or
A at a position corresponding to position 3907 of SEQ ID NO: 14;
or does not comprise or lacks
G at a position corresponding to position 3743 of SEQ ID NO: 14 in combination with one or more of
C at a position corresponding to position 410 of SEQ ID NO: 14;
A at a position corresponding to position 803 of SEQ ID NO: 14;
T at a position corresponding to position 1796 of SEQ ID NO: 14;
A at a position corresponding to position 2013 of SEQ ID NO: 14;
G at a position corresponding to position 3530 of SEQ ID NO: 14; and/or
C at a position corresponding to position 4895 of SEQ ID NO: 14.

In certain other embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) does not comprise or lacks one or more SNP which is
A at a position corresponding to position 804 of SEQ ID NO: 15;
G at a position corresponding to position 640 of SEQ ID NO: 15;
A at a position corresponding to position 49 of SEQ ID NO: 15; and/or
C at a position corresponding to position 1619 of SEQ ID NO: 15;
preferably does not comprise or lacks
A at a position corresponding to position 804 of SEQ ID NO: 15;
or does not comprise or lacks
G at a position corresponding to position 640 of SEQ ID NO: 15 in combination with one or more of
A at a position corresponding to position 49 of SEQ ID NO: 15; and/or
C at a position corresponding to position 1619 of SEQ ID NO: 15.

In certain other embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) does not comprise or lacks one or more SNP which is
A at a position corresponding to position 804 of a coding sequence encoding a protein having a sequence as set forth in SEQ ID NO: 16;
G at a position corresponding to position 640 of a coding sequence encoding a protein having a sequence as set forth in SEQ ID NO: 16;
A at a position corresponding to position 49 of a coding sequence encoding a protein having a sequence as set forth in SEQ ID NO: 16; and/or
C at a position corresponding to position 1619 of a coding sequence encoding a protein having a sequence as set forth in SEQ ID NO: 16;
preferably does not comprise or lacks
A at a position corresponding to position 804 of a coding sequence encoding a protein having a sequence as set forth in SEQ ID NO: 16;
or does not comprise or lacks
G at a position corresponding to position 640 of a coding sequence encoding a protein having a sequence as set forth in SEQ ID NO: 16 in combination with one or more of
A at a position corresponding to position 49 of a coding sequence encoding a protein having a sequence as set forth in SEQ ID NO: 16; and/or
C at a position corresponding to position 1619 of a coding sequence encoding a protein having a sequence as set forth in SEQ ID NO: 16.

In certain other embodiments, in the methods according to the invention as described herein (such as the methods for identifying or generating a barley plant or plant part) or in the polynucleic acids according to the invention as described herein, the (purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) does not comprise or lacks a SNP which is
C at a position corresponding to position 101 of SEQ ID NO: 13
G at a position corresponding to position 46 of SEQ ID NO: 20;
C at a position corresponding to position 96 of SEQ ID NO: 21;
T at a position corresponding to position 64 of SEQ ID NO: 22;
G at a position corresponding to position 73 of SEQ ID NO: 23;
T at a position corresponding to position 101 of SEQ ID NO: 24;
A at a position corresponding to position 72 of SEQ ID NO: 25;
G at a position corresponding to position 84 of SEQ ID NO: 26;
C at a position corresponding to position 76 of SEQ ID NO: 27;
A at a position corresponding to position 94 of SEQ ID NO: 28;
T at a position corresponding to position 88 of SEQ ID NO: 29;
A at a position corresponding to position 52 of SEQ ID NO: 30;
G at a position corresponding to position 80 of SEQ ID NO: 31; and/or
C at a position corresponding to position 70 of SEQ ID NO: 32;
preferably does not comprise or lacks one or more of
G at a position corresponding to position 46 of SEQ ID NO: 20;
C at a position corresponding to position 96 of SEQ ID NO: 21;
T at a position corresponding to position 64 of SEQ ID NO: 22;
G at a position corresponding to position 73 of SEQ ID NO: 23;
T at a position corresponding to position 101 of SEQ ID NO: 24; and/or
A at a position corresponding to position 72 of SEQ ID NO: 25;
or does not comprise or lacks
G at a position corresponding to position 84 of SEQ ID NO: 26 in combination with one or more of
C at a position corresponding to position 76 of SEQ ID NO: 27;
A at a position corresponding to position 94 of SEQ ID NO: 28;
T at a position corresponding to position 88 of SEQ ID NO: 29;
A at a position corresponding to position 52 of SEQ ID NO: 30;
G at a position corresponding to position 80 of SEQ ID NO: 31; and/or
C at a position corresponding to position 73 of SEQ ID NO: 32.

In certain embodiments, in the methods according to the invention as described herein (such as the methods for identifying or generating a barley plant or plant part) or in the polynucleic acids according to the invention as described herein, the (non-purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises one or more SNP which is
D, preferably T at a position corresponding to position 591324765 bp (on chromosome 2H) of reference genome Barley Morex v3.0;
H, preferably T at a position corresponding to position 593492855 bp (on chromosome 2H) of reference genome Barley Morex v3.0;
D, preferably T at a position corresponding to position 593493961 bp (on chromosome 2H) of reference genome Barley Morex v3.0;
V, preferably G at a position corresponding to position 593494314 bp (on chromosome 2H) of reference genome Barley Morex v3.0;
H, preferably A at a position corresponding to position 593494569 bp (on chromosome 2H) of reference genome Barley Morex v3.0;
V, preferably C at a position corresponding to position 593495307 bp (on chromosome 2H) of reference genome Barley Morex v3.0;
B, preferably C at a position corresponding to position 593496606 bp (on chromosome 2H) of reference genome Barley Morex v3.0;
H, preferably A at a position corresponding to position 593496442 bp (on chromosome 2H) of reference genome Barley Morex v3.0;
D, preferably G at a position corresponding to position 593492774 bp (on chromosome 2H) of reference genome Barley Morex v3.0;
B, preferably G at a position corresponding to position 593493167 bp (on chromosome 2H) of reference genome Barley Morex v3.0;
V, preferably C at a position corresponding to position 593494160 bp (on chromosome 2H) of reference genome Barley Morex v3.0;
B, preferably T at a position corresponding to position 593494377 bp (on chromosome 2H) of reference genome Barley Morex v3.0;
H, preferably A at a position corresponding to position 593495891 bp (on chromosome 2H) of reference genome Barley Morex v3.0; and/or
D, preferably G at a position corresponding to position 593497594 bp (on chromosome 2H) of reference genome Barley Morex v3.0;
preferably one or more of
H, preferably T at a position corresponding to position 593492855 bp (on chromosome 2H) of reference genome Barley Morex v3.0;
D, preferably T at a position corresponding to position 593493961 bp (on chromosome 2H) of reference genome Barley Morex v3.0;
V, preferably G at a position corresponding to position 593494314 bp (on chromosome 2H) of reference genome Barley Morex v3.0;
H, preferably A at a position corresponding to position 593494569 bp (on chromosome 2H) of reference genome Barley Morex v3.0;
V, preferably C at a position corresponding to position 593495307 bp (on chromosome 2H) of reference genome Barley Morex v3.0; and/or
B, preferably C at a position corresponding to position 593496606 bp (on chromosome 2H) of reference genome Barley Morex v3.0;
or
H, preferably A at a position corresponding to position 593496442 bp (on chromosome 2H) of reference genome Barley Morex v3.0; and (i.e., in combination with) one or more of
D, preferably G at a position corresponding to position 593492774 bp (on chromosome 2H) of reference genome Barley Morex v3.0;
B, preferably G at a position corresponding to position 593493167 bp (on chromosome 2H) of reference genome Barley Morex v3.0;
V, preferably C at a position corresponding to position 593494160 bp (on chromosome 2H) of reference genome Barley Morex v3.0;
B, preferably T at a position corresponding to position 593494377 bp (on chromosome 2H) of reference genome Barley Morex v3.0;
H, preferably A at a position corresponding to position 593495891 bp (on chromosome 2H) of reference genome Barley Morex v3.0; and/or
D, preferably G at a position corresponding to position 593497594 bp (on chromosome 2H) of reference genome Barley Morex v3.0.

In certain embodiments, in the methods according to the invention as described herein (such as the methods for identifying or generating a barley plant or plant part) or in the polynucleic acids according to the invention as described herein, the (non-purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises one or more SNP which is
H, preferably T at a position corresponding to position 491 of SEQ ID NO: 17;
D, preferably T at a position corresponding to position 1597 of SEQ ID NO: 17;
V, preferably G at a position corresponding to position 1950 of SEQ ID NO: 17;
H, preferably A at a position corresponding to position 2207 of SEQ ID NO: 17;
V, preferably C at a position corresponding to position 2946 of SEQ ID NO: 17;
B, preferably C at a position corresponding to position 3907 of SEQ ID NO: 17;
H, preferably A at a position corresponding to position 3743 of SEQ ID NO: 17;
D, preferably G at a position corresponding to position 410 of SEQ ID NO: 17;
B, preferably G at a position corresponding to position 803 of SEQ ID NO: 17;
V, preferably C at a position corresponding to position 1796 of SEQ ID NO: 17;
B, preferably T at a position corresponding to position 2013 of SEQ ID NO: 17;
H, preferably A at a position corresponding to position 3530 of SEQ ID NO: 17; and/or
D, preferably G at a position corresponding to position 4895 of SEQ ID NO: 17;
preferably one or more of
H, preferably T at a position corresponding to position 491 of SEQ ID NO: 17;
D, preferably T at a position corresponding to position 1597 of SEQ ID NO: 17;
V, preferably G at a position corresponding to position 1950 of SEQ ID NO: 17;
H, preferably A at a position corresponding to position 2207 of SEQ ID NO: 17;
V, preferably C at a position corresponding to position 2946 of SEQ ID NO: 17; and/or
B, preferably C at a position corresponding to position 3907 of SEQ ID NO: 17;
or
H, preferably A at a position corresponding to position 3743 of SEQ ID NO: 17 and (i.e., in combination with) one or more of
D, preferably G at a position corresponding to position 410 of SEQ ID NO: 17;
B, preferably G at a position corresponding to position 803 of SEQ ID NO: 17;
V, preferably C at a position corresponding to position 1796 of SEQ ID NO: 17;
B, preferably T at a position corresponding to position 2013 of SEQ ID NO: 17;
H, preferably A at a position corresponding to position 3530 of SEQ ID NO: 17; and/or
D, preferably G at a position corresponding to position 4895 of SEQ ID NO: 17.

The skilled person will understand that the corresponding positions can be easily identified on SEQ ID NO: 14.

In certain other embodiments, in the methods according to the invention as described herein (such as the methods for identifying or generating a barley plant or plant part) or in the polynucleic acids according to the invention as described herein, the (non-purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises or lacks one or more SNP which is
B, preferably C at a position corresponding to position 804 of SEQ ID NO: 18;
H, preferably A at a position corresponding to position 640 of SEQ ID NO: 18;
B, preferably G at a position corresponding to position 49 of SEQ ID NO: 18; and/or
D, preferably G at a position corresponding to position 1619 of SEQ ID NO: 18;
preferably
B, preferably C at a position corresponding to position 804 of SEQ ID NO: 18;
or
H, preferably A at a position corresponding to position 640 of SEQ ID NO: 18 and (i.e., in combination with) one or more of
B, preferably G at a position corresponding to position 49 of SEQ ID NO: 18; and/or
D, preferably G at a position corresponding to position 1619 of SEQ ID NO: 18.

The skilled person will understand that the corresponding positions can be easily identified on SEQ ID NO: 15.

In certain other embodiments, in the methods according to the invention as described herein (such as the methods for identifying or generating a barley plant or plant part) or in the polynucleic acids according to the invention as described herein, the (non-purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises one or more SNP which is
B, preferably C at a position corresponding to position 804 of a coding sequence encoding a protein having a sequence as set forth in SEQ ID NO: 19;
H, preferably A at a position corresponding to position 640 of a coding sequence encoding a protein having a sequence as set forth in SEQ ID NO: 19;
B, preferably G at a position corresponding to position 49 of a coding sequence encoding a protein having a sequence as set forth in SEQ ID NO: 19; and/or
D, preferably G at a position corresponding to position 1619 of a coding sequence encoding a protein having a sequence as set forth in SEQ ID NO: 19;
preferably
B, preferably C at a position corresponding to position 804 of a coding sequence encoding a protein having a sequence as set forth in SEQ ID NO: 19;
or
H, preferably A at a position corresponding to position 640 of a coding sequence encoding a protein having a sequence as set forth in SEQ ID NO: 19 and (i.e., in combination with) one or more of
B, preferably G at a position corresponding to position 49 of a coding sequence encoding a protein having a sequence as set forth in SEQ ID NO: 19; and/or
D, preferably G at a position corresponding to position 1619 of a coding sequence encoding a protein having a sequence as set forth in SEQ ID NO: 19.

The skilled person will understand that the corresponding positions can be easily identified on SEQ ID NO: 16.

In certain other embodiments, in the methods according to the invention as described herein (such as the methods for identifying or generating a barley plant or plant part) or in the polynucleic acids according to the invention as described herein, the (non-purple) (Ant2-associated) polynucleic acid, molecular marker (allele), Ant2 gene (allele) or coding sequence thereof or protein encoding sequence thereof, locus (allele), QTL (allele), SNP (allele) etc. or plant or plant part (homozygous or heterozygous, as described herein elsewhere) comprises one or more SNP which is
D, preferably T at a position corresponding to position 101 of SEQ ID NO: 13
H, preferably T at a position corresponding to position 46 of SEQ ID NO: 20;
D, preferably T at a position corresponding to position 96 of SEQ ID NO: 21;
V, preferably G at a position corresponding to position 64 of SEQ ID NO: 22;
H, preferably A at a position corresponding to position 73 of SEQ ID NO: 23;
V, preferably C at a position corresponding to position 101 of SEQ ID NO: 24;
B, preferably C at a position corresponding to position 72 of SEQ ID NO: 25;
H, preferably A at a position corresponding to position 84 of SEQ ID NO: 26;
D, preferably G at a position corresponding to position 76 of SEQ ID NO: 27;
B, preferably G at a position corresponding to position 94 of SEQ ID NO: 28;
V, preferably C at a position corresponding to position 88 of SEQ ID NO: 29;
B, preferably T at a position corresponding to position 52 of SEQ ID NO: 30;
H, preferably A at a position corresponding to position 80 of SEQ ID NO: 31; and/or
D, preferably G at a position corresponding to position 70 of SEQ ID NO: 32;
preferably one or more of
H, preferably T at a position corresponding to position 46 of SEQ ID NO: 20;
D, preferably T at a position corresponding to position 96 of SEQ ID NO: 21;
V, preferably G at a position corresponding to position 64 of SEQ ID NO: 22;
H, preferably A at a position corresponding to position 73 of SEQ ID NO: 23;
V, preferably C at a position corresponding to position 101 of SEQ ID NO: 24; and/or
B, preferably C at a position corresponding to position 72 of SEQ ID NO: 25;
or
H, preferably A at a position corresponding to position 84 of SEQ ID NO: 26 and (i.e., in combination with) one or more of
D, preferably G at a position corresponding to position 76 of SEQ ID NO: 27;
B, preferably G at a position corresponding to position 94 of SEQ ID NO: 28;
V, preferably C at a position corresponding to position 88 of SEQ ID NO: 29;
B, preferably T at a position corresponding to position 52 of SEQ ID NO: 30;
H, preferably A at a position corresponding to position 80 of SEQ ID NO: 31; and/or
D, preferably G at a position corresponding to position 73 of SEQ ID NO: 32.

The skilled person will understand that the position of nucleotides by reference to or at a position “corresponding” to a particular position, such as in a reference sequence, can be easily determined by suitable sequence alignment, as is known in the art.

In will be understood that in the context of QTLs, loci, gene or coding sequences and the like may and preferably do comprise all SNPs. In smaller sequences however, such as molecular marker sequences or primers or probes, only a single SNP as described herein is present. The skilled person will understand that the (purple or non-purple) (Ant1-associated or Ant2-associated) polynucleic acid, molecular marker (allele), Ant1 or Ant2 gene (allele) or coding sequence thereof or protein encoding sequence thereof, locus (allele), QTL (allele), SNP (allele) etc. may comprise additional mutations, such as SNPs or indels, besides the SNPs according to the invention described herein.

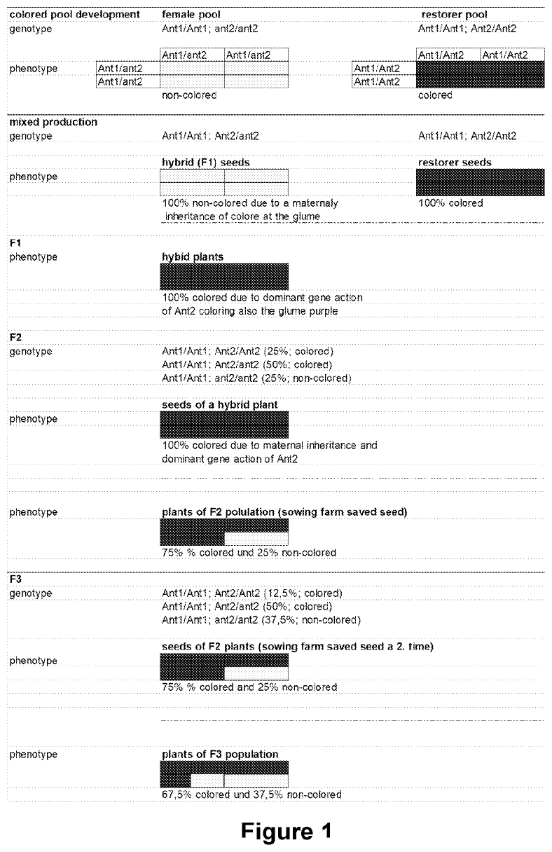

Preferably, a (Ant1-associated) purple allele of a polynucleic acid, QTL, locus, gene or gene comprises all purple Ant1 SNP alleles. In order to be capable to phenotypically manifest as purple seeds (pericarp/palea/lemma), a (Ant1-associated) purple allele of a polynucleic acid, QTL, locus, or gene preferably is homozygous (subject to the pericarp seed color and hull color being maternally inherited, and hence phenotypically corresponding to the maternal genotype, as described herein elsewhere). Accordingly, the purple color by Ant1 is a epistatic trait (and a purple phenotype is moreover additionally dependent on the allelic status of Ant2, and vice versa). A seed (in particular the embryo) carrying a homozygous (Ant1-associated) purple allele of a polynucleic acid, QTL, locus, or gene will grow into a plant producing purple seeds (if combined with homo- or heterozygosity of a purple Ant2 allele), irrespective of the genotype of the next generation or offspring seed (in particular the embryo), i.e., the seed produced by the plant grown from said seed. See also FIG. 1 in this context.

In order to be capable to phenotypically manifest as purple seeds (pericarp/lemma/palea), a (Ant2-associated) purple allele of a polynucleic acid, QTL, locus, or gene preferably is homozygous or heterozygous (subject to the pericarp seed color and hull color being maternally inherited, and hence phenotypically corresponding to the maternal genotype, as described herein elsewhere). Accordingly, the purple color by Ant2 is a dominant trait (and a purple phenotype is moreover additionally dependent on the allelic status of Ant1, and vice versa). A seed (in particular the embryo) carrying a homozygous or heterozygous (Ant2-associated) purple allele of a polynucleic acid, QTL, locus, or gene will grow into a plant producing purple seeds (if combined with homozygosity of a purple Ant1 allele), irrespective of the genotype of the next generation or offspring seed (in particular the embryo), i.e., the seed produced by the plant grown from said seed. See also FIG. 1 in this context.

In certain embodiments a (Ant1-associated) non-purple polynucleic acid, QTL, locus, gene or gene does not comprise or lacks at least one purple Ant1 SNP allele.

In certain embodiments a (Ant2-associated) non-purple polynucleic acid, QTL, locus, gene or gene does not comprise or lacks at least one purple Ant2 SNP allele.

In certain embodiments, the Ant1 gene as referred to herein has a genomic sequence which is at least 80%, preferably at least 90%, more preferably at least 95% identical to a sequence as set forth in SEQ ID NO: 1. Preferably, such Ant1 gene comprises one or more, preferably all, “Purple” Ant1 SNP of Table A. In certain embodiments, the Ant1 coding sequence as referred to herein has a genomic sequence which is at least 80%, preferably at least 90%, more preferably at least 95% identical to a sequence as set forth in SEQ ID NO: 2. Preferably, such Ant1 coding sequence comprises one or more, preferably all, “Purple” Ant1 SNP of Table A. In certain embodiments, the Ant1 gene or coding sequence as referred to herein encodes a protein having a sequence which is at least 80%, preferably at least 90%, more preferably at least 95% identical to a sequence as set forth in SEQ ID NO: 3. Preferably, such Ant1 gene or coding sequence comprises one or more, preferably all, “Purple” Ant1 SNP of Table A.

In certain embodiments, the Ant1 gene as referred to herein has a genomic sequence which is at least 80%, preferably at least 90%, more preferably at least 95% identical to a sequence as set forth in SEQ ID NO: 4. Preferably, such Ant1 gene does not comprise one or more “Purple” Ant1 SNP of Table A. In certain embodiments, the Ant1 coding sequence as referred to herein has a genomic sequence which is at least 80%, preferably at least 90%, more preferably at least 95% identical to a sequence as set forth in SEQ ID NO: 5. Preferably, such Ant1 coding sequence does not comprise one or more “Purple” Ant1 SNP of Table A. In certain embodiments, the Ant1 gene or coding sequence as referred to herein encodes a protein having a sequence which is at least 80%, preferably at least 90%, more preferably at least 95% identical to a sequence as set forth in SEQ ID NO: 6. Preferably, such Ant1 gene or coding sequence does not comprise one or more “Purple” Ant1 SNP of Table A.

In certain embodiments, the Ant1 gene as referred to herein has a genomic sequence which is at least 80%, preferably at least 90%, more preferably at least 95% identical to a sequence as set forth in SEQ ID NO: 4. Preferably, such Ant1 gene comprises one or more, preferably all, “Non-purple” Ant1 SNP of Table A. In certain embodiments, the Ant1 coding sequence as referred to herein has a genomic sequence which is at least 80%, preferably at least 90%, more preferably at least 95% identical to a sequence as set forth in SEQ ID NO: 5. Preferably, such Ant1 coding sequence comprises one or more, preferably all, “Non-purple” Ant1 SNP of Table A. In certain embodiments, the Ant1 gene or coding sequence as referred to herein encodes a protein having a sequence which is at least 80%, preferably at least 90%, more preferably at least 95% identical to a sequence as set forth in SEQ ID NO: 6. Preferably, such Ant1 gene or coding sequence comprises one or more, preferably all, “Non-purple” Ant1 SNP of Table A.

In certain embodiments, the Ant2 gene as referred to herein has a genomic sequence which is at least 80%, preferably at least 90%, more preferably at least 95% identical to a sequence as set forth in SEQ ID NO: 14. Preferably, such Ant2 gene comprises or is associated or linked with a “Purple” Ant2 SNP of Table A, preferably any one or more of Ant2_M2, Ant2_M3, Ant2_M4, Ant2_M5, Ant2_M6, and/or Ant2_M7 SNP; or Ant2_HapM1 and (i.e. in combination with) one or more of Ant2_HapM2, Ant2_HapM3, Ant2_HapM4, Ant2_HapM5, Ant2_HapM6, and/or Ant2_HapM7. In certain embodiments, the Ant2 coding sequence as referred to herein has a genomic sequence which is at least 80%, preferably at least 90%, more preferably at least 95% identical to a sequence as set forth in SEQ ID NO: 15. Preferably, such Ant2 coding sequence comprises or is associated or linked with one or more “Purple” Ant2 SNP of Table A, preferably Ant2_M7 or Ant2_HapM1 in combination with Ant2_HapM3 or Ant2_HapM7. In certain embodiments, the Ant2 gene or coding sequence as referred to herein encodes a protein having a sequence which is at least 80%, preferably at least 90%, more preferably at least 95% identical to a sequence as set forth in SEQ ID NO: 16. Preferably, such Ant2 gene or coding sequence comprises or is associated or linked with a, “Purple” Ant2 SNP of Table A, preferably Ant2_M7 or Ant2_HapM1 in combination with Ant2_HapM3 or Ant2_HapM7.

In certain embodiments, the Ant2 gene as referred to herein has a genomic sequence which is at least 80%, preferably at least 90%, more preferably at least 95% identical to a sequence as set forth in SEQ ID NO: 14. Preferably, such Ant2 gene does not comprise or is not associated or linked with a “Purple” Ant2 SNP of Table A, preferably lacking one or more of Ant2_M2, Ant2_M3, Ant2_M4, Ant2_M5, Ant2_M6, and/or Ant2_M7 SNP; or lacking Ant2_HapM1 or one or more of Ant2_HapM2, Ant2_HapM3, Ant2_HapM4, Ant2_HapM5, Ant2_HapM6, and/or Ant2_HapM7. In certain embodiments, the Ant2 coding sequence as referred to herein has a genomic sequence which is at least 80%, preferably at least 90%, more preferably at least 95% identical to a sequence as set forth in SEQ ID NO: 15. Preferably, such Ant2 coding sequence does not comprise or is not associated or linked with a “Purple” Ant2 SNP of Table A, preferably lacking Ant2_M7 or lacking Ant2_HapM1 or any one or more of Ant2_HapM3 or Ant2_HapM7. In certain embodiments, the Ant2 gene or coding sequence as referred to herein encodes a protein having a sequence which is at least 80%, preferably at least 90%, more preferably at least 95% identical to a sequence as set forth in SEQ ID NO: 16. Preferably, such Ant2 gene or coding sequence does not comprise′ or is not associated or linked with a “Purple” Ant2 SNP of Table A, preferably lacking Ant2_M7 or lacking Ant2_HapM1 or any one or more of Ant2_HapM3 or Ant2_HapM7.

In certain embodiments, the Ant2 gene as referred to herein has a genomic sequence which is at least 80%, preferably at least 90%, more preferably at least 95% identical to a sequence as set forth in SEQ ID NO: 14. Preferably, such Ant2 gene is associated or linked with the “Non-purple” Ant2 SNP of Table A. In certain embodiments, the Ant2 coding sequence as referred to herein has a genomic sequence which is at least 80%, preferably at least 90%, more preferably at least 95% identical to a sequence as set forth in SEQ ID NO: 15. Preferably, such Ant2 coding sequence is associated or linked with the “Non-purple” Ant2 SNP of Table A. In certain embodiments, the Ant2 gene or coding sequence as referred to herein encodes a protein having a sequence which is at least 80%, preferably at least 90%, more preferably at least 95% identical to a sequence as set forth in SEQ ID NO: 16. Preferably, such Ant2 gene or coding sequence is associated or linked with the “Non-purple” Ant2 SNP of Table A.

In an aspect, the invention relates to a method for identifying and/or selecting a barley plant or plant part, comprising screening for the presence of a polynucleotide or Ant1 gene having a sequence which is at least 80%, preferably at least 90%, more preferably at least 95% identical to a sequence as set forth in SEQ ID NO: 1 or 4 (in the genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant1 coding sequence having a sequence which is at least 80%, preferably at least 90%, more preferably at least 95% identical to a sequence as set forth in SEQ ID NO: 2 or 5 (in the genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant1 gene or coding sequence encoding a protein having a sequence which is at least 80%, preferably at least 90%, more preferably at least 95% identical to a sequence as set forth in SEQ ID NO: 3 or 6 (in the genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant1_HapM1 marker sequence having a sequence which is at least 80%, preferably at least 90%, more preferably at least 95% identical to a sequence as set forth in SEQ ID NO: 7 (in the Ant1 gene and/or genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant1_HapM2 marker sequence having a sequence which is at least 80%, preferably at least 90%, more preferably at least 95% identical to a sequence as set forth in SEQ ID NO: 8 (in the Ant1 gene and/or genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant1_HapM3 marker sequence having a sequence which is at least 80%, preferably at least 90%, more preferably at least 95% identical to a sequence as set forth in SEQ ID NO: 9 (in the Ant1 gene and/or genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant1_HapM4 marker sequence having a sequence which is at least 80%, preferably at least 90%, more preferably at least 95% identical to a sequence as set forth in SEQ ID NO: 10 (in the Ant1 gene and/or genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant1_HapM5 marker sequence having a sequence which is at least 80%, preferably at least 90%, more preferably at least 95% identical to a sequence as set forth in SEQ ID NO: 11 (in the Ant1 gene and/or genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant1_HapM6 marker sequence having a sequence which is at least 80%, preferably at least 90%, more preferably at least 95% identical to a sequence as set forth in SEQ ID NO: 12 (in the Ant1 gene and/or genome of the plant or plant part). Preferably, the sequence comprises one or more, preferably all, Ant1 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2 gene having a sequence which is at least 80%, preferably at least 90%, more preferably at least 95% identical to a sequence as set forth in SEQ ID NO: 14 or 17 (in the genome of the plant or plant part). Preferably, the sequence is associated or linked with the Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2 coding sequence having a sequence which is at least 80%, preferably at least 90%, more preferably at least 95% identical to a sequence as set forth in SEQ ID NO: 15 or 18 (in the genome of the plant or plant part). Preferably, the sequence is associated or linked with the Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2 gene or coding sequence encoding a protein having a sequence which is at least 80%, preferably at least 90%, more preferably at least 95% identical to a sequence as set forth in SEQ ID NO: 16 or 19 (in the genome of the plant or plant part). Preferably, the sequence is associated or linked with the Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_M1 marker sequence having a sequence which is at least 80%, preferably at least 90%, more preferably at least 95% identical to a sequence as set forth in SEQ ID NO: 13 (associated with or linked to the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_M2 marker sequence having a sequence which is at least 80%, preferably at least 90%, more preferably at least 95% identical to a sequence as set forth in SEQ ID NO: 20 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_M3 marker sequence having a sequence which is at least 80%, preferably at least 90%, more preferably at least 95% identical to a sequence as set forth in SEQ ID NO: 21 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_M4 marker sequence having a sequence which is at least 80%, preferably at least 90%, more preferably at least 95% identical to a sequence as set forth in SEQ ID NO: 22 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_M5 marker sequence having a sequence which is at least 80%, preferably at least 90%, more preferably at least 95% identical to a sequence as set forth in SEQ ID NO: 23 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_M6 marker sequence having a sequence which is at least 80%, preferably at least 90%, more preferably at least 95% identical to a sequence as set forth in SEQ ID NO: 24 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_M7 marker sequence having a sequence which is at least 80%, preferably at least 90%, more preferably at least 95% identical to a sequence as set forth in SEQ ID NO: 25 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_HapM1 marker sequence having a sequence which is at least 80%, preferably at least 90%, more preferably at least 95% identical to a sequence as set forth in SEQ ID NO: 26 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_HapM2 marker sequence having a sequence which is at least 80%, preferably at least 90%, more preferably at least 95% identical to a sequence as set forth in SEQ ID NO: 27 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_HapM3 marker sequence having a sequence which is at least 80%, preferably at least 90%, more preferably at least 95% identical to a sequence as set forth in SEQ ID NO: 28 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_HapM4 marker sequence having a sequence which is at least 80%, preferably at least 90%, more preferably at least 95% identical to a sequence as set forth in SEQ ID NO: 29 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_HapM5 marker sequence having a sequence which is at least 80%, preferably at least 90%, more preferably at least 95% identical to a sequence as set forth in SEQ ID NO: 30 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_HapM6 marker sequence having a sequence which is at least 80%, preferably at least 90%, more preferably at least 95% identical to a sequence as set forth in SEQ ID NO: 31 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

In an aspect, the invention relates to a method for identifying and/or selecting a barley plant or plant part, comprising screening for the presence of a polynucleotide or Ant2_HapM7 marker sequence having a sequence which is at least 80%, preferably at least 90%, more preferably at least 95% identical to a sequence as set forth in SEQ ID NO: 32 (in the Ant2 gene and/or in the genome of the plant or plant part). Preferably, the sequence comprises a Ant2 SNP of Table A. SNP identity allows for discrimination between “Purple” and “Non-Purple” alleles, as described herein elsewhere.

It will be understood that in the methods for identifying and/or selecting a barley plant according to the invention as described herein preferably encompass screening for the presence of both Ant1 and an Ant2 (associated) gene, coding sequence, polynucleotide, locus, marker, haplotype, SNP, etc.

In certain embodiments, the polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) is homozygous.

In certain embodiments, the polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) is heterozygous.

In certain embodiments, the methods for identifying and/or selecting a barley plant or plant part according to the invention as described herein include determining the zygosity of the polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele). In certain embodiments, the methods for identifying and/or selecting a barley plant or plant part according to the invention as described herein include screening for homozygosity of the polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele). In certain embodiments, the methods for identifying and/or selecting a barley plant or plant part according to the invention as described herein include screening for heterozygosity of the polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele). Screening for and identification of zygosity can be done by any means known in the art, such as for instance by allele-specific assays, as described herein elsewhere.

In certain embodiments, the methods for identifying and/or selecting a barley plant or plant part according to the invention as described herein are methods for identifying and/or selecting a barley plant or plant part producing or capable of producing a seed having a purple pericarp and/or hull (palea and lemma), and/or a plant grown therefrom and producing or capable of producing a seed having a purple pericarp and/or hull (palea and lemma).

In certain embodiments, the methods for identifying and/or selecting a barley plant or plant part according to the invention as described herein are methods for identifying and/or selecting a barley plant or plant part carrying an Ant1 and/or Ant2 allele responsible for or required for a purple color of the seed pericarp and/or hull (palea and lemma).

In certain embodiments, the methods for identifying and/or selecting a barley plant or plant part according to the invention as described herein are methods for identifying and/or selecting a barley plant or plant part producing or capable of producing a seed not having a purple pericarp and/or hull (palea and lemma), and/or a plant grown therefrom and producing or capable of producing a seed non having a purple pericarp and/or hull (palea and lemma).

In certain embodiments, the methods for identifying and/or selecting a barley plant or plant part according to the invention as described herein are methods for identifying and/or selecting a barley plant or plant part carrying an Ant1 and/or Ant2 allele responsible for or required for a non-purple color of the seed pericarp and/or hull (palea and lemma).

In certain embodiments, the methods for identifying and/or selecting a barley plant or plant part according to the invention as described herein are methods for identifying and/or selecting a barley plant or plant part not producing or not capable of producing a seed having a purple pericarp and/or hull (palea and lemma), and/or a plant grown therefrom and not producing or not capable of producing a seed non having a purple pericarp and/or hull (palea and lemma).

In certain embodiments, the methods for identifying and/or selecting a barley plant or plant part according to the invention as described herein are methods for identifying and/or selecting a barley plant or plant part lacking an Ant1 and/or Ant2 allele responsible for or required for a purple color of the seed pericarp and/or hull (palea and lemma).

In certain embodiments, the methods for identifying and/or selecting a barley plant or plant part according to the invention as described herein are methods for discriminating or distinguishing between a barley plant or plant part producing or capable of producing a seed having a purple pericarp and/or hull (palea and lemma) and a barley plant or plant part producing or capable of producing a seed having a non-purple pericarp and/or hull (palea and lemma), and/or a plant grown therefrom and producing or capable of producing a seed having a purple pericarp and/or hull (palea and lemma) or a non-purple pericarp and/or hull (palea and lemma).

In certain embodiments, the methods for identifying and/or selecting a barley plant or plant part according to the invention as described herein are methods for discriminating or distinguishing between a barley plant or plant part producing or capable of producing a seed having a purple pericarp and/or hull (palea and lemma) and a barley plant or plant part not producing or not capable of producing a seed having a purple pericarp and/or hull (palea and lemma), and/or a plant grown therefrom and producing or capable of producing a seed having a purple pericarp and/or hull (palea and lemma) or not producing or not capable of producing a purple pericarp and/or hull (palea and lemma).

In certain embodiments, the methods for identifying and/or selecting a barley plant or plant part according to the invention as described herein are methods for discriminating or distinguishing between a barley plant or plant part carrying an Ant1 and/or Ant2 allele responsible for or required for a purple color of the seed pericarp and/or hull (palea and lemma) and a plant or plant part carrying an Ant1 and/or Ant2 allele responsible for or required for a non-purple color of the seed pericarp and/or hull (palea and lemma).

In certain embodiments, the methods for identifying and/or selecting a barley plant or plant part according to the invention as described herein are methods for discriminating or distinguishing between a barley plant or plant part carrying an Ant1 and/or Ant2 allele responsible for or required for a purple color of the seed pericarp and/or hull (palea and lemma) and a plant or plant part not carrying an Ant1 and/or Ant2 allele responsible for or required for a purple color of the seed pericarp and/or hull (palea and lemma).

As used herein, the term “purple” refers to the generally purple color of plants or plant parts, in particular seeds, or more specific the pericarp and/or hull (i.e., lemma and palea) of seeds. “Purple” plants or plant parts can be easily distinguished from “non-purple” plants or plant parts, as is known in the art. Reference herein to “purple seeds” encompasses seeds having a purple pericarp/hull (i.e., lemma and palea), and the terms may be used interchangeably herein. Accordingly, as used herein, a purple seed may have a purple pericarp. Accordingly, as used herein, a purple seed may have a purple hull. Accordingly, as used herein, a purple seed may have a purple lemma. Accordingly, as used herein, a purple seed may have a purple palea. The term “purple grains” equally applies to such seeds. While purple seeds generally have a purple color, depending on the developmental stage and the climatological conditions (such as sunshine exposure) such seeds may also have a more greyish color and thus include grey tones. The skilled person will understand that also such greyish seeds or seeds having such grey tones or shades are encompassed by the term “purple seeds”. Generally, “purple seeds” are thus darker in appearance than “non-purple seeds”. Typically, the purple color is mostly visible in fresh plant material after the flowering of the ear, at the time when the grain is formed. As the ripening process begins, the color becomes deeply dark and changes to a greyish color as the grain dries out towards harvest. Truly dry seed is more or less grey. The skilled person will understand that the coloration preferably is but need not be uniform. The evaluation of the color is based on the overall appearance (it being uniform, non-uniform, patched, spotted, etc.), as is known in the art.

Purple color in plants commonly is the result of anthocyanin accumulation. In particular in grasses, such as barley, purple colored grains result from anthocyanin synthesis and accumulation in particular in the pericarp/hull. In barley, anthocyanin production, and hence purple coloration, is governed by the transcription factors Ant1 and Ant2. The present inventors have found that a purple color results from the allelic constellations of both Ant1 and Ant2 loci. In particular, a specific allelic state “haplotype” at Ant1 locus in combination with a dominant allele in homo- or heterozygous state at locus Ant2 (either comprising single markers or characterizable by single markers, or in the alternative also as a haplotype composed of multiple markers) are responsible for and required for a purple color. The specific Ant1 and Ant2 loci responsible for and required for the purple color are described herein elsewhere (for instance characterized by the “Purple” SNPs and polynucleotides as indicated in Table A). A confounding factor for the evaluation of the allelic status of Ant1 and Ant2 loci is that seed color (in particular pericarp/hull color) is maternally determined, as the seed pericarp and hull is maternally inherited. Accordingly, while a seed embryo may comprise the required allelic configuration of Ant1 and Ant2 loci associated with purple coloration, seed harboring such embryo may nevertheless not be purple, if the mother plant does not have the required allelic configuration of Ant1 and Ant2 associated with purple coloration. Plants grown from such seed, however, will produce seeds having a purple pericarp/hull. Consequently, plants grown from seed embryos which have an allelic configuration of Ant1 and Ant2 associated with purple coloration will always form colored seed regardless of the configuration of Ant1 and Ant2 loci associated with coloration in their seed. A schematic representation of the genotypic and phenotypic effects of the allelic Ant1/Ant2 configuration in plants and seeds is provided in FIG. 1.

As used herein, reference to an Ant1 gene (allele) preferably encompasses the Ant1 coding sequence or Ant1 protein sequence. As used herein, reference to an Ant2 gene (allele) preferably encompasses the Ant2 coding sequence or Ant2 protein sequence.

In certain embodiments, a barley plant or plant part is identified as a barley plant or plant part producing or capable of producing a seed having a purple pericarp/hull, and/or a plant grown therefrom and producing or capable of producing a seed having a purple pericarp/hull if the corresponding/required polynucleic acid, locus (allele), gene (allele), molecular marker (allele), haplotype, and/or SNP (allele) is present or detected, preferably in the required allelic constellation/configuration (as for instance according to Table A).

In certain embodiments, a barley plant or plant part is identified as a barley plant or plant part not producing or not capable of producing a seed having a purple pericarp/hull, and/or a plant grown therefrom and not producing or not capable of producing a seed having a purple pericarp/hull if the corresponding/required polynucleic acid, locus (allele), gene (allele), molecular marker (allele), haplotype, and/or SNP (allele) is not present or not detected, preferably lacking the required allelic constellation/configuration (as for instance according to Table A).

In certain embodiments, a barley plant or plant part is identified as a barley plant or plant part producing or capable of producing a seed having a non-purple pericarp/hull, and/or a plant grown therefrom and producing or capable of producing a seed having a non-purple pericarp/hull if the corresponding/required polynucleic acid, locus (allele), gene (allele), molecular marker (allele), haplotype, and/or SNP (allele) is present or detected, preferably in the required allelic constellation/configuration (as for instance according to Table A).

In certain embodiments, a barley plant or plant part is identified if the corresponding/required polynucleic acid, locus (allele), gene (allele), molecular marker (allele), haplotype, and/or SNP (allele) is present or detected, preferably in the required allelic constellation/configuration (as for instance according to Table A).

In certain embodiments, a barley plant or plant part is identified if the corresponding/required polynucleic acid, locus (allele), gene (allele), molecular marker (allele), haplotype, and/or SNP (allele) is not present or not detected, preferably lacking the required allelic constellation/configuration (as for instance according to Table A).

In certain embodiments, a barley plant or plant part is identified if the corresponding/required polynucleic acid, locus (allele), gene (allele), molecular marker (allele), haplotype, and/or SNP (allele) is present or detected, preferably in the required allelic constellation/configuration (as for instance according to Table A).

In certain embodiments, a barley plant or plant part is identified as a barley plant or plant part producing or capable of producing a seed having a purple pericarp/hull, and/or a plant grown therefrom and producing or capable of producing a seed having a purple pericarp/hull if the plant or plant part comprises a homozygous Ant1 purple color polynucleic acid, locus (allele), gene (allele), molecular marker (allele), haplotype, and/or SNP (allele) (as for instance according to Table A) and if the plant or plant part comprises a homozygous or heterozygous Ant2 purple color polynucleic acid, locus (allele), gene (allele), molecular marker (allele), haplotype, and/or SNP (allele) (as for instance according to Table A).

In certain embodiments, a barley plant or plant part is identified as a barley plant or plant part not producing or not capable of producing a seed having a purple pericarp/hull, and/or a plant grown therefrom and not producing or not capable of producing a seed having a purple pericarp/hull if the plant or plant part does not comprise a homozygous Ant1 purple color polynucleic acid, locus (allele), gene (allele), molecular marker (allele), haplotype, and/or SNP (allele) (as for instance according to Table A) and/or if the plant or plant part does not comprise a Ant2 purple color polynucleic acid, locus (allele), gene (allele), molecular marker (allele), haplotype, and/or SNP (allele) (as for instance according to Table A).

In certain embodiments, a barley plant or plant part is identified as a barley plant or plant part producing or capable of producing a seed having a non-purple pericarp/hull, and/or a plant grown therefrom and producing or capable of producing a seed having a non-purple pericarp/hull if the plant or plant part does not comprise a homozygous Ant1 purple color polynucleic acid, locus (allele), gene (allele), molecular marker (allele), haplotype, and/or SNP (allele) (as for instance according to Table A) and/or if the plant or plant part does not comprise a Ant2 purple color polynucleic acid, locus (allele), gene (allele), molecular marker (allele), haplotype, and/or SNP (allele) (as for instance according to Table A).

In certain embodiments, a barley plant or plant part is identified as a barley plant or plant part not producing or not capable of producing a seed having a purple pericarp/hull, and/or a plant grown therefrom and not producing or not capable of producing a seed having a purple pericarp/hull if the plant or plant part comprises a homozygous or heterozygous Ant1 non-purple color polynucleic acid, locus (allele), gene (allele), molecular marker (allele), haplotype, and/or SNP (allele) (as for instance according to Table A) and/or if the plant or plant part comprises a homozygous Ant2 non-purple color polynucleic acid, locus (allele), gene (allele), molecular marker (allele), haplotype, and/or SNP (allele) (as for instance according to Table A).

In certain embodiments, a barley plant or plant part is identified as a barley plant or plant part producing or capable of producing a seed having a non-purple pericarp/hull, and/or a plant grown therefrom and producing or capable of producing a seed having a non-purple pericarp/hull if the plant or plant part comprises a homozygous or heterozygous Ant1 non-purple color polynucleic acid, locus (allele), gene (allele), molecular marker (allele), haplotype, and/or SNP (allele) (as for instance according to Table A) and/or if the plant or plant part comprises a homozygous Ant2 non-purple color polynucleic acid, locus (allele), gene (allele), molecular marker (allele), haplotype, and/or SNP (allele) (as for instance according to Table A).

In certain embodiments, a barley plant or plant part is identified as a barley plant or plant part not producing or not capable of producing a seed having a purple pericarp/hull, and/or a plant grown therefrom and not producing or not capable of producing a seed having a purple pericarp/hull if the corresponding/required polynucleic acid, locus (allele), gene (allele), molecular marker (allele), haplotype, and/or SNP (allele) is not present or not detected, preferably lacking the required allelic constellation/configuration (as for instance according to Table A).

In certain embodiments, a barley plant or plant part is identified as a barley plant or plant part producing or capable of producing a seed having a non-purple pericarp/hull, and/or a plant grown therefrom and producing or capable of producing a seed having a non-purple pericarp/hull if the corresponding/required polynucleic acid, locus (allele), gene (allele), molecular marker (allele), haplotype, and/or SNP (allele) is present or detected, preferably in the required allelic constellation/configuration (as for instance according to Table A).

In certain embodiments, the methods for identifying and/or selecting a barley plant or plant part according to the invention as described herein further comprise isolating DNA from said plant or plant part, preferably prior to said screening. In certain embodiments, the methods for identifying and/or selecting a barley plant or plant part according to the invention as described herein further comprise isolating genomic DNA from said plant or plant part, preferably prior to said screening. In certain embodiments, the methods for identifying and/or selecting a barley plant or plant part according to the invention as described herein further comprise isolating and purifying DNA from said plant or plant part, preferably prior to said screening. In certain embodiments, the methods for identifying and/or selecting a barley plant or plant part according to the invention as described herein further comprise isolating and purifying genomic DNA from said plant or plant part, preferably prior to said screening.

In certain embodiments, the methods for identifying and/or selecting a barley plant or plant part according to the invention as described herein further comprise selecting the identified plant or plant part.

In certain embodiments, the methods for identifying and/or selecting a barley plant or plant part according to the invention as described herein further comprise selecting the non-identified plant or plant part, not selecting the identified plant or plant part, or discarding the identified plant or plant part.

In certain embodiments, purple seeds are identified and/or selected. In certain embodiments, non-purple seeds are identified and/or selected.

In certain embodiments, the methods for identifying and/or selecting a barley plant or plant part according to the invention as described herein further comprise selecting plants or plant parts comprising a homozygous Ant1 purple color polynucleic acid, locus (allele), gene (allele), molecular marker (allele), and/or SNP (allele) (as for instance according to Table A) and a homozygous or heterozygous Ant2 purple color polynucleic acid, locus (allele), gene (allele), molecular marker (allele), and/or SNP (allele) (as for instance according to Table A).

In certain embodiments, the methods for identifying and/or selecting a barley plant or plant part according to the invention as described herein further comprise not selecting or discarding plants or plant parts comprising a homozygous Ant1 purple color polynucleic acid, locus (allele), gene (allele), molecular marker (allele), and/or SNP (allele) (as for instance according to Table A) and a homozygous or heterozygous Ant2 purple color polynucleic acid, locus (allele), gene (allele), molecular marker (allele), and/or SNP (allele) (as for instance according to Table A).

In certain embodiments, the methods for identifying and/or selecting a barley plant or plant part according to the invention as described herein further comprise selecting plants or plant parts not comprising a homozygous Ant1 purple color polynucleic acid, locus (allele), gene (allele), molecular marker (allele), and/or SNP (allele) (as for instance according to Table A) and/or a homozygous or heterozygous Ant2 purple color polynucleic acid, locus (allele), gene (allele), molecular marker (allele), and/or SNP (allele) (as for instance according to Table A).

In certain embodiments, the methods for identifying and/or selecting a barley plant or plant part according to the invention as described herein further comprise selecting plants or plant parts comprising a homozygous or heterozygous Ant1 non-purple color polynucleic acid, locus (allele), gene (allele), molecular marker (allele), and/or SNP (allele) (as for instance according to Table A) and/or a homozygous Ant2 non-purple color polynucleic acid, locus (allele), gene (allele), molecular marker (allele), and/or SNP (allele) (as for instance according to Table A).

In an aspect, the invention relates to a barley plant or plant part identified and/or selected by the methods for identifying and/or selecting a barley plant or plant part according to the invention as described herein.

In an aspect, the invention relates to a barley plant or plant part comprising a polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a barley plant or plant part comprising a polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a “purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a “non-purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A.

In an aspect, the invention relates to a barley plant or plant part comprising a homozygous polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a barley plant or plant part comprising a homozygous polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a homozygous “purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a homozygous “non-purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A.

In an aspect, the invention relates to a barley plant or plant part comprising a heterozygous polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a barley plant or plant part comprising a heterozygous polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a heterozygous “purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a heterozygous “non-purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A.

In an aspect, the invention relates to a barley plant or plant part comprising a heterozygous Ant1-associated and Ant2-polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a barley plant or plant part comprising a heterozygous Ant1-associated and Ant2-associated polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a heterozygous Ant1-associated and Ant2-associated “purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a heterozygous Ant1-associated and Ant2-associated “non-purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A.

In an aspect, the invention relates to a barley plant or plant part comprising a homozygous Ant1-associated and Ant2-associated polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a barley plant or plant part comprising a homozygous Ant1-associated and Ant2-associated polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a homozygous Ant1-associated and Ant2-associated “purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a homozygous Ant1-associated and Ant2-associated “non-purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A.

In an aspect, the invention relates to a barley plant or plant part comprising a homozygous Ant1-associated and a heterozygous Ant2-associated polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a barley plant or plant part comprising a homozygous Ant1-associated and a heterozygous Ant2-associated polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a homozygous Ant1-associated and a heterozygous Ant2-associated “purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a homozygous Ant1-associated and a heterozygous Ant2-associated “non-purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A.

In an aspect, the invention relates to a barley plant or plant part comprising a heterozygous Ant1-associated and a homozygous Ant2-associated polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a barley plant or plant part comprising a heterozygous Ant1-associated and a homozygous Ant2-associated polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a heterozygous Ant1-associated and a homozygous Ant2-associated “purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a barley plant or plant part comprising a heterozygous Ant1-associated and a homozygous Ant2-associated “non-purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A.

In certain embodiments, the barley plants or plant parts according to the invention as described herein (such as the plants or plant parts identified or generated according to the methods of the invention) are sterile. In certain embodiments, the barley plants or plant parts according to the invention as described herein (such as the plants or plant parts identified or generated according to the methods of the invention) are male sterile. In certain embodiments, the barley plants or plant parts according to the invention as described herein (such as the plants or plant parts identified or generated according to the methods of the invention) are female sterile. In certain embodiments, the barley plants or plant parts according to the invention as described herein (such as the plants or plant parts identified or generated according to the methods of the invention) are genetically sterile. In certain embodiments, the barley plants or plant parts according to the invention as described herein (such as the plants or plant parts identified or generated according to the methods of the invention or one of the plants used in a cross or breeding according to the invention) are cytoplasmically sterile. In certain embodiments, the barley plants or plant parts according to the invention as described herein (such as the plants or plant parts identified or generated according to the methods of the invention or one of the plants used in a cross or breeding according to the invention) are genetically male sterile. In certain embodiments, the barley plants or plant parts according to the invention as described herein (such as the plants or plant parts identified or generated according to the methods of the invention or one of the plants used in a cross or breeding according to the invention) are genetically female sterile. In certain embodiments, the barley plants or plant parts according to the invention as described herein (such as the plants or plant parts identified or generated according to the methods of the invention or one of the plants used in a cross or breeding according to the invention) are cytoplasmically male sterile. In certain embodiments, the barley plants or plant parts according to the invention as described herein (such as the plants or plant parts identified or generated according to the methods of the invention or one of the plants used in a cross or breeding according to the invention) are cytoplasmically female sterile. Preferably, the plants or plant parts are cytoplasmically male sterile. Cytoplasmic male sterility is well known in the art.

As used herein, the term “sterile” plant (line, cultivar, or variety) has its ordinary meaning in the art. By means of further guidance, and without limitation, the term refers to a plant which is unable to produce offspring as a Pollen or oocyte donor, and may result from the failure to produce (functional) ovaries, oocytes, anthers, pollen, or gametes. As used herein, the term “female sterile” plant (line, cultivar, or variety)—has its ordinary meaning in the art. By means of further guidance, and without limitation, the term refers to a plant which is unable to produce offspring as a oocyte donor, and may result from the failure to produce (functional) ovaries, oocytes, or gametes. As used herein, the term “male sterile” plant (line, cultivar, or variety) has its ordinary meaning in the art. By means of further guidance, and without limitation, the term refers to a plant which is unable to produce offspring as a pollen donor, and may result from the failure to produce (functional) anthers, pollen, or gametes. Cytoplasmic male sterile plants have cytoplasmic genes, usually in the mitochondria, that encode factors that disrupt or prevent pollen development, making them male-sterile, with male sterility inherited maternally. The utilization of cytoplasmic male sterility for hybrid seed production typically requires three separate plant lines: the male-sterile line, an isogeneic male-fertile line for propagation (“maintainer line”) and a line for restoring fertility to the hybrid so that it can produce seed (“restorer line”). The male-sterile line is used as the receptive parent in a hybrid cross, the maintainer line is genetically identical to the male-sterile line, excepting that it lacks the cytoplasmic sterility factors, and the restorer line is any line that masks the cytoplasmic sterility factor. The restorer line is very important for those plants, such as grain sorghum or cotton, the useful crop of which is the seed itself or seed-associated structures. Genetic male sterility is similar to cytoplasmic male sterility but differs in that the sterility factors are encoded in nuclear DNA. Typically, genetic male sterility refers to a change in a plant's genetic structure which results in its ability to produce and/or spread viable pollen. Genetic male sterile plant lines may occur naturally. It is also possible to create a male-sterile plant line using recombinant techniques. Whether naturally occurring or transgenic, male-sterile lines still require the use of a sister maintainer line for their propagation, which of necessity leads to a minimum of 50% male-fertile plants in propagated seed. This is a result of the genetics of male-sterility and maintainer lines. If the male-sterility factor is recessive, as most are, a male-sterile plant would have to be homozygous recessive in order to display the trait. With the aim to inhibit self-pollination of female lines in hybrid production, CMS (cytoplasmic male sterility) is applied in plant breeding programs worldwide. CMS is characterized by maternally inherited mutations, which result in plants that are unable to produce pollen (see for instance Schnable, P. S., & Wise, R. P. (1998). “The molecular basis of cytoplasmic male sterility and fertility restoration”. Trends in plant science, 3 (5), 175-180). In order to ensure that the hybrid progeny is entirely fertile and able to produce seeds, male parental lines in hybrid production need to possess so called restorer genes, which cover and cancel the effects of CMS in the parental female as the result of pollination. Typically, restorer lines carry the restorer genes in homozygous state expressing full fertility. They are lines that can either lack the sterility factors (i.e., the maternally inherited CMS-causing mutations) or harbor them. The dominant character of the restorer gene alleles will override the effects of the CMS-causing mutations in heterozygous and homozygous state. Thus, a heterozygous allelic configuration at this locus in hybrid seed will result in the restoration of fertility in the hybrid plant.

It will be understood that in the context of a plant part, a reference to (male/female/cytoplasmic/genetic) “sterile” means that the plant part is derived from a sterile plant and/or that the plant part may be regenerated into or may produce a sterile plant.

In certain embodiments, the barley plants or plant parts according to the invention as described herein (such as the plants or plant parts identified or generated according to the methods of the invention) are transgenic. In certain embodiments, transgenesis comprises transforming a plant or plant part, preferably a plant cell, more preferably an immature or mature embryo, an inflorescence, a protoplast or callus, with a polynucleotide, molecular marker (allele), SNP, Ant1 gene or coding sequence thereof, and/or Ant2 gene or coding sequence thereof according to the invention as defined herein, such as provided in Table A, and optionally regenerating a plant from said plant cell, preferably immature or mature embryo, inflorescence, protoplast or callus. In certain embodiments, the barley plants or plant parts according to the invention as described herein (such as the plants or plant parts identified or generated according to the methods of the invention) are mutagenized. In certain embodiments, the barley plants or plant parts according to the invention as described herein (such as the plants or plant parts identified or generated according to the methods of the invention) are mutagenized, such as by chemical mutagenization or mutagenization by radiation. In certain embodiments, the barley plants or plant parts according to the invention as described herein (such as the plants or plant parts identified or generated according to the methods of the invention) are gene-edited. In certain embodiments, the barley plants or plant parts according to the invention as described herein (such as the plants or plant parts identified or generated according to the methods of the invention) are obtained by introgression of said one or more polynucleotide, molecular marker (allele), SNP, Ant1 gene and/or Ant2 gene, etc. according to the invention as described herein elsewhere, such as provided in Table A. In certain embodiments, the barley plants or plant parts according to the invention as described herein (such as the plants or plant parts identified or generated according to the methods of the invention) are not obtained by introgression of said one or more polynucleotide, molecular marker (allele), SNP, Ant1 gene and/or Ant2 gene, etc. according to the invention as described herein elsewhere.

In an aspect, the invention relates to a sterile, preferably male sterile, more preferably cytoplasmically male sterile barley plant or plant part comprising a polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a sterile, preferably male sterile, more preferably cytoplasmically male sterile barley plant or plant part comprising a polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a sterile, preferably male sterile, more preferably cytoplasmically male sterile barley plant or plant part comprising a “purple” polynucleotide, locus (allele), Ant1 gene (allele) (or coding sequence thereof. or Ant1 protein encoding sequence therefore), Ant2 gene (allele) (or coding sequence thereof, or Ant2 protein encoding sequence therefore), SNP (allele), haplotype, and/or molecular marker (allele) as described in Table A. In an aspect, the invention relates to a sterile, preferably male sterile, more preferably cytoplasmically male sterile barley plant or plant part comprising a “non-purple” polynucleotide, locus (allele), Ant1 gene (allele) (or coding sequence thereof, or Ant1 protein encoding sequence therefore), Ant2 gene (allele) (or coding sequence thereof, or Ant2 protein encoding sequence therefore), SNP (allele), haplotype, and/or molecular marker (allele) as described in Table A.

In an aspect, the invention relates to a sterile, preferably male sterile, more preferably cytoplasmically male sterile barley plant or plant part comprising a homozygous Ant1-associated polynucleotide, locus (allele), Ant1 gene (allele) (or coding sequence thereof, or Ant1 protein encoding sequence therefore), SNP (allele), haplotype, and/or molecular marker (allele) according to the invention as described herein elsewhere, as well as a homozygous or heterozygous Ant2-associated polynucleotide, locus (allele), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a sterile, preferably male sterile, more preferably cytoplasmically male sterile barley plant or plant part comprising a homozygous Ant1-associated polynucleotide, locus (allele), Ant1 gene (allele) (or coding sequence thereof, or Ant1 protein encoding sequence therefore), SNP (allele), haplotype, and/or molecular marker (allele) according to the invention, as well as a homozygous or heterozygous Ant2-associated polynucleotide, locus (allele), Ant2 gene (allele) (or coding sequence thereof, or Ant2 protein encoding sequence therefore), SNP (allele), haplotype, and/or molecular marker (allele), both as described in Table A. In an aspect, the invention relates to a sterile, preferably male sterile, more preferably cytoplasmically male sterile barley plant or plant part comprising a homozygous “purple” Ant1-associated polynucleotide, locus (allele), Ant1 gene (allele) (or coding sequence thereof, or Ant1 protein encoding sequence therefore), SNP (allele), haplotype, and/or molecular marker (allele), as well as a homozygous or heterozygous “purple” Ant2-associated polynucleotide, locus (allele), Ant2 gene (allele) (or coding sequence thereof, or Ant2 protein encoding sequence therefore), SNP (allele), haplotype, and/or molecular marker (allele), both as described in Table A.

In an aspect, the invention relates to a sterile, preferably male sterile, more preferably cytoplasmically male sterile barley plant or plant part comprising a homozygous Ant1-associated polynucleotide, locus (allele), Ant1 gene (allele) (or coding sequence thereof, or Ant1 protein encoding sequence therefore), SNP (allele), haplotype, and/or molecular marker (allele) according to the invention as described herein elsewhere. In an aspect, the invention relates to a sterile, preferably male sterile, more preferably cytoplasmically male sterile barley plant or plant part comprising a homozygous Ant1-associated polynucleotide, locus (allele), Ant1 gene (allele) (or coding sequence thereof, or Ant1 protein encoding sequence therefore), SNP (allele), haplotype, and/or molecular marker (allele) as described in Table A. In an aspect, the invention relates to a sterile, preferably male sterile, more preferably cytoplasmically male sterile barley plant or plant part comprising a homozygous “purple” Ant1-associated polynucleotide, locus (allele), Ant1 gene (allele) (or coding sequence thereof, or Ant1 protein encoding sequence therefore), SNP (allele), haplotype, and/or molecular marker (allele) as described in Table A.

In an aspect, the invention relates to a sterile, preferably male sterile, more preferably cytoplasmically male sterile barley plant or plant part comprising a homozygous or heterozygous Ant2-associated polynucleotide, locus (allele), Ant2 gene (allele) (or coding sequence thereof, or Ant2 protein encoding sequence therefore), SNP (allele), haplotype, and/or molecular marker (allele) according to the invention as described herein elsewhere. In an aspect, the invention relates to a sterile, preferably male sterile, more preferably cytoplasmically male sterile barley plant or plant part comprising a homozygous or heterozygous Ant2-associated polynucleotide, locus (allele), Ant2 gene (allele) (or coding sequence thereof, or Ant2 protein encoding sequence therefore), SNP (allele), haplotype, and/or molecular marker (allele) as described in Table A. In an aspect, the invention relates to a sterile, preferably male sterile, more preferably cytoplasmically male sterile barley plant or plant part comprising a homozygous “purple” Ant2-associated polynucleotide, locus (allele), Ant2 gene (allele) (or coding sequence thereof, or Ant2 protein encoding sequence therefore). SNP (allele), haplotype, and/or molecular marker (allele) as described in Table A.

Preferably, according to the invention, the barley plants or plant parts comprising the polynucleotide, Ant1 gene (allele), or coding sequence thereof, Ant2 gene (allele), or coding sequence thereof, molecular marker (allele), SNP (allele) according to the invention as described herein, such as provided in Table A, express, are configured to express, or are capable of expressing the Ant1 and/or Ant2 gene (allele) and/or protein.

In certain embodiments, the plant is not a plant variety.

In an aspect, the invention relates to a method for producing a barley plant or plant part, comprising
i) (recombinantly, transgenically, by gene-editing, or by mutagenesis) introducing into the genome of a barley plant or plant part a polynucleotide, molecular marker (allele), SNP (allele), haplotype, Ant1 gene (allele) or coding sequence thereof, and/or Ant2 gene (allele) or coding sequence thereof according to the invention as defined herein;
ii) crossing a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof according to the invention as defined herein; and selecting progeny seeds or plants comprising a polynucleotide, molecular marker (allele), SNP (allele), haplotype, Ant1 gene (allele) or coding sequence thereof, and/or Ant2 gene (allele) or coding sequence thereof according to the invention as defined herein; or
iii) providing a barley seed mixture harvested from a cross between a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof according to the invention as defined herein; and selecting seeds comprising a polynucleotide, molecular marker (allele), SNP (allele), haplotype, Ant1 gene (allele) or coding sequence thereof, and/or Ant2 gene (allele) or coding sequence thereof according to the invention as defined herein.

In an aspect, the invention relates to a method for producing a barley plant or plant part, comprising
i) (recombinantly, transgenically, by gene-editing, or by mutagenesis) introducing into the genome of a barley plant or plant part a polynucleotide, molecular marker (allele), SNP (allele), haplotype, Ant1 gene (allele) or coding sequence thereof, and/or Ant2 gene (allele) or coding sequence thereof as defined in Table A;
ii) crossing a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof as defined in Table A; and selecting progeny seeds or plants comprising a polynucleotide, molecular marker (allele), SNP (allele), haplotype, Ant1 gene (allele) or coding sequence thereof, and/or Ant2 gene (allele) or coding sequence thereof as defined in Table A; or
iii) providing a barley seed mixture harvested from a cross between a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof as defined in Table A; and selecting seeds comprising a polynucleotide, molecular marker (allele), SNP (allele), haplotype, Ant1 gene (allele) or coding sequence thereof, and/or Ant2 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part an Ant1-associated polynucleotide, molecular marker (allele), SNP (allele), haplotype, or Ant1 gene (allele) or coding sequence thereof as defined in Table A;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A; and selecting progeny seeds or plants comprising an Ant1-associated polynucleotide, molecular marker (allele), SNP (allele), haplotype, or Ant1 gene (allele) or coding sequence thereof as defined in Table A; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A; and selecting seeds comprising an Ant1-associated polynucleotide, molecular marker (allele), SNP (allele), haplotype, or Ant1 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part a “purple” Ant1-associated polynucleotide, molecular marker (allele), SNP (allele), haplotype, or Ant1 gene (allele) or coding sequence thereof as defined in Table A;
ii) crossing a first barley plant (population) and a second barley plant (population), wherein said first and/or second barley plant (population) comprises; and selecting progeny seeds or plants comprising a “purple” Ant1-associated polynucleotide, molecular marker (allele), SNP (allele), haplotype, or Ant1 gene (allele) or coding sequence thereof as defined in Table A; and selecting progeny seeds or plants comprising a “purple” Ant1-associated polynucleotide, molecular marker (allele), SNP (allele), haplotype, or Ant1 gene (allele) or coding sequence thereof as defined in Table A; or
iii) providing a barley seed mixture harvested from a cross between a first barley plant (population) and a second barley plant (population), wherein said first and/or second barley plant (population) comprises; and selecting progeny seeds or plants comprising a “purple” Ant1-associated polynucleotide, molecular marker (allele), SNP (allele), haplotype, or Ant1 gene (allele) or coding sequence thereof as defined in Table A; and selecting seeds comprising a “purple” Ant1-associated polynucleotide, molecular marker (allele), SNP (allele), haplotype, or Ant1 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant1-associated polynucleotide, molecular marker (allele), SNP (allele), haplotype, or Ant1 gene (allele) or coding sequence thereof as defined in Table A;
ii) crossing a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A; and selecting progeny seeds or plants comprising a “non-purple” Ant1-associated polynucleotide, molecular marker (allele), SNP (allele), haplotype, or Ant1 gene (allele) or coding sequence thereof as defined in Table A; or
iii) providing a barley seed mixture harvested from a cross between a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A; and selecting seeds comprising a “non-purple” Ant1-associated polynucleotide, molecular marker (allele), SNP (allele), haplotype, or Ant1 gene (allele) or coding sequence thereof as defined in Table A.

In an aspect, the invention relates to a method for producing a barley plant or plant part, comprising
i) (recombinantly, transgenically, by gene editing, or by mutagenesis) introducing into the genome of a barley plant or plant part an Ant2-associated polynucleotide, molecular marker (allele), SNP (allele), haplotype, or Ant2 gene (allele) or coding sequence thereof as defined in Table A;
ii) crossing a first barley plant (population) and a second barley plant (population), wherein said first and/or second barley plant (population) comprises; and selecting progeny seeds or plants comprising an Ant2-associated polynucleotide, molecular marker (allele), SNP (allele), haplotype, or Ant2 gene (allele) or coding sequence thereof as defined in Table A; and selecting progeny seeds or plants comprising an Ant2-associated polynucleotide, molecular marker (allele), SNP (allele), haplotype, or Ant2 gene (allele) or coding sequence thereof as defined in Table A; or
iii) providing a barley seed mixture harvested from a cross between a first barley plant (population) and a second barley plant (population), wherein said first and/or second barley plant (population) comprises; and selecting progeny seeds or plants comprising an Ant2-associated polynucleotide, molecular marker (allele), SNP (allele), haplotype, or Ant2 gene (allele) or coding sequence thereof as defined in Table A; and selecting seeds comprising an Ant2-associated polynucleotide, molecular marker (allele), SNP (allele), haplotype, or Ant2 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part a “purple” Ant2-associated polynucleotide, molecular marker (allele), SNP (allele), haplotype, or Ant2 gene (allele) or coding sequence thereof as defined in Table A;
ii) crossing a first barley plant (population) and a second barley plant (population), wherein said first and/or second barley plant (population) comprises a “purple” Ant2-associated polynucleotide, molecular marker (allele), SNP (allele), haplotype, or Ant2 gene (allele) or coding sequence thereof as defined in Table A; and selecting progeny seeds or plants comprising a “purple” Ant2-associated polynucleotide, molecular marker (allele), SNP (allele), haplotype, or Ant2 gene (allele) or coding sequence thereof as defined in Table A; or
iii) providing a barley seed mixture harvested from a cross between a first barley plant (population) and a second barley plant (population), wherein said first and/or second barley plant (population) comprises a “purple” Ant2-associated polynucleotide, molecular marker (allele), SNP (allele), haplotype, or Ant2 gene (allele) or coding sequence thereof as defined in Table A; and selecting seeds comprising a “purple” Ant2-associated polynucleotide, molecular marker (allele), SNP (allele), haplotype, or Ant2 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant2-associated polynucleotide, molecular marker (allele), SNP (allele), haplotype, or Ant2 gene (allele) or coding sequence thereof as defined in Table A;
ii) crossing a first barley plant (population) and a second barley plant (population), wherein said first and/or second barley plant (population) comprises a “non-purple” Ant2-associated polynucleotide, molecular marker (allele), SNP (allele), haplotype, or Ant2 gene (allele) or coding sequence thereof as defined in Table A; and selecting progeny seeds or plants comprising a “non-purple” Ant2-associated polynucleotide, molecular marker (allele), SNP (allele), haplotype, or Ant2 gene (allele) or coding sequence thereof as defined in Table A; or
iii) providing a barley seed mixture harvested from a cross between a first barley plant (population) and a second barley plant (population), wherein said first and/or second barley plant (population) comprises a “non-purple” Ant2-associated polynucleotide, molecular marker (allele), SNP (allele), haplotype, or Ant2 gene (allele) or coding sequence thereof as defined in Table A; and selecting seeds comprising a “non-purple” Ant2-associated polynucleotide, molecular marker (allele), SNP (allele), haplotype, or Ant2 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype. Ant1 gene (allele) or coding sequence thereof, and/or Ant2 gene (allele) or coding sequence thereof is homozygous; and selecting progeny seeds or plants comprising a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype. Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising a polynucleotide, molecular marker (allele), SNP (allele), haplotype, Ant1 gene (allele) or coding sequence thereof, and/or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising a polynucleotide, molecular marker (allele), SNP (allele), haplotype, Ant1 gene (allele) or coding sequence thereof, and/or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising an Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising a “purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising a “non-purple” Ant1-associated polynucleotide, molecular marker (allele), SNP (allele), haplotype, or Ant1 gene (allele) or coding sequence thereof as defined in Table A, wherein said polynucleotide, molecular marker (allele), SNP (allele), haplotype. Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype. Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising an Ant2-associated polynucleotide, molecular marker (allele). SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein; and selecting progeny seeds or plants comprising an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein; and selecting seeds comprising an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein.

In an aspect, the invention relates to a method for producing a barley plant or plant part, comprising
i) (recombinantly, transgenically, by gene-editing, or by mutagenesis) introducing into the genome of a barley plant or plant part an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A; and selecting progeny seeds or plants comprising an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A; and selecting seeds comprising an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A;
ii) crossing a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A; and selecting progeny seeds or plants comprising a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A; or
iii) providing a barley seed mixture harvested from a cross between a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A; and selecting seeds comprising a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A;
ii) crossing a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A; and selecting progeny seeds or plants comprising a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A; or
iii) providing a barley seed mixture harvested from a cross between a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A; and selecting seeds comprising a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A.

In an aspect, the invention relates to a method for producing a barley plant or plant part, comprising
i) (recombinantly, transgenically, by gene-editing, or by mutagenesis) introducing into the genome of a barley plant or plant part an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype. Ant1 gene (allele) or coding sequence thereof, and/or Ant2 gene (allele) or coding sequence thereof is heterozygous; and selecting progeny seeds or plants comprising an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype. Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous;
ii) crossing a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting progeny seeds or plants comprising a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; or
iii) providing a barley seed mixture harvested from a cross between a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous; and selecting seeds comprising a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is heterozygous.

In an aspect, the invention relates to a method for producing a barley plant or plant part, comprising
i) (recombinantly, transgenically, by gene-editing, or by mutagenesis) introducing into the genome of a barley plant or plant part an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype. Ant1 gene (allele) or coding sequence thereof, and/or Ant2 gene (allele) or coding sequence thereof is homozygous; and selecting progeny seeds or plants comprising an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising an Ant1-associated polynucleotide, molecular marker (allele), SNP (allele), haplotype, or Ant1 gene (allele) or coding sequence thereof according to the invention as defined herein, and an Ant2-associated polynucleotide, molecular marker (allele), SNP (allele), haplotype, or Ant2 gene (allele) or coding sequence thereof according to the invention as defined herein, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene editing, or by mutagenesis) introducing into the genome of a barley plant or plant part an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising an Ant1-associated polynucleotide, molecular marker (allele), SNP (allele), haplotype, or Ant1 gene (allele) or coding sequence thereof as defined in Table A, and an Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising a “purple” Ant1-associated polynucleotide, molecular marker (allele), SNP (allele), haplotype, or Ant1 gene (allele) or coding sequence thereof as defined in Table A, and a “purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype. Ant1 gene (allele) or coding sequence thereof, and/or Ant2 gene (allele) or coding sequence thereof is homozygous.

In an aspect, the invention relates to a method for producing a barley plant or plant part, comprising
i) (recombinantly, transgenically, by gene-editing, or by mutagenesis) introducing into the genome of a barley plant or plant part a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous;
ii) crossing a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting progeny seeds or plants comprising a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; or
iii) providing a barley seed mixture harvested from a cross between a first barley plant (population) and a second barley plant (population), wherein said first and/or second barley plant (population) comprises a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous; and selecting seeds comprising a “non-purple” Ant1-associated polynucleotide, molecular marker (allele), SNP (allele), haplotype, or Ant1 gene (allele) or coding sequence thereof as defined in Table A, and a “non-purple” Ant2-associated polynucleotide, molecular marker (allele), SNP (allele), haplotype, or Ant2 gene (allele) or coding sequence thereof as defined in Table A, wherein said polynucleotide, molecular marker (allele), SNP (allele), haplotype, Ant1 gene (allele) or coding sequence thereof, and/or Ant2 gene (allele) or coding sequence thereof is homozygous.

In an aspect, the invention relates to a method for generating or producing a (hybrid) barley plant or plant part, or for producing, obtaining, or selecting a (hybrid) barley seed (mixture), comprising crossing a first barley parent plant (population) and a second barley parent plant (population) or providing a (bulk or unselected) barley seed mixture harvested from a cross between a first barley parent plant (population) and a second barley parent plant (population), wherein said first and optionally second barley parent plant (population) comprises a polynucleotide, molecular marker (allele), SNP, haplotype, Ant1 gene, and/or Ant2 gene according to the invention as described herein;
selecting seeds comprising a polynucleotide, molecular marker (allele), SNP, haplotype, Ant1 gene, and/or Ant2 gene according to the invention as described herein;
optionally harvesting seeds resulting from said cross;
optionally sowing said selected seeds to grow barley plants;
optionally wherein said first or second barley plant (population) is sterile, preferably male sterile, more preferably cytoplasmic male sterile.

In certain embodiments, (only) said first barley parent plant (population) comprises a polynucleotide, molecular marker (allele), SNP, haplotype, Ant1 gene, and/or Ant2 gene according to the invention as described herein.

In certain embodiments, said first and optionally said second barley parent plant (population) comprises a Ant1-associated polynucleotide, molecular marker (allele), SNP (allele), haplotype, Ant1 gene (allele) or coding sequence thereof, and an Ant2-associated polynucleotide, molecular marker (allele), SNP (allele), haplotype, Ant2 gene (allele) or coding sequence thereof according to the invention as described herein.

In certain embodiments, (only) said first barley parent plant (population) comprises a Ant1-associated polynucleotide, molecular marker (allele), SNP (allele), haplotype, Ant1 gene (allele) or coding sequence thereof, and an Ant2-associated polynucleotide, molecular marker (allele), SNP (allele), haplotype, Ant2 gene (allele) or coding sequence thereof according to the invention as described herein.

In certain embodiments, Ant1-associated polynucleotide, molecular marker (allele), SNP (allele), haplotype, Ant1 gene (allele) or coding sequence thereof is homozygous.

In certain embodiments, said Ant2-associated polynucleotide, molecular marker (allele), SNP (allele), haplotype, Ant2 gene (allele) or coding sequence thereof is homozygous or heterozygous.

In certain embodiments, Ant1-associated polynucleotide, molecular marker (allele), SNP (allele), haplotype, Ant1 gene (allele) or coding sequence thereof is homozygous and said Ant2-associated polynucleotide, molecular marker (allele), SNP (allele), haplotype, Ant2 gene (allele) or coding sequence thereof is homozygous or heterozygous.

In certain embodiments, plants or plant parts are selected which comprise a homozygous Ant1-associated polynucleotide, molecular marker (allele), SNP (allele), haplotype, Ant1 gene (allele) or coding sequence thereof and a homozygous or heterozygous Ant2-associated polynucleotide, molecular marker (allele), SNP (allele), haplotype, Ant2 gene (allele) or coding sequence thereof.

In certain embodiments, plants or plant parts are selected which do not comprise a homozygous Ant1-associated polynucleotide, molecular marker (allele), SNP (allele), haplotype, Ant1 gene (allele) or coding sequence thereof or a homozygous or heterozygous Ant2-associated polynucleotide, molecular marker (allele), SNP (allele), haplotype, Ant2 gene (allele) or coding sequence thereof.

In certain embodiments, (only) said first barley parent plant (population) comprises a homozygous Ant1-associated polynucleotide, molecular marker (allele), haplotype, SNP, or Ant1 gene (or coding sequence thereof) according to the invention as described herein, and a homozygous or heterozygous Ant2-associated polynucleotide, molecular marker (allele), SNP, haplotype, or Ant2 gene (or coding sequence thereof) according to the invention as described herein.

In certain embodiments, only said first barley parent plant (population) comprises a homozygous Ant1-associated polynucleotide, molecular marker (allele), SNP, haplotype, or Ant1 gene (or coding sequence thereof) according to the invention as described herein, and a homozygous or heterozygous Ant2-associated polynucleotide, molecular marker (allele), SNP, haplotype, or Ant2 gene (or coding sequence thereof) according to the invention as described herein, said second barley plant (population) does not comprise a heterozygous Ant1-associated polynucleotide, molecular marker (allele), SNP, haplotype, or Ant1 gene according to the invention as described herein, and if said second barley plant (population) comprises a homozygous Ant1-associated polynucleotide, molecular marker (allele), SNP, or Ant1 gene according to the invention as described herein, said first barley plant (population) comprises a homozygous Ant2-associated polynucleotide, molecular marker (allele), SNP, haplotype, or Ant2 gene according to the invention as described herein.

In certain embodiments, only said first barley parent plant (population) comprises a Ant1-associated polynucleotide, molecular marker (allele), SNP, haplotype, or Ant1 gene according to the invention as described herein, said Ant1-associated polynucleotide, molecular marker (allele), SNP, or Ant1 gene is homozygous, and said first barley parent plant (population) further comprises a homozygous or heterozygous Ant2-associated polynucleotide, molecular marker (allele), SNP, haplotype, or Ant2 gene according to the invention as described herein.

In certain embodiments, said first barley parent plant (population) comprises a homozygous Ant1-associated polynucleotide, molecular marker (allele), SNP, haplotype, or Ant1 gene according to the invention as described herein, and a homozygous Ant2-associated polynucleotide, molecular marker (allele), SNP, haplotype, or Ant2 gene according to the invention as described herein; and said second barley parent plant (population) comprises a homozygous Ant1-associated polynucleotide, molecular marker (allele), SNP, haplotype, or Ant1 gene according to the invention as described herein, and does not comprise a Ant2-associated polynucleotide, molecular marker (allele), SNP, haplotype, or Ant2 gene according to the invention as described herein.

In certain embodiments, plants or plant parts are selected which have or are seeds having a purple pericarp and/or hull (lemma and palea).

In certain embodiments, plants or plant parts are selected which do not have or are not seeds having a purple pericarp and/or hull (lemma and palea).

In certain embodiments, the first or second plant is sterile, such as male or female sterile, preferably male sterile.

In certain embodiments, the first or second plant is genetic or cytoplasmic sterile, preferably cytoplasmic sterile.

In certain preferred embodiments, the first or second plant is cytoplasmic male sterile, preferably cytoplasmic sterile.

In certain embodiments, seeds having a purple pericarp and/or hull (lemma and palea) are selected if said first barley plant (population) is (cytoplasmic) (male) sterile and/or seeds not having a purple pericarp and/or hull (lemma and palea) are selected if said second barley plant (population) is (cytoplasmic) (male) sterile. In certain embodiments, seeds having a purple pericarp and/or hull (lemma and palea) are selected if said first barley plant (population) is (cytoplasmic) (male) sterile and seeds not having a purple pericarp and/or hull (lemma and palea) are selected if said second barley plant (population) is (cytoplasmic) (male) sterile. In certain embodiments, seeds having a purple pericarp and/or hull (lemma and palea) are selected if said first barley plant (population) is (cytoplasmic) (male) sterile or seeds not having a purple pericarp and/or hull (lemma and palea) are selected if said second barley plant (population) is (cytoplasmic) (male) sterile.

In an aspect, the invention relates to a method for producing or generating a (hybrid) barley plant or plant part, or for producing, obtaining, or selecting a (hybrid) barley seed (mixture), comprising crossing a first barley parent plant (population) and a second barley parent plant (population) or providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein only said first barley parent plant (population) comprises a homozygous Ant1-associated polynucleotide, molecular marker (allele). SNP, haplotype, or Ant1 gene (or coding sequence thereof) according to the invention as described herein, and a homozygous or heterozygous Ant2-associated polynucleotide, molecular marker (allele), SNP, haplotype, or Ant2 gene (or coding sequence thereof) according to the invention as described herein, and
selecting seeds having a purple pericarp and/or hull (lemma and palea) if said first barley plant (population) is (cytoplasmic) male sterile and selecting seeds not having a purple pericarp and/or hull (lemma and palea) if said second barley plant (population) is (cytoplasmic) male sterile;
optionally harvesting seeds resulting from said cross;
optionally sowing said selected seeds to grow barley plants.

In an aspect, the invention relates to a method for generating or producing a (hybrid) barley plant, or for producing, obtaining, or selecting a (hybrid) barley seed (mixture), comprising crossing a first barley parent plant (population) and a second barley parent plant (population) or providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein only said first barley parent plant (population) comprises a homozygous Ant1-associated polynucleotide, molecular marker (allele). SNP, haplotype, or Ant1 gene (or coding sequence thereof) according to the invention as described herein, and a homozygous or heterozygous Ant2-associated polynucleotide, molecular marker (allele), SNP, haplotype, or Ant2 gene (or coding sequence thereof) according to the invention as described herein,
wherein said second barley plant (population) does not comprise a heterozygous Ant1-associated polynucleotide, molecular marker (allele), haplotype, SNP, Ant1 gene according to the invention as described herein, and
wherein if said second barley plant (population) comprises a homozygous Ant1-associated polynucleotide, molecular marker (allele), SNP, haplotype, or Ant1 gene according to the invention as described herein, said first barley plant (population) comprises a homozygous Ant2-associated polynucleotide, molecular marker (allele), SNP, haplotype, or Ant2 gene according to the invention as described herein, and
selecting seeds having a purple pericarp and/or hull (lemma and palea) if said first barley plant (population) is (cytoplasmic) male sterile and selecting seeds not having a purple pericarp and/or hull (lemma and palea) if said second barley plant (population) is (cytoplasmic) male sterile,
optionally harvesting seeds resulting from said cross;
optionally sowing said selected seeds to grow barley plants.

In an aspect, the invention relates to a method for producing or generating a (hybrid) barley plant or plant part, or for producing, obtaining, or selecting a (hybrid) barley seed (mixture), comprising crossing a first barley parent plant (population) and a second barley parent plant (population) or providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein only said first barley parent plant (population) comprises a Ant1-associated polynucleotide, molecular marker (allele), haplotype, SNP, or Ant1 gene according to the invention as described herein,
wherein said Ant1-associated polynucleotide, molecular marker (allele), SNP, haplotype, or Ant1 gene is homozygous, and
wherein said first barley parent plant (population) further comprises a homozygous or heterozygous Ant2-associated polynucleotide, molecular marker (allele), SNP, haplotype, or Ant2 gene according to the invention as described herein, and
selecting seeds having a purple pericarp and/or hull (lemma and palea) if said first barley plant (population) is (cytoplasmic) male sterile and selecting seeds not having a purple pericarp and/or hull (lemma and palea) if said second barley plant (population) is (cytoplasmic) male sterile,
optionally harvesting seeds resulting from said cross;
optionally sowing said selected seeds to grow barley plants.

In an aspect, the invention relates to a method for producing or generating a (hybrid) barley plant or plant part, or for producing, obtaining, or selecting a (hybrid) barley seed (mixture), comprising crossing a first barley parent plant (population) and a second barley parent plant (population) or providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein said first barley parent plant (population) comprises a homozygous Ant1-associated polynucleotide, molecular marker (allele), SNP, haplotype, or Ant1 gene according to the invention as described herein, and a homozygous Ant2-associated polynucleotide, molecular marker (allele), SNP, haplotype, or Ant2 gene according to the invention as described herein; and wherein said second barley parent plant (population) comprises a homozygous Ant1-associated polynucleotide, molecular marker (allele), SNP, haplotype, or Ant1 gene according to the invention as described herein, and does not comprise a Ant2-associated polynucleotide, molecular marker (allele), SNP, haplotype, or Ant2 gene according to the invention as described herein, and
selecting seeds having a purple pericarp and/or hull (lemma and palea) if said first barley plant (population) is (cytoplasmic) male sterile and selecting seeds not having a purple pericarp and/or hull (lemma and palea) if said second barley plant (population) is (cytoplasmic) male sterile,
optionally harvesting seeds resulting from said cross;
optionally sowing said selected seeds to grow barley plants.

In an aspect, the invention relates to a method for producing or generating a (hybrid) barley plant or plant part, or for producing, obtaining, or selecting a (hybrid) barley seed (mixture), comprising crossing a first barley parent plant (population) and a second barley parent plant (population) or providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein only said first barley parent plant (population) comprises a homozygous Ant1 gene (or coding sequence thereof) comprising one or more of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences), and a homozygous or heterozygous (Ant2 gene (or coding sequence thereof) comprising or associated with or linked to one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e., in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences), and
selecting seeds having a purple pericarp and/or hull (lemma and palea) if said first barley plant (population) is (cytoplasmic) male sterile and selecting seeds not having a purple pericarp and/or hull (lemma and palea) if said second barley plant (population) is (cytoplasmic) male sterile,
optionally harvesting seeds resulting from said cross;
optionally sowing said selected seeds to grow barley plants.

In an aspect, the invention relates to a method for producing or generating a (hybrid) barley plant or plant part, or for producing, obtaining, or selecting a (hybrid) barley seed (mixture), comprising crossing a first barley parent plant (population) and a second barley parent plant (population) or providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein only said first barley parent plant (population) comprises a homozygous Ant1 gene (or coding sequence thereof) comprising one or more of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences), and a homozygous or heterozygous (Ant2 gene (or coding sequence thereof) comprising or associated with or linked to) one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences),
wherein said second barley plant (population) does not comprise a heterozygous Ant1 gene (or coding sequence thereof) comprising one or more of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences), and
wherein if said second barley plant (population) comprises a homozygous Ant1 gene (or coding sequence thereof) comprising one or more of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences), said first barley plant (population) comprises a homozygous (Ant2 gene (or coding sequence thereof) comprising or associated with or linked with one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences), and
selecting seeds having a purple pericarp and/or hull (lemma and palea) if said first barley plant (population) is (cytoplasmic) male sterile and selecting seeds not having a purple pericarp and/or hull (lemma and palea) if said second barley plant (population) is (cytoplasmic) male sterile,
optionally harvesting seeds resulting from said cross;
optionally sowing said selected seeds to grow barley plants.

In an aspect, the invention relates to a method for producing or generating a (hybrid) barley plant or plant part, or for producing, obtaining, or selecting a (hybrid) barley seed (mixture), comprising crossing a first barley parent plant (population) and a second barley parent plant (population) or providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein only said first barley parent plant (population) comprises a Ant1 gene (or coding sequence thereof) comprising one or more of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences),
wherein said Ant1 gene (or coding sequence thereof) comprising one or more of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences) is homozygous, and
wherein said first barley parent plant (population) further comprises a homozygous or heterozygous (Ant2 gene (or coding sequence thereof) comprising or associated with or linked with one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences), and
selecting seeds having a purple pericarp and/or hull (lemma and palea) if said first barley plant (population) is (cytoplasmic) male sterile and selecting seeds not having a purple pericarp and/or hull (lemma and palea) if said second barley plant (population) is (cytoplasmic) male sterile,
optionally harvesting seeds resulting from said cross;
optionally sowing said selected seeds to grow barley plants.

In an aspect, the invention relates to a method for producing or generating a (hybrid) barley plant or plant part, or for producing, obtaining, or selecting a (hybrid) barley seed (mixture), comprising crossing a first barley parent plant (population) and a second barley parent plant (population) or providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein said first barley parent plant (population) comprises a homozygous Ant1 gene (or coding sequence thereof) comprising one or more of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences), and a homozygous (Ant2 gene (or coding sequence thereof) comprising or associated with or linked with one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences); and
wherein said second barley parent plant (population) comprises a homozygous Ant1 gene (or coding sequence thereof) comprising one or more of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences), and does not comprise a (Ant2 gene (or coding sequence thereof) comprising or associated with or linked with one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences), and
selecting seeds having a purple pericarp and/or hull (lemma and palea) if said first barley plant (population) is (cytoplasmic) male sterile and selecting seeds not having a purple pericarp and/or hull (lemma and palea) if said second barley plant (population) is (cytoplasmic) male sterile,
optionally harvesting seeds resulting from said cross;
optionally sowing said selected seeds to grow barley plants.

In a further embodiment selecting seeds comprises the use of a seed sorter recognizing seed pigmentation or seed color expression.

In an aspect, the invention relates to a method for producing or generating a (hybrid) barley plant or plant part, comprising introducing into the genome of a barley plant or plant part a polynucleotide, such as an Ant1 gene (allele) or coding sequence thereof, having a sequence selected from
(1) a sequence having a sequence which is at least 80%, preferably at least 90%, more preferably at least 95%, identical to a sequence as set forth in SEQ ID NO: 1;
(2) a sequence having a sequence which is at least 80%, preferably at least 90%, more preferably at least 95%, identical to a sequence as set forth in SEQ ID NO: 2; and/or
(3) a sequence encoding a protein having a sequence which is at least 80%, preferably at least 90%, more preferably at least 95%, identical to a sequence as set forth in SEQ ID NO: 3;
preferably comprising one or more, preferably all, of molecular marker Ant1_HapM1 to Ant1_HapM6, having a SNP as indicated in Table B (at a position corresponding to the indicated position on one or more of the respective sequences).

In an aspect, the invention relates to a method for producing or generating a (hybrid) barley plant or plant part, comprising introducing into the genome of a barley plant or plant part a polynucleotide, such as an Ant2 gene (allele) or coding sequence thereof, having a sequence selected from
(1) a sequence having a sequence which is at least 80%, preferably at least 90%, more preferably at least 95%, identical to a sequence as set forth in SEQ ID NO: 14;
(2) a sequence having a sequence which is at least 80%, preferably at least 90%, more preferably at least 95%, identical to a sequence as set forth in SEQ ID NO: 15; and/or
(3) a sequence encoding a protein having a sequence which is at least 80%, preferably at least 90%, more preferably at least 95%, identical to a sequence as set forth in SEQ ID NO: 16.
preferably comprising or associated or linked with one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences).

In an aspect, the invention relates to a method for producing or generating a (hybrid) barley plant or plant part comprising
(A) introducing into the genome of a barley plant or plant part a polynucleotide, such as an Ant1 gene (allele) or coding sequence thereof, having a sequence selected from
(1) a sequence having a sequence which is at least 80%, preferably at least 90%, more preferably at least 95%, identical to a sequence as set forth in SEQ ID NO: 1;
(2) a sequence having a sequence which is at least 80%, preferably at least 90%, more preferably at least 95%, identical to a sequence as set forth in SEQ ID NO: 2; and/or
(3) a sequence encoding a protein having a sequence which is at least 80%, preferably at least 90%, more preferably at least 95%, identical to a sequence as set forth in SEQ ID NO: 3;
preferably comprising one or more, preferably all, of molecular marker Ant1_HapM1 to Ant1_HapM6, having a SNP as indicated in Table B (at a position corresponding to the indicated position on one or more of the respective sequences); and
(B) simultaneously or sequentially, in either order, introducing into the genome of a barley plant or plant part a polynucleotide, such as an Ant2 gene (allele) or coding sequence thereof, having a sequence selected from
(1) a sequence having a sequence which is at least 80%, preferably at least 90%, more preferably at least 95%, identical to a sequence as set forth in SEQ ID NO: 14;
(2) a sequence having a sequence which is at least 80%, preferably at least 90%, more preferably at least 95%, identical to a sequence as set forth in SEQ ID NO: 15; and/or
(3) a sequence encoding a protein having a sequence which is at least 80%, preferably at least 90%, more preferably at least 95%, identical to a sequence as set forth in SEQ ID NO: 16
preferably comprising or associated or linked with one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences).

In an aspect, the invention relates to a method for producing or generating a (hybrid) barley plant or plant part comprising
providing a plant or plant part having in the genome a polynucleotide, such as an Ant1 gene (allele) or coding sequence thereof, having a sequence selected from
(1) a sequence having a sequence which is at least 80%, preferably at least 90%, more preferably at least 95%, identical to a sequence as set forth in SEQ ID NO: 1;
(2) a sequence having a sequence which is at least 80%, preferably at least 90%, more preferably at least 95%, identical to a sequence as set forth in SEQ ID NO: 2; and/or
(3) a sequence encoding a protein having a sequence which is at least 80%, preferably at least 90%, more preferably at least 95%, identical to a sequence as set forth in SEQ ID NO: 3;
preferably comprising one or more, preferably all, of molecular marker Ant1_HapM1 to Ant1_HapM6, having a SNP as indicated in Table B (at a position corresponding to the indicated position on one or more of the respective sequences); and
introducing in the genome of said plant or plant part a polynucleotide, such as an Ant2 gene (allele) or coding sequence thereof, having a sequence selected from
(1) a sequence having a sequence which is at least 80%, preferably at least 90%, more preferably at least 95%, identical to a sequence as set forth in SEQ ID NO: 14;
(2) a sequence having a sequence which is at least 80%, preferably at least 90%, more preferably at least 95%, identical to a sequence as set forth in SEQ ID NO: 15; and/or
(3) a sequence encoding a protein having a sequence which is at least 80%, preferably at least 90%, more preferably at least 95%, identical to a sequence as set forth in SEQ ID NO: 16
preferably comprising or associated or linked with one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences).

In an aspect, the invention relates to a method for producing or generating a (hybrid) barley plant or plant part comprising
providing a plant or plant part having in the genome a polynucleotide, such as an Ant2 gene (allele) or coding sequence thereof, having a sequence selected from
(1) a sequence having a sequence which is at least 80%, preferably at least 90%, more preferably at least 95%, identical to a sequence as set forth in SEQ ID NO: 14;
(2) a sequence having a sequence which is at least 80%, preferably at least 90%, more preferably at least 95%, identical to a sequence as set forth in SEQ ID NO: 15; and/or
(3) a sequence encoding a protein having a sequence which is at least 80%, preferably at least 90%, more preferably at least 95%, identical to a sequence as set forth in SEQ ID NO: 16;
preferably comprising or associated or linked with one or more molecular marker selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably selected from Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences); and
introducing in the genome of said plant or plant part a polynucleotide, such as an Ant1 gene (allele) or coding sequence thereof, having a sequence selected from
(1) a sequence having a sequence which is at least 80%, preferably at least 90%, more preferably at least 95%, identical to a sequence as set forth in SEQ ID NO: 1;
(2) a sequence having a sequence which is at least 80%, preferably at least 90%, more preferably at least 95%, identical to a sequence as set forth in SEQ ID NO: 2; and/or
(3) a sequence encoding a protein having a sequence which is at least 80%, preferably at least 90%, more preferably at least 95%, identical to a sequence as set forth in SEQ ID NO: 3
preferably comprising one or more, preferably all, of molecular marker Ant1_HapM1 to Ant1_HapM6, having a SNP as indicated in Table B (at a position corresponding to the indicated position on one or more of the respective sequences).

In certain embodiments the Ant1 gene (allele) comprises or is comprised in a polynucleotide and/or comprises a molecular marker (allele) or SNP according to the invention as described herein, such as provided in Table B, preferably all.

In certain embodiments the Ant2 gene (allele) comprises or is comprised in a polynucleotide and/or is associated with or linked with a molecular marker (allele) or SNP according to the invention as described herein, such as provided in Table B, preferably one or more of Ant2_M2, Ant2_M3, Ant2_M4, Ant2_M5, Ant2_M6, and/or Ant2_M7; or Ant2_HapM1 and (i.e., in combination with) one or more of Ant2_HapM2, Ant2_HapM3, Ant2_HapM4, Ant2_HapM5, Ant2_HapM6, and/or Ant2_HapM7.

In certain embodiments the Ant1 gene (allele) or associated polynucleotides, markers, or SNPs are homozygous. In certain embodiments, the Ant1 gene (allele) or associated polynucleotides, markers, or SNPs are heterozygous. Preferably, the Ant1 gene (allele) or associated polynucleotides, markers, or SNPs are homozygous.

In certain embodiments the Ant2 gene (allele) or associated polynucleotides, markers, or SNPs are homozygous. In certain embodiments, the Ant2 gene (allele) or associated polynucleotides, markers, or SNPs are heterozygous.

In certain preferred embodiments, the Ant1 gene (allele) or associated polynucleotides, markers, or SNPs are homozygous and the Ant2 gene (allele) or associated polynucleotides, markers, or SNPs are homozygous or heterozygous.

In certain embodiments the Ant1-associated polynucleotide, molecular marker (allele), Ant1 gene (allele), or SNP (allele) is homozygous. In certain embodiments, the Ant1-associated polynucleotide, molecular marker (allele), Ant1 gene (allele), or SNP (allele) is heterozygous. Preferably, the Ant1 gene (allele) is homozygous.

In certain embodiments the Ant2-associated polynucleotide, molecular marker (allele), Ant2 gene (allele), or SNP (allele) is homozygous. In certain embodiments, the Ant2-associated polynucleotide, molecular marker (allele), Ant2 gene (allele), or SNP (allele) is heterozygous.

In certain preferred embodiments, the Ant1-associated polynucleotide, molecular marker (allele), Ant1 gene (allele), or SNP (allele) is homozygous and the Ant2-associated polynucleotide, molecular marker (allele), Ant2 gene (allele), or SNP (allele) is homozygous or heterozygous.

It will be understood that homozygosity can be achieved by any means in the art, such as backcrossing and selecting for homozygosity, as well as for instance through doubled haploid induction. Also, homozygosity may be achieved by (recombinant) introduction of the Ant1 gene simultaneously or sequentially on both homologous chromosomes.

A doubled haploid plant or plant part is one that is developed by the doubling of a haploid set of chromosomes. A plant or seed that is obtained from a doubled haploid plant that is selfed any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes. For example, a plant will be considered a doubled haploid plant if it contains viable gametes, even if it is chimeric.

Somatic haploid cells, haploid embryos, haploid seeds, or haploid seedlings produced from haploid seeds can be treated with a chromosome doubling agent. Homozygous plants can be regenerated from haploid cells by contacting the haploid cells, such as embryo cells or callus produced from such cells, with chromosome doubling agents, such as colchicine, pronamide, dithipyr, trifluralin, or another known anti-microtubule agent or anti-microtubule herbicide, or nitrous oxide to create homozygous doubled haploid cells. Treatment of a haploid seed or the resulting seedling generally produces a chimeric plant, partially haploid and partially doubled haploid. It may be beneficial to nick the seedling before treatment with colchicine. When reproductive tissue contains doubled haploid cells, then doubled haploid seed is produced.

In certain embodiments, the Ant1 gene comprises a polynucleotide having a sequence selected from
(1) a sequence having a sequence which is at least 80%, preferably at least 90%, more preferably at least 95%, identical to a sequence as set forth in SEQ ID NO: 1;
(2) a sequence having a sequence which is at least 80%, preferably at least 90%, more preferably at least 95%, identical to a sequence as set forth in SEQ ID NO: 2; and/or
(3) a sequence encoding a protein having a sequence which is at least 80%, preferably at least 90%, more preferably at least 95%, identical to a sequence as set forth in SEQ ID NO: 3.

Preferably, the Ant1 gene comprises one or more, preferably all, of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences).

Preferably, the Ant1 gene comprises or is comprised in a polynucleotide comprising one or more, preferably all, of molecular markers Ant1_HapM1, Ant1_HapM2, Ant1_HapM3, Ant1_HapM4, Ant1_HapM5, and Ant1_HapM6, preferably all, having a SNP as indicated in Table B (at a position corresponding to the indicated position on one or more of the respective sequences).

Preferably, the Ant1 gene comprises one or more, preferably all, polynucleic acid having a sequence as set forth in SEQ ID NOs: 7 to 12 (and comprising the respective SNPs as indicated in Table B).

Preferably, the Ant1 gene comprises or is comprised in a polynucleic acid comprising one or more, preferably all, polynucleic acid having a sequence as set forth in SEQ ID NOs: 7 to 12 (and comprising the respective SNPs as indicated in Table B).

In certain embodiments, the Ant2 gene comprises a polynucleotide having a sequence selected from
(1) a sequence having a sequence which is at least 80%, preferably at least 90%, more preferably at least 95%, identical to a sequence as set forth in SEQ ID NO: 14;
(2) a sequence having a sequence which is at least 80%, preferably at least 90%, more preferably at least 95%, identical to a sequence as set forth in SEQ ID NO: 15; and/or
(3) a sequence encoding a protein having a sequence which is at least 80%, preferably at least 90%, more preferably at least 95%, identical to a sequence as set forth in SEQ ID NO: 16.

Preferably, the Ant2 gene is associated or linked with molecular marker Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences).

Preferably, the Ant2 gene comprises or is comprised in a polynucleic acid associated or linked with, or comprising molecular marker Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and/or Ant2_HapM7, preferably Ant2_M2, Ant2_M3, Ant2_M4, Ant2_M5, Ant2_M6, and/or Ant2_M7; or Ant2_HapM1 and (i.e. in combination with) one or more of Ant2_HapM2, Ant2_HapM3, Ant2_HapM4, Ant2_HapM5, Ant2_HapM6, and/or Ant2_HapM7, having a SNP as indicated in Table B (at a position corresponding to the indicated position on one or more of the respective sequences).

Preferably, the Ant2 gene comprises or is associated or linked with a polynucleic acid having a sequence as set forth in any of SEQ ID NO: 13 or 20 to 32, preferably any one or more of SEQ ID NOs: 20 to 25 or SEQ ID NO: 26 in combination with any one or more of SEQ ID NOs: 27 to 32 (and comprising or associated or linked with the SNP as indicated in Table B).

Preferably, the Ant2 gene comprises or is comprised in a polynucleic acid comprising or associated or linked with a polynucleic acid having a sequence as set forth in SEQ ID NO: 13 or 20 to 32, preferably any one or more of SEQ ID NOs: 20 to 25 or SEQ ID NO: 26 in combination with any one or more of SEQ ID NOs: 27 to 32 (and comprising the SNP as indicated in Table B).

In certain embodiments, introducing into the genome comprises transformation of the plant or plant part with the respective polynucleotide(s), which may advantageously be provided on a vector as is known in the art.

In certain embodiments, the cross according to the invention as referred to herein is a mixed cross or mixed seed production cross. Such crosses are well known in the art. By means of further guidance and without limitation, a mixed (seed production) cross refers to a cross in which seeds of both parental lines are mixed prior to sowing. Hence paternal (i.e., the pollen donor) and maternal (i.e., the pollen acceptor) plants are randomly mixed in the field, as opposed to breeding schemes comprising discrete and separate regions of maternal and paternal plants (in which male and female plants can be distinguished based on their location). Such mixed (seed production) cross schemes are routinely used in hybrid seed production. Ratios of 15/85 pollen donor (paternal line)/pollen acceptor (maternal line) are routine. Advantageously, the maternal line can be male sterile in order to increase hybrid seed production.

In an aspect, the invention relates to a barley plant or plant part obtained or obtainable from the methods for generating or producing a barley plant or plant part according to the invention as described herein, or offspring thereof.

TABLE B
Positions on Morex v2/v3 for Ant1 markers are on chromosome 7H.
Positions on Morex v2/v3 for Ant2 markers are on chromosome 2H.
This table includes sequences which are at least 80%, preferably
at least 90%, more preferably at least 95% identical to the indicated
SEQ ID NOS, preferably either comprising one or more, preferably
all, of the respective Ant1 SNPs or Ant2-(associated) SNPs.

| | SNP | |
|---|---|---|
| Marker | identity | position |
| Ant1_HapM1 | T | 71238822 of Morex v3 |
| | | 68464237of Morex v2 |
| | | 95 of SEQ ID NO: 1 |
| | | 95 of SEQ ID NO: 7 |
| Ant1_HapM2 | C | 71238454 of Morex v3 |
| | | 68463869 of Morex v2 |
| | | 463 of SEQ ID NO: 1 |
| | | 224 of SEQ ID NO: 8 |
| Ant1_HapM3 | C | 71238052 of Morex v3 |
| | | 68463467 of Morex v2 |
| | | 865 of SEQ ID NO: 1 |
| | | 219 of SEQ ID NO: 2 |
| | | 206 of SEQ ID NO: 9 |
| Ant1_HapM4 | C | 71237825 of Morex v3 |
| | | 68463240 of Morex v2 |
| | | 1092 of SEQ ID NO: 1 |
| | | 446 of SEQ ID NO: 2 |
| | | 73 of SEQ ID NO: 10 |
| Ant1_HapM5 | C | 71237633 of Morex v3 |
| | | 68463048 of Morex v2 |
| | | 1284 of SEQ ID NO: 1 |
| | | 638 of SEQ ID NO: 2 |
| | | 145 of SEQ ID NO: 11 |
| Ant1_HapM6 | G | 71237608 of Morex v3 |
| | | 68463023 of Morex v2 |
| | | 1309 of SEQ ID NO: 1 |
| | | 663 of SEQ ID NO: 2 |
| | | 50 of SEQ ID NO: 12 |
| Ant2_M1 | C | 591324765 of Morex v3 |
| | | 599993462 of Morex v2 |
| | | 101 of SEQ ID NO: 13 |
| Ant2_M2 | G | 593492855 of Morex v3 |
| | | 491 of SEQ ID NO: 14 |
| | | 46 of SEQ ID NO: 20 |
| Ant2_M3 | C | 593493961 of Morex v3 |
| | | 1597 of SEQ ID NO: 14 |
| | | 96 of SEQ ID NO: 21 |
| Ant2_M4 | T | 593494314 of Morex v3 |
| | | 1950 of SEQ ID NO: 14 |
| | | 64 of SEQ ID NO: 22 |
| Ant2_M5 | G | 593494569 of Morex v3 |
| | | 2207 of SEQ ID NO: 14 |
| | | 73 of SEQ ID NO: 23 |
| Ant2_M6 | T | 593495307 of Morex v3 |
| | | 2946 of SEQ ID NO: 14 |
| | | 101 of SEQ ID NO: 24 |
| Ant2_M7 | A | 593496606 of Morex v3 |
| | | 3907 of SEQ ID NO: 14 |
| | | 804 of SEQ ID NO: 15 |
| | | 72 of SEQ ID NO: 25 |
| Ant2_HapM1 | G | 593496442 of Morex v3 |
| | | 3743 of SEQ ID NO: 14 |
| | | 640 of SEQ ID NO: 15 |
| | | 84 of SEQ ID NO: 26 |
| Ant2_HapM2 | C | 593492774 of Morex v3 |
| | | 410 of SEQ ID NO: 14 |
| | | 76 of SEQ ID NO: 27 |
| Ant2_HapM3 | A | 593493167 of Morex v3 |
| | | 803 of SEQ ID NO: 14 |
| | | 49 of SEQ ID NO: 15 |
| | | 94 of SEQ ID NO: 28 |
| Ant2_HapM4 | T | 593494160 of Morex v3 |
| | | 1796 of SEQ ID NO: 14 |
| | | 88 of SEQ ID NO: 29 |
| Ant2_HapM5 | A | 593494377 of Morex v3 |
| | | 2013 of SEQ ID NO: 14 |
| | | 52 of SEQ ID NO: 30 |
| Ant2_HapM6 | G | 593495891 of Morex v3 |
| | | 3530 of SEQ ID NO: 14 |
| | | 80 of SEQ ID NO: 31 |
| Ant2_HapM7 | C | 593497594 of Morex v3 |
| | | 4895 of SEQ ID NO: 14 |
| | | 1619 of SEQ ID NO: 15 |
| | | 73 of SEQ ID NO: 32 |

In an aspect, the invention relates to a (isolated) polynucleotide having a sequence comprised in, comprising, or consisting of a polynucleotide sequence, gene sequence, coding sequence, protein sequence, molecular marker sequence, or primer sequence according to the invention as described herein, a (unique) fragment thereof, or the complement or reverse complement thereof, or a (unique) fragment thereof.

In an aspect, the invention relates to a (isolated) polynucleotide comprising a polynucleotide, molecular marker (allele), SNP, Ant1 gene or coding sequence thereof, or Ant2 gene or coding sequence thereof according to the invention as described herein, the complement or the reverse complement thereof, or a (unique) fragment thereof or the complement or reverse complement thereof.

In an aspect, the invention relates to a (isolated) polynucleotide having a sequence comprised in, comprising, or consisting of a sequence as set forth in any of SEQ ID NOs: 1 to 32, a (unique) fragment thereof, or the complement or reverse complement thereof, or a (unique) fragment thereof, or the complement or reverse complement thereof.

In an aspect, the invention relates to a (isolated) polynucleotide having a sequence which is at least 80%, preferably at least 90%, more preferably at least 95% identical to a sequence comprised in, comprising, or consisting of a sequence as set forth in any of SEQ ID NOs: 1 to 32, a (unique) fragment thereof, or the complement or reverse complement thereof, or a (unique) fragment thereof, or the complement or reverse complement thereof.

In an aspect, the invention relates to a (isolated) polynucleotide having a sequence comprised in, comprising, or consisting of a sequence as set forth in any of SEQ ID NOs: 7 to 13 or 20 to 32, a (unique) fragment thereof, or the complement or reverse complement thereof, or a (unique) fragment thereof, or the complement or reverse complement thereof.

In an aspect, the invention relates to a (isolated) polynucleic acid comprising one or more molecular marker (marker allele) according to the invention as described herein, or a (unique) fragment thereof, and/or the complement or reverse complement thereof.

In an aspect, the invention relates to a (isolated) polynucleic acid comprising one or more SNP according to the invention as described herein, or a (unique) fragment thereof, and/or the complement or reverse complement thereof.

In an aspect, the invention relates to a (isolated) polynucleotide having a sequence which is at least 80%, preferably at least 90%, more preferably at least 95% identical to a sequence comprised in, comprising, or consisting of a sequence as set forth in any of SEQ ID NOs: 7 to 13 or 20 to 32, a (unique) fragment thereof, or the complement or reverse complement thereof, or a (unique) fragment thereof, or the complement or reverse complement thereof.

In an aspect, the invention relates to a (isolated) comprising at least 15 contiguous nucleotides, preferably at least 18 contiguous nucleotides, more preferably at least 20 contiguous nucleotides of a sequence as set forth in any of SEQ ID NOs: 7 to 13 or 20 to 32, preferably comprising the respective SNP as indicated in Table A or B, preferably as the most 3′ nucleotide, the complement or reverse complement thereof.

In an aspect, the invention relates to a (isolated) polynucleotide having a sequence which is at least 80%, preferably at least 90%, more preferably at least 95% identical to a sequence comprised in, comprising, or consisting of a sequence as set forth in any of SEQ ID NOs: 7 to 13 or 20 to 32, a (unique) fragment thereof, preferably comprising the respective SNP as indicated in Table A or B, preferably as the most 3′ nucleotide, or the complement or reverse complement thereof, or a (unique) fragment thereof, or the complement or reverse complement thereof.

In an aspect, the invention relates to a (isolated) polynucleic acid comprising, comprised in, or consisting of a sequence selected from
(1) a sequence having a sequence which is at least 80%, preferably at least 90%, more preferably at least 95%, identical to a sequence as set forth in SEQ ID NO: 1, the complement or reverse complement thereof, or a (unique) fragment thereof, the complement or reverse complement thereof;
(2) a sequence having a sequence which is at least 80%, preferably at least 90%, more preferably at least 95%, identical to a sequence as set forth in SEQ ID NO: 2, the complement or reverse complement thereof, or a (unique) fragment thereof, the complement or reverse complement thereof; and/or
(3) a sequence encoding a protein having a sequence which is at least 80%, preferably at least 90%, more preferably at least 95%, identical to a sequence as set forth in SEQ ID NO: 3, the complement or reverse complement thereof, or a (unique) fragment thereof, the complement or reverse complement thereof.

In certain embodiments, the (isolated) polynucleic acid of the invention as described herein comprises one or more, preferably, all of polynucleic acids having a sequence which is at least 80%, preferably at least 90%, more preferably at least 95%, identical to a sequence as set forth in SEQ ID NOs: 7 to 12.

In certain embodiments, the (isolated) polynucleic acid of the invention as described herein comprises one or more, preferably all, of the Ant1 SNPs of Table B.

In certain embodiments, the (isolated) polynucleic acid of the invention as described herein comprises one or more, preferably, all of polynucleic acids having a sequence which is at least 80%, preferably at least 90%, more preferably at least 95%, identical to a sequence as set forth in SEQ ID NOs: 7 to 12, and the (isolated) polynucleic acid of the invention as described herein comprises one or more, preferably all, of the Ant1 SNPs of Table B.

In an aspect, the invention relates to a (isolated) polynucleic acid comprising, comprised in, or consisting of a sequence selected from
(1) a sequence having a sequence which is at least 80%, preferably at least 90%, more preferably at least 95%, identical to a sequence as set forth in SEQ ID NO: 14, the complement or reverse complement thereof, or a (unique) fragment thereof, the complement or reverse complement thereof;
(2) a sequence having a sequence which is at least 80%, preferably at least 90%, more preferably at least 95%, identical to a sequence as set forth in SEQ ID NO: 15, the complement or reverse complement thereof, or a (unique) fragment thereof, the complement or reverse complement thereof; and/or
(3) a sequence encoding a protein having a sequence which is at least 80%, preferably at least 90%, more preferably at least 95%, identical to a sequence as set forth in SEQ ID NO: 16, the complement or reverse complement thereof, or a (unique) fragment thereof, the complement or reverse complement thereof.

In certain embodiments, the (isolated) polynucleic acid of the invention as described herein comprises a polynucleic acid having a sequence which is at least 80%, preferably at least 90%, more preferably at least 95%, identical to a sequence as set forth in SEQ ID NO: 13 or 20 to 32.

In certain embodiments, the (isolated) polynucleic acid of the invention as described herein comprises the Ant2 SNP of Table B.

In certain embodiments, the (isolated) polynucleic acid of the invention as described herein comprises a polynucleic acid having a sequence which is at least 80%, preferably at least 90%, more preferably at least 95%, identical to a sequence as set forth in SEQ ID NO: 13 or 20 to 32, and the (isolated) polynucleic acid of the invention as described herein comprises the Ant2 SNP of Table B.

In certain embodiments, the polynucleotide comprises at least 15, preferably at least 18, more preferably at least 20, contiguous nucleotides of any of SEQ ID NO: 7 to 13 or 20 to 32, or complementary to contiguous nucleotides of any of SEQ ID NO: 7 to 13 or 20 to 32, or reverse complementary to contiguous nucleotides of any of SEQ ID NO: 7 to 13 or 20 to 32.

In certain embodiments, the polynucleotide comprises at least 15, preferably at least 18, more preferably at least 20, contiguous nucleotides of any of SEQ ID NO: 7 to 13 or 20 to 32 and comprising the respective SNP; or complementary to contiguous nucleotides of any of SEQ ID NO: 7 to 13 or 20 to 32, or reverse complementary to contiguous nucleotides of any of SEQ ID NO: 7 to 13 or 20 to 32.

In certain embodiments, the polynucleotide comprises at least 15, preferably at least 18, more preferably at least 20, contiguous nucleotides of any of SEQ ID NO: 7 to 13 or 20 to 32 and comprising the respective SNP as the most 3′, second most 3′, or third most 3′ nucleotide; or complementary to contiguous nucleotides of any of SEQ ID NO: 7 to 13 or 20 to 32, or reverse complementary to contiguous nucleotides of any of SEQ ID NO: 7 to 13 or 20 to 32.

In certain embodiments, the (isolated) polynucleic acid according to the invention as described herein comprises one or more, optionally all, Ant1 or Ant2 SNP as defined in Table A.

In certain embodiments, the (isolated) polynucleic acid according to the invention as described herein comprises one or more, optionally all, Ant1 or Ant2 SNP as defined in Table B.

In certain embodiments, the (isolated) polynucleic acid according to the invention as described herein comprises one or more, optionally all, “purple” Ant1 or Ant2 SNP as defined in Table A.

In certain embodiments, the (isolated) polynucleic acid according to the invention as described herein comprises one or more, optionally all, “purple” Ant1 or Ant2 SNP as defined in Table B.

In certain embodiments, the (isolated) polynucleic acid according to the invention as described herein comprises one or more, optionally all, “non-purple” Ant1 or Ant2 SNP as defined in Table A.

In certain embodiments, the (isolated) polynucleic acid according to the invention as described herein comprises one or more, optionally all, “non-purple” Ant1 or Ant2 SNP as defined in Table B.

In certain embodiments, the (isolated) polynucleic acid according to the invention as described herein comprises one or more, optionally all, SNP of Ant1_HapM1, Ant1_HapM2, Ant1_HapM3, Ant1_HapM4, Ant1_HapM5, and Ant1_HapM6.

In certain embodiments, the (isolated) polynucleic acid according to the invention as described herein comprises one or more, optionally all, “purple” SNP of Ant1_HapM1, Ant1_HapM2, Ant1_HapM3, Ant1_HapM4, Ant1_HapM5, and Ant1_HapM6.

In certain embodiments, the (isolated) polynucleic acid according to the invention as described herein comprises one or more, optionally all, “non-purple” SNP of Ant1_HapM1, Ant1_HapM2, Ant1_HapM3, Ant1_HapM4, Ant1_HapM5, and Ant1_HapM6.

In certain embodiments, the (isolated) polynucleic acid according to the invention as described herein comprises one or more, optionally all SNP of Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably one or more SNP of Ant2_M2, Ant2_M3, Ant2_M4, Ant2_M5, Ant2_M6, and Ant2_M7, or the SNP of Ant2_HapM1 combined with any one or more SNP of Ant2_HapM2, Ant2_HapM3, Ant2_HapM4, Ant2_HapM5, Ant2_HapM6, or Ant2_HapM7.

In certain embodiments, the (isolated) polynucleic acid according to the invention as described herein comprises one or more, optionally all, “purple” SNP of Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably one or more SNP of Ant2_M2, Ant2_M3, Ant2_M4, Ant2_M5, Ant2_M6, and Ant2_M7, or the SNP of Ant2_HapM1 combined with any one or more SNP of Ant2_HapM2, Ant2_HapM3, Ant2_HapM4, Ant2_HapM5, Ant2_HapM6, or Ant2_HapM7.

In certain embodiments, the (isolated) polynucleic acid according to the invention as described herein comprises one or more, optionally all, “non-purple” SNP of Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably one or more SNP of Ant2_M2, Ant2_M3, Ant2_M4, Ant2_M5, Ant2_M6, and Ant2_M7, or the SNP of Ant2_HapM1 combined with any one or more SNP of Ant2_HapM2, Ant2_HapM3, Ant2_HapM4, Ant2_HapM5, Ant2_HapM6, or Ant2_HapM7.

In an aspect, the invention relates to a (isolated) polynucleic acid specifically hybridizing with a polynucleic acid according to the invention as described herein, the complement thereof, or the reverse complement thereof, in particular a primer or probe.

In certain embodiments, the (isolated) polynucleotide is a primer or a probe. In certain embodiments, the (isolated) polynucleotide is an allele-specific primer. In certain embodiments, the (isolated) polynucleotide is a KASP primer.

In certain aspects, the invention relates to (unique) fragments of the polynucleic acids of the invention, preferably of at least 15 nucleotides, more preferably of at least 18 nucleotides, most preferably of at least 20 nucleotides.

In an aspect, the invention relates to a primer or probe capable of specifically detecting the polynucleotide, molecular marker (allele), SNP, Ant1 gene or coding sequence thereof, or Ant2 gene or coding sequence thereof according to the invention as described herein. Such primer may be specific for the “purple” polynucleotide, Ant1 gene allele, Ant2 gene allele, SNP allele, or molecular marker allele. Alternatively, such primer may be specific for the “non-purple” polynucleotide, Ant1 gene allele, Ant2 gene allele, SNP allele, or molecular marker allele.

In certain embodiments, the polynucleic acid (e.g., primer or probe) is capable of discriminating between a molecular marker (allele) of the invention, such as a “purple” SNP, and a different molecular marker allele, such as a non-purple SNP, such as to specifically hybridize either with a molecular marker allele of the invention or with a different molecular marker allele.

In an aspect, the invention relates to a primer set or probe set capable of specifically detecting the polynucleotide, molecular marker (allele), SNP, Ant1 gene or coding sequence thereof, or Ant2 gene or coding sequence thereof according to the invention as described herein. Such primer set may comprise one or two primers specific for the “purple” polynucleotide, Ant1 gene allele, Ant2 gene allele, SNP allele, or molecular marker allele and/or one or two primers specific for the “non-purple” polynucleotide, Ant1 gene allele, Ant2 gene allele, SNP allele, or molecular marker allele. Such primer set may comprise a common primer and two allele-specific primers.

It will be understood that in embodiments relating to a set of forward and reverse primers, only one of both primers (forward or reverse) may need to be capable of discriminating between different marker alleles (or may be specific for one particular marker allele) of the invention, and hence may be unique (e.g., an allele-specific primer). The other primer may or may not be capable of discriminating between different marker alleles of the invention, and hence may or may not be unique (e.g., a common primer).

The skilled person will understand that the polynucleic acids, such as primers or probes, may further comprise (5′) additional nucleotides, which for instance may serve as a tag, tail, label, or barcode, etc.

In certain embodiments, the polynucleotides of the invention as described herein have a length of at most 1 Mb, preferably at most 500 kb, more preferably at most 200 kb, most preferably at most 100 kb. Such lengths are appropriate for describing polynucleotides in the context of introgression, such as introgression of a QTL or genomic locus.

In certain embodiments, the polynucleotides of the invention as described herein have a length of at least 10 kb, preferably at least 50 kb bp, more preferably at least 100 kb. Such lengths are appropriate for describing polynucleotides in the context of introgression, such as introgression of a QTL or genomic locus.

In certain embodiments, the polynucleotides of the invention as described herein have a length of at most 1 Mb, preferably at most 500 kb, more preferably at most 200 kb, most preferably at most 100 kb and have a length of at least 10 kb, preferably at least 50 kb bp, more preferably at least 100 kb. Such lengths are appropriate for describing polynucleotides in the context of introgression, such as introgression of a QTL or genomic locus.

In certain embodiments, the polynucleotides of the invention as described herein have a length of at most 20 kb, preferably at most 10 kb, more preferably at most 5 kb. Such lengths are appropriate for describing polynucleotides in the context of transgenesis, such as transgenesis of genes or markers.

In certain embodiments, the polynucleotides of the invention as described herein have a length of at least 1000 bp, preferably at least 3000 bp, more preferably at least 5000 bp. Such lengths are appropriate for describing polynucleotides in the context of transgenesis, such as transgenesis of genes or markers.

In certain embodiments, the polynucleotides of the invention as described herein have a length of at most 20 kb, preferably at most 10 kb, more preferably at most 5 kb and have a length of at least 1000 bp, preferably at least 3000 bp, more preferably at least 5000 bp. Such lengths are appropriate for describing polynucleotides in the context of transgenesis, such as transgenesis of genes or markers.

In certain embodiments, the polynucleotides of the invention as described herein have a length of at most 250 bp, preferably at most 100 bp, more preferably at most 50 bp. Such lengths are appropriate for describing polynucleotides in the context of primers or probes.

In certain embodiments, the polynucleotides of the invention as described herein have a length of at least 15 bp, preferably at least 18 bp, more preferably at least 20 bp. Such lengths are appropriate for describing polynucleotides in the context of primers or probes.

In certain embodiments, the polynucleotides of the invention as described herein have a length of at most 250 bp, preferably at most 100 bp, more preferably at most 50 bp and have a length of at least 15 bp, preferably at least 18 bp, more preferably at least 20 bp. Such lengths are appropriate for describing polynucleotides in the context of primers or probes.

In an aspect, the invention relates to a vector comprising a polynucleic acid according to the invention as described herein. In certain embodiments, the vector is a (plant) expression vector. In certain embodiments, the vector is an inducible (plant) expression vector. In certain embodiments, the expression is tissue- or organ-specific. In certain embodiments, the expression is developmentally specific. In certain embodiments, the expression is tissue- or organ-specific and developmentally specific.

As used herein, a “vector” has its ordinary meaning in the art, and may for instance be a plasmid, a cosmid, a phage or an expression vector, a transformation vector, shuttle vector, or cloning vector; it may be double- or single-stranded, linear or circular; or it may transform a prokaryotic or eukaryotic host, either via integration into its genome or extrachromosomally. The nucleic acid according to the invention is preferably operatively linked in a vector with one or more regulatory sequences which allow the transcription, and, optionally, the expression, in a prokaryotic or eukaryotic host cell. A regulatory sequence—preferably, DNA—may be homologous or heterologous to the nucleic acid according to the invention. For example, the nucleic acid is under the control of a suitable promoter or terminator. Suitable promoters may be promoters which are constitutively induced (example: 35S promoter from the “Cauliflower mosaic virus” (Odell et al., 1985); those promoters which are tissue-specific are especially suitable (example: Pollen-specific promoters, Chen et al., (2010), Zhao et al., (2006), or Twell et al., (1991)), or are development-specific (example: blossom-specific promoters). Suitable promoters may also be synthetic or chimeric promoters which do not occur in nature, are composed of multiple elements, and contain a minimal promoter, as well as—upstream of the minimum promoter—at least one cis-regulatory element which serves as a binding location for special transcription factors. Chimeric promoters may be designed according to the desired specifics and are induced or repressed via different factors. Examples of such promoters are found in Gurr & Rushton (2005) or Venter (2007). For example, a suitable terminator is the nos-terminator (Depicker et al., 1982). The vector may be introduced via conjugation, mobilization, biolistic transformation, agrobacteria-mediated transformation, transfection, transduction, vacuum infiltration, or electroporation. The vector may be a plasmid, a cosmid, a phage or an expression vector, a transformation vector, shuttle vector, or cloning vector; it may be double- or single-stranded, linear or circular. The vector may transform a prokaryotic or eukaryotic host, either via integration into its genome or extrachromosomally.

As used herein, the term “operatively linked” or “operably linked” means connected in a common nucleic acid molecule in such a manner that the connected elements are positioned and oriented relative to one another such that a transcription of the nucleic acid molecule may occur. A DNA which is operatively linked with a promoter is under the transcriptional control of this promoter. In an aspect, the invention relates to the use of the polynucleic acid or vector according to the invention as described herein for generating a barley plant or plant part.

In certain embodiments, the vector is an expression vector. The nucleic acid is preferably operatively linked in a vector with one or more regulatory sequences which allow the transcription, and optionally the expression, in a prokaryotic or eukaryotic host cell. A regulatory sequence may be homologous or heterologous to the nucleic acid. For example, the nucleic acid is under the control of a suitable promoter or terminator. Suitable promoters may be promoters which are constitutively induced, for example, the 35S promoter from the “Cauliflower mosaic virus” (Odell et al., 1985. Identification of DNA sequences required for activity of the cauliflower mosaic virus 35S promoter.) Tissue-specific promoters, e.g., pollen-specific promoters as described in Chen et al., (2010. Molecular Biology Reports 37 (2): 737-744), Zhao et al., (2006. Planta 224 (2): 405-412), or Twell et al., (1991. Genes & Development 5 (3): 496-507), are particularly suitable, as are development-specific promoters, e.g., blossom-specific promoters. Suitable promoters may also be synthetic or chimeric promoters which do not occur in nature, and which are composed of multiple elements. Such synthetic or chimeric promoter may contain a minimal promoter, as well as at least one cis-regulatory element which serves as a binding location for special transcription factors. Chimeric promoters may be designed according to the desired specifics and can be induced or repressed via different factors. Examples of such promoters are found in Gurr & Rushton (2005. Trends in Biotechnology 23 (6): 275-282) or Venter (2007. Trends in Plant Science: 12 (3): 118-124). For example, a suitable terminator is the nos-terminator (Depicker et al., 1982. Journal of Molecular and Applied Genetics 1 (6): 561-573).

In certain embodiments, the vector is a conditional expression vector. In certain embodiments, the vector is a constitutive expression vector. In certain embodiments, the vector is a tissue-specific expression vector, such as a pollen-specific expression vector. In certain embodiments, the vector is an inducible expression vector. All such vectors are well-known in the art. Methods for preparation of the described vectors are commonplace to the person skilled in the art (Sambrook et al., 2001).

Also envisaged herein is a host cell, such as a plant cell, which comprises a nucleic acid as described herein, or a vector as described herein. The host cell may contain the polynucleic acid as an extra-chromosomally (episomal) replicating molecule or comprises the polynucleic acid integrated in the nuclear or plastid genome of the host cell, or as introduced chromosome, e.g., minichromosome.

The host cell may be a prokaryotic (for example, bacterial) or eukaryotic cell (for example, a plant cell or a yeast cell). For example, the host cell may be an agrobacterium, such as Agrobacterium tumefaciens or Agrobacterium rhizogenes. Preferably, the host cell is a plant cell.

In an aspect, the invention relates to the use of the polynucleic acid or vector according to the invention as described herein for identifying a barley plant or plant part, such as a barley plant or plant part comprising or lacking an “purple” Ant1 and/or Ant2 gene allele, molecular marker allele, or SNP allele.

A polynucleic acid described herein or a vector described herein may be introduced in a host cell via well-known methods, which may depend on the selected host cell, including, for example, conjugation, mobilization, biolistic transformation, agrobacteria-mediated transformation, transfection, transduction, vacuum infiltration, or electroporation. In particular, methods for introducing a nucleic acid or a vector in an agrobacterium cell are well-known to the skilled person and may include conjugation or electroporation methods. Also, methods for introducing a nucleic acid or a vector into a plant cell are known (Sambrook et al., 2001) and may include diverse transformation methods such as biolistic transformation and agrobacterium-mediated transformation.

In particular embodiments, the present invention relates to a transgenic plant cell which comprises a nucleic acid as described herein as a transgene, or a vector as described herein. In further embodiments, the present invention relates to a transgenic plant or a part thereof which comprises the transgenic plant cell.

For example, such a transgenic plant cell or transgenic plant is a plant cell or plant which is, preferably stably, transformed with a polynucleic acid as described herein or a vector as described herein.

Preferably, the polynucleic acid in the transgenic plant cell is operatively linked with one or more regulatory sequences which allow the transcription, and optionally the expression, in the plant cell. A regulatory sequence may be homologous or heterologous to the nucleic acid. The total structure made up of the nucleic acid according to the invention and the regulatory sequence(s) may then represent the transgene.

The skilled person will understand that any of the sequences of the invention as described herein may be used for the identification of a barley plant or plant part and/or for the production of a barley plant or plant part. Accordingly, in an aspect, the invention relates to the use of a polynucleic acid, Ant1 gene (allele) or coding sequence thereof, or Ant2 gene (allele) or coding sequence thereof according to the invention as described herein for identifying (in particular primer or probe sequences) or generating (in particular gene sequences) a barley plant or plant part.

All aspects and embodiments relating to Ant1/Ant2 as described herein are equally applicable to Blp, mutatis mutandis.

### Blp Gene (Black Lemma Pericarp-Gen) Controlling Black/Dark Color in Seed Coat

Melanins are dark pigmented biopolymers, among which plant-based Melanins are classified as allomelanins (Shoeva et. al 2020, Glagoleva et. al 2020). The black melanin coloration of the barley ear is controlled by the dominant monogenic gene locus Blp1 (Black Lemmar Pericarp 1). Mapped was the locus on chromosome 1HL (Glagoleva et. al 2017, Long et. al 2019). Accumulation of melanins can occur in different tissues of the barley stalk e.g., awns, palea, lemma and pericarp (Glagoleva et. al 2017, Long et. al 2019, Shoeva et. al 2020). First black pigments could be observed on the grain at late milk maturity or early dough maturity. The color was expressed as a spot on the dorsal side of the grain or as a stripe on the palea. Further the lemma and the palea became colored in the early dough ripening. A barley plant or plant part producing or capable of producing a seed having a black/dark pericarp and/or hull is meant to include the abovementioned seed pigmentation which may be black or any shade of grey.

The present invention is in particular captured by any one or any combination of one or more of the below numbered items 1 to 13, with any other item, statement and/or embodiments.

1. A method for identifying and/or selecting a barley plant or plant part, comprising screening for the presence of a polynucleotide comprising the molecular marker (allele) Blp_M1 in the genome of the plant or plant part.

2. The method according to items 1, wherein
Blp_M1 is or comprises a SNP at a position corresponding to position 498773011 bp (on chromosome 1H) of reference genome sequence assembly of Barley cultivar Morex v3.0, or wherein Blp_M1 is or comprises a SNP at a position corresponding to position 101 of SEQ ID NO: 33.

3. The method according to item 2, wherein said SNP is C in Blp_M1.

4. The method according to any of items 1 to 3, which is a method for identifying a barley plant or plant part producing or capable of producing a seed having a black/dark pericarp and/or hull, and/or a plant grown therefrom is a plant producing or capable of producing a seed having a black/dark pericarp and/or hull.

5. The method according to any of items 1 to 4, wherein said barley plant or plant part is identified as producing or capable of producing a seed having a black/dark pericarp and/or hull if a SNP is identified which is C at a position corresponding to position 498773011 bp (on chromosome 1H) of reference genome sequence assembly of Barley cultivar Morex v3.0 and/or at a position corresponding to position 101 of SEQ ID NO: 33.

6. The method according to any of items 1 to 4, wherein said barley plant or plant part is identified as not producing or capable of producing a seed having a black/dark pericarp and/or hull if a SNP is identified which is A or which is not C at a position corresponding to position 498773011 bp (on chromosome 1H) of reference genome sequence assembly of Barley cultivar Morex v3.0 and/or at a position corresponding to position 101 of SEQ ID NO: 33.

7. The method according to any of items 1 to 6, wherein Blp_M1 comprises or is comprised in a polynucleotide having a sequence as set forth in SEQ ID NO: 33.

8. A male sterile barley plant or plant part (optionally identified according to the method of any of items 1 to 7), preferably a cytoplasmic male sterile barley plant or plant part, comprising (preferably a homozygous) molecular marker (allele) Blp_M1, preferably wherein said plant or plant has a SNP which is C at a position corresponding to position 498773011 bp (on chromosome 1H) of reference genome sequence assembly of Barley cultivar Morex v3.0 and/or at a position corresponding to position 101 of SEQ ID NO: 33.

9. A method for producing, obtaining, or selecting a (hybrid) barley seed (mixture), comprising
providing a barley (bulk or unselected) seed mixture harvested from a cross between a first barley parent plant (population) and a second barley parent plant (population),
wherein one of said first or second barley parent plant (population) is male sterile, preferably cytoplasmic male sterile,
wherein only said first barley parent plant (population) comprises the molecular marker (allele) Blp_M1, as defined in any of items 1 to 5 or 7 to 8, and
selecting seeds having a black palea and/or lemma and/or pericarp if said first barley plant (population) is (cytoplasmic) male sterile and selecting seeds not having a black palea and/or lemma and/or pericarp if said second barley plant (population) is (cytoplasmic) male sterile.

10. A (isolated) polynucleotide having a sequence comprising, comprised in, or consisting of a sequence as set forth in SEQ ID NO: 33, or a (unique) fragment thereof, or the complement or reverse complement of any thereof.

11. The fragment of the (isolated polynucleotide according to item 10, comprising at least 15, preferably at last 18, more preferably at least 20 contiguous nucleotides of SEQ ID NO: 33, including position 101 of SEQ ID NO: 33 (preferably as most 3′ nucleotide), the complement, or reverse complement thereof.

12. The fragment of the (isolated polynucleotide according to item 10 or 11, for use in producing hybrid barley seed having a black/dark pericarp and/or hull.

13. The barley plant or plant part according to any of item 1 to 7, wherein said plant or plant part is obtained by introgression of said molecular marker (allele), SNP and/or Blp gene.

In another aspect, the present invention comprises a method for identifying and selecting a barley plant or plant part, comprising screening for the presence of a polynucleotide comprising the molecular marker (allele) Blp_M1 in the genome of the barley plant or barley plant part and selecting a barley plant or plant part having the molecular marker (allele) Blp_M1 in the genome.

In yet another aspect, the present invention relates to a method for identifying and selecting a barley plant or plant part, comprising screening for the presence of a polynucleotide comprising the molecular marker (allele) Blp_M1 in the genome of the barley plant or barley plant part and not selecting a barley plant or plant part not having the molecular marker (allele) Blp_M1 in the genome.

Further, the present invention relates in another aspect to a method for identifying and selecting a barley plant or plant part, comprising screening for the presence of a polynucleotide comprising the molecular marker (allele) Blp_M1 in the genome of the barley plant or barley plant part and selecting a barley plant or plant part having the molecular marker (allele) Blp_M1 in the genome wherein Blp_M1 is or comprises a SNP at a position corresponding to position 498773011 bp (on chromosome 1H) of reference genome sequence assembly of Barley cultivar Morex v3.0, or wherein Blp_M1 is or comprises a SNP at a position corresponding to position 101 of SEQ ID NO: 33.

The method of the present invention comprises in another aspect a method for identifying and selecting a barley plant or plant part, comprising screening for the presence of a SNP at a position corresponding to position 498773011 bp (on chromosome 1H) of reference genome sequence assembly of Barley cultivar Morex v3.0.

In another aspect, the present invention comprises a method for identifying and selecting a barley plant or plant part, comprising screening for the presence of a C at a position corresponding to position 498773011 bp (on chromosome 1H) of reference genome sequence assembly of Barley cultivar Morex v3.0 or corresponding to position 101 of SEQ ID NO: 33.

The male sterile barley plant or plant part, is in another aspect of the present invention preferably a cytoplasmic male sterile barley plant or plant part, comprising a homozygous molecular marker (allele) Blp_M1, wherein said plant or plant part has a SNP which is C at a position corresponding to position 498773011 bp (on chromosome 1H) of reference genome sequence assembly of Barley cultivar Morex v3.0 and/or at a position corresponding to position 101 of SEQ ID NO: 33.

In yet another aspect, the present invention comprises a male sterile barley plant or plant part identified by screening for the presence of a SNP at a position corresponding to position 498773011 bp (on chromosome 1H) of reference genome sequence assembly of Barley cultivar Morex v3.0 and/or at a position corresponding to position 101 of SEQ ID NO: 33, preferably a cytoplasmic male sterile barley plant or plant part, comprising (preferably a homozygous) molecular marker (allele) Blp_M1, preferably wherein said SNP is a C.

Further, the present invention relates to a method for producing a hybrid barley seed, comprising providing an unselected barley seed mixture harvested from a cross between a first barley parent plant (population) and a second barley parent plant (population),
wherein said first barley parent plant (population) is male sterile, preferably cytoplasmic male sterile,
wherein only said first barley parent plant (population) comprises the molecular marker (allele) Blp_M1, wherein Blp_M1 is or comprises a SNP at a position corresponding to position 498773011 bp (on chromosome 1H) of reference genome sequence assembly of Barley cultivar Morex v3.0, or wherein Blp_M1 is or comprises a SNP at a position corresponding to position 101 of SEQ ID NO: 33, and
selecting seeds not having a black palea and/or lemma and/or pericarp.

In an aspect, the invention relates to a method for producing a hybrid barley seed, comprising providing an unselected barley seed mixture harvested from a cross between a first barley parent plant (population) and a second barley parent plant (population),
wherein said first barley parent plant (population) is male sterile, preferably cytoplasmic male sterile,
wherein only said first barley parent plant (population) comprises the molecular marker (allele) Blp_M1, wherein Blp_M1 is or comprises a SNP at a position corresponding to position 498773011 bp (on chromosome 1H) of reference genome sequence assembly of Barley cultivar Morex v3.0, or wherein Blp_M1 is or comprises a SNP at a position corresponding to position 101 of SEQ ID NO: 33, and
selecting seeds not having a black palea and/or lemma and/or pericarp, wherein the color selection is performed by a seed sorter.

A further aspect of the present invention comprises a method for producing a hybrid barley seed, comprising
providing an unselected barley seed mixture harvested from a cross between a first barley parent plant (population) and a second barley parent plant (population),
wherein said first barley parent plant (population) is male sterile, preferably cytoplasmic male sterile,
wherein only said first barley parent plant (population) comprises the molecular marker (allele) Blp_M1, wherein Blp_M1 is or comprises a SNP at a position corresponding to position 498773011 bp (on chromosome 1H) of reference genome sequence assembly of Barley cultivar Morex v3.0, or wherein Blp_M1 is or comprises a SNP at a position corresponding to position 101 of SEQ ID NO: 33, and
unselecting seeds having a black palea and/or lemma and/or pericarp, wherein the color selection is performed by a seed sorter.

In another aspect, the present invention comprises a method for producing a hybrid barley seed, comprising
providing an unselected barley seed mixture harvested from a cross between a first barley parent plant (population) and a second barley parent plant (population),
wherein said second barley parent plant (population) is male sterile, preferably cytoplasmic male sterile,
wherein only said first barley parent plant (population) comprises the molecular marker (allele) Blp_M1, wherein Blp_M1 is or comprises a SNP at a position corresponding to position 498773011 bp (on chromosome 1H) of reference genome sequence assembly of Barley cultivar Morex v3.0, or wherein Blp_M1 is or comprises a SNP at a position corresponding to position 101 of SEQ ID NO: 33, and
selecting seeds having a black palea and/or lemma and/or pericarp, wherein the color selection is performed by a seed sorter.

The skilled person will understand that the barley plants or plant parts of the invention as described herein may be used in the production of hybrid barley, in particular hybrid barley seeds. Accordingly, in an aspect, the invention relates to the use of the barley plants or plant parts according to the invention as described herein in hybrid barley (seed) production.

The present invention is not to be limited in scope by the specific embodiments described herein. Indeed, various modifications of the invention in addition to those described herein will become apparent to those skilled in the art from the foregoing description and the accompanying figures. Such modifications are intended to fall under the scope of the appended claims. It is further to be understood that all values may include approximates and are provided for description.

The aspects and embodiments of the invention are further supported by the following non-limiting examples. The following examples, including the experiments conducted and the results achieved, are provided for illustrative purposes only and are not constructed as limiting the present invention.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

A key feature of this invention is the development of genetic markers to introduce the colored seed coat traits from plant genetic resources into elite germplasm that are going to be used as restorer lines. This coloration enables inbred line seeds coming from restorer lines to be recognized on the basis of the seed/grain coat color from the hybrid seeds having non-colored seeds.

There are two color codes being interesting for the barley hybrid seed production due to visual seed sorting and marketing advantages. The “purple” barley is due to the coloration in the pericarp caused by the accumulation of anthocyanins. The color expression is visible very early already in the seedlings of colored plants. Melanins belong to dark pigmented biopolymers, among which plant-based Melanins are classified as allomelanins (Shoeva et al., 2020, Glagoleva et al., 2020). The plant melanin synthesis has not yet been comprehensively described. Black coloration of barley seed has been attributed to the accumulation of phytomelanins in the lemma and pericarp as well as awns and palea. The black color of barley grain is controlled by a single gene (Blp gene) located on barley chromosome one (1H) and the color can be grey to black and stays without fading.

### Example 1

A panel of 18 barley accessions having purple barley color-grains was used to develop segregating F2 mapping populations comprising about 200 plants. Plants of two populations (B20157×B9215 and Fantex×HOR10027) showed a purple phenotype during development. The accessions B9215 and HOR10027 (or HOR15455) were carrying the Ant1 and Ant2 loci controlling the purple barley color-grain trait—the normal non-colored grains crossing partner “B20157” (UK SeedBank) and “Fantex” (KWS line material) hold recessive loci of the anthocyanin pathway. The panel of barley accessions as well as normal line breeding material and both mapping populations were used for marker validation.

A barley public database called Bridge (bridge.ipk-gatersleben.de/#start) was used for the marker development of color codes “purple” barley grain traits. This database contains information of more than 20,000 barley accessions and landraces hosted in Gatersleben genebank which were genotyped. In addition, all germplasm accessions were phenotyped for some traits such as spike, awn and grain color and shape.

From this database, two groups of accessions were selected: one having purple grains and one having white or normal colors. By targeting the chromosomal region containing Ant1 and Ant2 loci, the polymorphism differentiating between the two selected groups were identified. Since Ant1 and Ant2 loci are located on two different chromosomal regions, each region was investigated separately. The flanking sequence of the identified one single SNP for each locus was extracted and used to design KASP marker assays. The developed Ant2 KASP marker was shown to be polymorphic on a tested panel, whereas the developed single marker for Ant1 was monomorphic across to the panel. This led to a hypothesis that instead of one single marker, a set of haplotype markers should be developed to answer the allelic status of the Ant1/Ant2 gene. By blasting the cDNAs against the barley reference genome and other reference assemblies derived from barley pangenome project, several polymorphisms between the accession with colored grain and barley reference genome (cv. Morex, with non-colored grain) were identified. The identified SNPs were used to develop a set of haplotype markers (6 markers) for the Ant1 locus and for the Ant2 locus.

The developed markers for the Ant1 gene and Ant2, single markers were initially tested on the panel consisting of interesting IPK and UK SeedBank accessions showing purple color and as well as several tested varieties (with non-colored grains) led to 100% matching phenotypes with genotypic calls. This result shows a successful validation of the developed markers.

Furthermore, the validated developed markers were applied for the screening of the Ant1 and Ant2 allelic status of the above mentioned two developed F2 mapping populations (B20157×B9215 and Fantex×HOR10027), demonstrating that the developed markers are perfectly functional, diagnostic and explaining the allelic status of all progeny with segregating colored seed coat phenotype in those F2 mapping population as well which could be counted as an additional round of validation for the developed markers (FIG. 1).

The genomic, coding, and protein sequence information of a representative HvMpc1-H1 (Ant1) “purple” and “non-purple” allele and a representative HvMyc2 (Ant2) gene is provided in Table 1. KASP markers for Ant1 and Ant2 genes are listed in Table 2. The KASP markers are based on competitive allele-specific PCR and enables bi-allelic scoring of single nucleotide polymorphisms (SNPs) and insertions and deletions (Indels) at specific loci. The SNP identity for the “purple” Ant1 and Ant2 alleles as well as a possible SNP identity of a representative “non-purple” allele are provided in Table 3. The purple phenotype manifests in case of a homozygous purple Ant1 haplotype (i.e., the combination of markers Ant1_HapM1 to Ant1_HapM6) and/or a homozygous or heterozygous purple Ant2 allele (i.e., Ant2_M1). In Table 4, SNP identity of the Ant1_HapM1 to Ant1_HapM6 markers as well as the Ant2_M1 marker is provided for a number of barley varieties. The seed (pericarp) color for each of these varieties is also provided. In Table 5, SNP identity of the Ant2_HapM1 to Ant2_HapM7 markers as well as the Ant2_M2 to Ant2_M7 markers is provided for a number of barley varieties. The seed (pericarp) color for each of these varieties is also provided.

It is clear that seed color corresponds with the genotype of Table 3.

The list of sequences of the present invention is provided in Table 6.

TABLE 1

| Gene name | Protein type | Chromosome | sequence | SEQ ID NO |
|---|---|---|---|---|
| HvMpc1-H1 | R2R3-Myb | 7HS | Genomic purple allele | 1 |
| (Ant1) | | | Coding purple allele | 2 |
| | | | Protein purple allele | 3 |
| | | | Genomic non-purple allele | 4 |
| | | | Coding non-purple allele | 5 |
| | | | Protein non-purple allele | 6 |
| HvMyc2 | Myc/bHLH | 2H | Genomic purple allele | 14 |
| (Ant2) | | | Coding purple allele | 15 |
| | | | Protein purple allele | 16 |
| | | | Genomic non-purple allele | 17 |
| | | | Coding non-purple allele | 18 |
| | | | Protein non-purple allele | 19 |

TABLE 2

| Locus | | position SNP | Marker sequence (SNP in square brackets - |
|---|---|---|---|
| name | Morex V3 | on Ant1 | donor allele (i.e., purple allele) underlined) |
| Ant1 | 71238822 | 95 of SEQ ID | Ant1_HapM1 |
| (Chr | | NO: 1 or 95 of | atccgtaggcttgccatcatttatatgcctatacatgtgcgtt |
| 7H) | | SEQ ID NO: 4 | gtcgtgtcgcattacaaacttgctacgaaaagtcaacatgttt |
| | | | cagttttt[g/t]tacgtgtagtacttttaatctttaccgttg |
| | | | atacatcttaaagtttagagtgtcaaatcatcaccattcgttt |
| | | | tctatcgttttaatagaagatatttaattattataaaggatgc |
| | | | taagagatgcctattataaacatgttttt |
| | | | (SEQ ID NO: 7; SNP at position |
| | | | 95) |
| | 71238454 | 463 of SEQ | Ant1_HapM2 |
| | | ID NO: 1 or | attatcatctctaaattaaatatgagttttaatataaaaccg |
| | | 464 of SEQ | tattatcaatcattgtacatgttcttagatatgtgctgatgc |
| | | ID NO: 4 | acacatggaagccttcccggccttttggtccatctgacactt |
| | | | gaggaaaggtgctaagcaggccacgtagtcgcgctgcgtcgc |
| | | | cggtggataaatacccccagcaacccgtacgtgctgcccaca |
| | | | gaccactccccct[c/g]tctaacaaagcacacacggcgaga |
| | | | tagagatagagggagagaggttgagaggatggggaggagggc |
| | | | gtgctgtgccaaggaaggggtgaagcgaggggcgtggac |
| | | | gagcaaggaggacgaaagcctggcttcctacgtcaaggcc |
| | | | catggcgaaggccggtggagggagctcccccagcgagct |
| | | | ggtacgtactga (SEQ ID NO: 8; SNP at position |
| | | | 224) |
| | 71238052 | 865 of SEQ | Ant1_HapM3 |
| | | ID NO: 1 or | tccatcctcatcccatatgttcgtcaaagttttgttcagtttt |
| | | 866 of SEQ | gagcattggagagctgcatggagagtgggtggtgacgcatgtt |
| | | ID NO: 4 | aagttgttaactgaatgaatctgcatgcatgcaggtctgcggc |
| | | | ggtgcggcaagagctgccggctgcggtggctgaactacctccg |
| | | | gccgaacatcaagcggggcaacatctccgacga[g/c]ga |
| | | | ggaggagctcatcgtcaggctccacggcctgctcggcaac |
| | | | aggtggtccatcatcgccggcaggctgcccggccgaacag |
| | | | acaacgaaatcaagaactactggaacagcacgctcggca |
| | | | ggaaggcgctccccgcccggcccgccattgccg (SEQ |
| | | | ID NO: 9; SNP at position 206) |
| | 71237825 | 1092 of SEQ | Ant1_HapM4 |
| | | ID NO: 1 or | cggcgaggaccatcgcctccggatcctccagctccacggg |
| | | 1093 of SEQ | gagcgccgcggcggcgctctccacctcagtcc[c/t]tgccgt |
| | | ID NO: 4 | cctccacgccgcggcgccttcgtcgtcgccggccgctgtgtg |
| | | | ggcgcccaagcccgtgaggtgcactggcggcctcttcttccg |
| | | | ccgggagacgccgccgc (SEQ ID NO: 10; SNP at |
| | | | position 73) |
| | 71237633 | 1284 of SEQ | Ant1_HapM5 |
| | | ID NO: 1 or | gggcgcccaagcccgtgaggtgcactggcggcctcttcttcc |
| | | 1285 of SEQ | gccgggagacgccgccgcccgcgccggtcgtcgaggag |
| | | ID NO: 4 | acgcggaccaggggagaagagccaccatttcatgcctgca |
| | | | gcggtagcagctcggcctcggagg[a/c]gtctccggccga |
| | | | gctttgctcgtcagggtccggcgggggagactggatggacg |
| | | | acgtgagagccctggcgtcgttcctcgagtccgacgaggaa |
| | | | t (SEQ ID NO: 11; SNP at position 145) |
| | 71237608 | 1309 of SEQ | Ant1_HapM6 |
| | | ID NO: 1 or | gcggtagcagctcggcctcggaggagtctccggccgagctt |
| | | 1310 of SEQ | tgctcgtc[a/g]gggtccggcgggggagactggatggacg |
| | | ID NO: 4 | acgtgagagccctggcgtcgttcctcgagtccgacgaggaa |
| | | | tggctcaagtccctgcacatggccggttaa (SEQ ID NO: |
| | | | 12; SNP at position 50) |
| Ant2 | 591324765 | | Ant2_M1 |
| (Chr | | | atgcgtctgcatcagctttaagttacttctgaacactaccacat |
| 2H) | | | gcctgcagttactactaccgcaaaaaaacacaagtcgagtgcc |
| | | | gtgttttcaggtt[c/t]agataaaggagggtgttaggtttca |
| | | | gtttgggttcactcacgtggtctgaccaggcgtgctccctgtt |
| | | | gtagatgatggccgcgccgaggctcctcgccg (SEQ ID |
| | | | NO: 13; SNP at position 101) |
| | 593492855 | 491 of SEQ | Ant2_M2 |
| | | ID NO: 14 or | gcttagtttatctcttactttcttggtttgttccagctttcgat |
| | | 1284 of SEQ | c[g/t]agtatatacagyatgttctttgtaatcttcgttgagaa |
| | | ID NO: 17 | atcttcccaggagtttcttaattagcaccagt |
| | | | (SEQ ID NO: 20, SNP at position 46) |
| | 593493961 | 1597 of SEQ | Ant2_M3 |
| | | ID NO: 14 or | gtctcgcaagcctcactctcatgtagtccctccgtaaataaat |
| | | 2381 of SEQ | ggagtataagagtgyttagatcactacttagtgatataaacgc |
| | | ID NO: 17 | ttttatgtt[c/t]ctttacgaagggagtactatctgcctaat |
| | | | atgtacccgatcttgactaataccatgttgtctactgtgtact |
| | | | tttggcgc |
| | | | (SEQ ID NO: 21, SNP at position 96) |
| | 593494314 | 1950 of SEQ | Ant2_M4 |
| | | ID NO: 14 or | gcacatgatattcacttacggtttaaaattaaacggaaaactc |
| | | 2734 of SEQ | actattagtaacgctttgtc[g/t]catcaagcacatcgaaat |
| | | ID NO: 17 | tgcaaaatttaacccgggttccatggagccttgttgttaagaa |
| | | | a |
| | | | (SEQ ID NO: 22, SNP at position 64) |
| | 593494569 | 2207 of SEQ | Ant2_M5 |
| | | ID NO: 14 or | tccaaaaccacacattgtcatcttgtgtagctcacatgcatgtg |
| | | 2989 of SEQ | ttatagtgtttattatcgaatgctacgt[a/g]tatgctagtat |
| | | ID NO: 17 | agatctatatatacatctaggtagacttgttttactgttttttt |
| | | | tcaaagttgtaaatattatttgatttttttt |
| | | | (SEQ ID NO: 23, SNP at position 73) |
| | 593495307 | 2946 of SEQ | Ant2_M6 |
| | | ID NO: 14 or | aagcataagttcttgtgccgccggtgcgaccactctttgcacc |
| | | 3729 of SEQ | gatgacactttcccatcaaaaactctagtgttgcgctctagcct |
| | | ID NO: 17 | tcattaatgtcag[c/t]cagccggtcttgaacgacaatgaagt |
| | | | cttccaagtcgcccacaacgtgaggttgcaaatctactttggt |
| | | | gaaggcacatgtgcg (SEQ ID NO: 24, SNP at |
| | | | position 101) |
| | 593496606 | 3907 of SEQ | Ant2_M7 |
| | | ID NO: 14 or | ggggaactcgagctaggggaggtcgagtgcctgtccgacg |
| | | 4690 of SEQ | acaacctcgagcggatcacgaaggagatcaa[a/c]sggtt |
| | | ID NO: 17 | ctacggcctctgcgacgagctggacgtcggcgctctcgacg |
| | | | aaaactggatcat (SEQ ID NO: 25, SNP at |
| | | | position 72) |
| | 593496442 | 3743 of SEQ | Ant2_HapM1 |
| | | ID NO: 14 or | gctaggttttggaggacagggacatggtgaaccggatcagc |
| | | 4526 of SEQ | acatctttctgggacctgaagatcccaacaagctcgaagcc |
| | | ID NO: 17 | g[a/g]aggagccctccagcccatcagcagacgacgctggt |
| | | | gaggccgacatcgtgttccaagac (SEQ ID NO: 26, |
| | | | SNP at position 84) |
| | 593492774 | 410 of SEQ | Ant2_HapM2 |
| | | ID NO: 14 or | gttggactccgggcttcttggtctccatagctcaaagtaaacct |
| | | 1203 of SEQ | ttaacctc[c/g]tcttagatggaaaattaatcctccactcatg |
| | | ID NO: 17 | tctagcttagtttatctcttactttcttggtttgtt |
| | | | (SEQ ID NO: 27, SNP at position 76) |
| | 593493167 | 803 of SEQ | Ant2_HapM3 |
| | | ID NO: 14 or | cgtctgcatcatgcatatgaaatgaaggaaggaaataatat |
| | | 1596 of SEQ | ggtaatggcgctaccaatagttcgtccgagccaggaagaa |
| | | ID NO: 17 | ccgccgacgggg[a/g]agcaattcagctaccagctcgctg |
| | | | ccgctgtgaggagcatcaactggagctacgccatattctggt |
| | | | ccatttcaaccagccg (SEQ ID NO: 28, SNP at |
| | | | position 94) |
| | 593494160 | 1796 of SEQ | Ant2_HapM4 |
| | | ID NO: 14 or | cgtctatccaggttagctagcgcatgtcatgcaccctatatctg |
| | | 2580 of SEQ | tgtatactgttcatgtttctgaccccagctgcacgggtgacaa[ |
| | | ID NO: 17 | c/t]wttttttcttcagacggtcgcctgcattcccttgatgggt |
| | | | ggtgtgcttgagctcgggacgacagataccgtgagttttcctcg |
| | | | catca (SEQ ID NO: 29, SNP at position 88) |
| | 593494377 | 2013 of SEQ | Ant2_HapM5 |
| | | ID NO: 14 or | tcgaaattgcaaaatttaacccgggttccatggagccttgttgt |
| | | 2797 of SEQ | taagaaa[a/t]acacaacagaagttgtgagcacctatatattt |
| | | ID NO: 17 | ccgttcagtacatcttgccatatgt (SEQ ID NO: 30, |
| | | | SNP at position 52) |
| | 593495891 | 3530 of SEQ | Ant2_HapM6 |
| | | ID NO: 14 or | tacttcttttaatgtaaaatatattcttatactatgtgacaacg |
| | | 4313 of SEQ | ggaaggcggtaccgtttgctggggacactcattaa[a/g]tcat |
| | | ID NO: 17 | gaagacacaatactactaaatcaatccatgaattgtttgag |
| | | | (SEQ ID NO: 31, SNP at position 80) |
| | 593497594 | 4895 of SEQ | Ant2_HapM7 |
| | | ID NO: 14 or | cggcggtaaaaaatgcagcattaattaacgctcgtatgcatgg |
| | | 5676 of SEQ | ctatggtttactgttgcagttcgccggtc[c/g]tggcgccgt |
| | | ID NO: 17 | ggagcccgggatgatcatcggagcgcttcagacagctacacgc |
| | | | gggcgctgaaaaaaaaggatgctgctgagaca (SEQ |
| | | | ID NO: 32, SNP at position 73) |

TABLE 3

| Ant2_M1 | Ant1_HapM1 | Ant1_HapM2 | Ant1_HapM3 | Ant1_HapM4 | Ant1_HapM5 | Ant1_HapM6 | phenotype |
|---|---|---|---|---|---|---|---|
| C | T | C | C | C | C | G | Purple |
| T | G | C | G | C | A | A | Non- |
| | | | | | | | purple |

TABLE 4

| | Ant2 | Ant1 | Ant1 | Ant1 | Ant1 | Ant1 | Ant1 | seed |
|---|---|---|---|---|---|---|---|---|
| Name | M1 | HapM1 | HapM2 | HapM3 | HapM4 | HapM5 | HapM6 | color |
| Titan | T | G | C | G | C | A | A | non- |
| | | | | | | | | purple |
| KWSInfinity | T | T | C | C | C | C | G | non- |
| | | | | | | | | purple |
| BorrealOlavi | T | T | G | C | T | C | G | non- |
| | | | | | | | | purple |
| B9215 | C | T | C | C | C | C | G | purple |
| HOR10021 | C | T | C | C | C | C | G | purple |
| (HOR 4271) | | | | | | | | |
| HOR10012 | T | T | C | G | C | A | A | non- |
| (HOR 11784) | | | | | | | | purple |
| B17726 | C | T | C | C | C | C | G | purple |
| Hor10023 | C | T | C | C | C | C | G | purple |
| (HOR 3523) | | | | | | | | |
| KWSIrina | T | T | C | C | C | C | G | non- |
| | | | | | | | | purple |
| 24285 | T | T | C | C | C | C | G | non- |
| | | | | | | | | purple |
| HOR10025 | T | T | C | C | C | C | G | non- |
| (HOR 3232) | | | | | | | | purple |
| Planet | T | T | C | C | C | C | G | non- |
| | | | | | | | | purple |
| Leibniz | T | T | C | C | C | C | G | non- |
| | | | | | | | | purple |
| Ven | T | T | G | C | T | C | G | non- |
| | | | | | | | | purple |
| HOR10027 | C | T | C | C | C | C | G | purple |
| (HOR 15455) | | | | | | | | |
| B17650 | C | T | C | C | C | C | G | purple |
| Eng_F3 | T | T | C/G | C | C/T | C | G | non- |
| | | | | | | | | purple |
| KWSTower | T | T | C | C | C | C | G | non- |
| | | | | | | | | purple |
| Gaute | T | T | G | C | T | C | G | non- |
| | | | | | | | | purple |
| B20157 | T | T | C | C | C | C | G | non- |
| | | | | | | | | purple |
| Lomerit | T | G | C | G | C | A | A | non- |
| | | | | | | | | purple |

TABLE 5

| Name | M2 | M3 | M4 | M5 | M6 | M7 | HapM1 | HapM2 | HapM3 | HapM4 | HapM5 | HapM6 | HapM7 | phenotype |
|---|---|---|---|---|---|---|---|---|---|---|---|---|---|---|
| KWSInfinity | T | T | G | A | C | C | A | | G | T | A | G | C | non- |
| | | | | | | | | | | | | | | purple |
| B9215 | T | T | G | | C | C | G | C | A | T | A | G | C | purple |
| KWSIrina | T | T | G | A | C | C | A | C | A | T | A | G | C | non- |
| | | | | | | | | | | | | | | purple |
| 24285 | T | T | G | A | C | C | A | C | A | T | A | G | C | non- |
| | | | | | | | | | | | | | | purple |
| HOR10025 | T | T | G | A | C | C | A | C | A | T | A | G | C | non- |
| | | | | | | | | | | | | | | purple |
| Planet | T | T | G | A | C | C | A | C | A | T | A | G | C | non- |
| | | | | | | | | | | | | | | purple |
| Leibniz | T | T | G | A | C | C | A | C | A | T | A | G | C | non- |
| | | | | | | | | | | | | | | purple |
| B17650 | T | H | G | | C | C | G | C | A | T | A | G | C | purple |
| KWSTower | T | T | G | A | | C | A | | G | T | A | G | C | non- |
| | | | | | | | | | | | | | | purple |
| B20157 | T | T | G | A | C | C | G | G | G | C | T | A | G | non- |
| | | | | | | | | | | | | | | purple |
| SEQ ID NO: 14 | G | C | T | G | T | A | A | C | A | T | A | G | C | purple |

HOR10025 may be listed under the reference HOR 3232.

TABLE 6

| SEQ | |
|---|---|
| ID NO | Annotation |
| 1 | Genomic DNA sequence of a representative purple Ant1 allele |
| 2 | cDNA sequence of a representative purple Ant1 allele |
| 3 | Protein sequence of a representative purple Ant1 allele |
| 4 | Genomic DNA sequence of a representative non-purple |
| | Ant1 allele |
| 5 | cDNA sequence of a representative non-purple Ant1 allele |
| 6 | Protein sequence of a representative non-purple Ant1 allele |
| 7 | Ant1_HapM1 marker sequence |
| 8 | Ant1_HapM2 marker sequence |
| 9 | Ant1_HapM3 marker sequence |
| 10 | Ant1_HapM4 marker sequence |
| 11 | Ant1_HapM5 marker sequence |
| 12 | Ant1_HapM6 marker sequence |
| 13 | Ant2_M1 marker sequence |
| 14 | Genomic DNA sequence of a representative purple Ant2 allele |
| 15 | cDNA sequence of a representative purple Ant2 allele |
| 16 | Protein sequence of a representative purple Ant2 allele |
| 17 | Genomic DNA sequence of a representative non-purple |
| | Ant2 allele |
| 18 | cDNA sequence of a representative non-purple Ant2 allele |
| 19 | Protein sequence of a representative non-purple Ant2 allele |
| 20 | Ant2_M2 marker sequence |
| 21 | Ant2_M3 marker sequence |
| 22 | Ant2_M4 marker sequence |
| 23 | Ant2_M5 marker sequence |
| 24 | Ant2_M6 marker sequence |
| 25 | Ant2_M7 marker sequence |
| 26 | Ant2_HapM1 marker sequence |
| 27 | Ant2_HapM2 marker sequence |
| 28 | Ant2_HapM3 marker sequence |
| 29 | Ant2_HapM4 marker sequence |
| 30 | Ant2_HapM5 marker sequence |
| 31 | Ant2_HapM6 marker sequence |
| 32 | Ant2_HapM7 marker sequence |
| 33 | Blp_M1 marker sequence |

### Example 2

A panel of barley accessions having black/dark barley color-grains were ordered. A barley public database called Bridge (bridge.ipk-gatersleben.de/#start) database was used for the marker development of both color codes “purple” and “black” barley grain traits. This database contains information of more than 20,000 barley accessions and landraces hosted in Gatersleben genebank which were genotyped. In addition, all germplasm accessions were phenotyped for some traits such as spike, awn and grain color and shape.

### Marker Development for Blp Gene (Black Lemma Pericarp-Gen) Controlling Black/Dark Color in Seed Coat:

For the Blp gene a similar approach was taken into consideration for marker development. In the Bridge database, two groups of accessions; one having dark grains and one having white or normal colors were selected. By targeting the chromosomal region containing the Blp gene, the polymorphism differentiating between the two selected groups were identified. The flanking sequence of the identified single SNP for the corresponding gene was extracted and used to design KASP marker assays.

TABLE 7
Overview on BLP-alleles and base combinations
used for marker analysis

| BLP-alleles | Base combination | Phenotype |
|---|---|---|
| blp/blp | AA | Light |
| Blp/blp | AC | Black |
| Blp/Blp | CC | Black |

The developed marker for the Blp gene was initially tested on the panel consisting of interesting IPK and UK SeedBank accessions showing dark color and as well as several varieties (with non-colored grains) led to 100% matching phenotypes with genotypic calls. This result shows a successful validation of the developed marker alleles (SEQ ID NO: 33 (non-colored allele comprises “A” at position 101 and colored/black allele comprises “C” at position 101)).

TABLE 8

| Locus | Marker sequence (SNP in |
|---|---|
| name | square brackets) |
| BLP | AGGGAGCAGCCAGCTAGCTATGCTCCTCTGTGC |
| (Chr | TGCTCTGCAGGTTGGCGCAGCCTCACGTCGACA |
| 1H) | ACACATGGCACACGGGGCCAACTGAACCACGAG |
| | G[A/C]GGTGCATGCATGCATGCCTCCTGCCTC |
| | ACCGGGTTTGCTCCATGGCCAGCGGCCACCGGT |
| | ATGTGCACGTACCACCCCAGAATTTTCTATTCC |
| | AATTTCC |
| | (SEQ ID NOs 33) |

A round of validation of the developed marker for Blp gene was performed by screening of the progeny of two F2 mapping populations (Fantex×HOR10027 and Titan×HOR10031 also named HOR 278). Phenotypic segregation for plants with black (pigmented) barley seeds was consistent with a 3:1 ratio this mapping populations, indicating the control of seed color trait by a single dominant gene.

The black color expression in the barley seed mediated by the Blp gene was used as color system to select restorer seed in the hybrid barley production as described herein.

### Example 3

Backcrossing was performed to confirm that the identified markers, particularly Ant2_M1 and blp_M1, were able to identify progeny that are pigmented. The SNP identity for the “purple” Ant1 and Ant2 alleles as well as a possible SNP identity of a representative “non-purple” allele, and for a dark/black Blp allele and a non-pigmented Blp allele, are provided in Tables 9-11. The data presented in Tables 9-11 also show the coloring of three different backcrosses in BC0 and BC1. The coloring for Ant1/2 related phenotypes is purple and the coloring for blp related phenotypes is dark/black. The identity of the backcrossing lines are listed below each table.

The purple phenotype manifests in case of a homozygous purple Ant1 haplotype (i.e., the combination of markers Ant1_HapM1 to Ant1_HapM6) and/or a homozygous or heterozygous purple Ant2 allele (i.e., Ant2_M1). In Table 9, SNP identity of the Ant1_HapM1 to Ant1_HapM6 markers as well as the Ant2_M1 marker is provided for a number of barley varieties. In Table 10, SNP identity of the blp_M1 marker is provided for a number of barley varieties. The seed (pericarp) color for each of these varieties is also provided. In Table 11, SNP identity of the Ant1_HapM1 to Ant1_HapM6 markers as well as the Ant2_M1 and blp_M1 markers is provided for a number of barley varieties. The seed (pericarp) color for each of these varieties is also provided.

TABLE 9

| | | | | | | | | | genetically |
|---|---|---|---|---|---|---|---|---|---|
| | | | | | | | | | verification of |
| | | | | | | | | | Ant1/Ant2 alleles |
| | | Ant2_{—} | Ant1_{—} | Ant1_{—} | Ant1_{—} | Ant1_{—} | Ant1_{—} | Ant1_{—} | spending purple |
| name | identity | M1 | HapM1 | HapM2 | HapM3 | HapM4 | HapM5 | HapM6 | color to the grains |
| restorer 1 | breeding | T | T | C | C | C | C | G | no |
| | line | | | | | | | | |
| restorer 3 | breeding | T | T | C | C | C | C | G | no |
| | line | | | | | | | | |
| gr23 | gene bank | C | T | C | C | C | C | G | yes |
| | accession | | | | | | | | |
| restorer 3 × gr23 | BCO | C/T | T | C | C | C | C | G | yes |
| | progeny | | | | | | | | |
| restorer 3 × gr23 | BC0 | C/T | T | C | C | C | C | G | yes |
| | progeny | | | | | | | | |
| restorer 3 × gr23 | BC0 | C/T | T | C | C | C | C | G | yes |
| | progeny | | | | | | | | |
| restorer 3 × gr23 | BCO | C/T | T | C | C | C | C | G | yes |
| | progeny | | | | | | | | |
| restorer 3 × gr23 | BC0 | C/T | T | C | C | C | C | G | yes |
| | progeny | | | | | | | | |
| restorer 3 × gr23 | BC0 | C/T | T | C | C | C | C | G | yes |
| | progeny | | | | | | | | |
| restorer 1 × (restorer | BC1 | T | T | C | C | C | C | G | no |
| 3 × gr23) | progeny | | | | | | | | |
| restorer 1 × (restorer | BC1 | T | T | C | C | C | C | G | no |
| 3 × gr23) | progeny | | | | | | | | |
| restorer 1 × (restorer | BC1 | T | | C | C | C | C | G | no |
| 3 × gr23) | progeny | | | | | | | | |
| restorer 1 × (restorer | BC1 | C/T | T | C | C | C | C | G | yes |
| 3 × gr23) | progeny | | | | | | | | |
| restorer 1 × (restorer | BC1 | C/T | T | C | C | C | C | G | yes |
| 3 × gr23) | progeny | | | | | | | | |
| restorer 1 × (restorer | BC1 | C/T | T | C | C | C | C | G | yes |
| 3 × gr23) | progeny | | | | | | | | |
| restorer 1 × (restorer | BC1 | C/T | T | C | C | C | C | G | yes |
| 3 × gr23) | progeny | | | | | | | | |
| restorer 1 × (restorer | BC1 | T | T | C | C | C | | G | no |
| 3 × gr23) | progeny | | | | | | | | |
| restorer 1 × (restorer | BC1 | T | T | C | C | C | C | G | no |
| 3 × gr23) | progeny | | | | | | | | |
| restorer 1 × (restorer | BC1 | C/T | T | C | C | C | C | G | yes |
| 3 × gr23) | progeny | | | | | | | | |
| restorer 1 × (restorer | BC1 | C/T | T | C | C | C | C | G | yes |
| 3 × gr23) | progeny | | | | | | | | |

*gr23 corresponds to B9280.

TABLE 10

| | | | genetically |
|---|---|---|---|
| | | | verification of |
| | | | blp allele |
| | | | spending dark |
| name | identity | Blp_M1 | color to grains |
| restorer 2 | breeding line | A | no |
| restorer 3 | breeding line | A | no |
| gr8 | gene bank | C | yes |
| | accession | | |
| restorer 3 × gr8 | BC0 progeny | C/A | Yes |
| restorer 3 × gr8 | BC0 progeny | C/A | Yes |
| restorer 3 × gr8 | BC0 progeny | C/A | Yes |
| restorer 3 × gr8 | BC0 progeny | C/A | Yes |
| restorer 3 × gr8 | BC0 progeny | C/A | Yes |
| restorer 3 × gr8 | BC0 progeny | C/A | Yes |
| restorer 3 × gr8 | BC0 progeny | C/A | Yes |
| restorer 3 × gr8 | BC0 progeny | C/A | Yes |
| restorer 3 × gr8 | BC0 progeny | C/A | Yes |
| restorer 3 × gr8 | BC0 progeny | C/A | Yes |
| restorer 2 × (restorer 3 × gr8) | BC1 progeny | A | no |
| restorer 2 × (restorer 3 × gr8) | BC1 progeny | A | no |
| restorer 2 × (restorer 3 × gr8) | BC1 progeny | A/C | Yes |
| restorer 2 × (restorer 3 × gr8) | BC1 progeny | A | no |
| restorer 2 × (restorer 3 × gr8) | BC1 progeny | A/C | Yes |
| restorer 2 × (restorer 3 × gr8) | BC1 progeny | A | no |
| restorer 2 × (restorer 3 × gr8) | BC1 progeny | A | no |
| restorer 2 × (restorer 3 × gr8) | BC1 progeny | A | no |
| restorer 2 × (restorer 3 × gr8) | BC1 progeny | A/C | Yes |
| restorer 2 × (restorer 3 × gr8) | BC1 progeny | A/C | Yes |
| restorer 2 × (restorer 3 × gr8) | BC1 progeny | A | no |
| restorer 2 × (restorer 3 × gr8) | BC1 progeny | A/C | Yes |
| restorer 2 × (restorer 3 × gr8) | BC1 progeny | A | no |
| restorer 2 × (restorer 3 × gr8) | BC1 progeny | A | no |
| restorer 2 × (restorer 3 × gr8) | BC1 progeny | A/C | Yes |
| restorer 2 × (restorer 3 × gr8) | BC1 progeny | A | no |
| restorer 2 × (restorer 3 × gr8) | BC1 progeny | A/C | Yes |
| restorer 2 × (restorer 3 × gr8) | BC1 progeny | A/C | Yes |
| restorer 2 × (restorer 3 × gr8) | BC1 progeny | A/C | Yes |
| restorer 2 × (restorer 3 × gr8) | BC1 progeny | A/C | Yes |

*gr8 corresponds to HOR 8273.

TABLE 11

| | | | | | | | | | genetically | | |
|---|---|---|---|---|---|---|---|---|---|---|---|
| | | | | | | | | | verification | | genetically |
| | | | | | | | | | of Ant1/Ant2 | | verification |
| | | | | | | | | | alleles spending | | of blp allele |
| | | Ant2_{—} | Ant1_{—} | Ant1_{—} | Ant1_{—} | Ant1_{—} | Ant1_{—} | Ant1_{—} | purple color | Blp_{—} | spending dark |
| name | identity | M1 | HapM1 | HapM2 | HapM3 | HapM4 | HapM5 | HapM6 | to the grains | M1 | color to grains |
| restorer | breeding | T | T | C | C | C | C | G | no | A | no |
| 1 | line | | | | | | | | | | |
| restorer | breeding | T | T | C | C | C | C | G | no | A | no |
| 3 | line | | | | | | | | | | |
| gr17 | gene bank | C | G | C | G | C | A | A | no | C | yes |
| | accession | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC0 | C/T | G | C | C/G | C | A/C | A/G | no | C/A | Yes |
| 3 × gr17 | progeny | | | | | | | | | | |
| restorer | BC1 | C/T | G/T | C | C/G | C | A/C | A/G | yes | A/C | Yes |
| 1 × (restorer | progeny | | | | | | | | | | |
| 3 × gr17) | | | | | | | | | | | |
| restorer | BC1 | T | T | C | C | C | C | G | no | A | no |
| 1 × (restorer | progeny | | | | | | | | | | |
| 3 × gr17) | | | | | | | | | | | |
| restorer | BC1 | T | G/T | C | C/G | C | A/C | A/G | no | A/C | Yes |
| 1 × (restorer | progeny | | | | | | | | | | |
| 3 × gr17) | | | | | | | | | | | |
| restorer | BC1 | T | T | C | C | C | C | G | no | A/C | Yes |
| 1 × (restorer | progeny | | | | | | | | | | |
| 3 × gr17) | | | | | | | | | | | |
| restorer | BC1 | T | T | C | C | C | C | G | no | A/C | Yes |
| 1 × (restorer | progeny | | | | | | | | | | |
| 3 × gr17) | | | | | | | | | | | |
| restorer | BC1 | T | G/T | C | C/G | C | A/C | A/G | no | A | no |
| 1 × (restorer | progeny | | | | | | | | | | |
| 3 × gr17) | | | | | | | | | | | |
| restorer | BC1 | T | G/T | C | C/G | C | A/C | A/G | no | A/C | Yes |
| 1 × (restorer | progeny | | | | | | | | | | |
| 3 × gr17) | | | | | | | | | | | |
| restorer | BC1 | C/T | G/T | C | C/G | C | A/C | A/G | yes | A/C | Yes |
| 1 × (restorer | progeny | | | | | | | | | | |
| 3 × gr17) | | | | | | | | | | | |
| restorer | BC1 | C/T | G/T | C | C/G | C | A/C | A/G | yes | A/C | Yes |
| 1 × (restorer | progeny | | | | | | | | | | |
| 3 × gr17) | | | | | | | | | | | |
| restorer | BC1 | T | G/T | C | C/G | C | A/C | A/G | no | A/C | Yes |
| 1 × (restorer | progeny | | | | | | | | | | |
| 3 × gr17) | | | | | | | | | | | |
| restorer | BC1 | C/T | G/T | C | C/G | C | A/C | A/G | yes | A | no |
| 1 × (restorer | progeny | | | | | | | | | | |
| 3 × gr17) | | | | | | | | | | | |
| restorer | BC1 | C/T | T | C | C | C | C | G | yes | A | no |
| 1 × (restorer | progeny | | | | | | | | | | |
| 3 × gr17) | | | | | | | | | | | |
| restorer | BC1 | T | G/T | C | C/G | C | A/C | A/G | no | A/C | Yes |
| 1 × (restorer | progeny | | | | | | | | | | |
| 3 × gr17) | | | | | | | | | | | |
| restorer | BC1 | C/T | T | C | C | C | C | G | yes | A | no |
| 1 × (restorer | progeny | | | | | | | | | | |
| 3 × gr17) | | | | | | | | | | | |
| restorer | BC1 | T | T | C | C | C | C | G | no | A/C | Yes |
| 1 × (restorer | progeny | | | | | | | | | | |
| 3 × gr17) | | | | | | | | | | | |
| restorer | BC1 | C/T | G/T | C | C/G | C | A/C | A/G | yes | A | no |
| 1 × (restorer | progeny | | | | | | | | | | |
| 3 × gr17) | | | | | | | | | | | |
| restorer | BC1 | C/T | G/T | C | C/G | C | A/C | A/G | yes | A/C | Yes |
| 1 × (restorer | progeny | | | | | | | | | | |
| 3 × gr17) | | | | | | | | | | | |
| restorer | BC1 | T | T | C | C | C | C | G | no | A | no |
| 1 × (restorer | progeny | | | | | | | | | | |
| 3 × gr17) | | | | | | | | | | | |
| restorer | BC1 | T | T | C | C | C | C | G | no | A | no |
| 1 × (restorer | progeny | | | | | | | | | | |
| 3 × gr17) | | | | | | | | | | | |
| restorer | BC1 | T | G/T | C | C/G | C | A/C | A/G | no | A | no |
| 1 × (restorer | progeny | | | | | | | | | | |
| 3 × gr17) | | | | | | | | | | | |

*gr17 corresponds to HOR 1429.

### Example 4

Additional data regarding the SNP identity of the Ant1_HapM1 to Ant1_HapM6 markers as well as the Ant2_M1 marker and the blp_M1 marker is provided for a number of barley varieties. KASP markers for Ant1 and Ant2 genes are listed in Table 2. The KASP markers are based on competitive allele-specific PCR and enables bi-allelic scoring of single nucleotide polymorphisms (SNPs) and insertions and deletions (Indels) at specific loci.

The SNP identity for the “purple” Ant1 and Ant2 alleles as well as a possible SNP identity of a representative “non-purple” allele, and for a dark/black Blp allele and a non-pigmented Blp allele, are provided in Tables 9-11. Again, the purple phenotype manifests in case of a homozygous purple Ant1 haplotype (i.e., the combination of markers Ant1_HapM1 to Ant1_HapM6) and/or a homozygous or heterozygous purple Ant2 allele (i.e., Ant2_M1). In Tables 12A-12B, SNP identity of the Ant1_HapM1 to Ant1_HapM6 markers as well as the Ant2_M1 marker is provided for a number of barley varieties. In Tables 13A-13B, SNP identity of the blp_M1 marker is provided for a number of barley varieties. The seed (pericarp) color for each of these varieties is also provided. In Tables 14A-14B, SNP identity of the Ant1_HapM1 to Ant1_HapM6 markers as well as the Ant2_M1 marker and blp_1 marker is provided for a number of barley varieties. The seed (pericarp) color for each of these varieties is also provided. The identity of each line is indicated in Table 15.

TABLE 12A

| name or | | | | | | | | | |
|---|---|---|---|---|---|---|---|---|---|
| accession | | | | | | | | type of | |
| number | Ant2_M1 | Ant1_HapM1 | Ant1_HapM2 | Ant1_HapM3 | Ant1_HapM4 | Ant1_HapM5 | Ant1_HapM6 | material | phenotype |
| Borreal | T | T | G | C | T | C | G | commercial | non- |
| Olavi | | | | | | | | line | purple |
| Borreal | T | T | G | C | T | C | G | commercial | non- |
| Olavi | | | | | | | | line | purple |
| Gaute | T | T | G | C | T | C | G | commercial | non- |
| | | | | | | | | line | purple |
| Gaute | T | T | G | C | T | C | G | commercial | non- |
| | | | | | | | | line | purple |
| cl 1 | T | T | C | C | | C | G | commercial | non- |
| | | | | | | | | line | purple |
| cl 1 | T | | C | C | C | C | G | commercial | non- |
| | | | | | | | | line | purple |
| cl 2 | T | T | C | C | C | C | G | commercial | non- |
| | | | | | | | | line | purple |
| cl 2 | T | T | C | C | C | C | G | commercial | non- |
| | | | | | | | | line | purple |
| cl 3 | T | | C | C | C | C | G | commercial | non- |
| | | | | | | | | line | purple |
| cl 3 | T | | C | C | C | C | G | commercial | non- |
| | | | | | | | | line | purple |
| cl 4 | T | | C | C | C | C | G | commercial | non- |
| | | | | | | | | line | purple |
| cl 4 | T | | C | C | C | C | G | commercial | non- |
| | | | | | | | | line | purple |
| cl 5 | T | | C | C | C | C | G | commercial | non- |
| | | | | | | | | line | purple |
| cl 5 | T | | C | C | C | C | G | commercial | non- |
| | | | | | | | | line | purple |
| KWS | T | T | C | C | C | C | G | commercial | non- |
| Infinity | | | | | | | | line | purple |
| KWS | T | T | C | C | C | C | G | commercial | non- |
| Infinity | | | | | | | | line | purple |
| KWS | T | T | C | C | C | C | G | commercial | non- |
| Irina | | | | | | | | line | purple |
| KWS | T | T | C | C | C | C | G | commercial | non- |
| Irina | | | | | | | | line | purple |
| KWS | T | T | C | C | C | C | G | commercial | non- |
| Tower | | | | | | | | line | purple |
| KWS | T | T | | C | C | C | G | commercial | non- |
| Tower | | | | | | | | line | purple |
| Lomerit | T | G | C | G | C | A | A | commercial | non- |
| | | | | | | | | line | purple |
| Lomerit | T | G | C | G | C | A | A | commercial | non- |
| | | | | | | | | line | purple |
| Planet | T | T | C | C | C | C | G | commercial | non- |
| | | | | | | | | line | purple |
| Planet | T | T | C | C | C | C | G | commercial | non- |
| | | | | | | | | line | purple |
| Titan | T | G | C | G | C | A | A | commercial | non- |
| | | | | | | | | line | purple |
| Titan | T | G | C | G | C | A | A | commercial | non- |
| | | | | | | | | line | purple |
| Ven | T | T | G | C | T | C | G | commercial | non- |
| | | | | | | | | line | purple |
| Ven | T | T | G | C | T | C | G | commercial | non- |
| | | | | | | | | line | purple |

*cl = commercial line

TABLE 12B

| name or | | | | | | | | | |
|---|---|---|---|---|---|---|---|---|---|
| accession | | | | | | | | type of | |
| number | Ant2_M1 | Ant1_HapM1 | Ant1_HapM2 | Ant1_HapM3 | Ant1_HapM4 | Ant1_HapM5 | Ant1_HapM6 | material | phenotype |
| restorer | T | T | C | C | C | C | G | breeding | non- |
| 1 | | | | | | | | line | purple |
| restorer | T | T | C | C | C | C | G | breeding | non- |
| 1 | | | | | | | | line | purple |
| restorer | T | T | C | C | C | C | G | breeding | non- |
| 1 | | | | | | | | line | purple |
| restorer | T | T | C | C | C | C | G | breeding | non- |
| 2 | | | | | | | | line | purple |
| restorer | T | T | C | C | C | C | G | breeding | non- |
| 2 | | | | | | | | line | purple |
| restorer | T | T | C | C | C | C | G | breeding | non- |
| 2 | | | | | | | | line | purple |
| restorer | T | T | C | C | C | C | G | breeding | non- |
| 3 | | | | | | | | line | purple |
| restorer | T | T | C | C | C | C | G | breeding | non- |
| 3 | | | | | | | | line | purple |
| restorer | T | T | C | C | C | C | G | breeding | non- |
| 3 | | | | | | | | line | purple |
| restorer | T | T | C | C | C | C | G | breeding | non- |
| 4 | | | | | | | | line | purple |
| restorer | T | T | C | C | C | C | G | breeding | non- |
| 4 | | | | | | | | line | purple |
| restorer | T | T | C | C | C | C | G | breeding | non- |
| 4 | | | | | | | | line | purple |
| restorer | T | T | C | C | C | C | G | breeding | non- |
| 5 | | | | | | | | line | purple |
| restorer | T | T | C | C | C | C | G | breeding | non- |
| 5 | | | | | | | | line | purple |
| restorer | T | T | C | C | C | C | G | breeding | non- |
| 5 | | | | | | | | line | purple |
| restorer | T | T | C | C | C | C | G | breeding | non- |
| 6 | | | | | | | | line | purple |
| restorer | T | T | C | C | C | C | G | breeding | non- |
| 6 | | | | | | | | line | purple |
| restorer | T | T | C | C | C | C | G | breeding | non- |
| 6 | | | | | | | | line | purple |
| restorer | T | T | C | C | C | C | G | breeding | non- |
| 7 | | | | | | | | line | purple |
| restorer | T | T | C | C | C | C | G | breeding | non- |
| 7 | | | | | | | | line | purple |
| restorer | T | T | C | C | C | C | G | breeding | non- |
| 7 | | | | | | | | line | purple |
| restorer | T | T | C | C | C | C | G | breeding | non- |
| 8 | | | | | | | | line | purple |
| restorer | T | T | C | C | C | C | G | breeding | non- |
| 8 | | | | | | | | line | purple |
| restorer | T | T | C | C | C | C | G | breeding | non- |
| 8 | | | | | | | | line | purple |
| restorer | T | T | C | C | C | C | G | breeding | non- |
| 9 | | | | | | | | line | purple |
| restorer | T | T | C | C | C | C | G | breeding | non- |
| 9 | | | | | | | | line | purple |
| restorer | T | T | C | C | C | C | G | breeding | non- |
| 9 | | | | | | | | line | purple |
| restorer | T | T | C | C | C | C | G | breeding | non- |
| 10 | | | | | | | | line | purple |
| restorer | T | T | C | C | C | C | G | breeding | non- |
| 10 | | | | | | | | line | purple |
| restorer | T | T | C | C | C | C | G | breeding | non- |
| 10 | | | | | | | | line | purple |
| restorer | T | T | C | C | C | C | G | breeding | non- |
| 11 | | | | | | | | line | purple |
| restorer | T | T | C | C | C | C | G | breeding | non- |
| 11 | | | | | | | | line | purple |
| restorer | T | T | C | C | C | C | G | breeding | non- |
| 11 | | | | | | | | line | purple |
| restorer | T | T | C | C | C | C | G | breeding | non- |
| 12 | | | | | | | | line | purple |
| restorer | T | T | C | C | C | C | G | breeding | non- |
| 12 | | | | | | | | line | purple |
| restorer | T | T | C | C | C | C | G | breeding | non- |
| 12 | | | | | | | | line | purple |
| gr1 | C | T | C | C | C | C | G | gene bank | Slightly |
| | | | | | | | | accession | purple |
| gr1 | C | T | C | C | C | C | G | gene bank | Slightly |
| | | | | | | | | accession | purple |
| gr1 | C | T | C | C | C | C | G | gene bank | Slightly |
| | | | | | | | | accession | purple |
| gr3 | C | T | C | C | C | C | G | gene bank | non- |
| | | | | | | | | accession | purple |
| gr3 | C | T | C | C | C | C | G | gene bank | non- |
| | | | | | | | | accession | purple |
| gr3 | C | T | | C | C | C | G | gene bank | non- |
| | | | | | | | | accession | purple |
| gr4 | C | T | C | C | C | C | G | gene bank | purple |
| | | | | | | | | accession | |
| gr4 | C | T | C | C | C | C | G | gene bank | purple |
| | | | | | | | | accession | |
| gr4 | C | T | C | C | C | C | G | gene bank | purple |
| | | | | | | | | accession | |
| gr20 | C | T | C | C | C | C | G | gene bank | purple |
| | | | | | | | | accession | |
| gr20 | C | T | C | C | C | C | G | gene bank | purple |
| | | | | | | | | accession | |
| gr20 | C | T | C | C | C | C | G | gene bank | purple |
| | | | | | | | | accession | |
| gr21 | C | T | C | C | C | C | G | gene bank | purple |
| | | | | | | | | accession | |
| gr21 | C | T | C | C | C | C | G | gene bank | purple |
| | | | | | | | | accession | |
| gr21 | C | T | C | C | C | C | G | gene bank | purple |
| | | | | | | | | accession | |
| gr22 | C | T | C | C | C | C | G | gene bank | purple |
| | | | | | | | | accession | |
| gr22 | C | T | C | C | C | C | G | gene bank | purple |
| | | | | | | | | accession | |
| gr22 | C | T | C | C | C | C | G | gene bank | purple |
| | | | | | | | | accession | |
| gr23 | C | T | C | C | C | C | G | gene bank | purple |
| | | | | | | | | accession | |
| gr23 | C | T | C | C | C | C | G | gene bank | purple |
| | | | | | | | | accession | |
| gr23 | C | T | C | C | C | C | G | gene bank | purple |
| | | | | | | | | accession | |
| gr24 | C | T | C | C | C | C | G | gene bank | purple |
| | | | | | | | | accession | |
| gr24 | C | T | C | C | C | C | G | gene bank | purple |
| | | | | | | | | accession | |
| gr24 | C | T | C | C | C | C | G | gene bank | purple |
| | | | | | | | | accession | |
| gr25 | C | T | C | C | C | C | G | gene bank | purple |
| | | | | | | | | accession | |
| gr25 | C | T | C | C | C | C | G | gene bank | purple |
| | | | | | | | | accession | |
| gr25 | C | T | C | C | C | C | G | gene bank | purple |
| | | | | | | | | accession | |
| gr26 | C | T | C | C | C | C | G | gene bank | purple |
| | | | | | | | | accession | |
| gr26 | C | T | C | C | C | C | G | gene bank | purple |
| | | | | | | | | accession | |
| gr26 | C | T | C | C | C | C | G | gene bank | purple |
| | | | | | | | | accession | |
| gr27 | C | T | C | C | C | C | G | gene bank | purple |
| | | | | | | | | accession | |
| gr27 | C | T | C | C | C | C | G | gene bank | purple |
| | | | | | | | | accession | |
| gr27 | C | T | C | C | C | C | G | gene bank | purple |
| | | | | | | | | accession | |

TABLE 13A

| | | type of | seed |
|---|---|---|---|
| name | Blp_M1 | material | phenotype |
| Borreal Olavi | A | commercial line | normal |
| Borreal Olavi | A | commercial line | normal |
| Gaute | A | commercial line | normal |
| Gaute | A | commercial line | normal |
| cl 1 | A | commercial line | normal |
| cl 1 | A | commercial line | normal |
| cl 2 | A | commercial line | normal |
| cl 2 | A | commercial line | normal |
| cl 3 | A | commercial line | normal |
| cl 3 | A | commercial line | normal |
| KWS Infinity | A | commercial line | normal |
| KWS Infinity | A | commercial line | normal |
| KWS Irina | A | commercial line | normal |
| KWS Irina | A | commercial line | normal |
| cl 4 | A | commercial line | normal |
| cl 4 | A | commercial line | normal |
| cl 5 | A | commercial line | normal |
| cl 5 | A | commercial line | normal |
| KWS Tower | A | commercial line | normal |
| KWS Tower | A | commercial line | normal |
| Lomerit | A | commercial line | normal |
| Lomerit | A | commercial line | normal |
| Planet | A | commercial line | normal |
| Planet | A | commercial line | normal |
| Titan | A | commercial line | normal |
| Titan | A | commercial line | normal |
| Ven | A | commercial line | normal |
| Ven | A | commercial line | normal |

*cl = commercial line

TABLE 13B

| | | type of | seed |
|---|---|---|---|
| name | Blp_M1 | material | phenotype |
| restorer 1 | A | breeding line | normal |
| restorer 1 | A | breeding line | normal |
| restorer 1 | A | breeding line | normal |
| restorer 2 | A | breeding line | normal |
| restorer 2 | A | breeding line | normal |
| restorer 2 | A | breeding line | normal |
| restorer 4 | A | breeding line | normal |
| restorer 4 | A | breeding line | normal |
| restorer 4 | A | breeding line | normal |
| restorer 5 | A | breeding line | normal |
| restorer 5 | A | breeding line | normal |
| restorer 5 | A | breeding line | normal |
| restorer 6 | A | breeding line | normal |
| restorer 6 | A | breeding line | normal |
| restorer 6 | A | breeding line | normal |
| restorer 7 | A | breeding line | normal |
| restorer 7 | A | breeding line | normal |
| restorer 7 | A | breeding line | normal |
| restorer 8 | A | breeding line | normal |
| restorer 8 | A | breeding line | normal |
| restorer 8 | A | breeding line | normal |
| restorer 9 | A | breeding line | normal |
| restorer 9 | A | breeding line | normal |
| restorer 9 | A | breeding line | normal |
| restorer 10 | A | breeding line | normal |
| restorer 10 | A | breeding line | normal |
| restorer 10 | A | breeding line | normal |
| restorer 11 | A | breeding line | normal |
| restorer 11 | A | breeding line | normal |
| restorer 11 | A | breeding line | normal |
| restorer 12 | A | breeding line | normal |
| restorer 12 | A | breeding line | normal |
| restorer 12 | A | breeding line | normal |
| gr17 | C | gene bank accession | strongly dark |
| gr17 | C | gene bank accession | strongly dark |
| gr17 | C | gene bank accession | strongly dark |
| gr11 | C | gene bank accession | strongly dark |
| gr11 | C | gene bank accession | strongly dark |
| gr11 | C | gene bank accession | strongly dark |
| gr19 | C | gene bank accession | dark |
| gr19 | C | gene bank accession | dark |
| gr19 | C | gene bank accession | dark |
| gr15 | C | gene bank accession | Slightly dark |
| gr15 | C | gene bank accession | Slightly dark |
| gr15 | C | gene bank accession | Slightly dark |
| gr14 | C | gene bank accession | dark |
| gr14 | C | gene bank accession | dark |
| gr14 | C | gene bank accession | dark |
| gr5 | C | gene bank accession | strongly dark |
| gr5 | C | gene bank accession | strongly dark |
| gr5 | C | gene bank accession | strongly dark |
| gr10 | C | gene bank accession | strongly dark |
| gr10 | C | gene bank accession | strongly dark |
| gr10 | C | gene bank accession | strongly dark |
| gr7 | C | gene bank accession | Slightly dark |
| gr7 | C | gene bank accession | Slightly dark |
| gr7 | C | gene bank accession | Slightly dark |
| gr8 | C | gene bank accession | Slightly dark |
| gr8 | C | gene bank accession | Slightly dark |
| gr8 | C | gene bank accession | Slightly dark |
| gr18 | C | gene bank accession | dark |
| gr18 | C | gene bank accession | dark |
| gr18 | C | gene bank accession | dark |

TABLE 14A

| | | | | | | | | | genetically | | | |
|---|---|---|---|---|---|---|---|---|---|---|---|---|
| | | | | | | | | | verification | | genetically | |
| | | | | | | | | | of Ant1/Ant2 | | verification | |
| name or | | | | | | | | | alleles spending | | of blp allele | |
| accession | Ant2_{—} | Ant1_{—} | Ant1_{—} | Ant1_{—} | Ant1_{—} | Ant1_{—} | Ant1_{—} | type of | purple color | Blp_{—} | spending darker | seed |
| number | M1 | HapM1 | HapM2 | HapM3 | HapM4 | HapM5 | HapM6 | material | to the grains | M1 | color to grains | phenotype |
| Borreal | T | T | G | C | T | C | G | commercial | no | A | no | non- |
| Olavi | | | | | | | | line | | | | colored |
| Borreal | T | T | G | C | T | C | G | commercial | no | A | no | non- |
| Olavi | | | | | | | | line | | | | colored |
| Gaute | T | T | G | C | T | C | G | commercial | no | A | no | non- |
| | | | | | | | | line | | | | colored |
| Gaute | T | T | G | C | T | C | G | commercial | no | A | no | non- |
| | | | | | | | | line | | | | colored |
| cl 1 | T | T | C | C | | C | G | commercial | no | A | no | non- |
| | | | | | | | | line | | | | colored |
| cl 1 | T | | C | C | C | C | G | commercial | no | A | no | non- |
| | | | | | | | | line | | | | colored |
| cl 2 | T | T | C | C | C | C | G | commercial | no | A | no | non- |
| | | | | | | | | line | | | | colored |
| cl 2 | T | T | C | C | C | C | G | commercial | no | A | no | non- |
| | | | | | | | | line | | | | colored |
| cl 3 | T | | C | C | C | C | G | commercial | no | A | no | non- |
| | | | | | | | | line | | | | colored |
| cl 3 | T | | C | C | C | C | G | commercial | no | A | no | non- |
| | | | | | | | | line | | | | colored |
| KWS | T | T | C | C | C | C | G | commercial | no | A | no | non- |
| Infinity | | | | | | | | line | | | | colored |
| KWS | T | T | C | C | C | C | G | commercial | no | A | no | non- |
| Infinity | | | | | | | | line | | | | colored |
| KWS | T | T | C | C | C | C | G | commercial | no | A | no | non- |
| Irina | | | | | | | | line | | | | colored |
| KWS | T | T | C | C | C | C | G | commercial | no | A | no | non- |
| Irina | | | | | | | | line | | | | colored |
| cl 4 | T | | C | C | C | C | G | commercial | no | A | no | non- |
| | | | | | | | | line | | | | colored |
| cl 4 | T | | C | C | C | C | G | commercial | no | A | no | non- |
| | | | | | | | | line | | | | colored |
| cl 5 | T | | C | C | C | C | G | commercial | no | A | no | non- |
| | | | | | | | | line | | | | colored |
| cl 5 | T | | C | C | C | C | G | commercial | no | A | no | non- |
| | | | | | | | | line | | | | colored |
| KWS | T | T | C | C | C | C | G | commercial | no | A | no | non- |
| Tower | | | | | | | | line | | | | colored |
| KWS | T | T | | C | C | C | G | commercial | no | A | no | non- |
| Tower | | | | | | | | line | | | | colored |
| Lomerit | T | G | C | G | C | A | A | commercial | no | A | no | non- |
| | | | | | | | | line | | | | colored |
| Lomerit | T | G | C | G | C | A | A | commercial | no | A | no | non- |
| | | | | | | | | line | | | | colored |
| Planet | T | T | C | C | C | C | G | commercial | no | A | no | non- |
| | | | | | | | | line | | | | colored |
| Planet | T | T | C | C | C | C | G | commercial | no | A | no | non- |
| | | | | | | | | line | | | | colored |
| Titan | T | G | C | G | C | A | A | commercial | no | A | no | non- |
| | | | | | | | | line | | | | colored |
| Titan | T | G | C | G | C | A | A | commercial | no | A | no | non- |
| | | | | | | | | line | | | | colored |
| Ven | T | T | G | C | T | C | G | commercial | no | A | no | non- |
| | | | | | | | | line | | | | colored |
| Ven | T | T | G | C | T | C | G | commercial | no | A | no | non- |
| | | | | | | | | line | | | | colored |

*cl = commercial line

TABLE 14B

| | | | | | | | | | genetically | | | |
|---|---|---|---|---|---|---|---|---|---|---|---|---|
| | | | | | | | | | verification | | genetically | |
| | | | | | | | | | of Ant1/Ant2 | | verification | |
| name or | | | | | | | | | alleles spending | | of blp allele | |
| accession | Ant2_{—} | Ant1_{—} | Ant1_{—} | Ant1_{—} | Ant1_{—} | Ant1_{—} | Ant1_{—} | type of | purple color | Blp_{—} | spending darker | seed |
| number | M1 | HapM1 | HapM2 | HapM3 | HapM4 | HapM5 | HapM6 | material | to the grains | M1 | color to grains | phenotype |
| restorer | T | T | C | C | C | C | G | breeding | no | A | no | non- |
| 1 | | | | | | | | line | | | | colored |
| restorer | T | T | C | C | C | C | G | breeding | no | A | no | non- |
| 1 | | | | | | | | line | | | | colored |
| restorer | T | T | C | C | C | C | G | breeding | no | A | no | non- |
| 1 | | | | | | | | line | | | | colored |
| restorer | T | T | C | C | C | C | G | breeding | no | A | no | non- |
| 2 | | | | | | | | line | | | | colored |
| restorer | T | T | C | C | C | C | G | breeding | no | A | no | non- |
| 2 | | | | | | | | line | | | | colored |
| restorer | T | T | C | C | C | C | G | breeding | no | A | no | non- |
| 2 | | | | | | | | line | | | | colored |
| restorer | T | T | C | C | C | C | G | breeding | no | A | no | non- |
| 3 | | | | | | | | line | | | | colored |
| restorer | T | T | C | C | C | C | G | breeding | no | A | no | non- |
| 3 | | | | | | | | line | | | | colored |
| restorer | T | T | C | C | C | C | G | breeding | no | A | no | non- |
| 3 | | | | | | | | line | | | | colored |
| restorer | T | T | C | C | C | C | G | breeding | no | A | no | non- |
| 4 | | | | | | | | line | | | | colored |
| restorer | T | T | C | C | C | C | G | breeding | no | A | no | non- |
| 4 | | | | | | | | line | | | | colored |
| restorer | T | T | C | C | C | C | G | breeding | no | A | no | non- |
| 4 | | | | | | | | line | | | | colored |
| restorer | T | T | C | C | C | C | G | breeding | no | A | no | non- |
| 5 | | | | | | | | line | | | | colored |
| restorer | T | T | C | C | C | C | G | breeding | no | A | no | non- |
| 5 | | | | | | | | line | | | | colored |
| restorer | T | T | C | C | C | C | G | breeding | no | A | no | non- |
| 5 | | | | | | | | line | | | | colored |
| restorer | T | T | C | C | C | C | G | breeding | no | A | no | non- |
| 6 | | | | | | | | line | | | | colored |
| restorer | T | T | C | C | C | C | G | breeding | no | A | no | non- |
| 6 | | | | | | | | line | | | | colored |
| restorer | T | T | C | C | C | C | G | breeding | no | A | no | non- |
| 6 | | | | | | | | line | | | | colored |
| restorer | T | T | C | C | C | C | G | breeding | no | A | no | non- |
| 7 | | | | | | | | line | | | | colored |
| restorer | T | T | C | C | C | C | G | breeding | no | A | no | non- |
| 7 | | | | | | | | line | | | | colored |
| restorer | T | T | C | C | C | C | G | breeding | no | A | no | non- |
| 7 | | | | | | | | line | | | | colored |
| restorer | T | T | C | C | C | C | G | breeding | no | A | no | non- |
| 8 | | | | | | | | line | | | | colored |
| restorer | T | T | C | C | C | C | G | breeding | no | A | no | non- |
| 8 | | | | | | | | line | | | | colored |
| restorer | T | T | C | C | C | C | G | breeding | no | A | no | non- |
| 8 | | | | | | | | line | | | | colored |
| restorer | T | T | C | C | C | C | G | breeding | no | A | no | non- |
| 9 | | | | | | | | line | | | | colored |
| restorer | T | T | C | C | C | C | G | breeding | no | A | no | non- |
| 9 | | | | | | | | line | | | | colored |
| restorer | T | T | C | C | C | C | G | breeding | no | A | no | non- |
| 9 | | | | | | | | line | | | | colored |
| restorer | T | T | C | C | C | C | G | breeding | no | A | no | non- |
| 10 | | | | | | | | line | | | | colored |
| restorer | T | T | C | C | C | C | G | breeding | no | A | no | non- |
| 10 | | | | | | | | line | | | | colored |
| restorer | T | T | C | C | C | C | G | breeding | no | A | no | non- |
| 10 | | | | | | | | line | | | | colored |
| restorer | T | T | C | C | C | C | G | breeding | no | A | no | non- |
| 11 | | | | | | | | line | | | | colored |
| restorer | T | T | C | C | C | C | G | breeding | no | A | no | non- |
| 11 | | | | | | | | line | | | | colored |
| restorer | T | T | C | C | C | C | G | breeding | no | A | no | non- |
| 11 | | | | | | | | line | | | | colored |
| restorer | T | T | C | C | C | C | G | breeding | no | A | no | non- |
| 12 | | | | | | | | line | | | | colored |
| restorer | T | T | C | C | C | C | G | breeding | no | A | no | non- |
| 12 | | | | | | | | line | | | | colored |
| restorer | T | T | C | C | C | C | G | breeding | no | A | no | non- |
| 12 | | | | | | | | line | | | | colored |
| gr2 | C | T | C | C | C | C | G | gene | yes | C | yes | colored |
| | | | | | | | | bank | | | | |
| | | | | | | | | accession | | | | |
| gr2 | C | T | C | C | C | C | G | gene | yes | C | yes | colored |
| | | | | | | | | bank | | | | |
| | | | | | | | | accession | | | | |
| gr2 | C | T | C | C | C | C | G | gene | yes | C | yes | colored |
| | | | | | | | | bank | | | | |
| | | | | | | | | accession | | | | |
| gr3 | T | T | C | C | C | C | G | gene | no | A | no | non- |
| | | | | | | | | bank | | | | colored |
| | | | | | | | | accession | | | | |
| gr3 | T | T | C | C | C | C | G | gene | no | A | no | non- |
| | | | | | | | | bank | | | | colored |
| | | | | | | | | accession | | | | |
| gr3 | T | T | | C | C | C | G | gene | no clear | A | no | non- |
| | | | | | | | | bank | marker | | | colored |
| | | | | | | | | accession | result | | | |
| gr 16 | C | T | C | C | C | C | G | gene | yes | C | yes | strongly |
| | | | | | | | | bank | | | | colored |
| | | | | | | | | accession | | | | |
| gr 16 | C | T | | C | C | C | G | gene | no clear | C | yes | strongly |
| | | | | | | | | bank | marker | | | colored |
| | | | | | | | | accession | result | | | |
| gr 16 | C | T | C | C | C | C | G | gene | yes | C | yes | strongly |
| | | | | | | | | bank | | | | colored |
| | | | | | | | | accession | | | | |
| gr 13 | C | T | C | C | C | C | G | gene | yes | C | yes | colored |
| | | | | | | | | bank | | | | |
| | | | | | | | | accession | | | | |
| gr 13 | C | T | C | C | C | C | G | gene | yes | C | yes | colored |
| | | | | | | | | bank | | | | |
| | | | | | | | | accession | | | | |
| gr 13 | C | T | C | C | C | C | G | gene | yes | C | yes | colored |
| | | | | | | | | bank | | | | |
| | | | | | | | | accession | | | | |
| gr 12 | C | T | C | C | C | C | G | gene | yes | C | yes | colored |
| | | | | | | | | bank | | | | |
| | | | | | | | | accession | | | | |
| gr 12 | C | T | C | C | C | C | G | gene | yes | C | yes | colored |
| | | | | | | | | bank | | | | |
| | | | | | | | | accession | | | | |
| gr 12 | C | T | C | C | C | C | G | gene | yes | C | yes | colored |
| | | | | | | | | bank | | | | |
| | | | | | | | | accession | | | | |

TABLE 15

| Name in | Accession number |
|---|---|
| Examples | at donor |
| gr1 | B17650 |
| gr2 | B17726 |
| gr3 | B20157 |
| gr4 | B9215 |
| gr5 | HOR 3474 |
| gr6 | HOR 4132 |
| gr7 | HOR 4605 |
| gr8 | HOR 8273 |
| gr9 | HOR 11784 |
| gr10 | HOR 3661 |
| gr11 | HOR 2678 |
| gr12 | HOR 4271 |
| gr13 | HOR 3523 |
| gr14 | HOR 3232 |
| gr15 | HOR 2988 |
| gr16 | HOR 15455 |
| gr17 | HOR 1429 |
| gr18 | HOR 962 |
| gr19 | HOR 278 |
| gr20 | B17447 |
| gr21 | B20225 |
| gr22 | B9235 |
| gr23 | B9280 |
| gr24 | HOR10114 |
| gr25 | HOR10151 |
| gr26 | HOR10154 |
| gr27 | HOR10159 |

1. A method for identifying and/or selecting a barley plant or plant part, comprising screening for the presence of a polynucleotide comprising one or more, preferably all, molecular marker allele selected from Ant1_HapM1, Ant1_HapM2, Ant1_HapM3, Ant1_HapM4, Ant1_HapM5, and Ant1_HapM6 in the genome of the plant or plant part, preferably wherein said molecular marker allele is homozygous.

2. The method according to claim 1, wherein Ant1_HapM1 is or comprises a SNP at a position corresponding to position 71238822 bp on chromosome 7H of reference genome sequence assembly of Barley cultivar Morex v3.0;
Ant1_HapM2 is or comprises a SNP at a position corresponding to position 71238454 bp on chromosome 7H of reference genome sequence assembly of Barley cultivar Morex v3.0;
Ant1_HapM3 is or comprises a SNP at a position corresponding to position 71238052 bp on chromosome 7H of reference genome sequence assembly of Barley cultivar Morex v3.0;
Ant1_HapM4 is or comprises a SNP at a position corresponding to position 71237825 bp on chromosome 7H of reference genome sequence assembly of Barley cultivar Morex v3.0;
Ant1_HapM5 is or comprises a SNP at a position corresponding to position 71237633 bp on chromosome 7H of reference genome sequence assembly of Barley cultivar Morex v3.0; and/or
Ant1_HapM6 is or comprises a SNP at a position corresponding to position 71237608 bp on chromosome 7H of reference genome sequence assembly of Barley cultivar Morex v3.0.

3. The method according to claim 2, wherein said SNP is
T in Ant1_HapM1;
Cin Ant1_HapM2;
Cin Ant1_HapM3;
Cin Ant1_HapM4;
Cin Ant1_HapM5; and/or
G in Ant1_HapM6.

4. The method according to claim 1, further comprising screening for the presence of a polynucleotide comprising one or more molecular marker allele selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2 M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7 in the genome of the plant or plant part.

5. A method for identifying and/or selecting a barley plant or plant part, comprising screening for the presence of a polynucleotide comprising one or more molecular marker allele selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7 in the genome of the plant or plant part.

6. The method according to claim 5, further comprising identifying and/or selecting a barley plant or plant part, comprising screening for the presence of a polynucleotide comprising one or more molecular marker allele selected from Ant1_HapM1, Ant1_HapM2, Ant1_HapM3, Ant1_HapM4, Ant1_HapM5, and Ant1_HapM6 in the genome of the plant or plant part, preferably as defined in claim 2 or 3, preferably wherein said molecular marker allele is homozygous, and
preferably wherein Ant1_HapM1 is or comprises a SNP at a position corresponding to position 71238822 bp on chromosome 7H of reference genome sequence assembly of Barley cultivar Morex v3.0;
Ant1 HapM2 is or comprises a SNP at a position corresponding to position 71238454 bp on chromosome 7H of reference genome sequence assembly of Barley cultivar Morex v3.0;
Ant1 HapM3 is or comprises a SNP at a position corresponding to position 71238052 bp on chromosome 7H of reference genome sequence assembly of Barley cultivar Morex v3.0;
Ant1 HapM4 is or comprises a SNP at a position corresponding to position 71237825 bp on chromosome 7H of reference genome sequence assembly of Barley cultivar Morex v3.0;
Ant1 HapM5 is or comprises a SNP at a position corresponding to position 71237633 bp on chromosome 7H of reference genome sequence assembly of Barley cultivar Morex v3.0; and/or
Ant1 HapM6 is or comprises a SNP at a position corresponding to position 71237608 bp on chromosome 7H of reference genome sequence assembly of Barley cultivar Morex v3.0.

7. The method according to claim 4, wherein
Ant2_M1 is or comprises a SNP at a position corresponding to position 591324765 bp on chromosome 2H of reference genome sequence assembly of Barley cultivar Morex v3.0;
Ant2_M2 is or comprises a SNP at a position corresponding to position 593492855 bp (on chromosome 2H) of reference genome sequence assembly of Barley cultivar Morex v3.0 July 2020 (Morex_v3);
Ant2_M3 is or comprises a SNP at a position corresponding to position 593493961 bp on chromosome 2H of reference genome sequence assembly of Barley cultivar Morex_v3;
Ant2_M4 is or comprises a SNP at a position corresponding to position 593494314 bp on chromosome 2H of reference genome sequence assembly of Barley cultivar Morex_v3;
Ant2_M5 is or comprises a SNP at a position corresponding to position 593494569 bp on chromosome 2H of reference genome sequence assembly of Barley cultivar Morex_v3;
Ant2_M6 is or comprises a SNP at a position corresponding to position 593495307 bp on chromosome 2H of reference genome sequence assembly of Barley cultivar Morex_v3;
Ant2_M7 is or comprises a SNP at a position corresponding to position 593496606 bp on chromosome 2H of reference genome sequence assembly of Barley cultivar Morex_v3;
Ant2_HapM1 is or comprises a SNP at a position corresponding to position 593496442 bp on chromosome 2H of reference genome sequence assembly of Barley cultivar Morex_v3;
Ant2_HapM2 is or comprises a SNP at a position corresponding to position 593492774 bp on chromosome 2H of reference genome sequence assembly of Barley cultivar Morex_v3;
Ant2_HapM3 is or comprises a SNP at a position corresponding to position 593493167 bp on chromosome 2H of reference genome sequence assembly of Barley cultivar Morex_v3;
Ant2_HapM4 is or comprises a SNP at a position corresponding to position 593494160 bp on chromosome 2H of reference genome sequence assembly of Barley cultivar Morex_v3;
Ant2_HapM5 is or comprises a SNP at a position corresponding to position 593494377 bp on chromosome 2H of reference genome sequence assembly of Barley cultivar Morex_v3;
Ant2_HapM6 is or comprises a SNP at a position corresponding to position 593495891 bp on chromosome 2H of reference genome sequence assembly of Barley cultivar Morex_v3; and/or
Ant2_HapM7 is or comprises a SNP at a position corresponding to position 593497594 bp on chromosome 2H of reference genome sequence assembly of Barley cultivar Morex_v3.

8. The method according to claim 7, wherein said SNP is
C in Ant2 M1
G in Ant2 M2
C in Ant2 M3
T in Ant2 M4
G in Ant2 M5
T in Ant2 M6
A in Ant2 M7
G in Ant2_HapM1
C in Ant2_HapM2
A in Ant2_HapM3
T in Ant2_HapM4
A in Ant2_HapM5
G in Ant2_HapM6 and/or
C in Ant2_HapM7.

9. The method according to claim 1, which is a method for identifying a barley plant or plant part producing or capable of producing a seed having a purple pericarp and/or hull, and/or a plant grown therefrom and producing or capable of producing a seed having a purple pericarp and/or hull.

10. A male sterile barley plant or plant part, preferably a cytoplasmic male sterile barley plant or plant part, comprising one or more, preferably all, homozygous molecular marker allele selected from Ant1_HapM1, Ant1_HapM2, Ant1_HapM3, Ant1_HapM4, Ant1_HapM5, and Ant1_HapM6 in the genome of the plant or plant part, and comprising one or more of a homozygous or heterozygous molecular marker allele selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7.

11. A method for producing, obtaining, or selecting a hybrid barley seed mixture, comprising
providing a barley bulk or unselected seed mixture harvested from a cross between a first barley parent plant population and a second barley parent plant population,
wherein one of said first or second barley parent plant population is male sterile, preferably cytoplasmic male sterile,
wherein only said first barley parent plant population comprises a homozygous Ant1 gene or coding sequence thereof comprising one or more of molecular marker allele selected from Ant1_HapM1, Ant1_HapM2, Ant1_HapM3, Ant1_HapM4, Ant1_HapM5, and Ant1_HapM6, preferably all, as defined in claim 1, and a homozygous or heterozygous Ant2 gene or coding sequence thereof comprising or associated or linked with one or more molecular marker allele selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7; and
selecting seeds having a purple pericarp and/or hull if said first barley plant population is cytoplasmic male sterile and selecting seeds not having a purple pericarp and/or hull if said second barley plant population is cytoplasmic male sterile.

12. The method according to claim 11,
wherein said second barley plant population does not comprise a heterozygous Ant1 gene or coding sequence thereof comprising one or more of molecular marker allele selected from Ant1_HapM1, Ant1_HapM2, Ant1_HapM3, Ant1_HapM4, Ant1_HapM5, and Ant1_HapM6, preferably all,
wherein if said second barley plant population comprises a homozygous Ant1 gene or coding sequence thereof comprising one or more of molecular marker allele selected from Ant1_HapM1, Ant1_HapM2, Ant1_HapM3, Ant1_HapM4, Ant1_HapM5, and Ant1_HapM6, preferably all, said first barley plant population comprises a homozygous Ant2 gene or coding sequence thereof comprising or associated or linked with one or more molecular marker allele selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7, and
selecting seeds having a purple pericarp and/or hull if said first barley plant population) is cytoplasmic male sterile and selecting seeds not having a purple pericarp and/or hull if said second barley plant population is cytoplasmic male sterile.

13. The method according to claim 11,
wherein only said first barley parent plant population comprises a Ant1 gene or coding sequence thereof comprising one or more of molecular marker allele selected from Ant1_HapM1, Ant1_HapM2, Ant1_HapM3, Ant1_HapM4, Ant1_HapM5, and Ant1_HapM6, preferably all,
wherein said Ant1 gene or coding sequence thereof comprising one or more of molecular marker allele selected from Ant1_HapM1, Ant1_HapM2, Ant1_HapM3, Ant1_HapM4, Ant1_HapM5, and Ant1_HapM6, preferably all, is homozygous,
wherein said first barley parent plant population further comprises a homozygous or heterozygous Ant2 gene or coding sequence thereof comprising or associated or linked with one or more molecular marker allele selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7, and
selecting seeds having a purple pericarp and/or hull if said first barley plant population is cytoplasmic male sterile and selecting seeds not having a purple pericarp and/or hull if said second barley plant population is cytoplasmic male sterile.

14. The method according to claim 11,
wherein said first barley parent plant population comprises a homozygous Ant1 gene or coding sequence thereof comprising one or more of molecular marker allele selected from Ant1_HapM1, Ant1_HapM2, Ant1_HapM3, Ant1_HapM4, Ant1_HapM5, and Ant1_HapM6, preferably all, and a homozygous Ant2 gene or coding sequence thereof comprising or associated or linked with one or more molecular marker allele selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7, and
wherein said second barley parent plant (population) comprises a homozygous Ant1 gene or coding sequence thereof comprising one or more of molecular marker allele selected from Ant1_HapM1, Ant1_HapM2, Ant1_HapM3, Ant1_HapM4, Ant1_HapM5, and Ant1_HapM6, preferably all, and does not comprise a Ant2 gene or coding sequence thereof comprising or associated or linked with one or more molecular marker allele selected from Ant2_M1, Ant2_M2, Ant2_M3, Ant2_M4, Ant2_M5, Ant2_M6, Ant2_M7, Ant2_HapM1, Ant2_HapM2, Ant2_HapM3, Ant2_HapM4, Ant2_HapM5, Ant2_HapM6, and Ant2_HapM7, preferably selected from Ant2_M2, Ant2_M3, Ant2_M4, Ant2_M5, Ant2_M6, and Ant2_M7, or selected from Ant2_HapM1 combined with any one or more of Ant2_HapM2, Ant2_HapM3, Ant2_HapM4, Ant2_HapM5, Ant2_HapM6, or Ant2_HapM7; and
selecting seeds having a purple pericarp and/or hull if said first barley plant population is cytoplasmic male sterile and selecting seeds not having a purple pericarp and/or hull if said second barley plant population is cytoplasmic male sterile.

15. An isolated polynucleotide having a sequence comprising, comprised in, or consisting of a sequence as set forth in any of SEQ ID NO: 1 to 32, or a unique fragment thereof, or the complement or reverse complement of any thereof.


SEQUENCE LISTING (34 sequences)

SEQ ID NO: 1 - DNA, 1408 bp, synthetic construct
  source 1..1408: mol_type = other DNA
atccgtaggcttgccatcttttatatgcctatacatgtgcgttgtcgtgtcgcattacaaacttgctacgaaaagtcaacatgtttcagtttttttacgtgtagtacttttaatctttaccgttgatacatcttaaagtttagagtgtcaaatcatcatcattcttttctatcgttttaatagaagatatttaattattataaaggatgctaagagatatctattataaacatgtttttgttgtcatctttaaattaaatatgagttttaatataaaaccgtattatcaatcattgtacatgttcttagatatgtgctgatgcacacatggaagccttcccggccttttggtccatctgacacttgaggaaaggtgctaagcaggccacgtagtcgcgctgcgtcgccggtgtataaatacttccagcaacccgtacgtgctgcccacagaccactccccctctctaacaaagcacacacggcgagatagagatagagggagagaggttgagaggatggggaggagggcgtgctgtgccaaggaaggggtgaagcgaggggcgtggacgagcaaggaggacgaaagcctggcttcctacgtcaaggcccatggcgaaggccggtggagggagctcccccagcgagctggtacgtactgatccatcctcatcccatatgttcgtcaaagttttgttcagttttgagcattggagagctgcatggagagtgggtggtgacgcatgttaagttgttaactgaatgaatctgcatgcatgcaggtctgcggcggtgcggcaagagctgccggctgcggtggctgaactacctccggccgaacatcaagcggggcaacatctccgacgacgaggaggagctcatcgtcaggctccacggcctgctcggcaacaggtggtccatcatcgccggcaggctgcccggccgaacagacaacgaaatcaagaactactggaacagcacgctcggcaggaaggcgctccccgcccggcccgccattgccgcggcgaggaccatcgcctccggatcctccagctccacggggagcgccgcggcggcgctctccacctcagtccctgccgtcctccacgccgcggcgccttcgtcgtcgccggccgctgtgtgggcgcccaagcccgtgaggtgcactggcggcctcttcttccgccgggagacgccgccgcccgcgccggtcgtcgaggagacgcggaccaggggagaagagccaccatttcatgcctgcagcggtagcagctcggcctcggaggcgtctccggccgagctttgctcgtcggggtccggcgggggagactggatggacgacgtgagagccctggcgtcgttcctcgagtccgacgaggaatggctcaagtccctgcacatggccggttaa

SEQ ID NO: 2 - DNA, 762 bp, synthetic construct
  source 1..762: mol_type = other DNA
atggggaggagggcgtgctgtgccaaggaaggggtgaagcgaggggcgtggacgagcaaggaggacgaaagcctggcttcctacgtcaaggcccatggcgaaggccggtggagggagctcccccagcgagctggtctgcggcggtgcggcaagagctgccggctgcggtggctgaactacctccggccgaacatcaagcggggcaacatctccgacgacgaggaggagctcatcgtcaggctccacggcctgctcggcaacaggtggtccatcatcgccggcaggctgcccggccgaacagacaacgaaatcaagaactactggaacagcacgctcggcaggaaggcgctccccgcccggcccgccattgccgcggcgaggaccatcgcctccggatcctccagctccacggggagcgccgcggcggcgctctccacctcagtccctgccgtcctccacgccgcggcgccttcgtcgtcgccggccgctgtgtgggcgcccaagcccgtgaggtgcactggcggcctcttcttccgccgggagacgccgccgcccgcgccggtcgtcgaggagacgcggaccaggggagaagagccaccatttcatgcctgcagcggtagcagctcggcctcggaggcgtctccggccgagctttgctcgtcggggtccggcgggggagactggatggacgacgtgagagccctggcgtcgttcctcgagtccgacgaggaatggctcaagtccctgcacatggccggttaa

SEQ ID NO: 3 - protein, 253 aa, synthetic construct
MGRRACCAKEGVKRGAWTSKEDESLASYVKAHGEGRWRELPQRAGLRRCGKSCRLRWLNYLRPNIKRGNISDDEEELIVRLHGLLGNRWSIIAGRLPGRTDNEIKNYWNSTLGRKALPARPAIAAARTIASGSSSSTGSAAAALSTSVPAVLHAAAPSSSPAAVWAPKPVRCTGGLFFRRETPPPAPVVEETRTRGEEPPFHACSGSSSASEASPAELCSSGSGGGDWMDDVRALASFLESDEEWLKSLHMAG

SEQ ID NO: 4 - DNA, 1409 bp, synthetic construct
  source 1..1409: mol_type = other DNA
atccgtaggcttgccatcatttatatgcctatacatgtgcgttgtcgtgtcgcattacaaacttgctacgaaaagtcaacatgtttcagtttttgtacgtgtagtacttttaatctttaccgttgatacatcttaaagtttagagtgtcaaatcatcaccattcgttttctatcgttttaatagaagatatttaattattataaaggatgctaagagatgcctattataaacatgtttttattatcatctctaaattaaatatgagttttaatataaaaccgtattatcaatcattgtacatgttcttagatatgtgctgatgcacacatggaagccttcccggccttttggtccatctgacacttgaggaaaggtgctaagcaggccacgtagtcgcgctgcgtcgccggtggataaatacccccagcaacccgtacgtgctgcccacagaccactccccctctctaacaaagcacacacggcgagatagagatagagggagagaggttgagaggatggggaggagggcgtgctgtgccaaggaaggggtgaagcgaggggcgtggacgagcaaggaggacgaaagcctggcttcctacgtcaaggcccatggcgaaggccggtggagggagctcccccagcgagctggtacgtactgatccatcctcatcccatatgttcgtcaaagttttgttcagttttgagcattggagagctgcatggagagtgggtggtgacgcatgttaagttgttaactgaatgaatctgcatgcatgcaggtctgcggcggtgcggcaagagctgccggctgcggtggctgaactacctccggccgaacatcaagcggggcaacatctccgacgaggaggaggagctcatcgtcaggctccacggcctgctcggcaacaggtggtccatcatcgccggcaggctgcccggccgaacagacaacgaaatcaagaactactggaacagcacgctcggcaggaaggcgctccccgcccggcccgccattgccgcggcgaggaccatcgcctccggatcctccagctccacggggagcgccgcggcggcgctctccacctcagtccctgccgtcctccacgccgcggcgccttcgtcgtcgccggccgctgtgtgggcgcccaagcccgtgaggtgcactggcggcctcttcttccgccgggagacgccgccgcccgcgccggtcgtcgaggagacgcggaccaggggagaagagccaccatttcatgcctgcagcggtagcagctcggcctcggaggagtctccggccgagctttgctcgtcagggtccggcgggggagactggatggacgacgtgagagccctggcgtcgttcctcgagtccgacgaggaatggctcaagtccctgcacatggccggttaa

SEQ ID NO: 5 - DNA, 762 bp, synthetic construct
  source 1..762: mol_type = other DNA
atggggaggagggcgtgctgtgccaaggaaggggtgaagcgaggggcgtggacgagcaaggaggacgaaagcctggcttcctacgtcaaggcccatggcgaaggccggtggagggagctcccccagcgagctggtctgcggcggtgcggcaagagctgccggctgcggtggctgaactacctccggccgaacatcaagcggggcaacatctccgacgaggaggaggagctcatcgtcaggctccacggcctgctcggcaacaggtggtccatcatcgccggcaggctgcccggccgaacagacaacgaaatcaagaactactggaacagcacgctcggcaggaaggcgctccccgcccggcccgccattgccgcggcgaggaccatcgcctccggatcctccagctccacggggagcgccgcggcggcgctctccacctcagtccctgccgtcctccacgccgcggcgccttcgtcgtcgccggccgctgtgtgggcgcccaagcccgtgaggtgcactggcggcctcttcttccgccgggagacgccgccgcccgcgccggtcgtcgaggagacgcggaccaggggagaagagccaccatttcatgcctgcagcggtagcagctcggcctcggaggagtctccggccgagctttgctcgtcagggtccggcgggggagactggatggacgacgtgagagccctggcgtcgttcctcgagtccgacgaggaatggctcaagtccctgcacatggccggttaa

SEQ ID NO: 6 - protein, 253 aa, synthetic construct
MGRRACCAKEGVKRGAWTSKEDESLASYVKAHGEGRWRELPQRAGLRRCGKSCRLRWLNYLRPNIKRGNISDEEEELIVRLHGLLGNRWSIIAGRLPGRTDNEIKNYWNSTLGRKALPARPAIAAARTIASGSSSSTGSAAAALSTSVPAVLHAAAPSSSPAAVWAPKPVRCTGGLFFRRETPPPAPVVEETRTRGEEPPFHACSGSSSASEESPAELCSSGSGGGDWMDDVRALASFLESDEEWLKSLHMAG

SEQ ID NO: 7 - DNA, 240 bp, synthetic construct
  source 1..240: mol_type = other DNA
atccgtaggcttgccatcatttatatgcctatacatgtgcgttgtcgtgtcgcattacaaacttgctacgaaaagtcaacatgtttcagtttttktacgtgtagtacttttaatctttaccgttgatacatcttaaagtttagagtgtcaaatcatcaccattcgttttctatcgttttaatagaagatatttaattattataaaggatgctaagagatgcctattataaacatgttttt

SEQ ID NO: 8 - DNA, 420 bp, synthetic construct
  source 1..420: mol_type = other DNA
attatcatctctaaattaaatatgagttttaatataaaaccgtattatcaatcattgtacatgttcttagatatgtgctgatgcacacatggaagccttcccggccttttggtccatctgacacttgaggaaaggtgctaagcaggccacgtagtcgcgctgcgtcgccggtggataaatacccccagcaacccgtacgtgctgcccacagaccactccccctstctaacaaagcacacacggcgagatagagatagagggagagaggttgagaggatggggaggagggcgtgctgtgccaaggaaggggtgaagcgaggggcgtggacgagcaaggaggacgaaagcctggcttcctacgtcaaggcccatggcgaaggccggtggagggagctcccccagcgagctggtacgtactga

SEQ ID NO: 9 - DNA, 360 bp, synthetic construct
  source 1..360: mol_type = other DNA
tccatcctcatcccatatgttcgtcaaagttttgttcagttttgagcattggagagctgcatggagagtgggtggtgacgcatgttaagttgttaactgaatgaatctgcatgcatgcaggtctgcggcggtgcggcaagagctgccggctgcggtggctgaactacctccggccgaacatcaagcggggcaacatctccgacgasgaggaggagctcatcgtcaggctccacggcctgctcggcaacaggtggtccatcatcgccggcaggctgcccggccgaacagacaacgaaatcaagaactactggaacagcacgctcggcaggaaggcgctccccgcccggcccgccattgccg

SEQ ID NO: 10 - DNA, 180 bp, synthetic construct
  source 1..180: mol_type = other DNA
cggcgaggaccatcgcctccggatcctccagctccacggggagcgccgcggcggcgctctccacctcagtccytgccgtcctccacgccgcggcgccttcgtcgtcgccggccgctgtgtgggcgcccaagcccgtgaggtgcactggcggcctcttcttccgccgggagacgccgccgc

SEQ ID NO: 11 - DNA, 240 bp, synthetic construct
  source 1..240: mol_type = other DNA
gggcgcccaagcccgtgaggtgcactggcggcctcttcttccgccgggagacgccgccgcccgcgccggtcgtcgaggagacgcggaccaggggagaagagccaccatttcatgcctgcagcggtagcagctcggcctcggaggmgtctccggccgagctttgctcgtcagggtccggcgggggagactggatggacgacgtgagagccctggcgtcgttcctcgagtccgacgaggaat

SEQ ID NO: 12 - DNA, 149 bp, synthetic construct
  source 1..149: mol_type = other DNA
gcggtagcagctcggcctcggaggagtctccggccgagctttgctcgtcrgggtccggcgggggagactggatggacgacgtgagagccctggcgtcgttcctcgagtccgacgaggaatggctcaagtccctgcacatggccggttaa

SEQ ID NO: 13 - DNA, 201 bp, synthetic construct
  source 1..201: mol_type = other DNA
atgcgtctgcatcagctttaagttacttctgaacactaccacatgcctgcagttactactaccgcaaaaaaacacaagtcgagtgccgtgttttcaggttyagataaaggagggtgttaggtttcagtttgggttcactcacgtggtctgaccaggcgtgctccctgttgtagatgatggccgcgccgaggctcctcgccg

SEQ ID NO: 14 - DNA, 5066 bp, synthetic construct
  source 1..5066: mol_type = other DNA
  misc_difference 3364: note = a, c, t, g, unknown, or other
  misc_difference 4992: note = a, c, t, g, unknown, or other
gccgtgtgtttccttagttgtcgtcgacgtcgattattctgtgcctgttaccccgtcacctcagcgtcaagcagagatcggtgaggattgcgtgcatctcactccatttttactaagcagtgagaagaccaagcaggcatgcacaccaaactaatcagaaagtgatgggcctaatatgaaactaatcagaagtaccatgttggagcactacagtagtgtccggacagtactttgcttttgcgtcgcagtctcgcttgttggctcgttgctcggagcctcggacagatctatataacatgccctggattctagatacattgcacatatagaagctccttcttcctctccggacgacaggttggactccgggcttcttggtctccatagctcaaagtaaacctttaacctcctcttagatggaaaattaatcctccactcatgtctagcttagtttatctcttactttcttggtttgttccagctttcgatcgagtatatacagtatgttctttgtaatcttcgttgagaaatcttcccaggagtttcttaattagcaccagtgcaacagagtggagggatttcgatcagctagcatgaactaaattatccaagaagatgcatcatgctcactataaacacaagagatggaaccatatatagcttcattgatttgtttatttttgcctccttgcttgctctaacttcacctcgtctgcatcatgcatatgaaatgaaggaaggaaataatatggtaatggcgctaccaatagttcgtccgagccaggaagaaccgccgacggggaagcaattcagctaccagctcgctgccgctgtgaggagcatcaactggagctacgccatattctggtccatttcaaccagccgtccagggtagggagtgcatcagattgatgatcacttggcactggccgtttcctttcctcttatgatccgtttgttatgtagggtactgacctggaaggacgggttctacaacggcgagataaagacgaggaaggtcaccagctcggcggacctcaccgccgaccagctcctcctgcagaggagcgagcagctgcgggagctctaccagtccctcctgtccggccagtgcgaccaccgggggaggcgcccggccgccgcgctctcgccggaggacctcggggacgccgaatggtactacgccgtctgcatgagctatgccttccgccctggccaagggtatgtactactccgtagtatgcttcaagaacaactccactgatccataacggccgatgcctgcgcacctacagtagttagcaatcatcttatcttgataagataagatttactttgctagagagtgaaaactacaacgtttgctatttcgtcatcaggttgccaggcagaagctttgcgagcaacgagcctgtttggctgtgcaatgctcagtgcgcagacaccaaaactttccagcgctcgctcttagcgaaggtacgtttgattgcaccgcccgagtgtctcgcaagcctcactctcatgtagtccctccgtaaataaatggagtataagagtgcttagatcactacttagtgatataaacgcttttatgttcctttacgaagggagtactatctgcctaatatgtacccgatcttgactaataccatgttgtctactgtgtacttttggcgcacatactatatctgtcggctaaatcagacgacgtctatccaggttagctagcgcatgtcatgcaccctatatctgtgtatactgttcatgtttctgaccccagctgcacgggtgacaatattttttcttcagacggtcgcctgcattcccttgatgggtggtgtgcttgagctcgggacgacagataccgtgagttttcctcgcatcaagcacatgatattcacttacggtttaaaattaaacggaaaactcactattagtaacgctttgtctcatcaagcacatcgaaattgcaaaatttaacccgggttccatggagccttgttgttaagaaaaacacaacagaagttgtgagcacctatatatttccgttcagtacatcttgccatatgtgggggtagacaacaaagacaaatatgcggatctacaaatagccaataatagatgtgctagtatttccaaaaccacacattgtcatcttgtgtagctcacatgcatgtgttatagtgtttattatcgaatgctacgtgtatgctagtatagatctatatatacatctaggtagacttgttttactgttttttttcaaagttgtaaatattatttgattttttttccaaaatttcagtctcactacaaccctgatctcccaattattcgctcaagctgccatgtcaactatggtacaagtttgaagtttgttaccctttcattctttccctgcccttgttgcttacaaatgcgagctttgctacaagtacagacgttttacaggatttttacaggcaaagttgaagtggcttattttaattggtacttagatgaggccggccctaagaatcaaggggagattagtgaggagaaaaacccatcagtctgtaatcttttgtaaaaaaggtccgtaggtctggcattttcctataaaatgcggacgaaaccaatatcttttgaggcctctgctacgttattctagtgtgttatttttcaagaacaagaaacacaatgctgcatgtcattgtcattaagaaggaaagggagttaattacaagcaaatagtcgtgacaaagcatgtagtgttgttataccacacgtgagaccaccgccaccacattcatatctaggcccactcctccctaatctttgtcactgactgcctagcctttttggaaagcataagttcttgtgccgccggtgcgaccactctttgcaccgatgacactttcccatcaaaaactctagtgttgcgctctagccttcattaatgtcagtcagccggtcttgaacgacaatgaagtcttccaagtcgcccacaacgtgaggttgcaaatctactttggtgaaggcacatgtgcgtatagccccaaatacgtgcacagaaatttggccacttgatttgcaagagaagcggtcagaacatccaaacaatccactccctcgcatcgggtaggtgaaatcacatgtttctaaagattaaccacaagaccaaaatcgccaccaaataacgtcaaagatctccttccttgccaacaactcgctttctctgggtttgaggtaacccgatagccggtgacgaacaccccatctatccaaaggttcagacacaccaaaatcctccactttattccttgtgcaggtggtaaagccccttgcctcgtggcatgacaataggttgtcctgattttgtttntcggattcgtcataaaggggaggcttttataccgtatgattgaaaattttgtattctatcagtaaattacgataaataaataaaaatacttcttttaatgtaaaatatattcttatactatgtgacaacgggaaggcggtaccgtttgctggggacactcattaagtcatgaagacacaatactactaaatcaatccatgaattgtttgagttcttcaccaaataaaatgctactgtgctgattaattcattcgaatataagttaaaaattaaccaaagcgctatcaatcgatttgctaggttttggaggacagggacatggtgaaccggatcagcacatctttctgggacctgaagatcccaacaagctcgaagccgaaggagccctccagcccatcagcagacgacgctggtgaggccgacatcgtgttccaagacctcgaccacaacaccatggccgcgatgatccccggggaactcgagctaggggaggtcgagtgcctgtccgacgacaacctcgagcggatcacgaaggagatcaaacggttctacggcctctgcgacgagctggacgtcggcgctctcgacgaaaactggatcataggcgggtctttcgaggtcatgtcctcgccggaagcgccaccggcgcctgcagccaccggcggcatcactgatggtattgtcactttaagcgccgctgcgtcctctctctcatcgtgctttacggcttggaagagatcgtgggactcagccgaagacatggctgcgccggtcgccgggcagtcgcagaagttactgaagaaagctttggccggtggtgtgtgggcgattaacggcggcggcggcggcggcacggcgagagctcaggaaagtagcaacaccaagaaccatgtcatatcggagaggcggcgccgggagaagctcaacgagatgttcctgattctcaagtcactggtgccgtccattcacaaggtaacacgcgcacatcaccggaaagcaaaagaaaaaatactttccagagatgtgtttctgaacttttgtttgcaggtggacaaggcatccatcctagctgagacgatagcctatctcagagagctggagcaaagggtggaggagctagaatccaacagggcgccgtcgcggccggccggagcagccgtccggagacaccatgacgccgcggcgaagaagatgttagctggatccaagagaaaggcgtcggagctcggcggggacgacggcccgaacagcgtcgtcaacgtcacggtgacggagaaagaggtgctcctggaggtgcaatgccggtggaaggagctgctgatgacgcaggtgttcgacgccttcaagagcctccgcctggacgtgctctccgtgcgcgcgtcgacgcccgacggcctcctcgctctcaagatacgagctcaggtccgtctggtagcagctgattaacttagctccatccggcggtaaaaaatgcagcattaattaacgctcgtatgcatggctatggtttactgttgcagttcgccggtcctggcgccgtggagcccgggatgatcatcggagcgcttcagacagctacacgcgggcgctgaaaaaaaaggatgctgctgagacactggtactgtacngtagatccgcgtcacgagctgggacatacatgaaagattgacaatgatggtggctcgttccgatgtgcagtgat

SEQ ID NO: 15 - DNA, 1680 bp, synthetic construct
  source 1..1680: mol_type = other DNA
atggcgctaccaatagttcgtccgagccaggaagaaccgccgacggggaagcaattcagctaccagctcgctgccgctgtgaggagcatcaactggagctacgccatattctggtccatttcaaccagccgtccaggggtactgacctggaaggacgggttctacaacggcgagataaagacgaggaaggtcaccagctcggcggacctcaccgccgaccagctcctcctgcagaggagcgagcagctgcgggagctctaccagtccctcctgtccggccagtgcgaccaccgggggaggcgcccggccgccgcgctctcgccggaggacctcggggacgccgaatggtactacgccgtctgcatgagctatgccttccgccctggccaagggttgccaggcagaagctttgcgagcaacgagcctgtttggctgtgcaatgctcagtgcgcagacaccaaaactttccagcgctcgctcttagcgaagacgacgtctatccagacggtcgcctgcattcccttgatgggtggtgtgcttgagctcgggacgacagataccgttttggaggacagggacatggtgaaccggatcagcacatctttctgggacctgaagatcccaacaagctcgaagccgaaggagccctccagcccatcagcagacgacgctggtgaggccgacatcgtgttccaagacctcgaccacaacaccatggccgcgatgatccccggggaactcgagctaggggaggtcgagtgcctgtccgacgacaacctcgagcggatcacgaaggagatcaaacggttctacggcctctgcgacgagctggacgtcggcgctctcgacgaaaactggatcataggcgggtctttcgaggtcatgtcctcgccggaagcgccaccggcgcctgcagccaccggcggcatcactgatggtattgtcactttaagcgccgctgcgtcctctctctcatcgtgctttacggcttggaagagatcgtgggactcagccgaagacatggctgcgccggtcgccgggcagtcgcagaagttactgaagaaagctttggccggtggtgtgtgggcgattaacggcggcggcggcggcggcacggcgagagctcaggaaagtagcaacaccaagaaccatgtcatatcggagaggcggcgccgggagaagctcaacgagatgttcctgattctcaagtcactggtgccgtccattcacaaggtggacaaggcatccatcctagctgagacgatagcctatctcagagagctggagcaaagggtggaggagctagaatccaacagggcgccgtcgcggccggccggagcagccgtccggagacaccatgacgccgcggcgaagaagatgttagctggatccaagagaaaggcgtcggagctcggcggggacgacggcccgaacagcgtcgtcaacgtcacggtgacggagaaagaggtgctcctggaggtgcaatgccggtggaaggagctgctgatgacgcaggtgttcgacgccttcaagagcctccgcctggacgtgctctccgtgcgcgcgtcgacgcccgacggcctcctcgctctcaagatacgagctcagttcgccggtcctggcgccgtggagcccgggatgatcatcggagcgcttcagacagctacacgcgggcgctga

SEQ ID NO: 16 - protein, 559 aa, synthetic construct
MALPIVRPSQEEPPTGKQFSYQLAAAVRSINWSYAIFWSISTSRPGVLTWKDGFYNGEIKTRKVTSSADLTADQLLLQRSEQLRELYQSLLSGQCDHRGRRPAAALSPEDLGDAEWYYAVCMSYAFRPGQGLPGRSFASNEPVWLCNAQCADTKTFQRSLLAKTTSIQTVACIPLMGGVLELGTTDTVLEDRDMVNRISTSFWDLKIPTSSKPKEPSSPSADDAGEADIVFQDLDHNTMAAMIPGELELGEVECLSDDNLERITKEIKRFYGLCDELDVGALDENWIIGGSFEVMSSPEAPPAPAATGGITDGIVTLSAAASSLSSCFTAWKRSWDSAEDMAAPVAGQSQKLLKKALAGGVWAINGGGGGGTARAQESSNTKNHVISERRRREKLNEMFLILKSLVPSIHKVDKASILAETIAYLRELEQRVEELESNRAPSRPAGAAVRRHHDAAAKKMLAGSKRKASELGGDDGPNSVVNVTVTEKEVLLEVQCRWKELLMTQVFDAFKSLRLDVLSVRASTPDGLLALKIRAQFAGPGAVEPGMIIGALQTATRGR

SEQ ID NO: 17 - DNA, 6419 bp, synthetic construct
  source 1..6419: mol_type = other DNA
atgtcgcatgagagccattacaaccaacgacgtcgcatgagagataacacaactcaacgacgtcgcatgggagctattcgcccgtcaaaaatgcaatgcatgtactccgatttagacgaaaaattatccaacttttaaggaagcagttcctctggtttgtttaaatttgcatcaattttgtttattctgttaaattccgttcgaaatgtcgtcatatatttacgactatggttggatggtcagctactatctgcgaattgatcctcacctgtcggcgcacagatgtggtgtcggattttggtgtgggcgatggagatgcccttaaggcggaaacctgtcgacctacgtgttccaatccccactaacctccttttgattttcactgagccaaccacctcatctgtaaatccagttcgtaatttctggattccttatgatgccgatggtagattccttggcaaaggaggtgctggaacacacgtacaagactttagcagttactccctctgtaaactaatataaaaacatttagaccactactttagttatctaaacgctcttccattagtttacagatggtgtaactatagaccgcatcgacagtgatgctcgccgtgtgtttccttagttgtcgttgacgttgattattctgtgcctgttaccccatcacctcagcgtcaagcagagatcggtgaggattgcgtacatctcactccatttttatcatagttttaaatagccggctatagtcccgctataaccttttcaatagggtgccgctaaatggtatcacgtacaaatatgccgctatagcccgatatagcccgctatagctccgctatagctgattttcaggcatgccgctatttgtcatagcccgctatttaaaacattgattttaactaagcactgagaagaccaagcaggcatgcacaccaaactaatcagaaagtgatgggcctaatatgaaactaatcagaagtaccatgttggagcactacagtagtgtccggacagtactttgcttttgcgtcgcagtctcgcttgttgttggctcgttgctcggagcctcggacagatctatataacatggcttggattctagatacattgcacatatagaagctccttcttcctctccggacgacaggttggactccgggcttcttggtctccatagctcaaagtaaacctttaacctcctcttagatggaaaattaatcctccactcatgtctagcttagtttatctcttactttcttggtttgttccagctttcgatctagtatatacagtatgttctttgtaatcttcgttgagaaatcttcccaggagtttcttaattagcaccagtgcaacagagtggagggatttcgatcagctagcatgaactaaattatccaagaagatgcatcatgctcactataaacacaagagatggaaccatatatagcttcattgatttgtttatttttgcctccttgcttgctctaacttcacctcgtctgcatcatgcatatgaaatgaaggaaggaaataatatggtaatggcgctaccaatagttcgtccgagccaggaagaaccgccgacggggaagcaattcagctaccagctcgctgccgctgtgaggagcatcaactggagctacgccatattctggtccatttcaaccagccgtccagggtagggagtgcatcagattgatgatcacttggcactggccgtttcctttcctcttatgatccgtttgttatgtagggtactgacctggaaggacgggttctacaacggcgagataaagacgaggaaggtcaccagctcggcggacctcaccgccgaccagctcgtcctgcagaggagcgagcagctgcgggagctctaccagtccctcttgtccggccagtgcgaccaccgggggaggcgcccggccgccgcgctctcgccggaggacctcggggacgccgaatggtactacgccgtctgcatgagctatgccttccgccctggccaagggtatgtactactccgtagtatgcttcaagaacaactcctctgatccacaagataacatttatcaatagttaacaatcatcttatcttgataagatgagagttattttgctagagagtgaaaactacaacgtttgctatttcgtcatcaggttgccaggcagaagctttgcgagcaacgagcctgtttggctgtgcaatgctcagtgcgcagacaccaaaactttccaacgctcgctcttagcgaaggtacgtttgattgcaccgcccgagtgtctcgcaagcctcactctcatgtagtccctccgtaaataaatggagtataagagtgtttagatcactacttagtgatctaaacgcttttatgtttctttacgaagggagtactatctgcctaatatgtacccgatcttgactaataccatgttgtctactgtgtacttttggcgcacatactatatctgtcggctaaatcagacgacgtctatccaggttagctagcgcatgtcatgcaccctatatctgtgtatactgttcatgtttctgaccccagctgcacgggtgacaattttttttcttcagacggtcgcctgcattcccttgatgggtggtgtgcttgagctcgggacgacagataccgtgagctttcctcgcatcaagcacatgatattcacttacggtttaaaattaaacggaaaactcactattagtaacgctttgtcgcatcaagcacatcgaaattgcaaaatttaacccgggttccatggagccttgttgttaagaaaaacacaacagaagttgtgagcacctatatatttccgttcagtacatcttgccatatgtgggtagacaacaaagacaaatatgaggatctacaaatagccaataatagatgtgctagtatttccaaaactacacattgtcatcttgtgtagctcacatgcatgtgttatagtgtttattatcgaatgctacgtatatgctagtatagatctatatatacatctaggtagacttgttttactgttttttttcaaagttgtaaatattatttgattttttttccaaaatttcagtctcactacaaccctgatctcccaattattcgctcaagctgccatgtcaactatggtacaagtttgaagtttgttaccctttcattctttccctgcccttgttgcttacaaatgcgagctttgctacaagtacggacgttttacaggatttttacaggcaaagttgaagtggcttattttaattgatacttagatgaggccggccctaaaaatcaaggggagattagtgaggagaaaaatccatcagtctgtaaccttctgtaaaaaaaaggtccgtaggtctggcattttcctacaaatgcggacgaaaccaatctcttttgaggcctctgctacgttattctagtgtgttatttttcaagaacaagaaacacaatgctgcatgtcattgtcattaagaaggaaagggagttaattagaagcaaatagtcgtgacaaagcatgtagtgttgttataccacacgtgagaccaccgccaccacattcatatctaggcccactcctccctaatctttgtcactgactgcctagcctttttggaaagcataagttcttgtgccgccggtgcgaccactctttgcaccgatgacactttcccatcaaaaactctagtgttgcgctctagccttcattaatgtcagccagccggtcttgaacgacaatgaagtcttccaagtcgcccacaacgtgaggttgcaaatctactttggtgaaggcacatgtgcgtatagccccaaatacgtgcacagaaatttggccacttgatttgcaagagaagcggtcagaacatccagacaatccactccctcgcatcgggtaggtgaaatcacatgtttctaaagattaaccacaagaccaaaatcgccaccaaataacgtcaaagatctccttccttgccaacaactcgctttctctgggtttgaggtaacccgatagccggtgacgaacaccccatctatccaaaggttcagacacaccaaaatcctccactttattccttgtgcaggtggtaaagccccttgcctcgtggcatgacaataggttgtcctgattttgtttctcggattcgtcataaaggggaggcttttataccgtatgattgaaaattttgtattctatcagtaaattacgataaataaataaaaatacttcttttaatgtaaaatatattcttatactatgtgacaatgggaaggctgtaccgtttgctcgggacactcattaagtcatgaagacacaatactactaaatcaatccatgaattgtttgagttcttcaccaaattaaatgctactgtgctgattaattcattcgaatataagttaaaaattaaccaaagcactatcaatcgatttgctaggttttggaggacagagacatggtgaaccgaatcagcacatctttctgggacctgaagatcccaacaagctcgaagccgaaggagccctccagcccatcagcagacgacgctggtgaggccgacatcgtgttccaagacctcgaccacaacaccatggccgcgatgatccccggggaactcgagctaggggaggtcgagtgcctgtccgacgacaacctcgagcggatcacgaaggagatcaacgggttctacggcctgtgcgacgagctggacgtcggcgctctcgacgaaaactggatcataggcgggtctttcgaggtcatgtcctcgccggaagcgccaccggcgcctgcagccaccggcggcatcactgatggtattgtcactttaagcgccgctgcgtcctctctctcatcgtgctttacggcttggaagagatcgtgggactcagccgaagacatggctgcgccggtcgccgggcagtcgcagaagttactgaagaaagctttggccggtggtgcgtgggcgattaacggcggcggcggcggcggcacggcgagagctcaggaaagtagcaacaccaagaaccatgtcatttcggagaggcggcgccgggagaagctcaacgagatgttcctgattctcaagtcactggtgccgtccattcacaaggtaacacgcgcacatcaccggaaagcaaaagaaaaatactgtccagagatgtgtctctgaacttttgtttgcaggtggacaaggcatccatcctagctgagacgatagcctatctcagagagctggagcaaagggtggaggagctagaatccaacagggcgccgtcgcggccggccggagcagccgtccggagacaccatgacgccgcggcgaagaagatgttagctggatccaagagaaaggcgtcggagctcggcggggacgacggcccgaacagcgtcgtcaacgtcacggtgatggagaaagaggtgctcctggaggtgcaatgccggtggaaggagctgctgatgacgcaggtgttcgacgccatcaagagcctccgcctggacgtgctctccgtgcgcgcgtcgacgcccgacggcctcctcgctctcaagatacgagctcaggtccgtctggtagcagctgattatttaagctccatccggcggtaaaaatgcagcattaattaacgctcgtatgcatggctatggtttactgttgcagttcgccggtcctggcgccgtggagcccgggatgatcatcggagcgcttcagacagctacacgagggcgctgaaaaagaagcatgctgctgagacactggtactgtactgtagatccgcgtcacgagctgggacatacatgaaagattgacaatgatggtggctatcttcttcggacatgacaattattttcattcgttccgatgtgcagtgatgtgggacgtgacaaatagatttccgatctttggatccacttgaaatcatgtgaaccgcagagctcgtgggtgcaggatgcgcgtcacgagcgcgcgcggtgcaactaccagtctccccacatgccggtcgtacaacggtgtatggcgcgggttgctcctcacctgacatcgcatctgtagactgtagcaaatctggacggcgcgacgggataccaccgcgagcgagcgcccggtcggccccgctcgctcggctgcgcgacagcgcgcgtgcgccggcgacgccgactggccagctcgtgctcgcccatgtcgcggccatacacgacgcaccgctgcgcgtcatgtaaccttttcctccgtactacgttcccgtcggatgcgcatgcgccgtgacgacatggccgaaggaattttaacgtggtccttcttgcctcatctctactattaaaagcacgaaaggcggatccaactcactatccaaaacgtctaatgagatgatcaatggattaaaatagcctgttaacgaaagtgatttaccaac

SEQ ID NO: 18 - DNA, 1680 bp, synthetic construct
  source 1..1680: mol_type = other DNA
atggcgctaccaatagttcgtccgagccaggaagaaccgccgacggggaagcaattcagctaccagctcgctgccgctgtgaggagcatcaactggagctacgccatattctggtccatttcaaccagccgtccaggggtactgacctggaaggacgggttctacaacggcgagataaagacgaggaaggtcaccagctcggcggacctcaccgccgaccagctcgtcctgcagaggagcgagcagctgcgggagctctaccagtccctcttgtccggccagtgcgaccaccgggggaggcgcccggccgccgcgctctcgccggaggacctcggggacgccgaatggtactacgccgtctgcatgagctatgccttccgccctggccaagggttgccaggcagaagctttgcgagcaacgagcctgtttggctgtgcaatgctcagtgcgcagacaccaaaactttccaacgctcgctcttagcgaagacgacgtctatccagacggtcgcctgcattcccttgatgggtggtgtgcttgagctcgggacgacagataccgttttggaggacagagacatggtgaaccgaatcagcacatctttctgggacctgaagatcccaacaagctcgaagccgaaggagccctccagcccatcagcagacgacgctggtgaggccgacatcgtgttccaagacctcgaccacaacaccatggccgcgatgatccccggggaactcgagctaggggaggtcgagtgcctgtccgacgacaacctcgagcggatcacgaaggagatcaacgggttctacggcctgtgcgacgagctggacgtcggcgctctcgacgaaaactggatcataggcgggtctttcgaggtcatgtcctcgccggaagcgccaccggcgcctgcagccaccggcggcatcactgatggtattgtcactttaagcgccgctgcgtcctctctctcatcgtgctttacggcttggaagagatcgtgggactcagccgaagacatggctgcgccggtcgccgggcagtcgcagaagttactgaagaaagctttggccggtggtgcgtgggcgattaacggcggcggcggcggcggcacggcgagagctcaggaaagtagcaacaccaagaaccatgtcatttcggagaggcggcgccgggagaagctcaacgagatgttcctgattctcaagtcactggtgccgtccattcacaaggtggacaaggcatccatcctagctgagacgatagcctatctcagagagctggagcaaagggtggaggagctagaatccaacagggcgccgtcgcggccggccggagcagccgtccggagacaccatgacgccgcggcgaagaagatgttagctggatccaagagaaaggcgtcggagctcggcggggacgacggcccgaacagcgtcgtcaacgtcacggtgatggagaaagaggtgctcctggaggtgcaatgccggtggaaggagctgctgatgacgcaggtgttcgacgccatcaagagcctccgcctggacgtgctctccgtgcgcgcgtcgacgcccgacggcctcctcgctctcaagatacgagctcagttcgccggtcctggcgccgtggagcccgggatgatcatcggagcgcttcagacagctacacgagggcgctga

SEQ ID NO: 19 - protein, 559 aa, synthetic construct
MALPIVRPSQEEPPTGKQFSYQLAAAVRSINWSYAIFWSISTSRPGVLTWKDGFYNGEIKTRKVTSSADLTADQLVLQRSEQLRELYQSLLSGQCDHRGRRPAAALSPEDLGDAEWYYAVCMSYAFRPGQGLPGRSFASNEPVWLCNAQCADTKTFQRSLLAKTTSIQTVACIPLMGGVLELGTTDTVLEDRDMVNRISTSFWDLKIPTSSKPKEPSSPSADDAGEADIVFQDLDHNTMAAMIPGELELGEVECLSDDNLERITKEINGFYGLCDELDVGALDENWIIGGSFEVMSSPEAPPAPAATGGITDGIVTLSAAASSLSSCFTAWKRSWDSAEDMAAPVAGQSQKLLKKALAGGAWAINGGGGGGTARAQESSNTKNHVISERRRREKLNEMFLILKSLVPSIHKVDKASILAETIAYLRELEQRVEELESNRAPSRPAGAAVRRHHDAAAKKMLAGSKRKASELGGDDGPNSVVNVTVMEKEVLLEVQCRWKELLMTQVFDAIKSLRLDVLSVRASTPDGLLALKIRAQFAGPGAVEPGMIIGALQTATRGR

SEQ ID NO: 20 - DNA, 116 bp, synthetic construct
  source 1..116: mol_type = other DNA
gcttagtttatctcttactttcttggtttgttccagctttcgatckagtatatacagyatgttctttgtaatcttcgttgagaaatcttcccaggagtttcttaattagcaccagt

SEQ ID NO: 21 - DNA, 176 bp, synthetic construct
  source 1..176: mol_type = other DNA
gtctcgcaagcctcactctcatgtagtccctccgtaaataaatggagtataagagtgyttagatcactacttagtgatataaacgcttttatgttyctttacgaagggagtactatctgcctaatatgtacccgatcttgactaataccatgttgtctactgtgtacttttggcgc

SEQ ID NO: 22 - DNA, 126 bp, synthetic construct
  source 1..126: mol_type = other DNA
gcacatgatattcacttacggtttaaaattaaacggaaaactcactattagtaacgctttgtckcatcaagcacatcgaaattgcaaaatttaacccgggttccatggagccttgttgttaagaaa

SEQ ID NO: 23 - DNA, 159 bp, synthetic construct
  source 1..159: mol_type = other DNA
tccaaaaccacacattgtcatcttgtgtagctcacatgcatgtgttatagtgtttattatcgaatgctacgtrtatgctagtatagatctatatatacatctaggtagacttgttttactgttttttttcaaagttgtaaatattatttgatttttttt

SEQ ID NO: 24 - DNA, 185 bp, synthetic construct
  source 1..185: mol_type = other DNA
aagcataagttcttgtgccgccggtgcgaccactctttgcaccgatgacactttcccatcaaaaactctagtgttgcgctctagccttcattaatgtcagycagccggtcttgaacgacaatgaagtcttccaagtcgcccacaacgtgaggttgcaaatctactttggtgaaggcacatgtgcg

SEQ ID NO: 25 - DNA, 131 bp, synthetic construct
  source 1..131: mol_type = other DNA
ggggaactcgagctaggggaggtcgagtgcctgtccgacgacaacctcgagcggatcacgaaggagatcaamsggttctacggcctctgcgacgagctggacgtcggcgctctcgacgaaaactggatcat

SEQ ID NO: 26 - DNA, 143 bp, synthetic construct
  source 1..143: mol_type = other DNA
gctaggttttggaggacagggacatggtgaaccggatcagcacatctttctgggacctgaagatcccaacaagctcgaagccgraggagccctccagcccatcagcagacgacgctggtgaggccgacatcgtgttccaagac

SEQ ID NO: 27 - DNA, 120 bp, synthetic construct
  source 1..120: mol_type = other DNA
gttggactccgggcttcttggtctccatagctcaaagtaaacctttaacctcstcttagatggaaaattaatcctccactcatgtctagcttagtttatctcttactttcttggtttgtt

SEQ ID NO: 28 - DNA, 176 bp, synthetic construct
  source 1..176: mol_type = other DNA
cgtctgcatcatgcatatgaaatgaaggaaggaaataatatggtaatggcgctaccaatagttcgtccgagccaggaagaaccgccgacggggragcaattcagctaccagctcgctgccgctgtgaggagcatcaactggagctacgccatattctggtccatttcaaccagccg

SEQ ID NO: 29 - DNA, 177 bp, synthetic construct
  source 1..177: mol_type = other DNA
cgtctatccaggttagctagcgcatgtcatgcaccctatatctgtgtatactgttcatgtttctgaccccagctgcacgggtgacaaywttttttcttcagacggtcgcctgcattcccttgatgggtggtgtgcttgagctcgggacgacagataccgtgagttttcctcgcatca

SEQ ID NO: 30 - DNA, 109 bp, synthetic construct
  source 1..109: mol_type = other DNA
tcgaaattgcaaaatttaacccgggttccatggagccttgttgttaagaaawacacaacagaagttgtgagcacctatatatttccgttcagtacatcttgccatatgt

SEQ ID NO: 31 - DNA, 125 bp, synthetic construct
  source 1..125: mol_type = other DNA
tacttcttttaatgtaaaatatattcttatactatgtgacaacgggaaggcggtaccgtttgctggggacactcattaartcatgaagacacaatactactaaatcaatccatgaattgtttgag

SEQ ID NO: 32 - DNA, 157 bp, synthetic construct
  source 1..157: mol_type = other DNA
cggcggtaaaaaatgcagcattaattaacgctcgtatgcatggctatggtttactgttgcagttcgccggtcstggcgccgtggagcccgggatgatcatcggagcgcttcagacagctacacgcgggcgctgaaaaaaaaggatgctgctgagaca

SEQ ID NO: 33 - DNA, 201 bp, synthetic construct
  source 1..201: mol_type = other DNA
agggagcagccagctagctatgctcctctgtgctgctctgcaggttggcgcagcctcacgtcgacaacacatggcacacggggccaactgaaccacgaggmggtgcatgcatgcatgcctcctgcctcaccgggtttgctccatggccagcggccaccggtatgtgcacgtaccaccccagaattttctattccaatttcc

SEQ ID NO: 34 - DNA, 201 bp, synthetic construct
  source 1..201: mol_type = other DNA
  misc_difference 101: note = a, c, t, g, unknown, or other
agggagcagccagctagctatgctcctctgtgctgctctgcaggttggcgcagcctcacgtcgacaacacatggcacacggggccaactgaaccacgaggnggtgcatgcatgcatgcctcctgcctcaccgggtttgctccatggccagcggccaccggtatgtgcacgtaccaccccagaattttctattccaatttcc